(19) United States
(12) Patent Application Publication
(10) Pub. No.: US 2007/0219234 A1
(43) Pub. Date: Sep. 20, 2007

(54) Thienopyridine Derivatives

(76) Inventors: Kiyoshi Oizumi, Tokyo (JP); Satoru Naito, Tokyo (JP); Akira Nakao, Tokyo (JP); Tsuyoshi Shinozuka, Tokyo (JP); Satoshi Matsui, Tokyo (JP); Kousei Shimada, Tokyo (JP)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 11/578,492

(22) PCT Filed: Apr. 11, 2005
(86) PCT No.: PCT/JP05/07025

§ 371 Date: Apr. 2, 2007

(30) Foreign Application Priority Data
Apr. 12, 2004 (JP) 2004-116366

(51) Int. Cl. A61K 31/4365; C07D 513/04

(52) U.S. Cl. 514/301; 546/114

(57) ABSTRACT

[Problem to be Solved]The present invention provides a compound promoting osteogenesis. [Solution]
The present invention provides a compound having the following general formula (I) 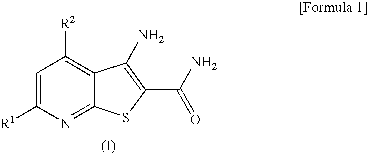
wherein R¹ is H or alkyl,
R² is R^{a}S—, R^{a}O—, R^{a}NH—, R^{a}(R^{b})N— or cyclic amino, and
R^{a} and R^{b} are alkyl which may be substituted, cycloalkyl which may be substituted, or the like, or a pharmacologically acceptable salt thereof.

### TECHNICAL FIELD

The present invention relates to a compound promoting osteogenesis.

### BACKGROUND ART

It is known that thienopyridine derivatives have IκB kinase complex inhibitory effect (see Patent Document 1). In addition, 3-amino-4-(dimethylamino)thieno[2,3-b]pyridine-2-carboxamide and 3-amino-4-anilinothieno[2,3-b]pyridine-2-carboxamide are known compounds (see Non-Patent Document 1).

However, the influence that these compounds give to bone has not been reported.
[Patent Document 1] WO03/103661
[Non-Patent Document 1] Pharm. Chem. J. (Engl. Transl.), 26, 870-874 (1992)

### DISCLOSURE OF THE INVENTION

### Problems to be Solved by the Invention

The present inventors have conducted intensive studies on compounds that promote osteogenesis, and consequently have found that thienopyridine derivatives have excellent pharmacological effect and thus completed the present invention.

### Means for Solving the Problems

The present invention relates to
(1) a compound having the following general formula (I) 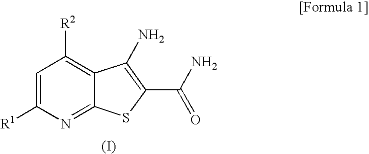
   [wherein
R¹ represents a hydrogen atom or a C₁-C₆ alkyl group,
R² represents R^{a}S—, R^{a}O—, R^{a}NH—, R^{a}(R^{b})N— or a group 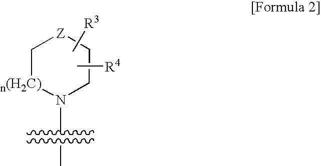
wherein R^{a} and R^{b} are the same or different and independently represent a C₁-C₆ alkyl group which may be substituted with one or more groups selected from Substituent Group α and Substituent Group γ; a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₆ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group δ and Substituent Group γ and which contains 1 to 3 sulfur, oxygen and/or nitrogen atoms,
R³ and R⁴ are the same or different and independently represent a hydrogen atom; a group selected from Substituent Group α, Substituent Group β and Substituent Group γ; a C₁-C₆ alkyl group substituted with one or more groups selected from Substituent Group γ; or a C₁-C₆ alkoxy group substituted with one or more groups selected from Substituent Group γ,
or when R³ and R⁴ are bonded to adjacent carbon atoms, R³ and R⁴ together with the carbon atoms to which they are bonded may form a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms,
Z represents a single bond; a double bond; an oxygen atom; a sulfur atom; sulfinyl; sulfonyl; or a group having the formula R⁵N<;
R⁵ represents a hydrogen atom; a C₁-C₆ alkyl group which may be substituted with one or more groups selected from Substituent Group α and Substituent Group γ; a C₂-C₆ alkenyl group which may be substituted with one or more groups selected from Substituent Group α and Substituent Group γ; a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; a 5- to 7-membered heteroaryl group which may be substituted with one or more groups selected from Substituent Group α, Substituent Group β and Substituent Group γ and which contains 1 to 3 sulfur, oxygen and/or nitrogen atoms; a formyl group; a C₂-C₇ alkylcarbonyl group which may be substituted with one or more groups selected from Substituent Group α and Substituent Group γ; a 5- to 7-membered heterocyclylcarbonyl group which may be substituted with one or more groups selected from Substituent Group α, Substituent Group β and Substituent Group γ and which contains 1 to 3 sulfur, oxygen and/or nitrogen atoms; a C₇-C₁₁ arylcarbonyl group which may be substituted with one or more groups selected from Substituent Group α, Substituent Group β and Substituent Group γ; a 5- to 7-membered heteroarylcarbonyl group which may be substituted with one or more groups selected from Substituent Group α, Substituent Group β and Substituent Group γ and which contains 1 to 3 sulfur, oxygen and/or nitrogen atoms; a C₁-C₆ alkylsulfonyl group which may be substituted with one or more groups selected from Substituent Group α and Substituent Group γ; a C₆-C₁₀ arylsulfonyl group which may be substituted with one or more groups selected from Substituent Group α, Substituent Group β and Substituent Group γ; a 5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms; a C₂-C₇ alkoxycarbonyl group which may be substituted with one or more groups selected from Substituent Group α and Substituent Group γ; a C₇-C₁₁, aryloxycarbonyl group which may be substituted with one or more groups selected from Substituent Group α, Substituent Group β and Substituent Group γ; or one or more groups having the formula R^{c}(R^{d})N—CO— (wherein R^{c} and R^{d} are the same or different and independently represent a hydrogen atom or a C₁-C₆ alkyl group which may be substituted with one or more groups selected from Substituent Group α and Substituent Group γ), and n represents an integer of 1 to 4,
Substituent Group α represents the group consisting of a halogen atom; a nitro group; a cyano group; a hydroxy group; a group having the formula R⁶—CO—, the formula R^{e}(R^{f})N— or the formula R^{e}(R^{f})N—CO— (wherein R⁶ represents a hydrogen atom, a C₁-C₆ alkyl group, a C₁-C₆ halogenated alkyl group, a hydroxy group, a C₁-C₆ alkoxy group or a C₆-C₁₀ aryloxy group and R^{e} and R^{f} are the same or different and independently represent a hydrogen atom; a C₁-C₆ alkyl group; a C₆-C₁₀ aryl group; a 5- to 7-membered heteroaryl group which contains 1 to 3 sulfur, oxygen and/or nitrogen atoms; a formyl group; a C₂-C₇ alkylcarbonyl group; a C₇-C₁₁ arylcarbonyl group; a 5- to 7-membered heteroarylcarbonyl group which contains 1 to 3 sulfur, oxygen and/or nitrogen atoms; a C₁-C₆ alkylsulfonyl group; a C₆-C₁₀ arylsulfonyl group; or a 5- to 7-membered heteroarylsulfonyl group which contains 1 to 3 sulfur, oxygen and/or nitrogen atoms, or alternatively R^{e} and R^{f} together with the nitrogen atom to which they are bonded form a 5- to 7-membered heterocylyl group which contains 1 to 3 sulfur, oxygen and/or nitrogen atoms); a C₁-C₆ alkoxy group; a C₁-C₆ alkylthio group; a C₁-C₆ alkylsulfinyl group; and a C₁-C₆ alkylsulfonyl group, Substituent Group β represents the group consisting of a C₁-C₆ alkyl group which may be substituted with one or more groups selected from Substituent Group α, and Substituent Group γ represents the group consisting of a C₁-C₆ alkoxy group substituted with one or more groups selected from Substituent Group α; a C₃-C₈ cycloalkyl group which may be substituted with one or more groups selected from Substituent Group α and Substituent Group β; a 5- to 7-membered heterocyclyl group which may be substituted with one or more groups selected from Substituent Group α and Substituent Group β and which contains 1 to 3 sulfur, oxygen and/or nitrogen atoms; a C₆-C₁₀ aryl group which may be substituted with one or more groups selected from Substituent Group α and Substituent Group β; a 5- to 7-membered heteroaryl group which may be substituted with one or more groups selected from Substituent Group α and Substituent Group γ and which contains 1 to 3 sulfur, oxygen and/or nitrogen atoms; a C₃-C₈ cycloalkyloxy group which may be substituted with one or more groups selected from Substituent Group α and Substituent Group β; a 5- to 7-membered heterocyclyloxy group which may be substituted with one or more groups selected from Substituent Group α and Substituent Group β and which contains 1 to 3 sulfur, oxygen and/or nitrogen atoms; a C₆-C₁₀ aryloxy group which may be substituted with one or more groups selected from Substituent Group α and Substituent Group β; a 5- to 7-membered heteroaryloxy group which may be substituted with one or more groups selected from Substituent Group α and Substituent Group β and which contains 1 to 3 sulfur, oxygen and/or nitrogen atoms; and a C₆-C₁₀ aryl-C₁-C₆ alkoxy group in which the aryl moiety may be substituted with one or more groups selected from Substituent Group α and Substituent Group β] or a pharmacologically acceptable salt thereof.

Preferred examples of the above compound are
(2) compounds in which R¹ is a hydrogen atom or a C₁-C₄ alkyl group,
(3) compounds in which R¹ is a hydrogen atom, methyl, ethyl or propyl,
(4) compounds in which R¹ is a hydrogen atom or methyl,
(5) compounds in which R² is a group R^{a}(R^{b})N—, and R^{a} and R^{b} are the same or different and independently represent a C₁-C₆ alkyl group which may be substituted with one or more groups selected from Substituent Group α and Substituent Group γ,
(6) compounds in which R^{a} is a C₁-C₆ alkyl group which may be substituted with one group selected from Substituent Group α and Substituent Group γ, R^{b} is a C₁-C₆ alkyl group, and Substituent Group α is the group consisting of a C₁-C₆ alkoxy group, and Substituent Group γ is the group consisting of a C₁-C₆ alkoxy group substituted with one or more groups selected from Substituent Group α; a C₆-C₁₀ aryloxy group which may be substituted with one or more groups selected from Substituent Group α and Substituent Group β; and a 5- to 7-membered heteroaryloxy group which may be substituted with one or more groups selected from Substituent Group α and Substituent Group β and which contains 1 to 3 sulfur, oxygen and/or nitrogen atoms,
(7) compounds in which R² is a group 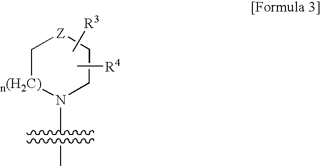
   wherein R⁴ is a hydrogen atom or together with R³ forms a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms,
(8) compounds in which R² is a group 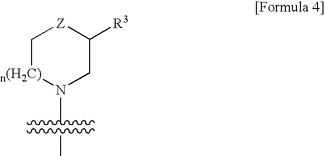
   wherein Z represents a single bond, an oxygen atom, a sulfur atom, sulfinyl or a group having the formula R⁵N<,
R⁵ represents a C₆-C₁₀ aryl group which may be substituted with one or more groups selected from Substituent Group α, Substituent Group β and Substituent Group γ; a 5- to 7-membered heteroaryl group which may be substituted with one or more groups selected from Substituent Group α, Substituent Group β and Substituent Group γ and which contains 1 to 3 sulfur, oxygen and/or nitrogen atoms; a formyl group; a C₂-C₇ alkylcarbonyl group which may be substituted with one or more groups selected from Substituent Group α and Substituent Group γ; a C₁-C₆ alkylsulfonyl group which may be substituted with one or more groups selected from Substituent Group α and Substituent Group γ; a C₆-C₁₀ arylsulfonyl group which may be substituted with one or more groups selected from Substituent Group α, Substituent Group β and Substituent Group γ; a 5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms; a C₂-C₇ alkoxycarbonyl group which may be substituted with one or more groups selected from Substituent Group α and Substituent Group γ; or one or more groups having the formula R^{c}(R^{d})N—CO—, and n is an integer of 1 to 3,
(9) compounds in which R³ is a C₁-C₆ alkoxy group; a C₁-C₆ alkyl group which may be substituted with one or more groups selected from Substituent Group α; a C₁-C₆ alkoxy group substituted with one or more groups selected from Substituent Group α; a C₆-C₁₀ aryloxy group which may be substituted with one or more groups selected from Substituent Group α, Substituent Group β and Substituent Group γ; a C₁-C₆ alkyl group substituted with one or more groups selected from Substituent Group γ; or a C₁-C₆ alkoxy group substituted with one or more groups selected from Substituent Group γ,
Z is a single bond, and n is 2,
(10) compounds in which R³ is a hydrogen atom, Z is a sulfur atom, and n is 1,and
(11) compounds in which R³ is a hydrogen atom, Z is a group having the formula R⁵N<, R⁵ represents a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms: n is 2
   and pharmacologically acceptable salts thereof, and particularly preferred compounds are
(12) any of the compounds selected from the following:
4-[4-(4-acetylphenyl)-1,4-diazepan-1-yl]-3-aminothieno[2,3-b]pyridine-2-carboxamide,
3-amino-4-{4-[4-(dimethylamino)phenyl]-1,4-diazepan-1-yl}thieno[2,3-b]pyridine-2-carboxamide,
3-amino-4-{4-[4-(methylsulfinyl)phenyl]-1,4-diazepan-1-yl}thieno[2,3-b]pyridine-2-carboxamide, and
3-amino-4-[4-(1,3-thiazol-2-yl)-1,4-diazepan-1-yl]thieno[2,3-b]pyridine-2-carboxamide or pharmacologically acceptable salts thereof.

The present invention further relates to
(13) a pharmaceutical composition which comprises a compound selected from any one of the above (1) to (12) or a pharmacologically acceptable salt thereof as an active ingredient {particularly a pharmaceutical composition for prevention or treatment of osteopathy [for example, osteoporosis (for example, postmenopausal osteoporosis, senile osteoporosis or secondary osteoporosis caused by the use of steroids or immunosuppressants), osteopenia or bone destruction associated with rheumatoid arthritis, Paget's disease of bone, bone fracture or dysostosis due to dwarfism] or osteoarthritis},
(14) use of a compound selected from any one of the above (1) to (12) or a pharmacologically acceptable salt thereof as an active ingredient for preparing a pharmaceutical composition {particularly a pharmaceutical composition for prevention or treatment of osteopathy [for example, osteoporosis (for example, postmenopausal osteoporosis, senile osteoporosis or secondary osteoporosis caused by the use of steroids or immunosuppressants), osteopenia or bone destruction associated with rheumatoid arthritis, Paget's disease of bone, bone fracture or dysostosis due to dwarfism] or osteoarthritis},
(15) a method for promoting osteogenesis, suppressing bone resorption and/or improving bone density comprising administering an effective amount of a compound selected from any one of the above (1) to (12) or a pharmacologically acceptable salt thereof to a mammal (for example, a human, horse, cow or pig, preferably a human) or a bird (preferably a chicken, more preferably a female chicken) {preferably a method for prevention or treatment of osteopathy [for example, osteoporosis (for example, postmenopausal osteoporosis, senile osteoporosis or secondary osteoporosis caused by the use of steroids or immunosuppressants), osteopenia or bone destruction associated with rheumatoid arthritis, Paget's disease of bone, bone fracture or dysostosis due to dwarfism] or osteoarthritis}.

In the above general formula (I), the “C₁-C₆ alkyl group” in the definitions of R¹, R⁶, R^{e} and R^{f}; the C₁-C₆ alkyl group of “C₁-C₆ alkyl group which may be substituted with one or more groups selected from Substituent Group α and Substituent Group γ” in the definitions of R^{a}, R^{b}, R⁵, R^{c} and R^{d}; the C₁-C₆ alkyl group of “C₁-C₆ alkyl group substituted with one or more groups selected from Substituent Group γ” in the definitions of R³ and R⁴; the alkyl moiety of “C₂-C₇ alkylcarbonyl group which may be substituted with one or more groups selected from Substituent Group α and Substituent Group γ” and “C₁-C₆ alkylsulfonyl group which may be substituted with one or more groups selected from Substituent Group a and Substituent Group γ” in the definition of R⁵; the alkyl moiety of “C₂-C₇ alkylcarbonyl group” and “C₁-C₆ alkylsulfonyl group” in the definition of R^{e} and R^{f}; the alkyl moiety of “C₁-C₆ alkylthio group”, “C₁-C₆ alkylsulfinyl group” and “C₁-C₆ alkylsulfonyl group” in the definition of Substituent Group α; and the C₁-C₆ alkyl group of “C₁-C₆ alkyl group which may be substituted with one or more groups selected from Substituent Group α” in the definition of Substituent Group β can be a linear or branched chain alkyl group such as a methyl, ethyl, propyl, isopropyl, butyl, isobutyl, s-butyl, tert-butyl, pentyl, isopentyl, 2-methylbutyl, neopentyl, 1-ethylpropyl, hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl or 2-ethylbutyl group, and preferably it is a C₁-C₄ linear or branched chain alkyl group, more preferably a C₁-C₃ linear or branched chain alkyl group and still more preferably a methyl, ethyl or isopropyl group.

The C₃-C₈ cycloalkyl group of “C₃-C₈ cycloalkyl group which may be substituted with one or more groups selected from Substituent Group α, Substituent Group β and Substituent Group γ” in the definitions of R^{a}, R^{b} and R⁵; the C₃-C₈ cycloalkyl group of “C₃-C₈ cycloalkyl group which may be substituted with one or more groups selected from Substituent Group α, Substituent Group β and Substituent Group γ” which R³ and R⁴ form together with the carbon atoms to which they are bonded; the C₃-C₈ cycloalkyl group of “C₃-C₈ cycloalkyl group which may be substituted with one or more groups selected from Substituent Group α and Substituent Group β” in the definition of Substituent Group γ; and the C₃-C₈ cycloalkyl moiety of “C₃-C₈ cycloalkyloxy group which may be substituted with one or more groups selected from Substituent Group α and Substituent Group β” in the definition of Substituent Group γ can be a cyclopropyl, cyclobutyl, cyclopentyl, cyclohexyl, cycloheptyl or cyclooctyl group and preferably it is a C₅-C₇ cycloalkyl group, and more preferably a cyclopentyl, cyclohexyl or cycloheptyl group.

The 5- to 7-membered heterocyclyl group of “5- to 7-membered heterocyclyl group which may be substituted with one or more groups selected from Substituent Group α, Substituent Group β and Substituent Group γ and which contains 1 to 3 sulfur, oxygen and/or nitrogen atoms” in the definitions of R^{a}, R^{b} and R⁵; the 5- to 7-membered heterocyclyl group of “5- to 7-membered heterocyclyl group which may be substituted with one or more groups selected from Substituent Group α, Substituent Group β and Substituent Group γ and which contains 1 to 3 sulfur, oxygen and/or nitrogen atoms” which R³ and R⁴ form together with the carbon atoms to which they are bonded; the 5- to 7-membered heterocyclyl moiety of “5- to 7-membered heterocyclylcarbonyl group which may be substituted with one or more groups selected from Substituent Group α, Substituent Group β and Substituent Group γ and which contains 1 to 3 sulfur, oxygen and/or nitrogen atoms” in the definition of R⁵; the “5- to 7-membered heterocyclyl group which contains 1 to 3 sulfur, oxygen and/or nitrogen atoms” which R^{e} and R^{f} form together with the nitrogen atoms to which they are bonded; the 5- to 7-membered heterocyclyl group of “5- to 7-membered heterocyclyl group which may be substituted with one or more groups selected from Substituent Group α and Substituent Group β and which contains 1 to 3 sulfur, oxygen and/or nitrogen atoms” in the definition of Substituent Group γ; and the 5- to 7-membered heterocyclyl moiety of “5- to 7-membered heterocyclyloxy group which may be substituted with one or more groups selected from Substituent Group α and Substituent Group β and which contains 1 to 3 sulfur, oxygen and/or nitrogen atoms” in the definition of Substituent Group γ can be, for example, azetidinyl, pyrrolidinyl, pyrrolinyl, imidazolidinyl, imidazolinyl, pyrazolidinyl, pyrazolinyl, oxazolidinyl, thiazolidinyl, oxadiazolidinyl, piperidyl, tetrahydropyridyl, dihydropyridyl, 2H-pyranyl, 2-oxo-2H-pyranyl, piperazinyl, morpholinyl, thiomorpholinyl or homopiperidyl, and preferably it is a 5- to 6-membered heterocyclyl group which contains 1 or 2 sulfur, oxygen and/or nitrogen atoms, and particularly preferably it is piperidyl, piperazinyl or morpholinyl.

Here, the above mentioned “5- to 7-membered heterocyclyl group which contains 1 to 3 sulfur, oxygen and/or nitrogen atoms” may be condensed with another cyclic group (for example, a phenyl group) and such a group can be, for example, tetrahydroquinolinyl, tetrahydroisoquinolinyl, chromanyl, indolinyl or isoindolinyl.

The C₆-C₁₀ aryl group of “C₆-C₁₀ aryl group which may be substituted with one or more groups selected from Substituent Group α, Substituent Group β and Substituent Group γ” in the definitions of R^{a}, R^{b} and R⁵; the C₆-C₁₀ aryl group of “C₆-C₁₀ aryl group which may be substituted with one or more groups selected from Substituent Group α, Substituent Group β and Substituent Group γ” which R³ and R⁴ form together with the carbon atoms to which they are bonded; the aryl moiety of “C₇-C₁₁ arylcarbonyl group which may be substituted with one or more groups selected from Substituent Group α, Substituent Group β and Substituent Group γ” in the definition of R⁵, the aryl moiety of “C₆-C₁₀ arylsulfonyl group which may be substituted with one or more groups selected from Substituent Group α, Substituent Group β and Substituent Group γ” and “C₇-C₁₁, aryloxycarbonyl group which may be substituted with one or more groups selected from Substituent Group α, Substituent Group β and Substituent Group γ”; the aryl moiety of “C₆-C₁₀ aryloxy group” in the definition of R⁶; the “C₆-C₁₀ aryl group” in the definition of R^{e} and R^{f}; the aryl moiety of “C₇-C₁₁ arylcarbonyl group” and “C₆-C₁₀ arylsulfonyl group” in the definition of R^{e} and R^{f}; the C₆-C₁₀ aryl group of “C₆-C₁₀ aryl group which may be substituted with one or more groups selected from Substituent Group α and Substituent Group β” in the definition of Substituent Group γ; the aryl moiety of “C₆-C₁₀ aryloxy group which may be substituted with one or more groups selected from Substituent Group α and Substituent Group β” in the definition of Substituent Group γ; and the aryl moiety of “C₆-C₁₀ aryl-C₁-C₆ alkoxy group in which the aryl moiety may be substituted with one or more groups selected from Substituent Group α and Substituent Group β” in the definition of Substituent Group γ can be, for example, phenyl or naphthyl and is preferably phenyl.

Here, the above mentioned “C₆-C₁₀ aryl group” may be condensed with a C₃-C₁₀ cycloalkyl group (preferably a C₅-C₆ cycloalkyl group), and examples of such a group include 5-indanyl.

The 5- to 7-membered heteroaryl group of “5- to 7-membered heteroaryl group which may be substituted with one or more groups selected from Substituent Group α, Substituent Group β and Substituent Group γ and which contains 1 to 3 sulfur, oxygen and/or nitrogen atoms” in the definitions of R^{a}, R^{b} and R⁵; the 5- to 7-membered heteroaryl group of “5- to 7-membered heteroaryl group which may be substituted with one or more groups selected from Substituent Group α, Substituent Group β and Substituent Group γ and which contains 1 to 3 sulfur, oxygen and/or nitrogen atoms” which R³ and R⁴ form together with the carbon atoms to which they are bonded; the 5- to 7-membered heteroaryl moiety of “5- to 7-membered heteroarylcarbonyl group which may be substituted with one or more groups selected from Substituent Group α, Substituent Group β and Substituent Group γ and which contains 1 to 3 sulfur, oxygen and/or nitrogen atoms” and “5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms” in the definition of R⁵; the “5-to 7-membered heteroaryl group which contains 1 to 3 sulfur, oxygen and/or nitrogen atoms” in the definitions of R^{e} and R^{f}; the 5- to 7-membered heteroaryl moiety of “5-to 7-membered heteroarylcarbonyl group which contains 1 to 3 sulfur, oxygen and/or nitrogen atoms” and “5- to 7-membered heteroarylsulfonyl group which contains 1 to 3 sulfur, oxygen and/or nitrogen atoms” in the definitions of R^{e} and R^{f}; the 5- to 7-membered heteroaryl group of “5- to 7-membered heteroaryl group which may be substituted with one or more groups selected from Substituent Group α and Substituent Group β and which contains 1 to 3 sulfur, oxygen and/or nitrogen atoms” in the definition of Substituent Group γ; and the 5- to 7-membered heteroaryl moiety of “5- to 7-membered heteroaryloxy group which may be substituted with one or more groups selected from Substituent Group α and Substituent Group β and which contains 1 to 3 sulfur, oxygen and/or nitrogen atoms” in the definition of Substituent Group γ can be, for example, furyl, thienyl, pyrrolyl, pyrazolyl, imidazolyl, oxazolyl, isoxazolyl, thiazolyl, isothiazolyl, triazolyl, thiadiazolyl, pyridyl, pyridazinyl, pyrimidinyl, pyrazinyl or azepinyl and preferably it is a 5- to 6-membered heteroaryl group which contains 1 or 2 sulfur, oxygen and/or nitrogen atoms such as furyl, thienyl, pyrrolyl, pyrazolyl, imidazolyl, oxazolyl, isoxazolyl, thiazolyl, isothiazolyl, pyridyl, pyridazinyl, pyrimidinyl or pyrazinyl.

Here, the above mentioned “5- to 7-membered heteroaryl group which contains 1 to 3 sulfur, oxygen and/or nitrogen atoms” may be condensed with another cyclic group [for example, a C₆-C₁₀ aryl or C₃-C₈ cycloalkyl group (preferably, a C₅-C₆ cycloalkyl group)], and such a group can be indolyl, benzofuranyl, benzothienyl, quinolyl, isoquinolyl, quinazolinyl, tetrahydroquinolyl or tetrahydroisoquinolyl.

The C₁-C₆ alkoxy group of “C₁-C₆ alkoxy group substituted with one or more groups selected from Substituent Group γ” in the definitions of R³ and R⁴; the alkoxy moiety of “C₂-C₇ alkoxycarbonyl group which may be substituted with one or more groups selected from Substituent Group α and Substituent Group γ” in the definition of R⁵; the “C₁-C₆ alkoxy group” in the definitions of R⁶ and Substituent Group α; and the C₁-C₆ alkoxy group of “C₁-C₆ alkoxy group substituted with one or more groups selected from Substituent Group a” in the definition of Substituent Group γ are one or more groups in which an oxygen atom is bonded to the above “C₁-C₆ alkyl group”, preferably it is a C₁-C₄ linear or branched chain alkoxy group, more preferably it is methoxy, ethoxy, propoxy, isopropoxy, butoxy, isobutoxy or tert-butoxy, still more preferably it is methoxy, ethoxy, propoxy, isopropoxy or butoxy, and particularly preferably it is methoxy or ethoxy.

The C₂-C₆ alkenyl group of “C₂-C₆ alkenyl group which may be substituted with one or more groups selected from Substituent Group α and Substituent Group γ” in the definition of R⁵ can be a linear or branched chain alkenyl group such as a vinyl, 2-propenyl, 1-methyl-2-propenyl, 2-methyl-2-propenyl, 2-ethyl-2-propenyl, 2-butenyl, 1-methyl-2-butenyl, 2-methyl-2-butenyl, 1-ethyl-2-butenyl, 3-butenyl, 1-methyl-3-butenyl, 2-methyl-3-butenyl, 1-ethyl-3-butenyl, 2-pentenyl, 1-methyl-2-pentenyl, 2-methyl-2-pentenyl, 3-pentenyl, 1-methyl-3-pentenyl, 2-methyl-3-pentenyl, 4-pentenyl, 1-methyl-4-pentenyl, 2-methyl-4-pentenyl, 2-hexenyl, 3-hexenyl, 4-hexenyl or 5-hexenyl group and preferably it is a C₂-C₄ linear or branched chain alkyl group, more preferably it is vinyl or 2-propenyl, and particularly preferably it is vinyl.

The “C₂-C₇ alkylcarbonyl group” in the definitions of R^{e} and R^{f}; and the C₂-C₇ alkylcarbonyl group of “C₂-C₇ alkylcarbonyl group which may be substituted with one or more groups selected from Substituent Group α and Substituent Group γ” in the definition of R⁵is a group in which carbonyl is bonded to the above “C₁-C₆ alkyl group”, and preferably it is a C₂-C₅ linear or branched chain alkylcarbonyl group, more preferably it is acetyl, propionyl, propylcarbonyl, isopropylcarbonyl, butylcarbonyl, isobutylcarbonyl or tert-butylcarbonyl, still more preferably it is acetyl, propionyl, propylcarbonyl, isopropylcarbonyl or butylcarbonyl, and particularly preferably it is acetyl or propionyl.

The 5- to 7-membered heterocyclylcarbonyl group of “5- to 7-membered heterocyclylcarbonyl group which may be substituted with one or more groups selected from Substituent Group α, Substituent Group β and Substituent Group γ and which contains 1 to 3 sulfur, oxygen and/or nitrogen atoms” in the definition of R⁵ is a group in which carbonyl is bonded to the above “5- to 7-membered heterocyclyl group”, and preferably it is a 5- or 6-membered heterocyclylcarbonyl group which contains 1 or 2 sulfur, oxygen and/or nitrogen atoms, and particularly preferably it is piperidylcarbonyl, piperazinylcarbonyl or morpholinylcarbonyl.

The “C₇-C₁₁ arylcarbonyl group” in the definitions of R^{e} and R^{f}; and the C₇-C₁₁ arylcarbonyl group of “C₇-C₁₁ arylcarbonyl group which may be substituted with one or more groups selected from Substituent Group α, Substituent Group β and Substituent Group γ” in the definition of R⁵ is a group in which carbonyl is bonded to the above “C₆-C₁₀ aryl group”, and preferably it is benzoyl.

The “5- to 7-membered heteroarylcarbonyl group which contains 1 to 3 sulfur, oxygen and/or nitrogen atoms” in the definitions of R^{e} and R^{f}; and the 5- to 7-membered heteroarylcarbonyl group of “5- to 7-membered heteroarylcarbonyl group which may be substituted with one or more groups selected from Substituent Group α, Substituent Group β and Substituent Group γ and which contains 1 to 3 sulfur, oxygen and/or nitrogen atoms” in the definition of R⁵is a group in which carbonyl is bonded to the above “5- to 7-membered heteroaryl group”, and preferably it is 5- to 6-membered heteroarylcarbonyl which contains 1 or 2 sulfur, oxygen and/or nitrogen atoms.

The “C₁-C₆ alkylsulfonyl group” in the definitions of Substituent Group α, R^{e}and R^{f}; and the C₁-C₆ alkylsulfonyl group of “C₁-C₆ alkylsulfonyl group which may be substituted with one or more groups selected from Substituent Group α and Substituent Group γ” in the definition of R⁵ is a group in which sulfonyl is bonded to the above “C₁-C₆ alkyl group”, and preferably it is a C₁-C₄ linear or branched chain alkylsulfonyl group, more preferably it is methylsulfonyl, ethylsulfonyl, propylsulfonyl, isopropylsulfonyl, butylsulfonyl, isobutylsulfonyl or tert-butylsulfonyl, still more preferably it is methylsulfonyl, ethylsulfonyl, propylsulfonyl, isopropylsulfonyl or butylsulfonyl, and particularly preferably it is methylsulfonyl or ethylsulfonyl.

The “C₆-C₁₀ arylsulfonyl group” in the definitions of R^{e} and R^{f}; and the C₆-C₁₀ arylsulfonyl group of “C₆-C₁₀ arylsulfonyl group which may be substituted with one or more groups selected from Substituent Group α, Substituent Group β and Substituent Group γ” in the definition of R⁵ is a group in which sulfonyl is bonded to the above “C₆-C₁₀ aryl group”, and preferably it is phenylsulfonyl.

The “5- to 7-membered heteroarylsulfonyl group which contains 1 to 3 sulfur, oxygen and/or nitrogen atoms” in the definitions of R^{e} and R^{f}; and the 5- to 7-membered heteroarylsulfonyl group of “5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms” in the definition R⁵ is a group in which sulfonyl is bonded to the above “5- to 7-membered heteroaryl group”, and preferably it is 5- to 6-membered heteroarylsulfonyl which contains 1 or 2 sulfur, oxygen and/or nitrogen atoms.

The C₂-C₇ alkoxycarbonyl group of “C₂-C₇ alkoxycarbonyl group which may be substituted with one or more groups selected from Substituent Group α and Substituent Group γ” in the definition of R⁵ is a group in which carbonyl is bonded to the above “C₁-C₆ alkoxy group”, and preferably it is a C₂-C₅ linear or branched chain alkoxycarbonyl group, more preferably it is methoxycarbonyl, ethoxycarbonyl, propoxycarbonyl, isopropoxycarbonyl, butoxycarbonyl, isobutoxycarbonyl or tert-butoxycarbonyl, still more preferably it is methoxycarbonyl, ethoxycarbonyl, propoxycarbonyl, isopropoxycarbonyl or tert-butoxycarbonyl, and particularly preferably it is ethoxycarbonyl or tert-butoxycarbonyl.

The “C₆-C₁₀ aryloxy group” in the definition of R⁶; and the C₆-C₁₀ aryloxy group of “C₆-C₁₀ aryloxy group which may be substituted with one or more groups selected from Substituent Group α and Substituent Group β” in the definition of Substituent Group γ is a group in which an oxygen atom is bonded to the above “C₆-C₁₀ aryl group”, and preferably it is phenoxy.

The C₇-C₁₁ aryloxycarbonyl group of “C₇-C₁₁ aryloxycarbonyl group which may be substituted with one or more groups selected from Substituent Group α, Substituent Group β and Substituent Group γ” in the definition of R⁵ is a group in which carbonyl is bonded to the above “C₆-C₁₀ aryloxy group”, and preferably it is phenoxycarbonyl.

The “halogen atom” in the definition of Substituent Group α is a fluorine atom, a chlorine atom, a bromine atom or an iodine atom and preferably it is a fluorine atom or a chlorine atom.

The “C₁-C₆ halogenated alkyl group” in the definition of R⁶ is a group in which one or two or more hydrogen atoms in the above “C₁-C₆ alkyl group” is substituted with a “halogen atom” mentioned above, and preferably it is a C₁-C₄ alkyl halide group, more preferably it is trifluoromethyl, trichloromethyl, difluoromethyl, dichloromethyl, dibromomethyl, fluoromethyl, 2,2,2-trichloroethyl, 2,2,2-trifluoroethyl, 2-bromoethyl, 2-chloroethyl, 2-fluoroethyl or 2,2-dibromoethyl, still more preferably it is trifluoromethyl, trichloromethyl, difluoromethyl or fluoromethyl, and the most preferably it is trifluoromethyl.

The “C₁-C₆ alkylthio group” in the definition of Substituent Group α is a group in which a sulfur atom is bonded to the above “C₁-C₆ alkyl group”, and preferably it is a C₁-C₄ linear or branched chain alkylthio group, more preferably it is methylthio, ethylthio, propylthio, isopropylthio, butylthio, isobutylthio or tert -butylthio, still more preferably it is methylthio, ethylthio, propylthio, isopropylthio or butylthio, and particularly preferably it is methylthio or ethylthio.

The “C₁-C₆ alkylsulfinyl group” in the definition of Substituent Group α is a group in which sulfinyl is bonded to the above “C₁-C₆ alkyl group”, and preferably it is a C₁-C₄ linear or branched chain alkylsulfinyl group, more preferably it is methylsulfinyl, ethylsulfinyl, propylsulphinyl, isopropylsulphinyl, butylsulfinyl, isobutylsulfinyl or tert-butylsulfinyl, still more preferably it is methylsulfinyl, ethylsulfinyl, propylsulphinyl, isopropylsulfinyl or butylsulfinyl, and particularly preferably it is methylsulfinyl or ethylsulfinyl.

The C₃-C₈ cycloalkyloxy group of “C₃-C₈ cycloalkyloxy group which may be substituted with one or more groups selected from Substituent Group α and Substituent Group β” in the definition of Substituent Group γ is a group in which an oxygen atom is bonded to the above “C₃-C₈ cycloalkyl group”, and preferably it is a C₅-C₇ cycloalkyloxy group, and more preferably it is cyclopentyloxy, cyclohexyloxy or cycloheptyloxy.

The 5- to 7-membered heterocyclyloxy group of “5- to 7-membered heterocyclyloxy group which may be substituted with one or more groups selected from Substituent Group α and Substituent Group β and which contains 1 to 3 sulfur, oxygen and/or nitrogen atoms” in the definition of Substituent Group γ is a group in which an oxygen atom is bonded to the above “5- to 7-membered heterocyclyl group”, and preferably it is a 5- or 6-membered heterocyclyloxy group which contains 1 or 2 sulfur, oxygen and/or nitrogen atoms, and particularly preferably it is piperidyloxy.

The 5- to 7-membered heteroaryloxy group of “5- to 7-membered heteroaryloxy group which may be substituted with one or more groups selected from Substituent Group α and Substituent Group β and which contains 1 to 3 sulfur, oxygen and/or nitrogen atoms” in the definition of Substituent Group γ is a group in which an oxygen atom is bonded to the above “5- to 7-membered heteroaryl which contains 1 to 3 sulfur, oxygen and/or nitrogen atoms”, and preferably it is a 5- to 6-member heteroaryloxy group which contains 1 or 2 sulfur, oxygen and/or nitrogen atoms, and more preferably it is furyloxy, thienyloxy, pyrrolyloxy, pyrazolyloxy, imidazolyloxy, oxazolyloxy, isoxazolyloxy, thiazolyloxy, isothiazolyloxy, pyridyloxy, pyridazinyloxy, pyrimidinyloxy or pyrazinyloxy.

The C₆-C₁₀ aryl-C₁-C₆ alkoxy group of “C₆-C₁₀ aryl-C₁-C₆ alkoxy group in which the aryl moiety may be substituted with one or more groups selected from Substituent Group α and Substituent Group β” in the definition of Substituent Group γ is the above “C₁-C₆ alkoxy group” substituted with a “C₆-C₁₀ aryl group” mentioned above, and preferably it is benzyloxy, phenethyloxy or 3-phenylpropyloxy, and particularly preferably it is benzyloxy.

Since compound (I) of the present invention can be converted to a salt by reacting with an acid when it has a basic functional group such as an amino group or with a base when it has an acidic functional group such as a carboxyl group, the “pharmacologically acceptable salt thereof” refers to such a salt.

The salt based on a basic functional group can be, for example, an inorganic acid salt such as a hydrohalide such as hydrochloride, hydrobromide or hydriodide, nitrate, perchlorate, sulfate or phosphate; an organic salt such as a lower alkanesulfonate such as methanesulfonate, trifluoromethanesulfonate or ethanesulfonate, an aryl sulfonate such as benzenesulfonate or p-toluenesulfonate, or a carboxylate such as acetate, malate, fumarate, succinate, citrate, ascorbate, tartrate, oxalate or maleate; or it can be an amino acid salt such as glycinate, lysinate, argininate, ornithinate, glutamate or aspartate.

The salt based on an acid functional group can be, for example, a metal salt such as an alkali metal salt such as a sodium salt, potassium salt or lithium salt, an alkaline earth metal salt such as a calcium salt or magnesium salt, an aluminium salt or an iron salt; an ammonium salt; an organic amine salt such as a t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt or tris(hydroxymethyl)aminomethane salt; or it can be an amino acid salt such as glycine salt, lysine salt, arginine salt, ornithine salt, glutamate or aspartate.

The compounds having general formula (I) of the present invention or pharmacologically acceptable salt thereof when allowed to stand in the atmosphere or to recrystallize may absorb water or adsorb water to form hydrates and such hydrates are also included in the present invention.

Among the compounds having general formula (I) of the present invention, there may be optical isomers due to an asymmetric centre in the molecule. For the compounds of the present invention, it should be noted that these isomers and mixtures thereof are represented by a single formula, i.e. general formula (I). Therefore, the present invention encompasses these isomers and mixtures of these isomers in any ratio.

Specific examples of the compound having general formula (I) of the present invention include compounds described in the following Table 1 to Table 3 for exemplified compounds.

Here, in Table 1 to Table 3 for exemplified compounds, “Ac” represents acetyl, “perhydro-1-Azep” represents perhydroazepin-1-yl, “perhydro-1-Azoc” represents perhydroazocin-1-yl, “Bn” represents benzyl, “Bu” represents butyl, “iBu” represents isobutyl, “tBu” represents tert-butyl, “BzDioxa” represents 1,4-benzodioxanyl, “BzDioxo” represents 1,3-benzodioxolanyl, “Bzhy” represents benzhydryl, “BzOxaz” represents benzoxazolyl, “BzThaz” represents benzothiazolyl, “Et” represents ethyl, “cHep” represents cycloheptyl, “cHx” represents cyclohexyl, “Iqui” represents isoquinolyl, “decahydro-2-Iqui” represents 1,2,3,4,5,6,7,8,9,10-decahydroisoquinolin-2-yl, “1,2,3,4-tetrahydro-2-Iqui” represents 1,2,3,4-tetrahydroisoquinolin-2-yl, “Me” represents methyl, “Mor” represents morpholin-4-yl, “Oxaz” represents oxazolyl, “1,4-Oxazep” represents 1,4-oxazepin-4-yl, “Ph” represents phenyl, “Phet” represents phenethyl, “Pip” represents piperidin-4-yl, “3,4-dehydro-Pip” represents 3,4-dehydropiperidin-4-yl, “cPn” represents cyclopentyl, “neoPn” represents neopentyl, “Pr” represents propyl, “iPr” represents isopropyl, “Py” represents pyridyl, “Pydz” represents pyridazinyl, “Pym” represents pyrimidyl, “Pyrld” represents pyrrolidin-1-yl, “Qunz” represents quinazolinyl, “Thaz” represents thiazolyl, “Thi” represents thienyl, “1,4-Thiazep” represents 1,4-thiazepin-4-yl, and “Thmor” represents thiomorpholin-4-yl.

TABLE 1
for exemplified compounds

| | |
|---|---|
| | (I) |
| 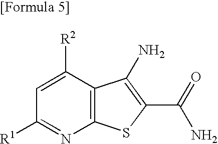 | |

| Compound | | |
|---|---|---|
| Number | R¹ | R² |
| 1-1 | H | MeO |
| 1-2 | H | EtO |
| 1-3 | H | iPrO |
| 1-4 | H | BnO |
| 1-5 | H | (HOOC)—CH₂O |
| 1-6 | H | cPnO |
| 1-7 | H | cHxO |
| 1-8 | H | cHepO |
| 1-9 | H | MeS |
| 1-10 | H | EtS |
| 1-11 | H | iPrS |
| 1-12 | H | BnS |
| 1-13 | H | cHxS |
| 1-14 | H | cHepS |
| 1-15 | H | Pyrld |
| 1-16 | H | Pip |
| 1-17 | H | 3-Me-Pip |
| 1-18 | H | 4-Me-Pip |
| 1-19 | H | 4-Bn-Pip |
| 1-20 | H | 3-(HO—CH₂)-Pip |
| 1-21 | H | 3-(MeO—CH₂)-Pip |
| 1-22 | H | 3-(EtO—CH₂)-Pip |
| 1-23 | H | 3-(PrO—CH₂)-Pip |
| 1-24 | H | 3-(BnO—CH₂)-Pip |
| 1-25 | H | 3-Ph-Pip |
| 1-26 | H | 4-Ph-Pip |
| 1-27 | H | 3-HO-Pip |
| 1-28 | H | 3-MeO-Pip |
| 1-29 | H | 3-EtO-Pip |
| 1-30 | H | 3-PrO-Pip |
| 1-31 | H | 3-BnO-Pip |
| 1-32 | H | 4-HO-Pip |
| 1-33 | H | 3-AcO-Pip |
| 1-34 | H | 4-AcO-Pip |
| 1-35 | H | 1,2,3,4-tetrahydro-2-Iqui |
| 1-36 | H | decahydro-2-Iqui |
| 1-37 | H | 3,4-dehydro-Pip |
| 1-38 | H | 4-Ph-3,4-dehydro-1-Pip |
| 1-39 | H | perhydro-1-Azep |
| 1-40 | H | perhydro-1-Azoc |
| 1-41 | H | Mor |
| 1-42 | H | 2,6-diMe-Mor |
| 1-43 | H | 1,4-Oxazep |
| 1-44 | H | Thmor |
| 1-45 | H | 1-Oxo-Thmor |
| 1-46 | H | 1,4-Thiazep |
| 1-47 | H | 1-Oxo-1,4-Thiazep |
| 1-48 | Me | MeO |
| 1-49 | Me | EtO |
| 1-50 | Me | iPrO |
| 1-51 | Me | BnO |
| 1-52 | Me | (HOOC)—CH₂O |
| 1-53 | Me | cPnO |
| 1-54 | Me | cHxO |
| 1-55 | Me | cHepO |
| 1-56 | Me | MeS |
| 1-57 | Me | EtS |
| 1-58 | Me | iPrS |
| 1-59 | Me | BnS |
| 1-60 | Me | cHxS |
| 1-61 | Me | cHepS |
| 1-62 | Me | Pyrld |
| 1-63 | Me | Pip |
| 1-64 | Me | 3-Me-Pip |
| 1-65 | Me | 4-Me-Pip |
| 1-66 | Me | 4-Bn-Pip |
| 1-67 | Me | 3-(HO—CH₂)-Pip |
| 1-68 | Me | 3-(MeO-CH₂)-Pip |
| 1-69 | Me | 3-(EtO-CH₂)-Pip |
| 1-70 | Me | 3-(PrO-CH₂)-Pip |
| 1-71 | Me | 3-(BnO-CH₂)-Pip |
| 1-72 | Me | 3-Ph-Pip |
| 1-73 | Me | 4-Ph-Pip |
| 1-74 | Me | 3-HO-Pip |
| 1-75 | Me | 3-MeO-Pip |
| 1-76 | Me | 3-EtO-Pip |
| 1-77 | Me | 3-PrO-Pip |
| 1-78 | Me | 3-BnO-Pip |
| 1-79 | Me | 4-HO-Pip |
| 1-80 | Me | 3-AcO-Pip |
| 1-81 | Me | 4-AcO-Pip |
| 1-82 | Me | 1,2,3,4-tetrahydro-2-Iqui |
| 1-83 | Me | decahydro-2-Iqui |
| 1-84 | Me | 3,4-dehydro-Pip |
| 1-85 | Me | 4-Ph-3,4-dehydro-1-Pip |
| 1-86 | Me | perhydro-1-Azep |
| 1-87 | Me | perhydro-1-Azoc |
| 1-88 | Me | Mor |
| 1-89 | Me | 2,6-diMe-Mor |
| 1-90 | Me | 1,4-Oxazep |
| 1-91 | Me | Thmor |
| 1-92 | Me | 1-Oxo-Thmor |
| 1-93 | Me | 1,4-Thiazep |
| 1-94 | Me | 1-Oxo-1,4-Thiazep |

TABLE 2
for exemplified compounds

| | |
|---|---|
| | (I-1) |
| 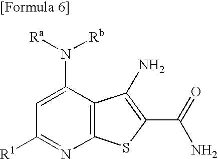 | |

| Compound | | | |
|---|---|---|---|
| Number | R¹ | R^{a} | R^{b} |
| 2-1 | H | H | Me |
| 2-2 | H | H | Et |
| 2-3 | H | H | Pr |
| 2-4 | H | H | iPr |
| 2-5 | H | H | Bu |
| 2-6 | H | H | iBu |
| 2-7 | H | H | neoPn |
| 2-8 | H | H | cPn |
| 2-9 | H | H | cHx |
| 2-10 | H | H | cHep |
| 2-11 | H | H | Bn |
| 2-12 | H | H | Phet |
| 2-13 | H | H | Ph-(CH₂)₃ |
| 2-14 | H | H | cHxCH₂ |
| 2-15 | H | H | MeOCH₂CH₂ |
| 2-16 | H | H | EtOCH₂CH₂ |
| 2-17 | H | Me | Me |
| 2-18 | H | Me | Et |
| 2-19 | H | Me | Pr |
| 2-20 | H | Me | iPr |
| 2-21 | H | Me | Bu |
| 2-22 | H | Me | iBu |
| 2-23 | H | Me | neoPn |
| 2-24 | H | Me | cPn |
| 2-25 | H | Me | cHx |
| 2-26 | H | Me | cHep |
| 2-27 | H | Me | Bn |
| 2-28 | H | Me | Phet |
| 2-29 | H | Me | Ph-(CH₂)₃ |
| 2-30 | H | Me | cHxCH₂ |
| 2-31 | H | Me | MeOCH₂CH₂ |
| 2-32 | H | Me | EtOCH₂CH₂ |
| 2-33 | H | Et | Et |
| 2-34 | H | Et | Pr |
| 2-35 | H | Et | iPr |
| 2-36 | H | Et | Bu |
| 2-37 | H | Et | iBu |
| 2-38 | H | Et | neoPn |
| 2-39 | H | Et | cPn |
| 2-40 | H | Et | cHx |
| 2-41 | H | Et | cHep |
| 2-42 | H | Et | Bn |
| 2-43 | H | Et | Phet |
| 2-44 | H | Et | Ph-(CH₂)₃ |
| 2-45 | H | Et | cHxCH₂ |
| 2-46 | H | Et | MeOCH₂CH₂ |
| 2-47 | H | Et | EtOCH₂CH₂ |
| 2-48 | H | Pr | Pr |
| 2-49 | H | Pr | iPr |
| 2-50 | H | Pr | Bu |
| 2-51 | H | Pr | iBu |
| 2-52 | H | Pr | neoPn |
| 2-53 | H | Pr | cPn |
| 2-54 | H | Pr | cHx |
| 2-55 | H | Pr | cHep |
| 2-56 | H | Pr | Bn |
| 2-57 | H | Pr | Phet |
| 2-58 | H | Pr | Ph-(CH₂)₃ |
| 2-59 | H | Pr | cHxCH₂ |
| 2-60 | H | Pr | MeOCH₂CH₂ |
| 2-61 | H | Pr | EtOCH₂CH₂ |
| 2-62 | H | MeOCH₂CH₂ | iPr |
| 2-63 | H | MeOCH₂CH₂ | Bu |
| 2-64 | H | MeOCH₂CH₂ | iBu |
| 2-65 | H | MeOCH₂CH₂ | neoPn |
| 2-66 | H | MeOCH₂CH₂ | cPn |
| 2-67 | H | MeOCH₂CH₂ | cHx |
| 2-68 | H | MeOCH₂CH₂ | cHep |
| 2-69 | H | MeOCH₂CH₂ | Bn |
| 2-70 | H | MeOCH₂CH₂ | Phet |
| 2-71 | H | MeOCH₂CH₂ | Ph-(CH₂)₃ |
| 2-72 | H | MeOCH₂CH₂ | cHxCH₂ |
| 2-73 | H | MeOCH₂CH₂ | MeOCH₂CH₂ |
| 2-74 | H | EtOCH₂CH₂ | iPr |
| 2-75 | H | EtOCH₂CH₂ | Bu |
| 2-76 | H | EtOCH₂CH₂ | iBu |
| 2-77 | H | EtOCH₂CH₂ | neoPn |
| 2-78 | H | EtOCH₂CH₂ | cPn |
| 2-79 | H | EtOCH₂CH₂ | cHx |
| 2-80 | H | EtOCH₂CH₂ | cHep |
| 2-81 | H | EtOCH₂CH₂ | Bn |
| 2-82 | H | EtOCH₂CH₂ | Phet |
| 2-83 | H | EtOCH₂CH₂ | Ph-(CH₂)₃ |
| 2-84 | H | EtOCH₂CH₂ | cHxCH₂ |
| 2-85 | H | EtOCH₂CH₂ | EtOCH₂CH₂ |
| 2-86 | Me | H | Me |
| 2-87 | Me | H | Et |
| 2-88 | Me | H | Pr |
| 2-89 | Me | H | iPr |
| 2-90 | Me | H | Bu |
| 2-91 | Me | H | iBu |
| 2-92 | Me | H | neoPn |
| 2-93 | Me | H | cPn |
| 2-94 | Me | H | cHx |
| 2-95 | Me | H | cHep |
| 2-96 | Me | H | Bn |
| 2-97 | Me | H | Phet |
| 2-98 | Me | H | Ph-(CH₂)₃ |
| 2-99 | Me | H | cHxCH₂ |
| 2-100 | Me | H | MeOCH₂CH₂ |
| 2-101 | Me | H | EtOCH₂CH₂ |
| 2-102 | Me | Me | Me |
| 2-103 | Me | Me | Et |
| 2-104 | Me | Me | Pr |
| 2-105 | Me | Me | iPr |
| 2-106 | Me | Me | Bu |
| 2-107 | Me | Me | iBu |
| 2-108 | Me | Me | neoPn |
| 2-109 | Me | Me | cPn |
| 2-110 | Me | Me | cHx |
| 2-111 | Me | Me | cHep |
| 2-112 | Me | Me | Bn |
| 2-113 | Me | Me | Phet |
| 2-114 | Me | Me | Ph-(CH₂)₃ |
| 2-115 | Me | Me | cHxCH₂ |
| 2-116 | Me | Me | MeOCH₂CH₂ |
| 2-117 | Me | Me | EtOCH₂CH₂ |
| 2-118 | Me | Et | Et |
| 2-119 | Me | Et | Pr |
| 2-120 | Me | Et | iPr |
| 2-121 | Me | Et | Bu |
| 2-122 | Me | Et | iBu |
| 2-123 | Me | Et | neoPn |
| 2-124 | Me | Et | cPn |
| 2-125 | Me | Et | cHx |
| 2-126 | Me | Et | cHep |
| 2-127 | Me | Et | Bn |
| 2-128 | Me | Et | Phet |
| 2-129 | Me | Et | Ph-(CH₂)₃ |
| 2-130 | Me | Et | cHxCH₂ |
| 2-131 | Me | Et | MeOCH₂CH₂ |
| 2-132 | Me | Et | EtOCH₂CH₂ |
| 2-133 | Me | Pr | Pr |
| 2-134 | Me | Pr | iPr |
| 2-135 | Me | Pr | Bu |
| 2-136 | Me | Pr | iBu |
| 2-137 | Me | Pr | neoPn |
| 2-138 | Me | Pr | cPn |
| 2-139 | Me | Pr | cHx |
| 2-140 | Me | Pr | cHep |
| 2-141 | Me | Pr | Bn |
| 2-142 | Me | Pr | Phet |
| 2-143 | Me | Pr | Ph-(CH₂)₃ |
| 2-144 | Me | Pr | cHxCH₂ |
| 2-145 | Me | Pr | MeOCH₂CH₂ |
| 2-146 | Me | Pr | EtOCH₂CH₂ |
| 2-147 | Me | MeOCH₂CH₂ | iPr |
| 2-148 | Me | MeOCH₂CH₂ | Bu |
| 2-149 | Me | MeOCH₂CH₂ | iBu |
| 2-150 | Me | MeOCH₂CH₂ | neoPn |
| 2-151 | Me | MeOCH₂CH₂ | cPn |
| 2-152 | Me | MeOCH₂CH₂ | cHx |
| 2-153 | Me | MeOCH₂CH₂ | cHep |
| 2-154 | Me | MeOCH₂CH₂ | Bn |
| 2-155 | Me | MeOCH₂CH₂ | Phet |
| 2-156 | Me | MeOCH₂CH₂ | Ph-(CH₂)₃ |
| 2-157 | Me | MeOCH₂CH₂ | cHxCH₂ |
| 2-158 | Me | MeOCH₂CH₂ | MeOCH₂CH₂ |
| 2-159 | Me | EtOCH₂CH₂ | iPr |
| 2-160 | Me | EtOCH₂CH₂ | Bu |
| 2-161 | Me | EtOCH₂CH₂ | iBu |
| 2-162 | Me | EtOCH₂CH₂ | neoPn |
| 2-163 | Me | EtOCH₂CH₂ | cPn |
| 2-164 | Me | EtOCH₂CH₂ | cHx |
| 2-165 | Me | EtOCH₂CH₂ | cHep |
| 2-166 | Me | EtOCH₂CH₂ | Bn |
| 2-167 | Me | EtOCH₂CH₂ | Phet |
| 2-168 | Me | EtOCH₂CH₂ | Ph-(CH₂)₃ |
| 2-169 | Me | EtOCH₂CH₂ | cHxCH₂ |
| 2-170 | Me | EtOCH₂CH₂ | EtOCH₂CH₂ |

TABLE 3
for exemplified compounds

| | |
|---|---|
| | (I-2) |
| 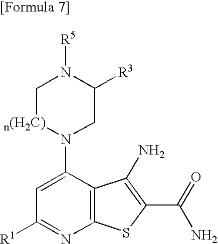 | |

| Compound | | | | |
|---|---|---|---|---|
| Number | R¹ | n | R³ | R⁵ |
| 3-1 | H | 1 | H | H |
| 3-2 | H | 1 | H | Me |
| 3-3 | H | 1 | H | Et |
| 3-4 | H | 1 | H | Pr |
| 3-5 | H | 1 | H | iPr |
| 3-6 | H | 1 | H | Bn |
| 3-7 | H | 1 | H | Bzhy |
| 3-8 | H | 1 | H | PhCH═CHCH₂ |
| 3-9 | H | 1 | H | Ph |
| 3-10 | H | 1 | H | 5-BzDioxo |
| 3-11 | H | 1 | H | 6-BzDioxa |
| 3-12 | H | 1 | H | 4-F-Ph |
| 3-13 | H | 1 | H | 3,4-diF-Ph |
| 3-14 | H | 1 | H | 3-Cl-4-F-Ph |
| 3-15 | H | 1 | H | 2-Cl-Ph |
| 3-16 | H | 1 | H | 3-Cl-Ph |
| 3-17 | H | 1 | H | 4-Cl-Ph |
| 3-18 | H | 1 | H | 4-Br-Ph |
| 3-19 | H | 1 | H | 3-NO₂-Ph |
| 3-20 | H | 1 | H | 4-NO₂-Ph |
| 3-21 | H | 1 | H | 4-CN-Ph |
| 3-22 | H | 1 | H | 4-Ac-Ph |
| 3-23 | H | 1 | H | 4-EtCO-Ph |
| 3-24 | H | 1 | H | 4-(HOOC)-Ph |
| 3-25 | H | 1 | H | 4-(MeOOC)-Ph |
| 3-26 | H | 1 | H | 4-(EtOOC)-Ph |
| 3-27 | H | 1 | H | 4-Me₂N-Ph |
| 3-28 | H | 1 | H | 4-(H₂NCO)-Ph |
| 3-29 | H | 1 | H | 4-(MeNHCO)-Ph |
| 3-30 | H | 1 | H | 4-(Me₂NCO)-Ph |
| 3-31 | H | 1 | H | 4-(Et₂NCO)-Ph |
| 3-32 | H | 1 | H | 2-Me-Ph |
| 3-33 | H | 1 | H | 3-Me-Ph |
| 3-34 | H | 1 | H | 4-Me-Ph |
| 3-35 | H | 1 | H | 4-Et-Ph |
| 3-36 | H | 1 | H | 4-Pr-Ph |
| 3-37 | H | 1 | H | 4-iPr-Ph |
| 3-38 | H | 1 | H | 4-tBu-Ph |
| 3-39 | H | 1 | H | 3,4-diMe-Ph |
| 3-40 | H | 1 | H | 3-F-4-Me-Ph |
| 3-41 | H | 1 | H | 4-F-3-Me-Ph |
| 3-42 | H | 1 | H | 3-NO₂-4-Me-Ph |
| 3-43 | H | 1 | H | 3-CF₃-Ph |
| 3-44 | H | 1 | H | 4-CF₃-Ph |
| 3-45 | H | 1 | H | 2-MeO-Ph |
| 3-46 | H | 1 | H | 3-MeO-Ph |
| 3-47 | H | 1 | H | 4-MeO-Ph |
| 3-48 | H | 1 | H | 3,4-diMeO-Ph |
| 3-49 | H | 1 | H | 3,4,5-triMeO-Ph |
| 3-50 | H | 1 | H | 4-EtO-Ph |
| 3-51 | H | 1 | H | 4-PrO-Ph |
| 3-52 | H | 1 | H | 4-iPrO-Ph |
| 3-53 | H | 1 | H | 4-CF₃O-Ph |
| 3-54 | H | 1 | H | 4-MeS-Ph |
| 3-55 | H | 1 | H | 4-MeSO-Ph |
| 3-56 | H | 1 | H | 4-MeSO₂-Ph |
| 3-57 | H | 1 | H | 4-BnO-Ph |
| 3-58 | H | 1 | H | 2-Oxaz |
| 3-59 | H | 1 | H | 2-Thaz |
| 3-60 | H | 1 | H | 2-BzOxaz |
| 3-61 | H | 1 | H | 2-BzThaz |
| 3-62 | H | 1 | H | 2-Py |
| 3-63 | H | 1 | H | 3-Py |
| 3-64 | H | 1 | H | 4-Py |
| 3-65 | H | 1 | H | 5-Ac-2-Py |
| 3-66 | H | 1 | H | 5-Me-2-Py |
| 3-67 | H | 1 | H | 6-MeO-3-Py |
| 3-68 | H | 1 | H | 2,3,5,6-tetraF-4-Py |
| 3-69 | H | 1 | H | 2-Pym |
| 3-70 | H | 1 | H | 4-Qunz |
| 3-71 | H | 1 | H | 6-Cl-3-Pydz |
| 3-72 | H | 1 | H | Ac |
| 3-73 | H | 1 | H | Ph-CO |
| 3-74 | H | 1 | H | MeSO₂ |
| 3-75 | H | 1 | H | tBuO-CO |
| 3-76 | Me | 1 | H | Ph |
| 3-77 | H | 1 | Me | 3-Me-Ph |
| 3-78 | H | 2 | H | H |
| 3-79 | H | 2 | H | Me |
| 3-80 | H | 2 | H | Et |
| 3-81 | H | 2 | H | Pr |
| 3-82 | H | 2 | H | iPr |
| 3-83 | H | 2 | H | Bn |
| 3-84 | H | 2 | H | Bzhy |
| 3-85 | H | 2 | H | PhCH═CHCH₂ |
| 3-86 | H | 2 | H | Ph |
| 3-87 | H | 2 | H | 5-BzDioxo |
| 3-88 | H | 2 | H | 6-BzDioxa |
| 3-89 | H | 2 | H | 4-F-Ph |
| 3-90 | H | 2 | H | 3,4-diF-Ph |
| 3-91 | H | 2 | H | 3-Cl-4-F-Ph |
| 3-92 | H | 2 | H | 2-Cl-Ph |
| 3-93 | H | 2 | H | 3-Cl-Ph |
| 3-94 | H | 2 | H | 4-Cl-Ph |
| 3-95 | H | 2 | H | 4-Br-Ph |
| 3-96 | H | 2 | H | 3-NO₂-Ph |
| 3-97 | H | 2 | H | 4-NO₂-Ph |
| 3-98 | H | 2 | H | 4-CN-Ph |
| 3-99 | H | 2 | H | 4-Ac-Ph |
| 3-100 | H | 2 | H | 4-EtCO-Ph |
| 3-101 | H | 2 | H | 4-(HOOC)-Ph |
| 3-102 | H | 2 | H | 4-(MeOOC)-Ph |
| 3-103 | H | 2 | H | 4-(EtOOC)-Ph |
| 3-104 | H | 2 | H | 4-Me₂N-Ph |
| 3-105 | H | 2 | H | 4-(H₂NCO)-Ph |
| 3-106 | H | 2 | H | 4-(MeNHCO)-Ph |
| 3-107 | H | 2 | H | 4-(Me₂NCO)-Ph |
| 3-108 | H | 2 | H | 4-(Et₂NCO)-Ph |
| 3-109 | H | 2 | H | 2-Me-Ph |
| 3-110 | H | 2 | H | 3-Me-Ph |
| 3-111 | H | 2 | H | 4-Me-Ph |
| 3-112 | H | 2 | H | 4-Et-Ph |
| 3-113 | H | 2 | H | 4-Pr-Ph |
| 3-114 | H | 2 | H | 4-iPr-Ph |
| 3-115 | H | 2 | H | 4-tBu-Ph |
| 3-116 | H | 2 | H | 3,4-diMe-Ph |
| 3-117 | H | 2 | H | 3-F-4-Me-Ph |
| 3-118 | H | 2 | H | 4-F-3-Me-Ph |
| 3-119 | H | 2 | H | 3-NO₂-4-Me-Ph |
| 3-120 | H | 2 | H | 3-CF₃-Ph |
| 3-121 | H | 2 | H | 4-CF₃-Ph |
| 3-122 | H | 2 | H | 2-MeO-Ph |
| 3-123 | H | 2 | H | 3-MeO-Ph |
| 3-124 | H | 2 | H | 4-MeO-Ph |
| 3-125 | H | 2 | H | 3,4-diMeO-Ph |
| 3-126 | H | 2 | H | 3,4,5-triMeO-Ph |
| 3-127 | H | 2 | H | 4-EtO-Ph |
| 3-128 | H | 2 | H | 4-PrO-Ph |
| 3-129 | H | 2 | H | 4-iPrO-Ph |
| 3-130 | H | 2 | H | 4-CF₃O-Ph |
| 3-131 | H | 2 | H | 4-MeS-Ph |
| 3-132 | H | 2 | H | 4-MeSO-Ph |
| 3-133 | H | 2 | H | 4-MeSO₂-Ph |
| 3-134 | H | 2 | H | 4-BnO-Ph |
| 3-135 | H | 2 | H | 2-Oxaz |
| 3-136 | H | 2 | H | 2-Thaz |
| 3-137 | H | 2 | H | 2-BzOxaz |
| 3-138 | H | 2 | H | 2-BzThaz |
| 3-139 | H | 2 | H | 2-Py |
| 3-140 | H | 2 | H | 3-Py |
| 3-141 | H | 2 | H | 4-Py |
| 3-142 | H | 2 | H | 5-Ac-2-Py |
| 3-143 | H | 2 | H | 5-Me-2-Py |
| 3-144 | H | 2 | H | 6-MeO-3-Py |
| 3-145 | H | 2 | H | 2,3,5,6-tetraF-4-Py |
| 3-146 | H | 2 | H | 2-Pym |
| 3-147 | H | 2 | H | 4-Qunz |
| 3-148 | H | 2 | H | 6-Cl-3-Pydz |
| 3-149 | H | 2 | H | Ac |
| 3-150 | H | 2 | H | Ph-CO |
| 3-151 | H | 2 | H | MeSO₂ |
| 3-152 | H | 2 | H | tBuO-CO |
| 3-153 | Me | 2 | H | H |
| 3-154 | Me | 2 | H | Me |
| 3-155 | Me | 2 | H | Et |
| 3-156 | Me | 2 | H | Pr |
| 3-157 | Me | 2 | H | iPr |
| 3-158 | Me | 2 | H | Bn |
| 3-159 | Me | 2 | H | Bzhy |
| 3-160 | Me | 2 | H | PhCH═CHCH₂ |
| 3-161 | Me | 2 | H | Ph |
| 3-162 | Me | 2 | H | 5-BzDioxo |
| 3-163 | Me | 2 | H | 6-BzDioxa |
| 3-164 | Me | 2 | H | 4-F-Ph |
| 3-165 | Me | 2 | H | 3,4-diF-Ph |
| 3-166 | Me | 2 | H | 3-Cl-4-F-Ph |
| 3-167 | Me | 2 | H | 2-Cl-Ph |
| 3-168 | Me | 2 | H | 3-Cl-Ph |
| 3-169 | Me | 2 | H | 4-Cl-Ph |
| 3-170 | Me | 2 | H | 4-Br-Ph |
| 3-171 | Me | 2 | H | 3-NO₂-Ph |
| 3-172 | Me | 2 | H | 4-NO₂-Ph |
| 3-173 | Me | 2 | H | 4-CN-Ph |
| 3-174 | Me | 2 | H | 4-Ac-Ph |
| 3-175 | Me | 2 | H | 4-EtCO-Ph |
| 3-176 | Me | 2 | H | 4-(HOOC)-Ph |
| 3-177 | Me | 2 | H | 4-(MeOOC)-Ph |
| 3-178 | Me | 2 | H | 4-(EtOOC)-Ph |
| 3-179 | Me | 2 | H | 4-Me₂N-Ph |
| 3-180 | Me | 2 | H | 4-(H₂NCO)-Ph |
| 3-181 | Me | 2 | H | 4-(MeNHCO)-Ph |
| 3-182 | Me | 2 | H | 4-(Me₂NCO)-Ph |
| 3-183 | Me | 2 | H | 4-(Et₂NCO)-Ph |
| 3-184 | Me | 2 | H | 2-Me-Ph |
| 3-185 | Me | 2 | H | 3-Me-Ph |
| 3-186 | Me | 2 | H | 4-Me-Ph |
| 3-187 | Me | 2 | H | 4-Et-Ph |
| 3-188 | Me | 2 | H | 4-Pr-Ph |
| 3-189 | Me | 2 | H | 4-iPr-Ph |
| 3-190 | Me | 2 | H | 4-tBu-Ph |
| 3-191 | Me | 2 | H | 3,4-diMe-Ph |
| 3-192 | Me | 2 | H | 3-F-4-Me-Ph |
| 3-193 | Me | 2 | H | 4-F-3-Me-Ph |
| 3-194 | Me | 2 | H | 3-NO₂-4-Me-Ph |
| 3-195 | Me | 2 | H | 3-CF₃-Ph |
| 3-196 | Me | 2 | H | 4-CF₃-Ph |
| 3-197 | Me | 2 | H | 2-MeO-Ph |
| 3-198 | Me | 2 | H | 3-MeO-Ph |
| 3-199 | Me | 2 | H | 4-MeO-Ph |
| 3-200 | Me | 2 | H | 3,4-diMeO-Ph |
| 3-201 | Me | 2 | H | 3,4,5-triMeO-Ph |
| 3-202 | Me | 2 | H | 4-EtO-Ph |
| 3-203 | Me | 2 | H | 4-PrO-Ph |
| 3-204 | Me | 2 | H | 4-iPrO-Ph |
| 3-205 | Me | 2 | H | 4-CF₃O-Ph |
| 3-206 | Me | 2 | H | 4-MeS-Ph |
| 3-207 | Me | 2 | H | 4-MeSO-Ph |
| 3-208 | Me | 2 | H | 4-MeSO₂-Ph |
| 3-209 | Me | 2 | H | 4-BnO-Ph |
| 3-210 | Me | 2 | H | 2-Oxaz |
| 3-211 | Me | 2 | H | 2-Thaz |
| 3-212 | Me | 2 | H | 2-BzOxaz |
| 3-213 | Me | 2 | H | 2-BzThaz |
| 3-214 | Me | 2 | H | 2-Py |
| 3-215 | Me | 2 | H | 3-Py |
| 3-216 | Me | 2 | H | 4-Py |
| 3-217 | Me | 2 | H | 5-Ac-2-Py |
| 3-218 | Me | 2 | H | 5-Me-2-Py |
| 3-219 | Me | 2 | H | 6-MeO-3-Py |
| 3-220 | Me | 2 | H | 2,3,5,6-tetraF-4-Py |
| 3-221 | Me | 2 | H | 2-Pym |
| 3-222 | Me | 2 | H | 4-Qunz |
| 3-223 | Me | 2 | H | 6-Cl-3-Pydz |
| 3-224 | Me | 2 | H | Ac |
| 3-225 | Me | 2 | H | Ph-CO |
| 3-226 | Me | 2 | H | MeSO₂ |
| 3-227 | Me | 2 | H | tBuO-CO |
| 3-228 | H | 2 | Me | H |
| 3-229 | H | 2 | Me | Me |
| 3-230 | H | 2 | Me | Et |
| 3-231 | H | 2 | Me | Pr |
| 3-232 | H | 2 | Me | iPr |
| 3-233 | H | 2 | Me | Bn |
| 3-234 | H | 2 | Me | Bzhy |
| 3-235 | H | 2 | Me | PhCH═CHCH₂ |
| 3-236 | H | 2 | Me | Ph |
| 3-237 | H | 2 | Me | 5-BzDioxo |
| 3-238 | H | 2 | Me | 6-BzDioxa |
| 3-239 | H | 2 | Me | 4-F-Ph |
| 3-240 | H | 2 | Me | 3,4-diF-Ph |
| 3-241 | H | 2 | Me | 3-Cl-4-F-Ph |
| 3-242 | H | 2 | Me | 2-Cl-Ph |
| 3-243 | H | 2 | Me | 3-Cl-Ph |
| 3-244 | H | 2 | Me | 4-Cl-Ph |
| 3-245 | H | 2 | Me | 4-Br-Ph |
| 3-246 | H | 2 | Me | 3-NO₂-Ph |
| 3-247 | H | 2 | Me | 4-NO₂-Ph |
| 3-248 | H | 2 | Me | 4-CN-Ph |
| 3-249 | H | 2 | Me | 4-Ac-Ph |
| 3-250 | H | 2 | Me | 4-EtCO-Ph |
| 3-251 | H | 2 | Me | 4-(HOOC)-Ph |
| 3-252 | H | 2 | Me | 4-(MeOOC)-Ph |
| 3-253 | H | 2 | Me | 4-(EtOOC)-Ph |
| 3-254 | H | 2 | Me | 4-Me₂N-Ph |
| 3-255 | H | 2 | Me | 4-(H₂NCO)-Ph |
| 3-256 | H | 2 | Me | 4-(MeNHCO)-Ph |
| 3-257 | H | 2 | Me | 4-(Me₂NCO)-Ph |
| 3-258 | H | 2 | Me | 4-(Et₂NCO)-Ph |
| 3-259 | H | 2 | Me | 2-Me-Ph |
| 3-260 | H | 2 | Me | 3-Me-Ph |
| 3-261 | H | 2 | Me | 4-Me-Ph |
| 3-262 | H | 2 | Me | 4-Et-Ph |
| 3-263 | H | 2 | Me | 4-Pr-Ph |
| 3-264 | H | 2 | Me | 4-iPr-Ph |
| 3-265 | H | 2 | Me | 4-tBu-Ph |
| 3-266 | H | 2 | Me | 3,4-diMe-Ph |
| 3-267 | H | 2 | Me | 3-F-4-Me-Ph |
| 3-268 | H | 2 | Me | 4-F-3-Me-Ph |
| 3-269 | H | 2 | Me | 3-NO₂-4-Me-Ph |
| 3-270 | H | 2 | Me | 3-CF₃-Ph |
| 3-271 | H | 2 | Me | 4-CF₃-Ph |
| 3-272 | H | 2 | Me | 2-MeO-Ph |
| 3-273 | H | 2 | Me | 3-MeO-Ph |
| 3-274 | H | 2 | Me | 4-MeO-Ph |
| 3-275 | H | 2 | Me | 3,4-diMeO-Ph |
| 3-276 | H | 2 | Me | 3,4,5-triMeO-Ph |
| 3-277 | H | 2 | Me | 4-EtO-Ph |
| 3-278 | H | 2 | Me | 4-PrO-Ph |
| 3-279 | H | 2 | Me | 4-iPrO-Ph |
| 3-280 | H | 2 | Me | 4-CF₃O-Ph |
| 3-281 | H | 2 | Me | 4-MeS-Ph |
| 3-282 | H | 2 | Me | 4-MeSO-Ph |
| 3-283 | H | 2 | Me | 4-MeSO₂-Ph |
| 3-284 | H | 2 | Me | 4-BnO-Ph |
| 3-285 | H | 2 | Me | 2-Oxaz |
| 3-286 | H | 2 | Me | 2-Thaz |
| 3-287 | H | 2 | Me | 2-BzOxaz |
| 3-288 | H | 2 | Me | 2-BzThaz |
| 3-289 | H | 2 | Me | 2-Py |
| 3-290 | H | 2 | Me | 3-Py |
| 3-291 | H | 2 | Me | 4-Py |
| 3-292 | H | 2 | Me | 5-Ac-2-Py |
| 3-293 | H | 2 | Me | 5-Me-2-Py |
| 3-294 | H | 2 | Me | 6-MeO-3-Py |
| 3-295 | H | 2 | Me | 2,3,5,6-tetraF-4-Py |
| 3-296 | H | 2 | Me | 2-Pym |
| 3-297 | H | 2 | Me | 4-Qunz |
| 3-298 | H | 2 | Me | 6-Cl-3-Pydz |
| 3-299 | H | 2 | Me | Ac |
| 3-300 | H | 2 | Me | Ph-CO |
| 3-301 | H | 2 | Me | MeSO₂ |
| 3-302 | H | 2 | Me | tBuO-CO |
| 3-303 | Me | 2 | Me | H |
| 3-304 | Me | 2 | Me | Me |
| 3-305 | Me | 2 | Me | Et |
| 3-306 | Me | 2 | Me | Pr |
| 3-307 | Me | 2 | Me | iPr |
| 3-308 | Me | 2 | Me | Bn |
| 3-309 | Me | 2 | Me | Bzhy |
| 3-310 | Me | 2 | Me | PhCH═CHCH₂ |
| 3-311 | Me | 2 | Me | Ph |
| 3-312 | Me | 2 | Me | 5-BzDioxo |
| 3-313 | Me | 2 | Me | 6-BzDioxa |
| 3-314 | Me | 2 | Me | 4-F-Ph |
| 3-315 | Me | 2 | Me | 3,4-diF-Ph |
| 3-316 | Me | 2 | Me | 3-Cl-4-F-Ph |
| 3-317 | Me | 2 | Me | 2-Cl-Ph |
| 3-318 | Me | 2 | Me | 3-Cl-Ph |
| 3-319 | Me | 2 | Me | 4-Cl-Ph |
| 3-320 | Me | 2 | Me | 4-Br-Ph |
| 3-321 | Me | 2 | Me | 3-NO₂-Ph |
| 3-322 | Me | 2 | Me | 4-NO₂-Ph |
| 3-323 | Me | 2 | Me | 4-CN-Ph |
| 3-324 | Me | 2 | Me | 4-Ac-Ph |
| 3-325 | Me | 2 | Me | 4-EtCO-Ph |
| 3-326 | Me | 2 | Me | 4-(HOOC)-Ph |
| 3-327 | Me | 2 | Me | 4-(MeOOC)-Ph |
| 3-328 | Me | 2 | Me | 4-(EtOOC)-Ph |
| 3-329 | Me | 2 | Me | 4-Me₂N-Ph |
| 3-330 | Me | 2 | Me | 4-(H₂NCO)-Ph |
| 3-331 | Me | 2 | Me | 4-(MeNHCO)-Ph |
| 3-332 | Me | 2 | Me | 4-(Me₂NCO)-Ph |
| 3-333 | Me | 2 | Me | 4-(Et₂NCO)-Ph |
| 3-334 | Me | 2 | Me | 2-Me-Ph |
| 3-335 | Me | 2 | Me | 3-Me-Ph |
| 3-336 | Me | 2 | Me | 4-Me-Ph |
| 3-337 | Me | 2 | Me | 4-Et-Ph |
| 3-338 | Me | 2 | Me | 4-Pr-Ph |
| 3-339 | Me | 2 | Me | 4-iPr-Ph |
| 3-340 | Me | 2 | Me | 4-tBu-Ph |
| 3-341 | Me | 2 | Me | 3,4-diMe-Ph |
| 3-342 | Me | 2 | Me | 3-F-4-Me-Ph |
| 3-343 | Me | 2 | Me | 4-F-3-Me-Ph |
| 3-344 | Me | 2 | Me | 3-NO₂-4-Me-Ph |
| 3-345 | Me | 2 | Me | 3-CF₃-Ph |
| 3-346 | Me | 2 | Me | 4-CF₃-Ph |
| 3-347 | Me | 2 | Me | 2-MeO-Ph |
| 3-348 | Me | 2 | Me | 3-MeO-Ph |
| 3-349 | Me | 2 | Me | 4-MeO-Ph |
| 3-350 | Me | 2 | Me | 3,4-diMeO-Ph |
| 3-351 | Me | 2 | Me | 3,4,5-triMeO-Ph |
| 3-352 | Me | 2 | Me | 4-EtO-Ph |
| 3-353 | Me | 2 | Me | 4-PrO-Ph |
| 3-354 | Me | 2 | Me | 4-iPrO-Ph |
| 3-355 | Me | 2 | Me | 4-CF₃O-Ph |
| 3-356 | Me | 2 | Me | 4-MeS-Ph |
| 3-357 | Me | 2 | Me | 4-MeSO-Ph |
| 3-358 | Me | 2 | Me | 4-MeSO₂-Ph |
| 3-359 | Me | 2 | Me | 4-BnO-Ph |
| 3-360 | Me | 2 | Me | 2-Oxaz |
| 3-361 | Me | 2 | Me | 2-Thaz |
| 3-362 | Me | 2 | Me | 2-BzOxaz |
| 3-363 | Me | 2 | Me | 2-BzThaz |
| 3-364 | Me | 2 | Me | 2-Py |
| 3-365 | Me | 2 | Me | 3-Py |
| 3-366 | Me | 2 | Me | 4-Py |
| 3-367 | Me | 2 | Me | 5-Ac-2-Py |
| 3-368 | Me | 2 | Me | 5-Me-2-Py |
| 3-369 | Me | 2 | Me | 6-MeO-3-Py |
| 3-370 | Me | 2 | Me | 2,3,5,6-tetraF-4-Py |
| 3-371 | Me | 2 | Me | 2-Pym |
| 3-372 | Me | 2 | Me | 4-Qunz |
| 3-373 | Me | 2 | Me | 6-Cl-3-Pydz |
| 3-374 | Me | 2 | Me | Ac |
| 3-375 | Me | 2 | Me | Ph-CO |
| 3-376 | Me | 2 | Me | MeSO₂ |
| 3-377 | Me | 2 | Me | tBuO-CO |

The compounds which are preferred in the above Table 1 to Table 3 for exemplified compounds are the exemplified compounds designated as numbers 1-7, 1-8, 1-13, 1-15 to 1-18, 1-20, 1-21, 1-27 to 1-29, 1-32, 1-36, 1-2-9, 2-33 and 2-102, and 3-9, 3-34, 3-62, 3-76, 3-86 to 3-90, 3-93, 3-94, 3-96 to 3-99, 3-104, 3-110 to 3-112, 3-115 to 3-119, 3-123 to 3-129, 3-131, 3-132, 3-136 to 3-141, 3-143, 3-144, 3-148 and 3-161, and

more preferred compounds are the exemplified compounds designated as numbers 1-16, 1-17, 1-20, 1-21, 1-28 and 1-44, and 3-86, 3-88, 3-89, 3-97, 3-99, 3-104, 3-110 to 3-112, 3-116, 3-123 to 3-125, 3-127, 3-131, 3-132, 3-136, 3-138 to 3-140, 3-143, 3-144 and 3-161.

Of these, particularly preferred compounds are
4-[4-(4-acetylphenyl)-1,4-diazepan-1-yl]-3-aminothieno[2,3-b]pyridine-2-carboxamide (exemplary compound No. 3-99),
3-amino-4-{4-[4-(dimethylamino)phenyl]-1,4-diazepan-1-yl}thieno[2,3-b]pyridine -2-carboxamide (exemplary compound No. 3-104),
3-amino-4-{4-[4-(methylsulfinyl)phenyl]-1,4-diazepan-1-yl}thieno[2,3-b]pyridine -2-carboxamide (exemplary compound No. 3-132), and
3-amino-4-[4-(1,3-thiazol-2-yl)-1,4-diazepan-1-yl]thieno[2,3-b]pyridine-2-carboxamide (exemplary compound No. 3-136).

### EMBODIMENTS OF THE INVENTION

The compounds having a general formula (I) of the present invention can be produced by the processes mentioned below.

<Process A>

The compound wherein R¹ is a hydrogen atom; R² is a group R^{a}NH—, R^{a}(R^{b})N— or 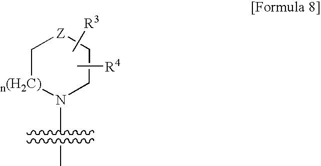
in general formula (I) can be produced according to Process A. 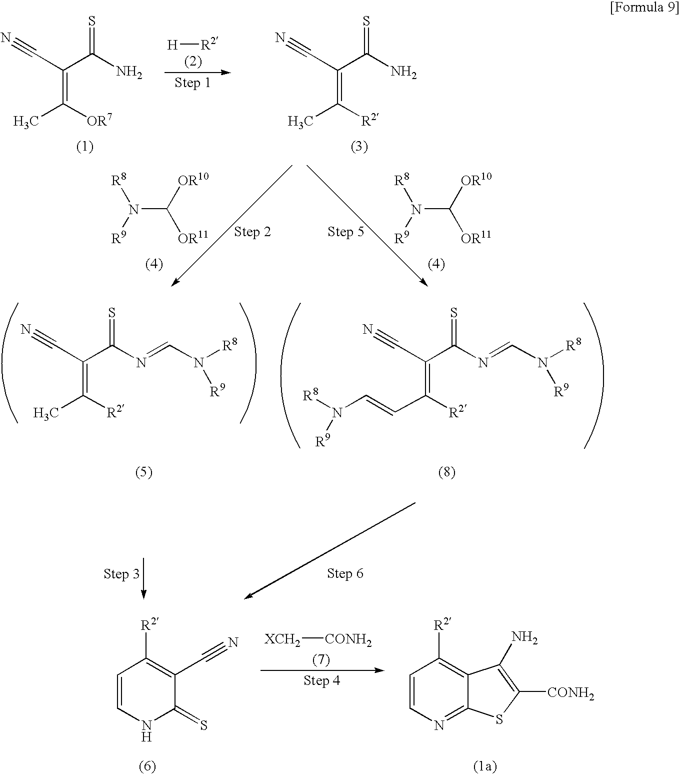
[wherein R^{2′}represents a group R^{a}NH—, R^{a}(R^{b})N— or 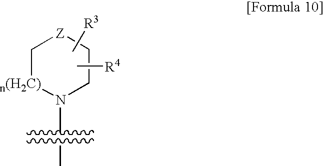
in the definition of R²,
R⁷ represents methyl or ethyl,
R⁸, R⁹, R¹⁰ and R¹¹ independently represent a C₁-C₆ alkyl group (preferably methyl, ethyl or isopropyl, particularly preferably methyl), and
X represents a halogen atom (preferably a chlorine atom or a bromine atom, particularly preferably a chlorine atom)].

Step 1 is a step for reacting compound (1) and amine compound (2) in an inert solvent to produce compound (3), and it can be carried out following a method described in J. Org. Chem, (1962) 27, 2433-2439.

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a halogenated hydrocarbon such as methylene chloride, chloroform, carbon tetrachloride, dichloroethane, chlorobenzene, o-dichlorobenzene, m-dichlorobenzene, fluorobenzene, trichloromethylbenzene or trifluoromethyl benzene, and preferably it is ethanol or N,N-dimethylformamide.

Reaction temperature varies depending on the raw material compounds or solvent used, but it is usually 0° C. to reflux temperature of the reaction mixture and preferably it is room temperature to reflux temperature of the reaction mixture.

Reaction time differs according to the raw material compounds, solvent or reaction temperature used, but it is usually from 30 minutes to 96 hours, preferably from 30 minutes to 24 hours.

The deposit obtained by filtering the reaction liquid or the residue obtained by evaporating the solvent after the reaction terminates can be used in the next step (Step 2) without being particularly purified. In addition, the reaction solution can be used as it is in the next step when an amide is used as inert solvent.

Step 2 is a step for reacting compound (3) and N,N-dialkylformrnamide dialkyl acetal (4) in an inert solvent to produce amidine derivative (5).

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a halogenated hydrocarbon such as methylene chloride, chloroform, carbon tetrachloride, dichloroethane, chlorobenzene, o-dichlorobenzene, m-dichlorobenzene, fluorobenzene, trichloromethylbenzene or trifluoromethyl benzene, and preferably it is ethanol or N,N-dimethylformamide.

The amount of N,N-dialkylformamide dialkyl acetal (4) used for the reaction is preferably 1 to 2 equivalent for one equivalent of compound (3).

Reaction temperature varies depending on the raw material compounds or solvent used, but it is usually 0° C. to reflux temperature of the reaction mixture, and preferably it is room temperature.

Reaction time differs according to the raw material compounds, solvent or reaction temperature used, but it is usually from 30 minutes to 96 hours, preferably from 30 minutes to 24 hours.

The deposit obtained by filtering the reaction liquid or the residue obtained by evaporating the solvent after the reaction terminates can be used in the next step (Step 3) without being particularly purified. In addition, the reaction solution can be used as it is in the next step when an amide is used as inert solvent.

Step 3 is a step for treating amidine derivative (5) in an inert solvent to produce thiopyridone derivatives (6).

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a halogenated hydrocarbon such as methylene chloride, chloroform, carbon tetrachloride, dichloroethane, chlorobenzene, o-dichlorobenzene, m-dichlorobenzene, fluorobenzene, trichloromethylbenzene or trifluoromethyl benzene, preferably it is ethanol or N,N-dimethylformamide, and particularly preferably it is N,N-dimethylformamide.

Reaction temperature varies depending on the raw material compounds or solvent used, but it is usually room temperature to reflux temperature of the reaction mixture and preferably it is 50° C. to 120° C.

Reaction time differs according to the raw material compounds, solvent or reaction temperature used, but it is usually from 10 minutes to six hours, preferably from 10 minutes to two hours.

After the reaction terminates, the object compound is collected from the reaction mixture according to a conventional method (extraction, column chromatography, filtration and concentration) as required. In addition, the reaction solution can be used as it is in the next step (Step 4) when an amide is used as inert solvent.

Step 4 is a step for reacting thiopyridone derivative (6) and a-haloacetamide (7) in the presence of a base in an inert solvent to produce thienopyridine derivatives (Ia).

The inert solvent to be used is, for example, an alcohol such as methanol, ethanol, propanol, 2-propanol or butanol; or an amide such as N,N-dimethylformamide, N,N-dimethylacetamide, N-methyl-2-pyrrolidone, N-methylpyrrolidinone or hexamethylphosphorotriamide, and preferably it is ethanol or N,N-dimethylformamide.

The base to be used is, for example, an alkali metal alkoxide such as sodium methoxide, sodium ethoxide, potassium tert-butoxide or lithium methoxide; or an alkali metal hydroxide such as sodium hydroxide, potassium hydroxide or lithium hydroxide, and preferably it is sodium ethoxide or sodium hydroxide.

Reaction temperature varies depending on the raw material compounds, solvent or base used, but it is usually 0° C. to reflux temperature of the reaction mixture and preferably it is room temperature to reflux temperature of the reaction mixture.

Reaction time differs according to the raw material compounds, solvent, base or reaction temperature used, but it is usually from 10 minutes to six hours, preferably from 30 minutes to two hours.

After the reaction terminates, the object compound is collected from the reaction mixture according to a conventional method as required.

For example, the object compound can be obtained by adding water to the reaction mixture and filtering the separated object compound; or after neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 5 is a step for reacting compound (3) and N,N-dialkylformamide dialkyl acetal (4) in an inert solvent to produce amidine derivative (8).

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a halogenated hydrocarbon such as methylene chloride, chloroform, carbon tetrachloride, dichloroethane, chlorobenzene, o-dichlorobenzene, m-dichlorobenzene, fluorobenzene, trichloromethylbenzene or trifluoromethyl benzene, preferably it is an aromatic hydrocarbon, and particularly preferably it is toluene.

The amount of N,N-dialkylformamide dialkyl acetal (4) used for the reaction is preferably 2 to 3 equivalents for one equivalent of compound (3).

Reaction temperature varies depending on the raw material compounds or solvent used, but it is usually 0° C. to reflux temperature of the reaction mixture and preferably it is room temperature to reflux temperature of the reaction mixture.

Reaction time differs according to the raw material compounds, solvent or reaction temperature used, but it is usually from three minutes to six hours, preferably from three minutes to two hours.

The residue obtained by evaporating the solvent under reduced pressure after the reaction terminates can be used in the next step (Step 6) without being particularly purified.

Step 6 is a step for treating amidine derivative (8) with an alkaline aqueous solution to produce thiopyridone derivatives (6).

The alkaline aqueous solution to be used is, for example, an aqueous solution of an alkali metal hydroxide (for example, sodium hydroxide, potassium hydroxide or lithium hydroxide) and preferably an aqueous solution of sodium hydroxide.

Reaction temperature varies depending on the raw material compounds or solvent used, but it is usually room temperature to reflux temperature of the reaction mixture and preferably it is reflux temperature of the reaction mixture.

Reaction time differs according to the raw material compounds, solvent or reaction temperature used, but it is usually from 10 minutes to two hours, preferably from 30 minutes to one hour.

After the reaction terminates, the object compound is collected from the reaction mixture according to a conventional method (extraction, column chromatography, filtration and concentration) as required.

<Process B>

The compound wherein R¹ is a C₁-C₆ alkyl group; R² is a group R^{a}NH—, R^{a}(R^{b})N— or 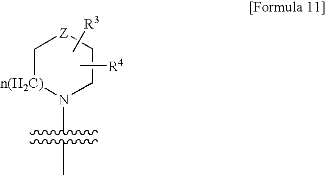
in general formula (I) can be produced according to Process B. 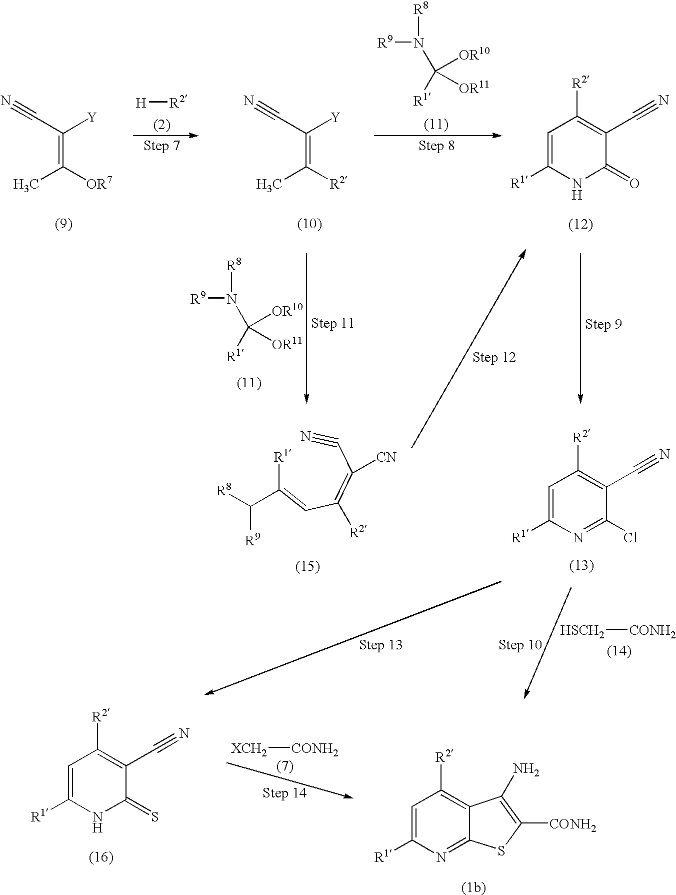
(wherein R^{2′}, R⁸, R⁹, R¹⁰, R¹¹ and X represent the same as defined above, and R^{1′} represents a C₁-C₆ alkyl group in the definition of R¹, and Y represents CONH₂ or CN.)

Step 7 is a step for reacting compound (9) and amine compound (2) in an inert solvent to produce compound (10), and it can be carried out following a similar method as described in Step 1.

Step 8 is a step for reacting a compound (10) in which Y is CONH₂ and (N,N-dialkyl)alkylamide dialkyl acetal (11) in an inert solvent to produce pyridone derivative (12), and it can be carried out following a method described in Pharm. Chem. J. (Engl. Transl.) 25, (1991), 623-628.

The inert solvent to be used is, for example, an aromatic hydrocarbon such as benzene, toluene or xylene; or an amide such as N,N-dimethylformamide, N,N-dimethylacetamide, N-methyl-2-pyrrolidone, N-methylpyrrolidinone or hexamethylphosphorotriamide, and preferably it is an amide and particularly preferably it is N,N-dimethylformamide.

Reaction temperature varies depending on the raw material compounds or solvent used, but it is usually room temperature to reflux temperature of the reaction mixture and preferably it is 50° C. to reflux temperature of the reaction mixture.

Reaction time differs according to the raw material compounds, solvent or reaction temperature used, but it is usually from one hour to 24 hours, preferably from one hour to five hours.

After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 9 is a step for halogenating pyridone derivative (12) in the presence of a base with a halogenating agent to produce chloropyridine derivative (13).

When the reaction is carried out in an inert solvent, for example, an aromatic hydrocarbon such as benzene, toluene or xylene; or an ether such as diethyl ether, diisopropyl ether, tetrahydrofuran, dioxane or 1,2-dimethoxyethane, and preferably toluene or dioxane, is used as solvent.

The base to be used can be, for example, an organic amine such as triethylamine, tributylamine, diisopropylethylamine, N-methylmorpholine, pyridine, 4-(N,N-dimethylamino)pyridine, N,N-dimethylaniline, N,N-diethylaniline, 1,5-diazabicyclo[4.3.0]non-5-ene, 1,4-diazabicyclo[2.2.2]octane (DABCO) or 1,8-diazabicyclo[5.4.0]-7-undecene (DBU), and particularly preferably N,N-dimethylaniline.

The halogenating agent to be used can be, for example, a phosphorus chloride such as phosphorus trichloride, phosphorous pentachloride or phosphorus oxychloride; or thionyl chloride, and preferably it is phosphorous pentachloride, phosphorus oxychloride or thionyl chloride.

Reaction temperature varies depending on the raw material compounds, solvent, base or halogenating agent, but it is usually room temperature to reflux temperature of the reaction mixture, and preferably it is 50° C. to reflux temperature of the reaction mixture.

Reaction time differs according to the raw material compounds, solvent, base, halogenating agent or reaction temperature, but it is usually from one hour to 24 hours, preferably from one hour to eight hours.

After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 10 is a step for reacting chloropyridine derivative (13) and 2-mercaptoacetamide (14) in the presence of a base in an inert solvent to produce thienopyridine derivative (Ib).

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; or an amide such as N,N-dimethylformamide, N,N-dimethylacetamide, N-methyl-2-pyrrolidone, N-methylpyrrolidinone or hexamethylphosphorotriamide, preferably it is an alcohol or an amide and more preferably it is ethanol or N,N-dimethylformamide.

The base to be used is, for example, an alkali metal alkoxide such as sodium methoxide, sodium ethoxide, potassium tert-butoxide or lithium methoxide; or an alkali metal hydroxide such as sodium hydroxide, potassium hydroxide or lithium hydroxide, and preferably it is sodium ethoxide or sodium hydroxide.

Reaction temperature varies depending on the raw material compounds, solvent or base used, but it is usually room temperature to reflux temperature of the reaction mixture.

Reaction time differs according to the raw material compounds, solvent, base or reaction temperature used, but it is usually from one hour to 24 hours, preferably from one hour to two hours.

After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 11 is a step for reacting a compound (10) in which Y is CN and (N,N-dialkyl)alkylamide dialkyl acetal (11) in an inert solvent to produce enamine derivative (15).

The inert solvent to be used is, for example, an aromatic hydrocarbon such as benzene, toluene or xylene; or an amide such as N,N-dimethylformamide, N,N-dimethylacetamide, N-methyl-2-pyrrolidone, N-methylpyrrolidinone or hexamethylphosphorotriamide, preferably it is an aromatic hydrocarbon, and particularly preferably it is xylene.

Reaction temperature varies depending on the raw material compounds or solvent used, but it is usually room temperature to reflux temperature of the reaction mixture and preferably it is 100° C. to reflux temperature of the reaction mixture.

Reaction time differs according to the raw material compounds, solvent or reaction temperature used, but it is usually from one hour to 24 hours, preferably from one hour to eight hours.

After the reaction terminates, the object compound is collected according to a conventional method as required from the reaction mixture.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 12 is a step for treating enamine derivative (15) with an acid to produce pyridone derivative (12).

The acid to be used can be, for example, an organic acid such as formic acid, acetic acid, trifluoroacetic acid or polyphosphoric acid, and preferably it is acetic acid or polyphosphoric acid.

When the reaction is carried out in an inert solvent, the solvent can be, for example, an aromatic hydrocarbon such as benzene, toluene or xylene; an amide such as N,N-dimethylformamide, N,N-dimethylacetamide, N-methyl-2-pyrrolidone, N-methylpyrrolidinone or hexamethylphosphorotriamide; an alcohol such as methanol or ethanol; or water or a mixed solvent of water and a solvent mentioned above, and particularly preferably it is water.

Reaction temperature varies depending on the raw material compounds or acid used, but it is usually room temperature to reflux temperature of the reaction mixture and preferably it is 50° C. to reflux temperature of the reaction mixture.

Reaction time differs according to the raw material compounds, acid or reaction temperature used, but it is usually from one hour to 24 hours, preferably from one hour to eight hours.

Step 13 is a step for reacting chloropyridine derivative (13) and thiourea or sodium sulfide (preferably thiourea) in an inert solvent to produce thiopyridone derivative (16).

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; or a mixture of solvents mentioned above, preferably it is an alcohol, an aromatic hydrocarbon or a mixture of an alcohol and aromatic hydrocarbon, and more preferably it is ethanol, toluene or a mixture of ethanol and toluene.

Reaction temperature varies depending on the raw material compounds or solvent used, but it is usually room temperature to reflux temperature of the reaction mixture and preferably it is 50° C. to reflux temperature of the reaction mixture.

Reaction time differs according to the raw material compounds, solvent or reaction temperature used, but it is usually from one hour to 48 hours, preferably from one hour to 24 hours.

After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

Step 14 is a step for reacting thiopyridone derivative (16) and α-haloacetamide (7) in the presence of a base in an inert solvent to produce thienopyridine derivatives (Ib) and can be carried out by a similar method as described in Step 4.

<Process C>

The compound wherein R² is R^{a}O— in general formula (I) can be produced according to Process C. 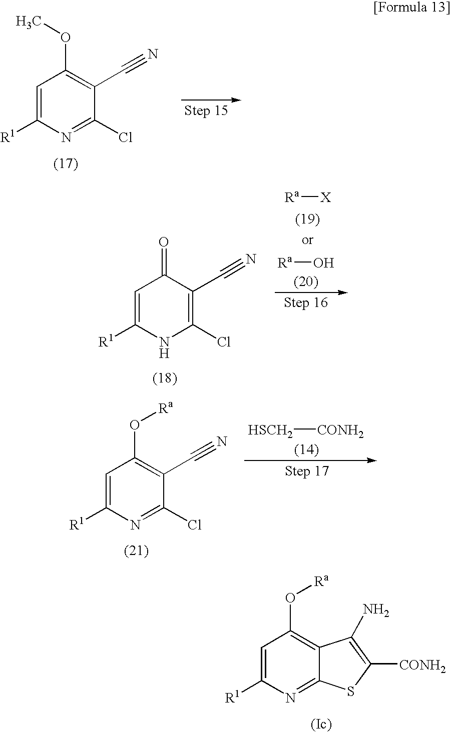
(wherein R¹, R^{a} and X represent the same as defined above.)

Step 15 is a step for demethylating methoxypyridine derivative (17) to produce compound (18) and, for example, it can be carried out by heating a methoxypyridine derivative (17) and concentrated hydrochloric acid under reflux in acetic acid solvent.

Step 16 is a step for (a) reacting compound (18) and halogen compound (19) in the presence of a base in an inert solvent or (b) performing a Mitsunobu reaction using compound (18) and alcohol derivative (20), to produce 4-alkoxypyridine derivative (21).

(a) A Method Using Halogen Compound (19) (Etherification Reaction)

The inert solvent to be used is, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a sulfoxide such as dimethylsulfoxide or sulfolane, preferably it is an ether or an amide, and particularly preferably it is tetrahydrofuran or N,N-dimethylacetamide.

The base to be used can be, for example, an alkali metal carbonate such as sodium carbonate or potassium carbonate; an alkali metal hydride such as lithium hydride, sodium hydride or potassium hydride; an alkali metal alkoxide such as sodium methoxide, sodium ethoxide, potassium tert-butoxide or lithium methoxide; or an alkali metal hydroxide such as sodium hydroxide, potassium hydroxide or lithium hydroxide, preferably it is an alkali metal carbonate or alkali metal hydride, and more preferably it is potassium carbonate or sodium hydride.

Reaction temperature varies depending on the raw material compounds, solvent or base used, but it is usually 0° C. to reflux temperature of the reaction mixture and preferably it is 0° C. to room temperature.

Reaction time differs according to the raw material compounds, solvent, base or reaction temperature used, but it is usually from one hour to 48 hours, preferably from one hour to 24 hours.

(b) A Method Using Alcohol Derivative (20) (Mitsunobu Reaction)

The reaction is usually performed in an inert solvent, and the inert solvent used can be, for example, an aromatic hydrocarbon such as benzene, toluene or xylene; or an ether such as diethylether, diisopropylether, tetrahydrofuran, dioxane or 1,2-dimethoxyethane, and preferably it is toluene or tetrahydrofuran.

The reagent used for the Mitsunobu reaction is a combination of, for example, an azo compound such as a di(C₁-C₆ alkyl) azodicarboxylate such as diethyl azodicarboxylate or diisopropyl azodicarboxylate or an azodicarbonyl such as 1,1′-(azodicarbonyl)dipiperidine, and a phosphine such as a tri(C₆-C₁₀ aryl)phosphine such as triphenylphosphine or a tri(C₁-C₆ alkyl)phosphine such as tri-n-butylphosphine, more preferably it is a combination of di(C₁-C₆ alkyl) azodicarboxylate and tri(C₆-C₁₀ aryl)phosphine, and most preferably it is a combination of diethyl azodicarboxylate and triphenylphosphine.

Reaction temperature varies depending on the raw material compounds, solvent or chemical reagent used, but it is usually 0° C. to reflux temperature of the solvent and preferably it is 0° C. to room temperature.

Reaction time differs according to the raw material compounds, solvent, reagent or reaction temperature used, but it is usually from one hour to 48 hours, preferably from one hour to 24 hours. After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

It is length of time.

Step 17 is a step for reacting 4-alkoxypyridine derivative (21) and 2-mercaptoacetamide (14) in the presence of a base in an inert solvent to produce thienopyridine derivative (Ic) and it can be carried out by a similar method as described in Step 10.

<Process D>

The compound wherein R² is R^{a}S— in general formula (I) can be produced according to Process D. 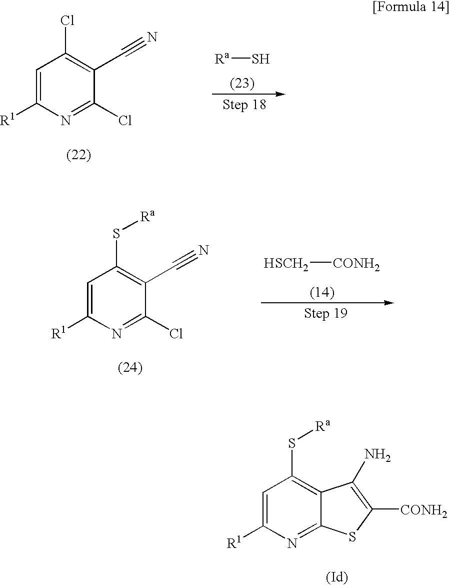
(wherein R¹, R^{a} and X represent the same as defined above.)

Step 18 is a step for reacting dichloropyridine compound (22) and thiol compound (23) or an alkali metal salt thereof (for example, sodium salt) in the presence or absence of a base in an inert solvent to produce 4-alkylthio pyridine derivative (24).

The inert solvent to be used can be, for example, an alcohol such as methanol, ethanol, propanol, 2-propanol or butanol; an aromatic hydrocarbon such as benzene, toluene or xylene; an ether such as diethyl ether, diisopropyl ether, tetrahydrofuran, dioxane or 1,2-dimethoxyethane; an amide such as N,N-dimethylformamide, N,N-dimethylacetamide, N-methyl-2-pyrrolidone, N-methylpyrrolidinone or hexamethylphosphorotriamide; or a sulfoxide such as dimethylsulfoxide or sulfolane, preferably it is an ether or an amide, and particularly preferably it is tetrahydrofuran or N,N-dimethylacetamide.

The base to be used can be, for example, an alkali metal hydride such as lithium hydride, sodium hydride or potassium hydride; an alkali metal alkoxide such as sodium methoxide, sodium ethoxide, potassium tert-butoxide or lithium methoxide; or an alkali metal hydroxide such as sodium hydroxide, potassium hydroxide or lithium hydroxide, and preferably it is sodium hydride or sodium hydroxide.

Reaction temperature varies depending on the raw material compounds, solvent or base used, but it is usually 0° C. to reflux temperature of the reaction mixture and preferably it is 0° C. to room temperature.

Reaction time differs according to the raw material compounds, solvent, base or reaction temperature used, but it is usually from one hour to 24 hours, preferably from one hour to six hours.

After the reaction terminates, the object compound is collected from the reaction mixture according to a conventional method as required.

For example, the object compound can be obtained by neutralizing the reaction mixture appropriately and removing by filtration insolubles if present, adding water, extracting with a water-immiscible organic solvent such as ethyl acetate or toluene and washing with water and the like, and evaporating the solvent after drying over anhydrous magnesium sulfate and the like.

If necessary, the obtained compound can be separated and purified by a conventional method, for example by silica gel column chromatography.

In addition, 4-alkoxypyridine derivatives corresponding to compound (24) can be produced by substituting an alcohol derivative (20) for thiol compound (23).

Step 19 is a step for reacting 4-alkylthiopyridine derivative (24) and 2-mercaptoacetamide (14) in the presence of a base in an inert solvent to produce thienopyridine derivative (Id), and it is carried out by a method similar to the method described in Step 10.

Since the compounds having a general formula (I) of the present invention or pharmacologically acceptable salts thereof have effects of promoting osteogenesis, suppressing bone resorption and/or improving bone density, they are useful as a pharmaceutical composition {particularly a pharmaceutical composition for prevention or treatment of osteopathy [for example, osteoporosis (for example, postmenopausal osteoporosis, senile osteoporosis or secondary osteoporosis caused by use of steroid or immunosuppressant), osteopenia or bone destruction associated with rheumatoid arthritis, Paget's disease of bone, bone fracture or dysostosis due to dwarfism] or osteoarthritis}. The mode for administration can be, for example, oral administration by tablets, capsules, granules, powders or syrup, or parenteral administration by injection or suppository, etc., and preparations for those purposes can be produced by well-known methods using additives such as excipients, lubricants, binders, disintegrants, stabilizing agents, corrigents, diluents.

Excipients can be, for example, organic excipients such as a sugar derivative such as lactose, sucrose, glucose, mannitol or sorbitol, a starch derivative such as corn starch, potato starch, α-starch, dextrin or carboxymethyl starch, a cellulose derivative such as crystalline cellulose, low substituted hydroxypropyl cellulose, hydroxypropyl methyl cellulose, carboxymethyl cellulose, calcium carboxymethyl cellulose or intemally crosslinked sodium carboxymethyl cellulose, gum arabic, dextran or pullulan; or inorganic excipients such as a silicate derivative such as light anhydrous silicic acid, synthetic aluminium silicate or magnesium aluminometasilicate, a phosphate such as calcium phosphate, a carbonate such as calcium carbonate, or a sulphate such as calcium sulphate.

Lubricants can be, for example, stearic acid or a metal stearate such as calcium stearate or magnesium stearate; talc; colloidal silica; wax such as bee gum or spermaceti; boric acid; adipic acid; sulfate such as sodium sulfate; glycol; fumaric acid; sodium benzoate; DL-leucine; sodium salt of fatty acid; a lauryl sulfate such as sodium lauryl sulfate or magnesium lauryl sulfate; silicic acid such as silicic anhydride or silicic acid hydrate; or a starch derivative as mentioned above.

Binders can be, for example, polyvinylpyrrolidone or macrogol or a compound which is similar to the above excipients.

Disintegrants can be, for example, a compound which is similar to the above excipients or a chemically modified starch and/or cellulose such as cross sodium carmellose, sodium carboxymethyl starch or crosslinked polyvinylpyrrolidone.

Stabilizing agents can be, for example, a paraoxybenzoic acid ester such as methylparaben or propylparaben; an alcohol such as chlorobutanol, benzyl alcohol or phenylethyl alcohol; benzalkonium chloride; a phenol such as phenol or cresol; thimerosal; dehydroacetic acid; or sorbic acid.

Corrigents can be, for example, a sweetener, acidulant or flavouring agent usually used.

The amount of the compound having a general formula (I) of the present invention or its pharmacologically acceptable salt varies depending on the symptoms, age, administration method, but it is desirable to administer, for example, 0.1 mg (preferably, 1 mg) for the lower limit and 1000 mg (preferably, 100 mg) for the upper limit, in a single dose or divided into multiple doses per day for an adult, depending on the symptoms, in the case of oral administration. In the case of intravenous administration, it is desirable to administrate 0.01 mg (preferably, 0.1 mg) for the lower limit and 100 mg (preferably, 10 mg) for the upper limit, in a single dose or divided into multiple doses, per day for an adult depending on the symptoms.

[Advantages of the Invention]

Since the compound having a general formula (I) of the present invention or pharmacologically acceptable salts thereof have effects of promoting osteogenesis, suppressing bone resorption and/or improving bone density, they are useful as a pharmaceutical composition {particularly a pharmaceutical composition for prevention or treatment of osteopathy [for example, osteoporosis (for example, postmenopausal osteoporosis, senile osteoporosis or secondary osteoporosis caused by the use of steroids or immunosuppressants), osteopenia or bone destruction associated with rheumatoid arthritis, Paget's disease of bone, bone fracture or dysostosis due to dwarfism] or osteoarthritis}.

[Best Mode for Carrying Out the Invention]

The present invention will be described in more detail by way of the Examples, Preparation Examples and Test Examples below but the present invention is not limited to these.

### EXAMPLES

### Example 1

### 3-amino-4-(dimethylamino)thieno[2,3-b]pyridine-2-carboxamide

(Exemplified Compound No. 2-17)

The compound was produced by the following method with reference to a method described in Pharm. Chem. J. (Engl. Transl.), 26, (1992), 870-874.

### (1a) (2Z)-2-cyano-3-(dimethylamino)but-2-enethioamide

Cyanothioacetamide (1.00 g, 10 mmol) and N,N-dimethylacetamide dimethylacetal (1.73 g, 13 mmol) were dissolved in acetonitrile (5 mL) and the mixture was stirred at room temperature for one hour. The deposited crystal was filtered and the crystal was further washed with acetonitrile and 1.05 g (yield 62%) of the title compound was obtained.

Mp 155-158° C.;

¹H NMR (DMSO-d₆, 400MHz) δ 2.27 (3H, s), 3.03 (6H, s), 8.08 (1H, br), 8.83 (1H, br).

### (1b) 4-(dimethylamino)-2-thioxo-1,2-dihydropyridine-3-carbonitrile

(2Z)-2-cyano-3-(dimethylamino)but-2-enethioamide (1.05 g, 6.2 mmol) produced in Example 1 (1a) and N,N-dimethylformamide dimethylacetal (2.22 g, 18.6 mmol) were dissolved in toluene (10 mL) and the mixture was stirred under heat reflux for two hours. The residue obtained by concentrating the mixture under reduced pressure was suspended in 1N aqueous solution of sodium hydroxide (10 mL) and heated under reflux for 30 minutes. After the reaction mixture was cooled to room temperature, 1N hydrochloric acid (15 mL) was added. The deposited solid was filtered and washed with water and ethanol and 0.87 g (yield 78%) of the title compound was obtained.

Mp 246-250° C.;

¹H NMR (DMSO-d₆, 400 MHz)δ3.12 (6H, s), 6.25 (1H, d, J=7.3 Hz), 7.29-7.33 (1H, m), 12.40 (1H, br).

### (1c) 3-amino-4-(dimethylamino)thieno[2,3-b]pyridine-2-carboxamide

8N aqueous solution of sodium hydroxide (2 mL) and 2-chloroacetamide (0.54 g, 5.8 mmol) were added to an N,N-dimethylformamide (10 mL) solution of 4-(dimethylamino)-2-thioxo-1,2-dihydropyridine-3-carbonitrile (0.87 g) produced in Example 1 (1b), and the mixture was stirred at room temperature for one hour. Water (100 mL) was added to the reaction mixture, and the deposited crystal was filtered and further washed with water and ethanol and 0.79 g (yield 54%) of the title compound was obtained.

Mp 208-211° C.;

IR (KBr) νₘₐₓ 3430, 3296, 3132, 1673, 1582, 1372, 979 cm⁻¹;

¹H NMR (DMSO-d₆, 400 MHz) δ 2.80 (6H, s), 6.96 (1H, d, J=5.5 Hz), 6.97 (2H, br), 7.04 (2H, br), 8.36 (1H, d, J=5.5 Hz);

MS (FAB) m/z: 237 [M+H]⁺;

Anal. Calcd. for C₁₀H₁₂N₄SO: C, 50.83; H, 5.12; N, 23.71; S, 13.57. Found: C, 50.70; H, 4.98; N, 23.53; S, 13.38.

### Example 2

### 3-amino-4-(diethylamino)thieno[2,3-b]pyridine-2-carboxamide

(Exemplified Compound No. 2-33)

### (2a) (2Z)-2-cyano-3-(diethylamino)but-2-enethioamide

(2Z)-2-cyano-3-ethoxybut-2-enethioamide (J. Org. Chem., (1962), 27,2433-2439) (406 mg, 2.38 mmol) and diethylamine (0.36 mL, 3.53 mmol) were suspended in ethanol (5 mL) and the mixture was stirred at room temperature for two hours. After the solvent was evaporated, the obtained residue was purified by silica gel column chromatography (ethyl acetate/hexane=2:1) and the title compound was obtained (237 mg, yield 50%).

¹H NMR(CDCl₃, 400 MHz) δ 1.32 (6H, t, J=7.04 Hz), 2.71 (3H, s), 3.65 (4H, q, J=7.05 Hz), 6.69 (2H, br s).

### (2b) 4-(diethylamino)-2-thioxo-1,2-dihydropyridine-3-carbonitrile

(2Z)-2-cyano-3-(diethylamino)but-2-enethioamide produced in Example 2 (2a) was used in place of (2Z)-2-cyano-3-(dimethylamino)but-2-enethioamide and reacted in a similar method as described in Example 1 (1b) and the title compound was obtained. Yield 54%.

¹H NMR(DMSO-d₆, 400 MHz) δ 1.19 (6H, t, J=6.8 Hz), 3.21 (1H, s), 3.615 (4H, q, J=6.8 Hz), 6.34 (1H, d, J=7.8 Hz), 7.36 (1H, t, J=7.8 Hz).

### (2c) 3-amino-4-(diethylamino)thieno[2,3-b]pyridine-2-carboxamide

4-(diethylamino)-2-thioxo-1,2-dihydropyridine-3-carbonitrile produced in Example 2 (2b) was used in place of 4-(dimethylamino)-2-thioxo-1,2-dihydropyridine-3-carbonitrile and reacted in a similar method as described in Example 1 (1c) and the title compound was obtained. Yield 65%.

Mp 127-129° C.;

IR (KBr) νₘₐₓ 3426, 3304, 3143, 2974, 1672, 1647, 1581, 1504, 1376, 1345, 1262, 1158, 1050, 790, 616 cm⁻¹;

¹H NMR(DMSO-d₆, 400 MHz) δ 0.98 (6H, t, J=7.0 Hz), 3.17 (4H, q, J=7.0 Hz), 7.06 (1H, d, J=5.1 Hz), 7.09 (2H, s), 7.36 (1H, d, J=5.1 Hz);

MS (FAB) m/z: 264.10 [M+H]⁺;

Anal. Calcd. for C₁₂H₁₆N₄OS: C, 54.52; H, 6.10; N, 21.19; S, 12.13. Found: C, 54.18; H, 5.86; N, 21.34; S, 12.18.

### Example 3

### 3-amino-4-(dimethylamino)-6-methylthieno[2,3-b]pyridine-2-carboxamide

(Exemplified Compound No. 2-102)

### (3a) 4-(dimethylamino) -6-methyl-2-thioxo-1,2-dihydropyridine-3-carbonitrile

2-chloro-4-(dimethylamino)-6-methylnicotinonitrile (Pharm. Chem. J., (Engl. Transl.), 25, (1991), 623-628.) (1.46 g, 7.5 mmol) and thiourea (0.74 g, 9.7 mmol) were suspended in toluene (25 mL) and the mixture was stirred under heat reflux for four hours. Ethanol (40 mL) was added to the reaction mixture and further heated under reflux for 30 minutes. The solid which deposited after allowing to stand overnight at room temperature was filtered and washed with ethanol, water, ethanol sequentially and the title compound was obtained as a crude product (0.64 g).

¹H NMR (DMSO-d₆, 400 MHz) δ 2.20 (3H, s), 3.18 (6H, s), 6.23 (1H, s), 12.41 (1H, br).

### (3b) 3-amino-4-(dimethylamino)-6-methylthieno[2,3-b]pyridine-2-carboxamide

4-(dimethylamino)-6-methyl-2-thioxo-1,2-dihydropyridine-3-carbonitrile (0.19 g, 1.0 mmol) produced in Example 3 (3a) was dissolved in N,N-dimethylformamide (3 mL) and 8N aqueous solution of sodium hydroxide (0.5 mL) and 2-chloroacetamide (0.11 g, 1.2 mmol) were added. After the mixture was stirred at room temperature for one hour, water (50 mL) was added. The aqueous layer was extracted with ethyl acetate (3×50 mL) and the extract was concentrated after drying over sodium sulfate under reduced pressure. The residue was purified by silica gel column chromatography (100% ethyl acetate) and 0.16 g of the title compound (yield 62%) was obtained.

Mp 167-170° C.;

IR (KBr) νₘₐₓ 3442, 3327, 3170, 1647, 1580, 1368, 992 cm⁻¹;

¹H NMR (DMSO-d₆, 400 MHz) δ 2.48 (3H, s), 2.78 (6H, s), 6.84 (1H, s), 6.93 (2H, br), 6.95 (2H, br);

MS (EI) m/z: 250 [M⁺], 218, 205, 190;

Anal. Calcd. for C₁₁H₁₄N₄SO.0.5 H₂O: C, 50.95; H, 5.83; N, 21.60; S, 12.36. Found: C, 50.84; H, 5.94; N, 21.51; S, 12.19.

### Example 4

### 4-(isobutylamino)-2-thioxo-1,2-dihydropyridine-3-carbonitrile

(Exemplified Compound No. 3-3)

### (4a) (2Z)-2-cyano-3-(propylamino)but-2-enethioamide

(2Z)-2-cyano-3-ethoxybut-2-enethioamide (J. Org. Chem., (1962), 27,2433-2439) (0.34 g, 2.0 mmol) and propylamine (0.15 g, 2.5 mmol) were suspended in ethanol (5 mL) and the mixture was stirred at room temperature for 15 hours. The deposited solid was separated by filtration and further washed with ethanol and 0.29 g of the title compound (yield 79%) was obtained.

Mp 149-151° C.;

IR (KBr) νₘₐₓ 3400, 3287, 3187, 2190, 1612 cm⁻¹;

¹H NMR (DMSO-d₆, 400 MHz) δ 0.97 (3H, t, J=7.4 Hz), 1.55-1.64 (2H, m), 2.30 (3H, s), 3.35-3.40 (2H, m), 7.65 (1H, br), 8.45 (1H, br), 12.74 (1H, br);

MS (FAB) m/z: 184 [M+H]⁺;

Anal. Calcd. for C₈H₁₃N₃S: C, 52.43; H, 7.15; N, 22.93; S, 17.49. Found: C, 52.59; H, 7.25; N, 22.83; S, 17.51.

### (4b) 3-amino-4-(propylamino)thieno[2,3-b]pyridine-2-carboxamide

(2Z)-2-cyano-3-(propylamino)but-2-enethioamide (0.29 g, 1.6 mmol) which was produced in Example 4 (4a) and N,N-dimethylformamide dimethylacetal (0.57 g, 4.7 mmol) were mixed with toluene (3 mL) and the mixture was stirred under heat reflux for two hours. 1N aqueous solution of sodium hydroxide (5 mL) was added to the residue which was obtained by evaporating the solvent under reduced pressure and the mixture was stirred under heat reflux for 30 minutes. After the reaction mixture was cooled to room temperature, ether (20 mL) was added and the liquid was partitioned. Furthermore, the organic layer was extracted with 1N aqueous solution of sodium hydroxide (5 mL). The combined aqueous layer was neutralized with 1N hydrochloric acid, and the deposited solid was separated by filtration and further washed with water and a little amount of ethanol and a solid (0.17 g) mainly containing 2-thioxo-1,2-dihydropyridine derivative was obtained.

The obtained solid was dissolved in N,N-dimethylformamide (3 mL) and 8N aqueous solution of sodium hydroxide (0.5 mL) and 2-chloroacetamide (0.10 g, 1.1 mmol) were added. After the mixture was stirred at room temperature for one hour, water (3 mL) was added. The deposited solid was separated by filtration and washed with water and ethanol and 106 mg of the title compound was obtained. Yield 27% from (2Z)-2-cyano-3-(propylamino)but-2-enethioamide.

Mp 214-215° C.;

IR (KBr) νₘₐₓ 3348, 3319, 3189, 1650, 1592, 1504, 1364 cm⁻¹;

¹H NMR (DMSO-d₆, 400 MHz) δ 0.95 (3H, t, J=7.4 Hz), 1.59-1.68 (2H, m), 3.16-3.21 (2H, m), 6.41 (1H, d, J=5.6 Hz), 6.44 (1H, brt, J=5.4 Hz), 6.81 (2H, br), 7.02 (2H, br), 8.05 (1H, d, J=5.6 Hz);

MS (EI) m/z: 250 [M⁺], 204;

Anal. Calcd. for C₁₁H₁₄N₄OS.1.1H₂O: C, 48.91; H, 6.04; N, 20.74; S, 11.87. Found: C, 49.06; H, 5.92; N, 20.71; S, 11.90.

### Example 5

### 3-amino-4-(isobutylamino)thieno[2,3-b]pyridine-2-carboxamide

(Exemplified Compound No. 2-6)

### (5a) (2Z)-2-cyano-3-(isobutylamino)but-2-enethioamide

(2Z)-2-cyano-3-ethoxybut-2-enethioamide (J. Org. Chem., (1962), 27,2433-2439) (340 mg, 2.0 mmol) and isobutylamine (183 mg, 2.5 mmol) were suspended in ethanol (3 mL) and the mixture was stirred at room temperature for six hours. After the deposited solid was separated by filtration, it was washed with ethanol and 378 mg of the title compound (96%) was obtained.

Mp 165-167° C.;

IR (KBr) νₘₐₓ 3373, 3291, 3198, 2190, 1608 cm⁻¹;

¹H NMR (DMSO-d₆, 400 MHz) δ 0.97 (6H, d, J=6.7 Hz), 1.82-1.92 (1H, m), 2.30 (3H, s), 3.25-3.28 (2H, m), 7.67 (1H, br), 8.48 (1H, br), 12.76 (1H, br);

MS (FAB) m/z: 198 [M+H]⁺;

Anal. Calcd. for C₉H₁₅N₃S: C, 54.79; H, 7.66; N, 21.30; S, 16.25. Found: C, 54.73; H, 7.84; N, 21.24; S, 16.18.

### (5b) 4-(isobutylamino)-2-thioxo-1,2-dihydropyridine-3-carbonitrile

(2Z)-2-cyano-3-(isobutylamino)but-2-enethioamide (360 mg, 1.8 mmol) which was produced in Example 5 (5a) and N,N-dimethylformamide dimethylacetal (652 mg, 5.5 mmol) were mixed with toluene (5 mL) and the mixture was stirred under heat reflux for two hours. 1N aqueous solution of sodium hydroxide (5 mL) was added to the residue which was obtained by evaporating the solvent under reduced pressure, and it was stirred under heat reflux for 30 minutes. After the reaction mixture was cooled to room temperature, the liquid was partitioned with ether (50 mL) and water (20 mL). The obtained aqueous layer was neutralized with 1N hydrochloric acid (5 mL) and the deposited solid was separated by filtration and further washed with water and a little amount of ethanol and 217 mg of a solid mainly containing the title compound was obtained.

¹H NMR (DMSO-d₆, 400 MHz) δ 0.87 (6H, d, J=6.7 Hz), 1.78-1.88 (1H, m), 3.06-3.12 (2H, m), 6.33 (1H, d, J=7.4 Hz), 7.41 (1H, br), 7.50 (1H, br), 12.35 (1H, br).

### (5c) 3-amino-4-(isobutylamino)thieno[2,3-b]pyridine-2-carboxamide

Crude product (215 mg, 1.0 mmol) of 4-(isobutylamino)-2-thioxo-1,2-dihydropyridine-3-carbonitrile which was produced in Example 5 (5b) was dissolved in N,N-dimethylformamide (3 mL) and 8N aqueous solution of sodium hydroxide (0.3 mL) and 2-chloroacetamide (126 mg, 1.3 mmol) were added. Water (3 mL) was added after the mixture was stirred at room temperature for one hour. The deposited solid was separated by filtration and washed with water and ethanol and 109 mg of the title compound was obtained. Yield 23% from 4-(isobutylamino)-2-thioxo-1,2-dihydropyridine-3-carbonitrile.

Mp 117-119° C.;

IR (KBr) νₘₐₓ 3399, 3352, 3250, 3121, 1676, 1595, 860 cm⁻¹;

¹H NMR (DMSO-d₆, 400 MHz) δ 0.95 (6H, d, J=6.7 Hz), 1.90-2.01 (1H, m), 3.04-3.08 (2H, m), 6.43 (1H, d, J=5.5 Hz), 6.52 (1H, br), 6.83 (2H, br), 7.08 (2H, br), 8.07 (1H, d, J=5.5 Hz);

MS (EI) m/z: 264 [M⁺], 204;

Anal. Calcd. for C₁₂H₁₆N₄OS. 0.1H₂O: C, 54.15; H, 6.14; N, 21.05; S, 12.05. Found: C, 54.11; H, 5.94; N, 21.06; S, 12.17.

### Example 6

### 3-amino-4-(neopentylamino)thieno[2,3-b]pyridine-2-carboxamide

(Exemplified Compound No. 2-7)

### (6a) (2Z)-2-cyano-3-(neopentylamino)but-2-enethioamide

Neopentylamine was used in place of propylamine and the reaction was performed in a similar method as described in Example 4 (4a) and the title compound was obtained. Yield 54%.

Mp 143-144° C.;

IR (KBr) νₘₐₓ 3376, 3297, 3202, 2188, 1607 cm⁻¹;

¹H NMR (DMSO-d₆, 400 MHz) δ 0.99 (9H, s), 2.30 (3H, s), 3.23 (2H, d, J=5.5 Hz), 7.67 (1H, br), 8.49 (1H, br), 12.78 (1H, br);

MS (FAB) m/z: 212 [M+H]⁺;

Anal. Calcd. for C₁₀H₁₇N₃S: C, 56.84; H, 8.11; N, 19.88; S, 15.17. Found: C, 56.59; H, 8.09; N, 19.76; S, 14.90.

### (6b) 3-amino-4-(neopentylamino)thieno[2,3-b]pyridine-2-carboxamide

(2Z)-2-cyano-3-(neopentylamino)but-2-enethioamide which was produced in Example 6 (6a) was used in place of (2Z)-2-cyano-3-(propylamino)but-2-enethioamide and the reaction was performed in a similar method as described in Example 4 (4b) and the title compound was obtained. Yield 16%.

Mp 238-240° C.;

IR (KBr) νₘₐₓ 3318, 3190, 1653, 1587, 1105 cm⁻¹;

¹H NMR (DMSO-d₆, 400 MHz) δ 0.97 (9H, s), 3.08 (2H, d, J=6.1 Hz), 6.46 (1H, brt, J=6.1 Hz), 6.54 (1H, d, J=5.7 Hz), 6.63 (2H, br), 7.14 (2H, br), 8.06 (1H, d, J=5.7 Hz);

MS (FAB) m/z: 279 [M+H]⁺;

Anal. Calcd. for C₁₃H₁₈N₄OS.1.2H₂O: C, 52.05; H, 6.85; N, 18.68; S, 10.69. Found: C, 52.31; H, 6.69; N, 18.68; S, 10.67.

### Example 7

### 3-amino-4-(benzylamino)thieno[2,3-b]pyridine-2-carboxamide

(Exemplified Compound No. 2-11)

### (7a) (2Z)-3-(benzylamino) -2-cyanobut-2-enethioamide

Benzylamine was used in place of propylamine and the reaction was performed in a similar method as described in Example 4 (4a) and the title compound was obtained. Yield 97%.

Mp 157-159° C.;

IR (KBr) νₘₐₓ 3355, 3287, 3193, 2193, 1627, 1608, 853, 739 cm⁻¹;

¹H NMR (DMSO-d₆, 400 MHz) δ 2.34 (3H, s), 4.69 (2H, d, J=5.6 Hz), 7.31-7.43 (5H, m), 7.74 (1H, br), 8.55 (1H, br), 13.02 (1H, br);

MS (FAB) m/z: 232 [M+H]⁺;

Anal. Calcd. for C₁₂H₁₃N₃S: C, 62.31; H, 5.66; N, 18.17; S, 13.86. Found: C, 62.19; H, 5.88; N, 18.27; S, 13.66.

### (7b) 4-(benzylamino)-2-thioxo-1,2-dihydropyridine-3-carbonitrile

(2Z)-3-(benzylamino) -2-cyanobut-2-enethioamide (0.41 g) which was produced in Example 7 (7a) was used in place of (2Z)-2-cyano-3-(dimethylamino)but-2-enethioamide and the reaction was performed in a similar method as described in Example 1 (1b) and a crude product (0.38 g) of the title compound was obtained.

¹H NMR (DMSO-d₆, 400 MHz) δ 4.54 (2H, d, J=6.3 Hz), 6.19 (1H, d, J=7.4 Hz), 7.24-7.44 (5H, m), 8.12 (1H, br), 12.45 (1H, br).

### (7c) 3-amino-4-(benzylamino)thieno[2,3-b]pyridine-2-carboxamide

A crude product (0.38 g) of 4-(benzylamino)-2-thioxo-1,2-dihydropyridine-3-carbonitrile which was produced in Example 7 (7b) was used in place of 4-(dimethylamino)-2-thioxo-1,2-dihydropyridine-3-carbonitrile and the reaction was performed in a similar method as described in Example 1 (1c), and 0.39 g of the title compound was obtained. Yield 73% from (2Z)-3-(benzylamino)-2-cyanobut-2-enethioamide.

Mp 260-262° C.;

IR (KBr) νₘₐₓ 3460, 3394, 3351, 3112, 1654, 1627, 1598 cm⁻¹;

¹H NMR (DMSO-d6, 400 MHz) δ 4.52 (2H, d, J=5.8 Hz), 6.28 (1H, d, J=5.7 Hz), 6.95 (2H, br), 7.07 (2H, br), 7.16 (1H, brt, J=5.8 Hz), 7.23-7.42 (5H, m), 8.00 (1H, d, J=5.7 Hz);

MS (FAB) m/z: 299 [M+H]⁺;

Anal. Calcd. for C₁₅H₁₄N₄OS: C, 60.38; H, 4.73; N, 18.78; S, 10.75. Found: C, 60.37; H, 4.85; N, 18.87; S, 10.65.

### Example 8

### 3-amino-4-[(2-phenethyl)amino]thieno[2,3-b]pyridine-2-carboxamide

(Exemplified Compound No. 2-12)

### (8a) (2Z)-2-cyano-3-[(2-phenethyl)amino]but-2-enethioamide

Phenethylamine was used in place of propylamine, the reaction was performed in a similar method as described in Example 4 (4a) and the title compound was obtained. Yield 75%.

Mp 95-96° C.;

IR (KBr) νₘₐₓ 3353, 3300, 3203, 2189, 1626, 1595, 749, 699 cm⁻¹;

¹H NMR (DMSO-d₆, 400 MHz) δ 2.22 (3H, s), 2.89 (2H, t, J=7.2 Hz), 3.64-3.69 (1H, m), 7.18-7.31 (5H, m), 7.60 (1H, br), 8.40 (1H, br), 12.70 (1H, br);

MS (FAB) m/z: 246 [M+H]⁺;

Anal. Calcd. for C₁₃H₁₅N₃S: C, 63.64; H, 6.16; N, 17.13; S, 13.07. Found: C, 63.73; H, 6.11; N, 17.20; S, 13.14.

### (8b) 3-amino-4-[(2-phenethyl)amino]thieno[2,3-b]pyridine-2-carboxamide

(2Z)-2-cyano-3-[(2-phenethyl)amino]but-2-enethioamide (0.37 g, 1.5 mmol) which was produced in Example 8 (8a) was used in place of (2Z)-2-cyano-3-(dimethylamino)but-2-enethioamide and the reaction was performed in a similar method as described in Example 1 (1b) and 0.34 g of a crude product of 2-thioxo -1,2-dihydropyridine derivative was obtained.

The reaction was performed in a similar method as described in Example 1 (1c) using the obtained 2-thioxo-1,2-dihydropyridine derivative and 0.25 g of the title compound was obtained (yield 53%).

Mp 206-208° C.;

IR (KBr) νₘₐₓ 3449, 3358, 3121, 1656, 1598, 1514, 1103, 753, 703 cm⁻¹;

¹H NMR (DMSO-d₆, 400 MHz) δ 2.93 (2H, t, J=7.5 Hz), 3.44-3.49 (2H, m), 6.49 (1H, d, J=5.7 Hz), 6.50 (1H, br), 6.76 (2H, br), 7.04 (2H, br), 7.18-7.32 (5H, m), 8.07 (1H, d, J=5.7 Hz);

MS (FAB) m/z: 313 [M+H]⁺;

Anal. Calcd. for C₁₆H₁₆N₄0S: C, 61.52; H, 5.16; N, 17.93; S, 10.26. Found: C, 61.47; H, 5.25; N, 17.95; S, 10.33.

### Example 9

### 3-amino-4-[(3-phenylpropyl)amino]thieno[2,3-b]pyridine-2-carboxamide

(Exemplified Compound No. 2-13)

### (9a) (2Z)-2-cyano-3-[(3-phenylpropyl)amino]but-2-enethioamide

3-phenyl propylamine was used in place of propylamine, the reaction was performed in a similar method as described in Example 4 (4a) and the title compound was obtained. Yield 94%.

Mp 125-126° C.;

IR (KBr) νₘₐₓ 3399, 3285, 3173, 2189, 1612, 1603, 754, 531 cm⁻¹;

¹H NMR (DMSO-d₆, 400 MHz) δ 1.85-1.92 (2H, m), 2.27 (3H, s), 2.70 (2H, t, J=7.7 Hz), 3.37-3.42 (2H, m), 7.16-7.29 (5H, m), 7.64 (1H, br), 8.45 (1H, br), 12.79 (1H, br);

MS (FAB) m/z: 260 [M+H]⁺;

Anal. Calcd. for C₁4H₁₇N₃S: C, 64.85; H, 6.61; N, 16.20; S, 12.36. Found: C, 65.02; H, 6.52; N, 16.28; S, 12.37.

### (9b) 3-amino-4-[(3-phenylpropyl)amino]thieno[2,3-b]pyridine-2-carboxamide

(2Z)-2-cyano-3-[(3-phenylpropyl)amino]but-2-enethioamide (0.49 g, 1.9 mmol) which was produced in Example 9 (9a) was used in place of (2Z)-2-cyano-3-(dimethylamino)but-2-enethioamide and the reaction was performed in a similar method as described in Example 1 (1b) and a crude product of 2-thioxo-1,2-dihydropyridine derivative (0.45 g) was obtained.

The reaction was performed in a similar method as described in Example 1 (1c) using the obtained 2-thioxo-1,2-dihydropyridine derivative and 0.30 g of the title compound was obtained (yield 49%).

Mp 232-234° C.;

IR (KBr) νₘₐₓ 3424, 3326, 3125, 1659, 1603, 1517, 1362 cm⁻¹;

¹H NMR (DMSO-d₆, 400 MHz) δ 1.89-1.97 (2H, m), 2.69 (2H, t, J=7.6 Hz), 3.21-3.26 (2H, m), 6.37 (1H, d, J=5.6 Hz), 6.47 (1H, brt, J=5.3 Hz), 6.81 (2H, br), 7.02 (2H, br), 7.15-7.29 (5H, m), 8.04 (1H, d, J=5.6 Hz);

MS (FAB) m/z: 327 [M+H]⁺;

Anal. Calcd for C₁₇H₁₈N₄OS: C, 62.55; H, 5.56; N, 17.16; S, 9.82. Found: C, 62.33; H, 5.76; N, 17.32; S, 9.68.

### Example 10

### 3-amino-4-(cyclopentylamino)thieno[2,3-b]pyridine-2-carboxamide

(Exemplified Compound No. 2-8)

### (10a) (2Z)-2-cyano-3-(cyclopentylamino)but-2-enethioamide

Cyclopentylamine was used in place of isobutylamine, the reaction was performed in a similar method as described in Example 5 (5a) and the title compound was obtained. Yield 89%.

Mp 162-163° C.;

IR (KBr) νₘₐₓ 3368, 3287, 3196, 2192, 1612 cm⁻¹;

¹H NMR (DMSO-d₆, 400 MHz) δ 1.53-1.74 (6H, m), 1.92-2.00 (2H, m), 2.33 (3H, s), 4.11-4.19 (1H, m), 7.63 (1H, br), 8.44 (1H, br), 12.94 (1H, brd, J=7.5 Hz);

MS (FAB) m/z: 210 [M+H]⁺;

Anal. Calcd for C₁₀H₁₅N₃S: C, 57.38; H, 7.22; N, 20.08; S, 15.32. Found: C, 57.39; H, 7.21; N, 19.98; S, 15.26.

### (10b) 4-(cyclopentylamino)-2-thioxo-1,2-dihydropyridine-3-carbonitrile

(2Z)-2-cyano-3-(cyclopentylamino)but-2-enethioamide (358 mg) which was produced in Example 10 (10a) was used in place of (2Z)-2-cyano-3-(dimethylamino)but-2-enethioamide and the reaction was performed in a similar method as described in Example 1 (1b) and a crude product (318 mg) of the title compound was obtained.

¹H NMR (DMSO-d₆, 400MHz) δ 1.51-1.73 (6H, m), 1.90-1.98 (2H, m), 3.94-4.04 (1H, m), 6.37 (1H, d, J=7.8 Hz), 7.44-7.49 (1H, m), 12.42 (1H, br).

### (10c) 3-amino-4-(cyclopentylamino)thieno[2,3-b]pyridine-2-carboxamide

A crude product (318 mg) of 4-(cyclopentylamino)-2-thioxo-1,2-dihydropyridine-3-carbonitrile which was produced in Example 10 (1b) was used in place of 4-(dimethylamino)-2-thioxo-1,2-dihydropyridine-3-carbonitrile, and the reaction was performed in a similar method as described in Example 1 (1c) and 261 mg of the title compound was obtained. Yield 55% from (2Z)-3-(cyclopentylamino)-2-cyanobut-2-enethioamide.

Mp 212-213° C.;

IR (KBr) νₘₐₓ 3311, 3177, 1649, 1594, 1513, 1497 cm⁻¹;

H NMR (DMSO-d₆, 400 MHz) δ 1.52-1.73 (6H, m), 2.01-2.08 (2H, m), 3.85-3.92 (1H, m), 6.21 (1H, brd, J=6.4 Hz), 6.43 (1H, d, J=5.6 Hz), 6.72 (2H, br), 7.07 (2H, br), 8.06 (1H, d, J=5.6 Hz);

MS (EI) m/z: 276 [M⁺], 231;

Anal. Calcd for C₁₃H₁₆N₄OS: C, 56.50; H, 5.84; N, 20.27; S, 11.60. Found: C, 56.31; H, 5.73; N, 20.13; S, 11.33.

### Example 11

### 3-amino-4-(cyclohexylamino)thieno[2,3-b]pyridine-2-carboxamide

(Exemplified Compound No. 2-9)

### (11a) (2Z)-2-cyano-3-(cyclohexylamino)but-2-enethioamide

Cyclohexylamine was used in place of isobutylamine, the reaction was performed in a similar method as described in Example 5 (5a) and the title compound was obtained. Yield 76%.

Mp 142-144° C.;

IR (KBr) νₘₐₓ 3412, 3297, 3188, 2187, 1613, 1493, 1410 cm⁻¹;

¹H NMR (DMSO-d₆, 400 MHz) δ 1.33-1.61 (6H, m), 1.81-1.93 (4H, m), 2.37 (3H, s), 3.55-3.64 (1H, m), 6.38 (1H, br), 6.69 (1H, br), 12.92 (1H, br);

MS (EI) m/z: 223 [M⁺], 190.

### (11b) 3-amino-4-(cyclohexylamino)thieno[2,3-b]pyridine-2-carboxamide

(2Z)-2-cyano-3-(cyclohexylamino)but-2-enethioamide (0.33 g, 1.5 mmol) which was produced in Example 11 (11a) was used in place of (2Z)-2-cyano-3-(dimethylamino)but-2-enethioamide and the reaction was performed in a similar method as described in Example 1 (1b) and a crude product (0.11 g) of 2-thioxo-1,2-dihydropyridine derivative was obtained.

The reaction was performed in a similar method as described in Example 1 (1c) using the obtained 2-thioxo-1,2-dihydropyridine derivative and 0.06 g of the title compound was obtained (yield 14%).

Mp 244-247° C.;

IR (KBr) νₘₐₓ 3141, 1664, 1598, 1513, 1497, 1108 cm⁻¹;

¹H NMR (DMSO-d₆, 400 MHz) δ 1.20-1.98 (10H, m), 3.43-3.50 (1H, m), 6.16 (1H, d, J=7.5 Hz), 6.46 (1H, d, J=5.7 Hz), 6.71 (2H, br), 7.08 (2H, br), 8.06 (1H, d, J=5.7 Hz);

MS (FAB) m/z: 291 [M+H]⁺;

Anal. Calcd for C₁₄H₁₈N₄OS.0.3H₂O: C, 56.85; H, 6.34; N, 18.94; S, 10.85. Found: C, 57.00; H, 6.27; N, 18.95; S, 10.61.

### Example 12

### 3-amino-4-(cycloheptylamino)thieno[2,3-b]pyridine-2-carboxamide

(Exemplified Compound No. 2-10)

### (12a) (2Z)-2-cyano-3-(cycloheptylamino)but-2-enethioamide

Cycloheptylamine was used in place of isobutylamine and the reaction was performed in a similar method as described in Example 5 (5a) and the title compound was obtained. Yield 74%.

Mp 132-134° C.;

IR (KBr) νₘₐₓ 3380, 3295, 3200, 2189, 1609, 853 cm⁻¹;

¹H NMR (DMSO-d₆, 400 MHz) δ 1.48-1.63 (10H, m), 1.85-1.92 (2H, m), 2.32 (3H, s), 3.90-3.97 (1H, m), 7.62 (1H, br), 8.43 (1H, br), 12.94 (1H, brd, J=8.9 Hz);

MS (FAB) m/z: 238 [M+H]⁺;

Anal. Calcd for C₁₂H₁₉N₃S: C, 60.72; H, 8.07; N, 17.70; S, 13.51. Found: C, 60.69; H, 8.12; N, 17.68; S, 13.41.

### (12b) 4-(cycloheptylamino)-2-thioxo-1,2-dihydropyridine-3-carbonitrile

(2Z)-2-cyano-3-(cycloheptylamino)but-2-enethioamide (339 mg) which was produced in Example 12 (12a) was used in place of (2Z)-2-cyano-3-(dimethylamino)but-2-enethioamide and the reaction was performed in a similar method as described in Example 1 (1b) and a crude product (306 mg) of the title compound was obtained.

¹H NMR (DMSO-d₆, 400 MHz) δ 1.40-1.72 (10H, m), 1.76-1.85 (2H, m), 3.61-3.73 (1H, m), 6.30 (1H, d, J=7.4 Hz), 6.82 (1H, br), 7.43 (1H, br), 12.35 (1H, br).

### (12c) 3-amino-4-(cycloheptylamino)thieno[2,3-b]pyridine-2-carboxamide

A crude product (306 mg) of 4-(cycloheptylamino)-2-thioxo-1,2-dihydropyridine-3-carbonitrile which was produced in Example 12 (12b) was used in place of 4-(isobutylamino)-2-thioxo-1,2-dihydropyridine-3-carbonitrile and the reaction was performed in a similar method as described in Example 5 (5c) and 201 mg of the title compound was obtained. Yield 46% from (2Z)-2-cyano-3-(cycloheptylamino)but-2-enethioamide.

Mp 206-208° C.;

IR (KBr) ν^{max} 3334, 1652, 1594, 1514, 1498, 1365 cm⁻¹;

¹H NMR (DMSO-d₆, 400 MHz) δ 1.49-1.71 (10H, m), 1.93-1.98 (2H, m), 3.60-3.63 (1H, m), 6.21 (1H, d, J=7.5 Hz), 6.35 (1H, d, J=5.8 Hz), 6.70 (2H, br), 7.10 (2H, br), 8.08 (1H, d, J=5.8 Hz);

MS (FAB) m/z: 305 [M+H]⁺;

Anal. Calcd for C₁₅H₂₀N₄OS.0.1H₂O: C, 58.84; H, 6.65; N, 18.30; S, 10.47. Found: C, 58.77; H, 6.48; N, 18.22; S, 10.34.

### Example 13

### 3-amino-4-pyrrolidin-1-ylthieno[2,3-b]pyridine-2-carboxamide

(Exemplified Compound No. 1-15)

### (13a) (2Z)-2-cyano-3-pyrrolidin-1-ylbut-2-enethioamide

Pyrrolidine was used in place of propylamine, the reaction was performed in a similar method as described in Example 4 (4a) and the title compound was obtained. Yield 86%

¹H NMR(CD₃OD, 400 MHz) δ 2.00 (4H, s), 2.47 (3H, s), 3.63 (4H, s).

### (13b) 4-pyrrolidin-1-yl-2-thioxo-1,2-dihydropyridine-3-carbonitrile

(2Z)-2-cyano-3-pyrrolidin-1-ylbut-2-enethioamide which was produced in Example 13 (13a) was used in place of (2Z)-2-cyano-3-(dimethylamino)but-2-enethioamide and the reaction was performed in a similar method as described in Example 1 (1b) and the title compound was obtained. Yield 80%.

¹H NMR(DMSO-d₆, 400 MHz) δ 1.91 (4H, s), 3.55-3.75 (4H, br s), 6.21 (1H, d, J=7.3 Hz), 7.365 (1H, dd, J=5.9, 7.3 Hz).

### (13c) 3-amino-4-pyrrolidin-1-yl thieno[2,3-b]pyridine-2-carboxamide

4-pyrrolidin-1-yl-2-thioxo-1,2-dihydropyridine-3-carbonitrile which was produced in Example 13 (13b) was used in place of 4-(dimethylamino)-2-thioxo-1,2-dihydropyridine-3-carbonitrile, and the reaction was performed in a similar method as described in Example 1 (1c) and the title compound was synthesized. Yield 85%.

Mp 275-283° C.;

IR (KBr) νₘₐₓ 3429, 3297, 3138, 2999, 2877, 1673, 1611, 1584, 1503, 1372, 1341, 1273, 1113, 1056, 1002, 618, 486 cm⁻¹;

¹H NMR(DMSO-d₆, 400 MHz) δ 1.91 (4H, s), 3.30 (4H, s), 6.87 (1H, d, J=5.8 Hz), 6.88 (2H, s), 7.02 (2H, br s), 8.255 (1H, d, J=5.8 Hz);

MS (FAB) m/z: 262.08 [M+H]⁺;

Anal. Calcd for C₁₂H₁₄N₄OS: C, 54.94; H, 5.38; N, 21.36; S, 12.22. Found: C, 54.54; H, 5.15; N, 21.10; S, 12.03.

### Example 14

### 3-amino-4-piperidin-1-ylthieno[2,3-b]pyridine-2-carboxamide

(Exemplified Compound No. 1-16)

### (14a) (2Z)-2-cyano-3-piperidin-1-ylbut-2-enethioamide

An ethanol (30 mL) solution of (2Z)-2-cyano-3-ethoxybut-2-enethioamide (1.70 g, 10 mmol) and piperidine (1.02 g, 12 mmol) was stirred for two hours under heat reflux and allowed to stand overnight at room temperature. After the deposited solid was separated by filtration, it was washed with ethanol (3×3 mL) and 1.36 g of the title compound was obtained (yield 65%).

Mp 161-166° C.;

IR (KBr) νₘₐₓ 3381, 3268, 3170, 2182, 1600, 1534, 873, 838, 636 cm⁻¹;

1H NMR (DMSO-d₆, 400 MHz) δ 1.58-1.63 (6H, m), 2.27 (3H, s), 3.31-3.36 (4H, m), 8.15 (1H, br), 8.86 (1H, br);

MS (EI) m/z: 209 [M⁺], 150, 44;

Anal. Calcd for C₁₀H₁₅N₃S: C, 57.38; H, 7.22; N, 20.08; S, 15.32. Found: C, 57.27; H, 7.16; N, 20.03; S, 15.47.

### (14b) 4-piperidin-1-yl-2-thioxo-1,2-dihydropyridine-3-carbonitrile

(2Z)-2-cyano-3-piperidin-1-ylbut-2-enethioamide (0.42 g, 2.0 mmol) which was produced in Example 14 (14a) and N,N-dimethylformamide dimethylacetal (0.72 g, 6.0 mmol) were mixed with toluene (3 mL) and the mixture was stirred under reflux for two hours. 1N aqueous solution of sodium hydroxide (3 mL) was added to the residue which was obtained by evaporating the solvent under reduced pressure and the mixture was stirred under heat reflux for 30 minutes. After the reaction mixture was cooled to room temperature, 1N hydrochloric acid (4 mL) was added and the deposited solid was separated by filtration and further washed with water and a little amount of ethanol and 0.30 g of a solid mainly containing the title compound was obtained.

¹H NMR (DMSO-d₆, 400 MHz) δ 1.50-1.55 (6H, m), 3.48-3.52 (4H, m), 6.38 (1H, d, J=7.3 Hz), 7.35 (1H, d, J=7.3 Hz).

### (14c) 3-amino-4-piperidin-1-ylthieno[2,3-b]pyridine-2-carboxamide

4-piperidin-1-yl-2-thioxo-1,2-dihydropyridine-3-carbonitrile (0.30 g) which was produced in Example 14 (14b) was dissolved in N,N-dimethylformamide (3 mL) and 8N aqueous solution of sodium hydroxide (0.6 mL) and 2-chloroacetamide (0.15 g, 1.6 mmol)were added. Water (5 mL) was added after the mixture was stirred at room temperature for one hour. The deposited solid was separated by filtration and washed with water and ethanol and 0.30 g of a solid was obtained. The obtained solid was purified by silica gel column chromatography (100% ethyl acetate) and 0.21 g of the title compound was obtained. Yield 38% from (2Z)-2-cyano-3-piperidin-1-ylbut-2-enethioamide.

Mp 191-192° C.;

IR (KBr) νₘₐₓ 3460, 3329, 3176, 1651, 1589, 1501, 1378, 963 cm⁻¹;

¹H NMR (DMSO-d₆, 400MHz) δ 1.72-1.79 (6H, m), 2.55-3.40 (4H, m), 6.93 (2H, br), 6.99 (1H, d, J=5.1 Hz), 7.07 (2H, br), 8.40 (1H, d, J=5.1 Hz);

MS (FAB) m/z: 277 [M+H]⁺;

Anal. Calcd for C₁₃H₁₆N₄SO.0.5 H₂O: C, 54.72; H, 6.00; N, 19.63; S, 11.24. Found: C, 54.68; H, 6.03; N, 19.67; S, 11.05.

### Example 15

### 3-amino-4-(4-methylpiperidin-1-yl)thieno[2,3-b]pyridine-2-carboxamide

(Exemplified Compound No. 1-18)

### (15a) (2Z)-2-cyano-3-(4-methylpiperidin-1-yl)but-2-enethioamide

4-methylpiperidine was used in place of isobutylamine, the reaction was performed in a similar method as described in Example 5 (5a) and the title compound was obtained. Yield 87%.

Mp 156-159° C.;

IR (KBr) νₘₐₓ 3387, 3262, 3162, 2177, 1604, 1540, 868 cm⁻¹;

¹H NMR (DMSO-d₆, 400 MHz) δ 0.91 (3H, d, J=6.1 Hz), 1.15-1.25 (2H, m), 1.62-1.73 (3H, m), 2.27 (3H, s), 3.04-3.10 (2H, m), 3.62 (2H, brd, J=13.5 Hz), 8.14 (1H, br), 8.85 (1H, br);

MS (EI) m/z: 223 [M⁺], 190;

Anal. Calcd for C₁₁H₁₇N₃S.0.1H₂O: C, 58.68; H, 7.70; N, 18.66; S, 14.27. Found: C, 58.92; H, 7.73; N, 18.77; S, 14.06.

### (15b) 4-(4-methylpiperidin-1-yl)-2-thioxo-1,2-dihydropyridine-3-carbonitrile

(2Z)-2-cyano-3-(4-methylpiperidin-1-yl)but-2-enethioamide (0.39 g) which was produced in Example 15 (15a) was used in place of (2Z)-2-cyano-3-(dimethylamino)but-2-enethioamide and the reaction was performed in a similar method as described in Example 1 (1b). After the reaction terminated, toluene was added and the liquid was partitioned, and then the aqueous layer was neutralized with 1N hydrochloric acid and the deposited solid was separated by filtration and further washed with water and ethanol and a crude product (0.24 g) of the title compound was obtained.

¹H NMR (DMSO-d₆, 400MHz) δ 0.93 (3H, d, J=6.4 Hz), 1.14-1.24 (2H, m), 1.65-1.76 (3H, m), 3.10-3.16 (2H, m), 4.08 (2H, brd, J=13.2 Hz), 6.49 (1H, d, J=7.5 Hz), 7.44 (1H, d, J=7.5 Hz), 12.60 (1H, br).

### (15c) 3-amino-4-(4-methylpiperidin-1-yl)thieno[2,3-b]pyridine-2-carboxamide

A crude product (0.24 g) of 4-(4-methylpiperidin-1-yl)-2-thioxo-1,2-dihydropyridine-3-carbonitrile which was produced in Example 15 (15b) was used in place of 4-(isobutylamino)-2-thioxo-1,2-dihydropyridine-3-carbonitrile, and the reaction was performed in a similar method as described in Example 5 (5c) and 0.24 g of the title compound was obtained. Yield 41% from (2Z)-2-cyano-3-(4-methylpiperidin-1-yl)but-2-enethioamide.

Mp 252-254° C.;

IR (KBr) νₘₐₓ 3430, 3311, 3159, 1667, 1611, 1580, 1504, 1367, 1341 cm⁻¹;

¹H NMR (DMSO-d₆, 400 MHz) δ 0.98 (3H, d, J=6.0 Hz), 1.40-1.76 (5H, m), 2.66-2.80 (2H, m), 3.27-3.30 (2H, m), 6.91 (2H, br), 6.99 (1H, d, J=5.2 Hz), 7.05 (2H, br), 8.39 (1H, d, J=5.2 Hz);

MS (FAB) m/z: 291 [M+H]⁺;

Anal. Calcd for C₁₄H₁₈N₄SO: C, 57.91; H, 6.25; N, 19.29; S, 11.04. Found: C, 57.89; H, 6.31; N, 19.29; S, 11.06.

### Example 16

### 3-amino-4-(3-methylpiperidin-1-yl)thieno[2,3-b]pyridine-2-carboxamide

(Exemplified Compound No. 1-17)

### (16a) 4-(3-methylpiperidin-1-yl)-2-thioxo-1,2-dihydropyridine-3-carbonitrile

(2Z)-2-cyano-3-ethoxybut-2-enethioamide (340 mg, 2.0 mmol) and 3-methylpiperidine (248 mg, 2.5 mmol) were mixed with ethanol (2 mL) and the mixture was stirred at room temperature for two hours. The residue which was obtained by concentrating the reaction mixture under reduced pressure was dissolved in toluene (6 mL) and was blended with N,N-dimethylformamide dimethylacetal (714 mg, 6.0 mmol) and the mixture was stirred under heat reflux for two hours. 1N aqueous solution of sodium hydroxide (4 mL) was added to the residue after the solvent was evaporated under reduced pressure. The mixture was stirred under heat reflux for one hour and after it was cooled, 1N hydrochloric acid (5 mL) was added. After the deposited solid was separated by filtration, it was washed with water and ethanol and 211 mg of a solid mainly containing the title compound was obtained.

¹H NMR (DMSO-d₆, 400 MHz) δ 0.89 (3H, d, J=6.7 Hz), 1.14-1.25 (1H, m), 1.47-1.83 (4H, m), 2.82 (1H, dd, J=10.6, 13.3 Hz), 3.08-3.15 (1H, m), 3.96-4.05 (2H, m), 6.50 (1H, d, J=6.5 Hz), 7.42-7.45 (1H, m), 12.59 (1H, br).

### (16b) 3-amino-4-(3-methylpiperidin-1-yl)thieno[2,3-b]pyridine-2-carboxamide

A solid (211 mg) mainly containing 4-(3-methylpiperidin-1-yl)-2-thioxo-1,2-dihydropyridine-3-carbonitrile which was produced in Example 16 (16a) was dissolved in N,N-dimethylformamide (2 mL) and 8N aqueous solution of sodium hydroxide (0.2 mL) and 2-chloroacetamide (122 mg, 1.3 mmol) were added and the mixture was stirred at room temperature for one hour. Water (2 mL) was added to the reaction mixture and the deposited solid was separated by filtration and further washed with water and ethanol and 158 mg of the title compound was obtained. Yield 27% from (2Z)-2-cyano-3-ethoxybut-2-enethioamide.

Mp 201-204° C.;

IR (KBr) νₘₐₓ 3422, 3325, 3162, 1657, 1580, 1501, 1371 cm⁻¹;

¹H NMR (DMSO-d₆, 400 MHz) δ 0.91 (3H, d, J=6.3 Hz), 1.70-1.95 (4H, m), 2.23-2.70 (2H, m), 3.20-3.30 (2H, m), 6.90 (2H, br), 6.99 (1H, d, J=5.1 Hz), 7.08 (2H, br), 8.40 (1H, d, J=5.1 Hz);

MS (FAB) m/z: 291 [M+H]⁺;

Anal. Calcd for C₁₄H₁₈N₄SO.0.1 H₂O: C, 57.55; H, 6.28; N, 19.17; S, 10.97. Found: C, 57.45; H, 6.10; N, 19.35; S, 11.12.

### Example 17

### 3-amino-4-[3-(hydroxymethyl)piperidin-1-yl]thieno[2,3-b]pyridine-2-carboxamide

(Exemplified Compound No. 1-20)

(2Z)-2-cyano-3-ethoxybut-2-enethioamide (340 mg, 2.0 mmol) and 3-(hydroxymethyl)piperidine (288 mg, 2.5 mmol) were mixed with ethanol (3 mL) and the mixture was stirred at room temperature for 1.5 hours. The residue which was obtained by concentrating the reaction mixture under reduced pressure was dissolved in N,N-dimethylformamide (3 mL) and was blended with N,N-dimethylformamide dimethylacetal (250 mg, 2.1 mmol) and the mixture was stirred at room temperature for one hour and further at 100° C. for one hour. The reaction mixture was cooled to room temperature and was blended with 8N aqueous solution of sodium hydroxide (0.4 mL) and 2-chloroacetamide (281 mg, 3.0 mmol) and the mixture was stirred at room temperature for one hour. Water (50 mL) and ethyl acetate (50 mL) were added to the reaction mixture and the liquid was partitioned, and further the aqueous layer was extracted with ethyl acetate (30 mL). Combined with the organic layer, the solvent was evaporated under reduced pressure after drying over sodium sulfate. The residue was purified by silica gel column chromatography (methylene chloride/methanol=10:1) and 146 mg of the title compound was obtained. Yield 24% from (2Z)-2-cyano-3-ethoxybut-2-enethioamide.

Mp 125-130° C.;

IR (KBr) νₘₐₓ 3441, 3335, 3183, 1661, 1595, 1583, 1503, 1375, 1037 cm⁻¹;

¹H NMR (DMSO-d₆, 400 MHz) δ 0.95-1.15 (1H, m), 1.70-1.99 (4H, m), 2.26-2.70 (2H, m), 3.21-3.43 (4H, m), 4.56 (1H, t, J=5.3 Hz), 6.94 (2H, br), 7.02 (1H, d, J=5.1 Hz), 7.11 (2H, br), 8.44 (1H, d, J=5.1 Hz);

MS (FAB) m/z: 307 [M+H]⁺;

Anal. Calcd for C₁₄H₁₈N₄SO₂.1 H₂O: C, 51.84; H, 6.21; N, 17.27; S, 9.88. Found: C, 51.72; H, 6.15; N, 17.37; S, 9.70.

### Example 18

### 3-amino-4-(4-benzylpiperidin-1-yl)thieno[2,3-b]pyridine-2-carboxamide

(Exemplified Compound No. 1-19)

### (18a) (2Z)-3-(4-benzylpiperidin-1-yl) -2-cyanobut-2-enethioamide

4-benzylpiperidine was used in place of isobutylamine, the reaction was performed in a similar method as described in Example 5 (5a) and the title compound was obtained. Yield 88%.

Mp 151-154° C.;

IR (KBr) νₘₐₓ 3283, 3173, 2187, 1605, 1542, 867, 749, 702 cm⁻¹;

¹H NMR (DMSO-d₆, 400 MHz) δ 1.26-1.32 (2H, m), 1.63-1.66 (2H, m), 1.80-1.90 (1H, m), 2.53 (2H, d, J=7.2 Hz), 3.02-3.08 (2H, m), 3.65 (2H, brd, J=13.6 Hz), 7.17-7.31 (1H, m), 8.18 (1H, br), 8.88 (1H, br);

MS (EI) m/z: 299 [M⁺], 240, 91;

Anal. Calcd for C₁₇H₂₁N₃S.0.3 H₂O: C, 66.98; H, 7.14; N, 13.78; S, 10.52. Found: C, 67.03; H, 6.97; N, 13.78; S, 10.36.

### (18b) 4-(4-benzylpiperidin-1-yl)-2-thioxo-1,2-dihydropyridine-3-carbonitrile

(2Z)-2-cyano-3-(4-benzylpiperidin-1-yl)but-2-enethioamide (0.53 g) which was produced in Example 18 (18a) was used in place of (2Z)-2-cyano-3-(dimethylamino)but-2-enethioamide and the reaction was performed in a similar method as described in Example 1 (1b). The aqueous layer was neutralized with 1N hydrochloric acid after was added toluene after the reaction terminated, the liquid was partitioned, and the deposited solid was separated by filtration and further washed with water and ethanol and a crude product (0.23 g) of the title compound was obtained.

¹H NMR (DMSO-d₆, 400 MHz) δ 1.20-1.31 (2H, m), 1.69 (2H, brd, J=10.9 Hz), 1.82-1.88 (1H, m), 2.54 (2H, d, J=7.1 Hz), 3.05-3.11 (2H, m), 4.07 (2H, brd, J=13.2 Hz), 6.45 (1H, d, J=7.5 Hz), 7.40-7.44 (1H, m), 12.57 (1H, br).

### (18c) 3-amino-4-(4-benzylpiperidin-1-yl)thieno[2,3-b]pyridine-2-carboxamide

A crude product (0.53 g) of 4-(4-benzylpiperidin-1-yl)-2-thioxo-1,2-dihydropyridine-3-carbonitrile which was produced in Example 18 (18b) was used in place of 4-(isobutylamino)-2-thioxo-1,2-dihydropyridine-3-carbonitrile, the reaction was performed in a similar method as described in Example 5 (5c) and the title compound was obtained (0.20 g). Yield 27% from (2Z)-2-cyano-3-(4-benzylpiperidin-1-yl)but-2-enethioamide.

Mp 220-221° C.;

IR (KBr) νₘₐₓ 3446, 3330, 3156, 1649, 1579, 1502, 1367, 958, 748, 700 cm⁻¹;

¹H NMR (DMSO-d₆, 400 MHz) δ 1.48-1.72 (5H, m), 2.52-2.73 (2H, m), 2.59 (2H, d, J=6.4 Hz), 3.30-3.33 (2H, m), 6.94 (2H, br), 6.99 (1H, d, J=5.3 Hz), 7.09 (2H, br), 7.18-7.32 (5H, m), 8.41 (1H, d, J=5.3 Hz);

MS (FAB) m/z: 367 [M+H]⁺;

Anal. Calcd for C₂₀H₂₂N₄SO.0.1 H₂O: C, 65.23; H, 6.08; N, 15.21; S, 8.71. Found: C, 65.18; H, 6.15; N, 15.35; S, 8.54.

### Example 19

### 3-amino-4-(4-hydroxypiperidin-1-yl)thieno[2,3-b]pyridine-2-carboxamide

(Exemplified Compound No. 1-32)

### (19a) (2Z)-2-cyano-3-(4-hydroxypiperidin-1-yl)but-2-enethioamide

4-hydroxypiperidine was used in place of isobutylamine, the reaction was performed in a similar method as described in Example 5 (5a) and the title compound was obtained. Yield 93%.

Mp 160-163° C.;

IR (KBr) νₘₐₓ 3368, 3167, 2180, 1634, 1536, 1415 cm⁻¹;

¹H NMR (DMSO-d_{6, 400} MHz) δ 1.44-1.52 (2H, m), 1.78-1.83 (2H, m), 2.27 (3H, s), 3.15-3.22 (2H, m), 3.50-3.56 (2H, m), 3.75-3.79 (1H, m), 4.82 (1H, d, J=3.8 Hz), 8.18 (1H, br), 8.88 (1H, br);

MS (FAB) m/z: 226 [M+H]⁺;

Anal. Calcd for C₁₀H₁₅N₃SO: C, 53.31; H, 6.71; N, 18.65; S, 14.23. Found: C, 53.14; H, 6.65; N, 18.54; S, 14.03.

### (19b) (2Z)-2-cyano-N-[(1E)-(dimethylamino)methylene]-3-(4-hydroxypiperidin-1-yl)but-2-enethioamide

(2Z)-2-cyano-3-(4-hydroxypiperidin-1-yl)but-2-enethioamide (0.22 g, 1.0 mmol) which was produced in Example 19 (19a) and N,N-dimethylformamide dimethylacetal (0.13 g, 1.1 mmol) were mixed with ethanol (3 mL) and the mixture was stirred at room temperature for three hours. The deposited solid was separated by filtration and further washed with ethanol and 0.27 g of the title compound was obtained (yield 96%).

¹H NMR (DMSO-d₆, 400 MHz) δ 1.49-1.57 (2H, m), 1.83-1.90 (2H, m), 2.50 (3H, s), 2.99 (3H, s), 3.15 (3H, s), 3.32-3.39 (2H, m), 3.63-3.69 (2H, m), 3.80-3.86 (1H, m), 4.88 (1H, d, J=3.9 Hz), 8.50 (1H, s);

### (19c) 3-amino-4-(4-hydroxypiperidin-1-yl)thieno[2,3-b]pyridine-2-carboxamide

(2Z)-2-cyano-N-[(1E)-(dimethylamino)methylene]-3-(4-hydroxypiperidin-1-yl)but-2-enethioamide (0.27 g) which was produced in Example 19 (19b) was dissolved in N,N-dimethylformamide (5 mL) and the mixture was stirred at 120° C. for one hour. The reaction liquid was cooled to room temperature and 8N aqueous solution of sodium hydroxide (0.3 mL) and 2-chloroacetamide (0.11 g, 1.2 mmol) were added. Water (5 mL) was added after the mixture was stirred at room temperature for one hour. The deposited solid was separated by filtration and washed with water and ethanol and 0.05 g of the title compound was obtained (yield 18%).

Mp 239-241° C.;

IR (KBr) νₘₐₓ 3445, 3425, 3330, 1645, 1592, 1376, 1045 cm⁻¹;

¹H NMR (DMSO-d₆, 400 MHz) δ 1.63-1.71 (2H, m), 1.90-1.98 (2H, m), 2.70-3.30 (4H, m), 3.50-3.80 (1H, m), 4.77 (1H, br), 6.91 (2H, br), 7.00 (1H, d, J=5.3 Hz), 7.06 (2H, br), 8.39 (1H, d, J=5.3 Hz);

MS (FAB) m/z: 293 [M+H]⁺;

Anal. Calcd for C₁₃H₁₆N₄SO₂.0.1 H₂O: C, 53.08; H, 5.55; N, 19.05; S, 10.90. Found: C, 53.09; H, 5.37; N, 18.77; S, 10.99.

### Example 20

### 3-amino-4-(4-acetoxypiperidin-1-yl)thieno[2,3-b]pyridine-2-carboxamide

(Exemplified Compound No. 1-34)

3-amino-4-(4-hydroxypiperidin-1-yl)thieno[2,3-b]pyridine-2-carboxamide (0.10 g, 0.3 mmol) which was produced in Example 19 (19c) and acetic acid anhydride (0.2 mL) were reacted overnight in tetrahydrofuran (5 mL) solvent at room temperature in the presence of a catalytic amount of N,N-dimethylaminopyridine. A saturated sodium bicarbonate aqueous solution (30 mL) was added to the reaction mixture and the aqueous layer was extracted with ethyl acetate (50 mL). The extract was dried over sodium sulfate and the solvent was evaporated under reduced pressure. The obtained solid was washed with ethyl acetate and 0.04 g of the title compound was obtained (yield 35%).

Mp 231-235° C.;

IR (KBr) νₘₐₓ 3439, 3422, 3323, 1710, 1645, 1580, 1268, 1035, 963 cm⁻¹;

¹H NMR (DMSO-d₆, 400 MHz) δ 1.82-2.10 (4H, m), 2.04 (3H, s), 2.70-3.45 (4H, m), 4.65-5.00 (1H, m), 6.91 (2H, br), 7.02 (1H, d, J=5.3 Hz), 7.09 (2H, br), 8.41 (1H, d, J=5.3 Hz);

MS (FAB) m/z: 335 [M+H]⁺;

Anal. Calcd for C₁₅H₁₈N₄SO₃.0.24 H₂O: C, 53.19; H, 5.50; N, 16.54; S, 9.47. Found: C, 53.47; H, 5.27; N, 16.20; S, 9.22.

### Example 21

### 3-amino-4-(4-phenyl-3,6-dihydropyridin-1(2H)-yl)thieno[2,3-b]pyridine-2-carboxamide

(Exemplified Compound No. 1-38)

### (21a) (2Z)-2-cyano-3-(4-phenyl-3,6-dihydropyridin-1(2H)-yl)but-2-enethioamide

4-phenyl-1,2,3,6-tetrahydropyridine was used in place of isobutylamine, the reaction was performed in a similar method as described in Example 5 (5a) and the title compound was obtained. Yield 99%.

Mp 170-171° C.;

IR (KBr) νₘₐₓ 3342, 3293, 3189, 2176, 1637, 1555, 1410, 872, 750 cm⁻¹;

¹H NMR (DMSO-d₆, 400 MHz) δ 2.36 (3H, s), 2.64-2.71 (2H, m), 3.64 (2H, t, J=5.5 Hz), 3.98-4.04 (2H, m), 6.16 (1H, brs), 7.24-7.45 (5H, m), 8.33 (1H, br), 9.02 (1H, br);

MS (FAB) m/z: 284 [M+H]⁺;

Anal. Calcd for C₁₆H₁₇N₃S.0.2 H₂O: C, 66.96; H, 6.11; N, 14.64; S, 11.17. Found: C, 67.02; H, 6.12; N, 14.62; S, 10.81.

### (21b) 4-phenyl-2′-thioxo-1′,2′,3,6-tetrahydro-2H-1,4′-bipyridine-3′-carbonitrile

(2Z)-2-cyano-3-(4-phenyl-3,6-dihydropyridine-1(2H)-yl)but-2-enethioamide (1.13 g) which was produced in Example 21 (21a) was used in place of (2Z)-2-cyano-3-(dimethylamino)but-2-enethioamide and the reaction was performed in a similar method as described in Example 1 (1b) and a crude product (0.13 g) of the title compound was obtained.

¹H NMR (DMSO-d₆, 400 MHz) δ 2.66-2.72 (2H, m), 3.88-3.91 (2H, m), 4.26-4.30 (2H, m), 6.24 (1H, brs), 6.56 (1H, d, J=7.8 Hz), 7.27-7.53 (6H, m), 12.70 (1H, br).

### (21c) 3-amino-4-(4-phenyl-3,6-dihydropyridin-1(2H)-yl)thieno[2,3-b]pyridine-2-carboxamide

A crude product (0.13 g) of 4-phenyl-2′-thioxo-1′,2′,3,6-tetrahydro-2H-1,4′-bipyridine-3′-carbonitrile which was produced in Example 21 (21b) was used in place of 4-(isobutylamino)-2-thioxo-1,2-dihydropyridine-3-carbonitrile, and the reaction was performed in a similar method as described in Example 5 (5c) and the title compound (0.06 g) was obtained. Yield 4% from (2Z)-2-cyano-3-(4-phenyl-3,6-dihydropyridine-1(2H)-yl)but-2-enethioamide

Mp 235° C. (decomposition);

IR (KBr) νₘₐₓ 3435, 3325, 1646, 1583, 1369, 954, 747 cm⁻¹;

¹H NMR (DMSO-d₆, 400 MHz) δ 2.62-2.76 (2H, m), 3.30-3.46 (2H, m), 3.77-3.86 (2H, m), 6.35 (1H, brs), 6.90 (2H, br), 7.09 (1H, d, J=5.3 Hz), 7.10 (2H, br), 7.25-7.51 (5H, m), 8.43 (1H, d, J=5.3 Hz);

MS (FAB) m/z: 351 [M+H]⁺;

Anal. Calcd for C₁₉H₁₈N₄SO.0.7 H₂O: C, 62.86; H, 5.39; N, 15.43; S, 8.87. Found: C, 63.16; H, 5.04; N, 15.23; S, 8.45.

### Example 22

### 3-amino-4-morpholin-4-ylthieno[2,3-b]pyridine-2-carboxamide

(Exemplified Compound No. 1-41)

### (22a) (2Z)-2-cyano-3-morpholin-1-ylbut-2-enethioamide

Morpholine was used in place of propylamine, the reaction was performed in a similar method as described in Example 4 (4a) and the title compound was obtained. Yield 85%.

Mp 156-160° C.;

IR (KBr) νₘₐₓ 3374, 3255, 3165, 2177, 1605, 1540, 1119, 985, 884 cm⁻¹;

¹H NMR (DMSO-d₆, 400 MHz) δ 2.26 (3H, s), 3.36-3.39 (4H, m), 3.64-3.67 (4H, m), 8.38 (1H, br), 9.06 (1H, br);

MS (FAB) m/z: 212 [M+H]⁺;

Anal. Calcd for C₉H₁₃N₃SO: C, 51.16; H, 6.20; N, 19.89; S, 15.18. Found: C, 51.15; H, 6.14; N, 19.73; S, 15.17.

### (22b) 4-morpholin-1-yl-2-thioxo-1,2-dihydropyridine-3-carbonitrile

4-(dimethoxymethyl) morpholine (Nucleosides and Nucleotides, 12, 1033-1046, (1993)) was used in place of N,N-dimethylformamide dimethylacetal and (2Z)-2-cyano-3-morpholin-1-ylbut-2-enethioamide was used in place of (2Z)-2-cyano-3-piperidin-1-ylbut-2-enethioamide and the reaction was performed in a similar method as described in Example 14 (14b). The aqueous solution obtained by removing insolubles by filtration from the reaction mixture was neutralized with 1N hydrochloric acid. The deposited solid was separated by filtration and washed with water and ethanol and a crude product of the title compound was obtained.

Yield 43%.

¹H NMR (DMSO-d₆, 400 MHz) δ 3.62-3.65 (4H, m), 3.69-3.71 (4H, m), 6.51 (1H, d, J=7.8 Hz), 7.52 (1H, d, J=7.8 Hz) 12.75 (1H, br).

### (22c) 3-amino-4-morpholin-4-ylthieno[2,3-b]pyridine-2-carboxamide

4-morpholin-1-yl-2-thioxo-1,2-dihydropyridine-3-carbonitrile which was produced in Example 22 (22b) was used in place of 4-piperidin-1-yl-2-thioxo-1,2-dihydropyridine-3-carbonitrile, the reaction was performed in a similar method as described in Example 14 (14c) and the title compound was obtained. Yield 64%.

Mp 232-234° C.;

IR (KBr) νₘₐₓ 3427, 3377, 3170, 1670, 1579, 1501, 1373, 1112, 969 cm⁻¹;

¹H NMR (DMSO-d₆, 400 MHz) δ 2.90-3.15 (4H, m), 3.84-3.86 (4H, m), 6.95 (2H, br), 7.05 (1H, d, J=5.3 Hz), 7.13 (2H, br), 8.46 (1H, d, J=5.3 Hz);

MS (FAB) m/z: 279 [M+H]⁺;

Anal. Calcd for C₁₂H₁₄N₄SO₂.0.3 H₂O: C, 50.80; H, 5.19; N, 19.75; S, 11.30. Found: C, 50.95; H, 4.86; N, 19.72; S, 11.28.

### Example 23

### 3-amino-4-(cis-2,6-dimethylmorpholin-4-yl)thieno[2,3-b]pyridine-2-carboxamide

(Exemplified Compound No. 1-42)

(2Z)-2-cyano-3-ethoxybut-2-enethioamide (340 mg, 2.0 mmol) and cis-2,6-dimethylmorpholine (288 mg, 2.5 mmol) were mixed with ethanol (3 mL) and the mixture was stirred at room temperature for four hours. The obtained residue by evaporating the solvent under reduced pressure was dissolved in N,N-dimethylformamide (3 mL) and N,N-dimethylformamide dimethylacetal (262 mg, 2.2 mmol) was added. The reaction mixture was stirred at 100° C. for one hour after stirring at room temperature for one hour. After cooled to room temperature, 8N aqueous solution of sodium hydroxide (0.4 mL) and 2-chloroacetamide (243 mg, 2.6 mmol) were added to the reaction mixture. The reaction mixture was partitioned with water (50 mL) and ethyl acetate (50 mL) after stirring at room temperature for one hour. The solvent was evaporated under reduced pressure after the organic layer was dried over sodium sulfate. Residue was purified by silica gel column chromatography (ethyl acetate/ethanol/triethylamine=30:1:1) and 100 mg of the title compound was obtained (16%).

Mp 242-244° C.;

IR (KBr) νₘₐₓ 3441, 3326, 3172, 1650, 1584, 1502, 1373, 1082, 1010 cm⁻¹;

¹H NMR (DMSO-d₆, 400 MHz) δ 1.13 (6H, d, J=6.3 Hz), 2.40-2.50 (2H, m), 3.24 (2H, brd, J=11.3 Hz), 3.90-3.98 (2H, m), 6.92 (2H, br), 7.03 (1H, d, J=5.1 Hz), 7.16 (2H, br), 8.45 (1H, d, J=5.1 Hz);

MS (EI) m/z: 306 [M⁺], 202;

Anal. Calcd for C₁₄H₁₈N₄SO₂: C, 54.88; H, 5.92; N, 18.29; S, 10.47. Found: C, 54.88; H, 5.99; N, 18.37; S, 10.43.

### Example 24

### 3-amino-4-thiomorpholin-4-ylthieno[2,3-b]pyridine-2-carboxamide

(Exemplified Compound No. 1-44)

### (24a) (2Z)-2-cyano-3-thiomorpholin-4-ylbut-2-enethioamide

Thiomorpholine was used in place of isobutylamine, the reaction was performed in a similar method as described in Example 5 (5a) and the title compound was obtained. Yield 73%.

Mp 157-160° C. (decomposition);

IR (KBr) νₘₐₓ 3346, 3285, 3180, 2183, 1534, 953, 865 cm⁻¹;

¹H NMR (DMSO-d₆, 400 MHz) δ 2.26 (3H, s), 2.72-2.74 (4H, m), 3.58-3.61 (4H, m), 8.43 (1H, br), 9.11 (1H, br);

MS (FAB) m/z: 228 [M+H]⁺;

Anal. Calcd for C₉H₁₃N₃S₂.0.1 H₂O: C, 47.17; H, 5.81; N, 18.34; S, 27.98. Found: C, 47.27; H, 5.82; N, 18.62; S, 27.84.

### (24b) 4-thiomorpholin-4-yl-2-thioxo-1,2-dihydropyridine-3-carbonitrile

(2Z)-2-cyano-3-thiomorpholin-4-ylbut-2-enethioamide (250 mg) which was produced in Example 24 (24a) was used in place of (2Z)-2-cyano-3-(dimethylamino)but-2-enethioamide and the reaction was performed in a similar method as described in Example 1 (1b) and a crude product (157 mg) of the title compound was obtained.

¹H NMR (DMSO-d₆, 400 MHz) δ 2.74-2.77 (4H, m), 3.84-3.87 (4H, m), 6.49 (1H, d, J=7.4 Hz), 7.48 (1H, brd, J=7.4 Hz), 12.73 (1H, br).

### (24c) 3-amino-4-thiomorpholin-4-ylthieno[2,3-b]pyridine-2-carboxamide

A crude product (157 mg) of 4-thiomorpholin-4-yl-2-thioxo-1,2-dihydropyridine-3-carbonitrile which was produced in Example 24 (24b) was dissolved in N,N-dimethylformamide (2 mL) and 8N aqueous solution of sodium hydroxide (0.2 mL) and 2-chloroacetamide (74 mg, 0.8 mmol) were added. Water (2 mL) was added to the reaction mixture after stirring at room temperature for one hour. The deposited solid was separated by filtration and washed with water and ethanol. The obtained crystal was further heated and the mixture was stirred in ethyl acetate. After cooling, crystal was separated by filtration and 37 mg of the title compound was obtained. Yield 11% from (2Z)-2-cyano-3-thiomorpholin-4-ylbut-2-enethioamide.

Mp 255-258° C.;

IR (KBr) νₘₐₓ 3439, 3322, 3166, 1656, 1582, 1502, 1374, 1344, 935 cm⁻¹;

¹H NMR (DMSO-d₆, 400 MHz) δ 2.50-3.60 (8H, m), 6.93 (2H, br), 7.04 (1H, d, J=5.3 Hz), 7.10 (2H, br), 8.43 (1H, d, J=5.3 Hz);

MS (EI) m/z: 294 [M⁺], 202;

Anal. Calcd for C₁₂H₁₄N₄S₂O: C, 48.96; H, 4.79; N, 19.03; S, 21.78. Found: C, 48.90; H, 4.70; N, 19.04; S, 21.73.

### Example 25

### 3-amino-4-azepan-1-ylthieno[2,3-b]pyridine-2-carboxamide

(Exemplified Compound No. 1-39)

### (25a) (2Z)-3-azepan-1-yl-2-cyanobut-2-enethioamide

Hexamethyleneimine was used in place of isobutylamine and the reaction was performed in a similar method as described in Example 5 (5a) and the title compound was obtained. Yield 94%.

Mp 158-161° C.;

IR (KBr) νₘₐₓ 3405, 3292, 3184, 2192, 1607, 1517, 1009, 870 cm⁻¹;

¹H NMR (CDCl₃, 400MHz) δ 1.61-1.70 (4H, m), 1.87-1.95 (4H, m), 2.74 (3H, s), 3.70-3.73 (4H, m), 6.69 (2H, br);

MS (EI) m/z: 223 [M⁺], 190;

Anal. Calcd for C₁₁H₁₇N₃S: C, 59.16; H, 7.67; N, 18.81; S, 14.36. Found: C, 59.10; H, 7.72; N, 18.67; S, 14.07.

### (25b) 4-azepan-1-yl-2-thioxo-1,2-dihydropyridine-3-carbonitrile

(2Z)-3-azepan-1-yl-2-cyanobut-2-enethioamide (0.42 g) which was produced in Example 25 (25a) was used in place of (2Z)-2-cyano-3-(dimethylamino)but-2-enethioamide and the reaction was performed in a similar method as described in Example 1 (1b) and a crude product (0.38 g) of the title compound was obtained.

¹H NMR (DMSO-d₆, 400 MHz) δ 1.49-1.54 (4H, m), 1.73-1.80 (4H, m), 3.75-3.78 (4H, m), 6.39 (1H, d, J=7.8 Hz), 7.37 (1H, dd, J=6.0, 7.8 Hz), 12.46 (1H, br).

### (25c) 3-amino-4-azepan-1-ylthieno[2,3-b]pyridine-2-carboxamide

A crude product (0.38 g) of 4-azepan-1-yl-2-thioxo-1,2-dihydropyridine-3-carbonitrile which was produced in Example 25 (25b) was used in place of 4-(isobutylamino)-2-thioxo-1,2-dihydropyridine-3-carbonitrile, the reaction was performed in a similar method as described in Example 5 (5c) and 0.39 g of the title compound was obtained. Yield 71% from (2Z)-3-azepan-1-yl-2-cyanobut-2-enethioamide.

Mp 219-221° C.;

IR (KBr) νₘₐₓ 3436, 3298, 3131, 1673, 1582, 1371, 930 cm⁻¹;

¹H NMR (DMSO-d₆, 400 MHz) δ 1.65-1.78 (8H, m), 3.25-3.28 (4H, m), 7.04 (4H, br), 7.05 (1H, d, J=5.4 Hz), 8.38 (1H, d, J=5.4 Hz);

MS (EI) m/z: 290 [M⁺], 202.

### Example 26

### 3-amino-4-azocan-1-ylthieno[2,3-b]pyridine-2-carboxamide

(Exemplified Compound No. 1-40)

### (26a) (2Z)-3-azocan-1-yl-2-cyanobut-2-enethioamide

Heptamethyleneimine was used in place of isobutylamine, the reaction was performed in a similar method as described in Example 5 (5a) and the title compound was obtained. Yield 85%.

Mp 149-150° C.;

IR (KBr) νₘₐₓ 3340, 3276, 3164, 2176, 1631, 1558, 848 cm⁻¹;

¹H NMR (DMSO-d₆, 400 MHz) δ 1.45-1.52 (6H, m), 1.72-1.75 (4H, m), 2.34 (3H, s), 3.55-3.58 (4H, m), 8.30 (1H, br), 8.92 (1H, br);

MS (FAB) m/z: 238 [M+H]⁺;

Anal. Calcd for C₁₂H₁₉N₃S.0.06 H₂O: C, 60.45; H, 8.08; N, 17.62; S, 13.45. Found: C, 60.43; H, 7.93; N, 17.67; S, 13.58.

### (26b) 4-azocan-1-yl-2-thioxo-1,2-dihydropyridine-3-carbonitrile

(2Z)-3-azocan-1-yl-2-cyanobut-2-enethioamide (0.39 g) which was produced in Example 26 (26a) was used in place of (2Z)-2-cyano-3-(dimethylamino)but-2-enethioamide and the reaction was performed in a similar method as described in Example 1 (1b) and a crude product (0.35 g) of the title compound was obtained.

¹H NMR (DMSO-d₆, 400 MHz) δ 1.44-1.57 (6H, m), 1.71-1.74 (4H, m), 3.80-3.83 (4H, m), 6.38 (1H, d, J=7.7 Hz), 7.35-8.38 (1H, m), 12.48 (1H, br).

### (26c) 3-amino-4-azocan-1-ylthieno[2,3-b]pyridine-2-carboxamide

A crude product (0.35 g) of 4-azocan-1-yl-2-thioxo-1,2-dihydropyridine-3-carbonitrile which was produced in Example 26 (26b) was used in place of 4-(isobutylamino)-2-thioxo-1,2-dihydropyridine-3-carbonitrile, and the reaction was performed in a similar method as described in Example 5 (5c) and 0.36 g of the title compound was obtained. Yield 72% from (2Z)-3-azocan-1-yl-2-cyanobut-2-enethioamide.

Mp 222-224° C.;

IR (KBr) νₘₐₓ 3440, 3300, 3132, 1667, 1579, 1499, 1372, 990 cm⁻¹;

¹H NMR (DMSO-d₆, 400 MHz) δ 1.60-1.75 (10H, m), 3.34-3.36 (4H, m), 7.03 (2H, br), 7.04 (2H, br), 7.08 (1H, d, J=5.4 Hz), 8.35 (1H, d, J=5.4 Hz);

MS (FAB) m/z: 305 [M+H]⁺;

Anal. Calcd for C₁₅H₂₀N₄SO: C, 58.84; H, 6.65; N, 18.30; S, 10.47. Found: C, 58.84; H, 6.53; N, 18.30; S, 10.37.

### Example 27

### 3-amino-4-(3,4-dihydroisoquinoline-2(1H)-yl)thieno[2,3-b]pyridine-2-carboxamide

(Exemplified Compound No. 1-35)

### (27a) (2Z) 2-cyano-3-(3,4-dihydroisoquinoline-2(1H)-yl)but-2-enethioamide

1,2,3,4-tetrahydroisoquinoline was used in place of isobutylamine, the reaction was performed in a similar method as described in Example 5 (5a) and the title compound was obtained. Yield 93%.

IR (KBr) νₘₐₓ 3284, 3167, 2186, 1609, 1538, 878, 752 cm⁻¹;

¹H NMR (DMSO-d₆, 400 MHz) δ 2.38 (3H, s), 2.95 (2H, t, J=5.8 Hz), 3.62 (2H, t, J=5.8 Hz), 4.54 (2H, s), 7.10-7.21 (4H, m), 8.32 (1H, br) 8.98 (1H, br);

MS (EI) m/z: 257 [M⁺], 224.

### (27b) 4-(3,4-dihydroisoquinoline-2(1H)-yl)-2-thioxo-1,2-dihydropyridine-3-carbonitrile

(2Z)-2-cyano-3-(3,4-dihydroisoquinoline-2(1H)-yl)but-2-enethioamide (470 mg) which was produced in Example 27 (27a) was used in place of (2Z)-2-cyano-3-(dimethylamino)but-2-enethioamide and the reaction was performed in a similar method as described in Example 1 (1b) and a crude product (175 mg) of the title compound was obtained.

¹H NMR (DMSO-d₆, 400 MHz) δ 3.00 (2H, t, J=5.9 Hz), 3.88 (2H, t, J=5.9 Hz), 4.76 (2H, s), 6.58 (1H, d, J=7.4 Hz), 7.22-7.25 (4H, m), 7.51-7.54 (1H, m) 12.72 (1H, br).

### (27c) 3-amino-4-(3,4-dihydroisoquinoline-2(1H)-yl)thieno[2,3-b]pyridine-2-carboxamide

A crude product (175 mg) of 4-(3,4-dihydroisoquinoline-2(1H)-yl)-2-thioxo-1,2-dihydropyridine-3-carbonitrile which was produced in Example 27 (27b) was used in place of 4-(isobutylamino)-2-thioxo-1,2-dihydropyridine-3-carbonitrile, and the reaction was performed in a similar method as described in Example 5 (5c) and the title compound was obtained (71 mg). Yield 12% from (2Z)-2-cyano-3-(3,4-dihydroisoquinoline-2(1H)-yl)but-2-enethioamide.

Mp 244-245° C.;

IR (KBr) νₘₐₓ 3395, 3325, 3144, 1659, 1585, 1502, 1378, 739 cm⁻¹;

¹H NMR (DMSO-d₆, 400 MHz) δ 2.94-3.10 (2H, m), 3.33-3.50 (2H, m), 4.28 (2H, brs), 6.85 (2H, br), 7.08 (1H, d, J=5.5 Hz), 7.09 (2H, br), 7.17-7.19 (4H, m), 8.44 (1H, d, J=5.5 Hz);

MS (FAB) m/z: 325 [M+H]⁺;

Anal. Calcd for C₁₇H₁₆N₄SO: C, 62.94; H, 4.97; N, 17.27; S, 9.88. Found: C, 62.69; H, 5.24; N, 17.21; S, 9.85.

### Example 28

### 3-amino-4-(trans-perhydroisoquinolin-2-yl)thieno[2,3-b]pyridine-2-carboxamide

(Exemplified Compound No. 1-36)

### (28a) (2Z)-2-cyano-3-(trans-perhydroisoquinolin-2-yl)but-2-enethioamide

Trans-perhydroisoquinoline was used in place of isobutylamine, the reaction was performed in a similar method as described in Example 5 (5a) and the title compound was obtained. Yield 41%.

Mp 142-147° C.;

IR (KBr) νₘₐₓ 3375, 3277, 3163, 2184, 1606, 1537, 872 cm⁻¹;

¹H NMR (DMSO-d₆, 400 MHz) δ 0.88-1.72 (12H, m), 2.27 (3H, s), 2.71-2.77 (1H, m), 3.04-3.11 (1H, m), 3.49-3.51 (1H, m), 3.64-3.68 (1H, m), 8.15 (1H, br), 8.87 (1H, br);

MS (FAB) m/z: 264 [M+H]⁺;

Anal. Calcd for C₁₄H₂₁N₃S: C, 63.84; H, 8.04; N, 15.95; S, 12.17. Found: C, 63.92; H, 7.98; N, 15.93; S, 11.91.

### (28b) 4-(trans-perhydroisoquinolin-2-yl)-2-thioxo-1,2-dihydropyridine-3-carbonitrile

(2Z)-2-cyano-3-(trans-perhydroisoquinolin-2-yl)but-2-enethioamide (210 mg) which was produced in Example 28 (28a) was used in place of (2Z)-2-cyano-3-(dimethylamino)but-2-enethioamide and the reaction was performed in a similar method as described in Example 1 (1b) and a crude product (153 mg) of the title compound was obtained.

¹H NMR (DMSO-d₆, 400 MHz) δ 0.91-1.74 (12H, m), 2.75-2.81 (1H, m), 3.09-3.15 (1H, m), 3.94-4.00 (1H, m), 4.10-4.17 (1H, m), 6.48 (1H, d, J=7.4 Hz), 7.39-7.43 (1H, m), 12.56 (1H, br).

### (28c) 3-amino-4-(trans-perhydroisoquinolin-2-yl)thieno[2,3-b]pyridine-2-carboxamide

A crude product (153 mg) of 4-(trans-perhydroisoquinolin-2-yl)-2-thioxo-1,2-dihydropyridine-3-carbonitrile which was produced in Example 28 (28b) was used in place of 4-(isobutylamino)-2-thioxo-1,2-dihydropyridine-3-carbonitrile and the reaction was performed in a similar method as described in Example 5 (5c) and the title compound was obtained (115 mg). Yield 44% from (2Z)-2-cyano-3-(trans-perhydroisoquinolin-2-yl)but-2-enethioamide.

Mp 285-288° C.;

IR (KBr) νₘₐₓ 3406, 3323, 3143, 1656, 1584, 1502, 1377, 951 cm⁻¹;

¹H NMR (DMSO-d₆, 400 MHz) δ 0.92-1.76 (12H, m), 2.34-2.45 (1H, m), 2.66-2.79 (1H, m), 3.14-3.22 (1H, m), 3.30-3.38 (1H, m), 6.92 (2H, br), 7.01 (1H, d, J=5.1 Hz), 7.10 (2H, br), 8.42 (1H, d, J=5.1 Hz);

MS (FAB) m/z: 331 [M+H]⁺;

Anal. Calcd for C₁₇H₂₂N₄SO: C, 61.79; H, 6.71; N, 16.95; S, 9.70. Found: C, 61.63; H, 6.71; N, 16.94; S, 9.74.

### Example 29

### 3-amino-4-(cis-perhydroisoquinolin-2-yl)thieno[2,3-b]pyridine-2-carboxamide

(Exemplified Compound No. 1-36)

### (29a) (2Z)-2-cyano-3-(cis-perhydroisoquinolin-2-yl)but-2-enethioamide

cis-perhydroisoquinoline was used in place of isobutylamine, the reaction was performed in a similar method as described in Example 5 (5a) and the title compound was obtained. Yield 95%.

Mp 173-175° C.;

IR (KBr) νₘₐₓ 3444, 3250, 3160, 2188, 1603, 1542, 867 cm⁻¹;

¹H NMR (DMSO-d₆, 400 MHz) δ 1.23-1.95 (12H, m), 2.27 (3H, s), 3.12-3.25 (2H, m), 3.41-3.54 (2H, m), 8.12 (1H, br), 8.85 (1H, br);

MS (FAB) m/z: 264 [M+H]⁺;

Anal. Calcd for C₁₄H₂₁N₃S: C, 63.84; H, 8.04; N, 15.95; S, 12.17. Found: C, 63.53; H, 8.10; N, 15.67; S, 12.32.

### (29b) 4-(cis-perhydroisoquinolin-2-yl)-2-thioxo-1,2-dihydropyridine-3-carbonitrile

(2Z)-2-cyano-3-(cis-perhydroisoquinolin-2-yl)but-2-enethioamide (490 mg) which was produced in Example 29 (29a) was used in place of (2Z)-2-cyano-3-(dimethylamino)but-2-enethioamide and the reaction was performed in a similar method as described in Example 1 (1b) and a crude product (370 mg) of the title compound was obtained.

¹H NMR (DMSO-d₆, 400 MHz) δ 1.22-1.95 (12H, m), 3.10-3.43 (2H, m), 3.78-3.94 (2H, m), 6.47 (1H, d, J=7.4 Hz), 7.40 (1H, d, J=7.4 Hz), 12.40 (1H, br).

### (29c) 3-amino-4-(cis-perhydroisoquinolin-2-yl)thieno[2,3-b]pyridine-2-carboxamide

A crude product (370 mg) of 4-(cis-perhydroisoquinolin-2-yl)-2-thioxo-1,2-dihydropyridine-3-carbonitrile which was produced in Example 29 (29b) was used in place of 4-(isobutylamino)-2-thioxo-1,2-dihydropyridine-3-carbonitrile, the reaction was performed in a similar method as described in Example 5 (5c) and the title compound was obtained (200 mg). Yield 33% from (2Z)-2-cyano-3-(cis-perhydroisoquinolin-2-yl)but-2-enethioamide.

Mp 224-226° C.;

IR (KBr) νₘₐₓ 3443, 3323, 3179, 1646, 1582, 1503, 1370, 957 cm⁻¹;

¹H NMR (DMSO-d₆, 400 MHz) δ 1.20-2.25 (12H, m), 2.92-3.46 (4H, m), 6.93 (2H, br), 7.03 (1H, d, J=5.1 Hz), 7.08 (2H, br), 8.40 (1H, d, J=5.1 Hz);

MS (EI) m/z: 330 [M⁺], 285;

Anal. Calcd for C₁₇H₂₂N₄SO.0.1H₂O: C, 61.46; H, 6.73; N, 16.86; S, 9.65. Found: C, 61.37; H, 6.76; N, 16.80; S, 9.68.

### Example 30

### 3-amino-4-(1,4-oxazepan-4-yl)thieno[2,3-b]pyridine-2-carboxamide

(Exemplified Compound No. 1-43)

### (30a) (2Z)-2-cyano-3-(1,4-oxazepan-4-yl)but-2-enethioamide

Homomorpholine was used in place of isobutylamine, the reaction was performed in a similar method as described in Example 5 (5a) and the title compound was synthesized.

Pale yellow powder

Mp 151-156° C.;

IR (KBr) νₘₐₓ 3348, 3285, 3174, 2184, 1632, 1518, 1417, 1127, 1075, 878, 814 cm⁻¹;

¹H NMR (DMSO-d₆, 400 MHz) δ 1.86-1.92 (2H, m), 2.31 (3H, s), 3.57 (2H, t, J=5.5 Hz), 3.62 (2H, t, J=5.5 Hz), 3.66 (2H, t, J=5.5 Hz), 3.77 (2H, t, J=5.5 Hz), 8.29 (1H, brs), 8.94 (1H, brs);

HRMS m/z calcd for C₁₀H₁₆ON₃S 226.1014. Found: 226.1019;

MS (FAB) m/z: 226 [M+H]⁺, 209, 192, 165, 65, 51.

### (30b) 4-(1,4-oxazepan-4-yl)-2-thioxo-1,2-dihydropyridine-3-carbonitrile

(2Z)-2-cyano-3-(1,4-oxazepan-4-yl)but-2-enethioamide which was produced in Example 30 (30a) was used in place of (2Z)-2-cyano-3-(isobutylamino)but-2-enethioamide and the reaction was performed in a similar method as described in Example 5 (5b) and the title compound was obtained.

White powder

Mp 228-233° C. (decomposition);

IR (KBr) νₘₐₓ 3119, 2952, 2210, 1625, 1520, 1252, 1117, 928, 780 cm⁻¹;

¹H NMR (DMSO-d₆, 400 MHz) δ 1.89-1.94 (2H, m), 3.67 (2H, t, J=5.5 Hz), 3.77-3.79 (2H, m), 3.33-3.37 (4H, m), 6.44 (1H, d, J=7.8 Hz), 7.42 (1H, d, J=7.8 Hz), 12.59 (1H, brs); HRMS m/z calcd for C₁₁H₁₃ON₃S 235.0779. Found: 235.0790;

MS (EI) m/z: 235 [M⁺], 204, 190, 177, 164, 150, 136, 108, 76, 70, 41.

### (30c) 3-amino-4-(1,4-oxazepan-4-yl)thieno[2,3-b]pyridine-2-carboxamide

4-(1,4-oxazepan-4-yl)-2-thioxo-1,2-dihydropyridine-3-carbonitrile which was produced in Example 30 (30b) was used in place of 4-(isobutylamino)-2-thioxo-1,2-dihydropyridine-3-carbonitrile and the reaction was performed in a similar method as described in Example 5 (5c) and the title compound was obtained.

White powder

Mp 175-176° C.;

IR (KBr) νₘₐₓ 3445, 3301, 3141, 1671, 1585, 1497, 1370, 1153, 1060, 940 cm⁻¹;

¹H NMR(DMSO-d₆, 400 MHz) δ 1.99-2.05 (2H, m), 3.30-3.34 (4H, m), 3.78-3.84 (4H, m), 7.05 (2H, brs), 7.08-7.10 (3H, m), 8.41 (1H, d, J=5.5 Hz);

HRMS m/z calcd for C₁₃H₁₇O₂N₄S 293.1073. Found: 293.1067;

MS (FAB) m/z: 293 [M+H]⁺, 276, 237, 183, 165, 120, 65;

Anal. Calcd for C₁₃H₁₆N₄O₂S.0.28 H₂O: C, 52.50; H, 5.61; N, 18.84; S, 10.78. Found: C, 52.32; H, 5.41; N, 19.03, S, 10.77.

### Example 31

### 3-amino-4-(4-phenylpiperidin-1-yl)thieno[2,3-b]pyridine-2-carboxamide

(Exemplified Compound No. 1-26)

### (31a) (2Z)-2-cyano-3-(4-phenylpiperidin-1-yl)but-2-enethioamide

4-phenylpiperidine was used in place of isobutylamine and the reaction was performed in a similar method as described in Example 5 (5a) and the title compound was synthesized.

Pale yellow powder

Mp 171-172° C.;

IR (KBr) νₘₐₓ 3389, 3263, 3162, 2185, 1599, 1535, 1364, 1232, 980, 855, 764, 701 cm⁻¹;

¹H NMR(DMSO-d₆, 400 MHz) δ 1.69-1.86 (4H, m), 2.33 (3H, s), 2.83-2.91 (1H, m), 3.22 (2H, t, J=12.1 Hz), 3.76 (2H, d, J=12.1 Hz), 7.19-7.83 (5H, m), 8.28 (1H, brs), 8.98 (1H, brs);

HRMS m/z calcd for C₁₆H₂₀N₃S 286.1378. Found: 286.1372;

MS (FAB) m/z: 286 [M+H]⁺, 252, 227, 186, 80, 56, 41;

Anal. Calcd for C₁₆H₁₉N₃S: C, 67.33; H, 6.71; N, 14.72; S, 11.23. Found: C, 67.10; H, 6.75; N, 14.65, S, 11.17.

### (31b) (2Z)-2-cyano-N-[(1E)-(dimethylamino)methylene]-3-(4-phenylpiperidin-1-yl)but-2-enethioamide

(2Z)-2-cyano-3-(4-phenylpiperidin-1-yl)but-2-enethioamide (373 mg, 1.3 mmol) which was produced in Example 31 (31a) and N,N-dimethylformamide dimethylacetal (312 mg, 2.6 mmol) were mixed with ethanol (12 mL) and the mixture was stirred for 18 hours. The deposited solid was separated by filtration and 446 mg of the title compound was obtained (yield 70%).

Yellow powder

Mp 151-152° C.;

IR (KBr) νₘₐₓ 2920, 2182, 1614, 1520, 1333, 1289, 1191, 975, 767, 704 cm⁻¹;

¹H NMR(DMSO-d₆, 500 MHz) δ 1.79-1.91 (4H, m), 2.55 (3H, s), 2.94-3.00 (1H, m), 3.02 (3H, s) 3.18 (3H, s), 3.30-3.36 (2H, m), 3.93 (2H, d, J=13.7 Hz), 7.20-7.83 (5H, m), 8.55 (1H, s);

HRMS m/z calcd for C₁₉H₂₅N₄S 341.1800. Found: 341.1797;

MS (FAB) m/z: 341 [M+H]⁺, 273, 246, 200, 165, 63;

Anal. Calcd for C₁₉H₂₄N₄S.0.26 H₂O: C, 66.11; H, 7.16; N, 16.23; S, 9.29. Found: C, 66.15; H, 6.97; N, 16.14, S, 8.99.

### (31c) 3-amino-4-(4-phenylpiperidin-1-yl)thieno[2,3-b]pyridine-2-carboxamide

N,N-dimethylformamide (1.8 mL) solution of (2Z)-2-cyano-N-[(1E)-(dimethylamino) methylene]-3-(4-phenylpiperidin-1-yl)but-2-enethioamide (299 mg, 0.88 mmol) which was produced in Example 31 (31b) was stirred at 80° C. for 15 minutes. 8N aqueous solution of sodium hydroxide (0.37 mL) and 2-chloroacetamide (99 mg, 1.1 mmol) were added after the reaction mixture was cooled to room temperature. Water and ethyl acetate were added after the mixture was stirred at room temperature for one hour. The deposited solid was separated by filtration and 310 mg of the title compound was obtained (yield 58%).

Pale yellow crystal

Mp 226-233° C.;

IR (KBr) νₘₐₓ 3445, 3315, 3130, 1647, 1580, 1501, 1371, 1229, 1052, 959, 701 cm⁻¹;

¹H NMR(DMSO-d₆, 500 MHz) δ 1.90 (2H, d, J=11.7 Hz), 2.03 (2H, q, J=11.7 Hz), 2.70 (1H, t, J=11.7 Hz), 2.82-2.91 (2H, m), 3.45 (2H, d, J=11.7 Hz), 7.02 (2H, brs), 7.07 (1H, d, J=5.4 Hz), 7.10 (2H, brs), 7.22 (1H, t, J=7.3 Hz), 7.33 (2H, t, J=7.3 Hz), 7.38 (2H, d, J=7.3 Hz), 8.46 (1H, d, J=5.4 Hz);

HRMS m/z calcd for C₁₉H₂₀ON₄S 352.1358. found: 352.1358;

MS (FAB) m/z: 353 [M+H]⁺, 273, 246, 200, 165, 63.

### Example 32

### 3-amino-4-(1-oxothiomorpholine-4-yl)thieno[2,3-b]pyridine-2-carboxamide

(Exemplified Compound No. 1-45)

3-amino-4-(thiomorpholine-4-yl)thieno[2,3-b]pyridine-2-carboxamide (73 mg, 0.25 mmol) which was produced in Example 24 (24c) was dissolved in methanol and an aqueous solution (1 mL) of sodium periodate (59 mg, 0.28 mmol) was added. A saturated saline solution was added to the reaction liquid after stirring at room temperature for one hour and the aqueous layer was extracted with a mixed solvent of methylene chloride/2-propanol (4:1). The extract was concentrated after drying over sodium sulfate under reduced pressure. The residue was washed with ether and 78 mg of the title compound was obtained (yield 92%).

Pale yellow powder

Mp 165-172° C.;

IR (KBr) νₘₐₓ 3431, 3323, 3183, 1649, 1589, 1500, 1375, 1057, 933 cm⁻¹;

¹H NMR(DMSO-d₆, 500 MHz) δ 2.95-3.04 (2H, m), 3.21-3.32 (4H, m), 3.52-3.57 (2H, m), 6.99 (2H, brs), 7.15 (3H, brs), 8.48 (1H, d, J=5.4 Hz);

HRMS m/z calcd for C₁₂H₁₄O₂N₄S₂ 310.0558. found 310.0555;

MS (EI) m/z: 310 [M⁺], 278, 261, 244, 230, 202, 189, 176, 148, 122, 101, 76.

### Example 33

### 3-amino-4-(1,4-thiazepan-4-yl)thieno[2,3-b]pyridine-2-carboxamide

(Exemplified Compound No. 1-46)

### (33a) (2Z)-2-cyano-3-(1,4-thiazepan-4-yl)but-2-enethioamide

Homothiomorpholine (J. Org. Chem., 25, 1953-1956 (1960)) was used in place of isobutylamine and the reaction was performed in a similar method as described in Example 5 (5a) and the title compound was synthesized.

White powder

Mp 138-142° C.;

IR (KBr) νₘₐₓ 3438, 23284, 3173, 1597, 1533, 1409, 1251, 867, 569 cm⁻¹;

¹H NMR(DMSO-d₆, 400 MHz) δ 1.95-2.01 (2H, m), 2.33 (3H, s), 2.67 (2H, t, J=5.9 Hz), 2.94 (2H, t, J=5.5 Hz), 3.65 (2H, t, J=5.9 Hz), 3.70 (2H, t, J=5.5 Hz), 8.46 (1H, brs), 9.07 (1H, brs);

HRMS m/z calcd for C₁₀H₁₅N₃S₂ 241.0707. found 241.0707;

MS (EI) m/z: 241 [M⁺], 208, 182, 142, 135, 121, 96, 68, 59, 43;

Anal. Calcd for C₁₀H₁₅N₃S₂: C, 49.76; H, 6.26; N, 17.41; S, 26.57. Found: C, 49.47; H, 6.32; N, 17.14, S, 26.48.

### (33b) 4-(1,4-thiazepan-4-yl)-2-thioxo-1,2-dihydropyridine-3-carbonitrile

(2Z)-2-cyano-3-(1,4-thiazepan-4-yl)but-2-enethioamide which was produced in Example 33 (33a) was used in place of (2Z)-2-cyano-3-(isobutylamino)but-2-enethioamide and the reaction was performed in a similar method as described in Example 5 (5b) and the title compound was obtained.

Yellow powder

Mp 265-270° C.;

IR (KBr) νₘₐₓ 3115, 2949, 2207, 1625, 1524, 1256, 1136, 943, 788 cm⁻¹;

¹H NMR(DMSO-d₆, 400 MHz) δ 1.96 (2H, quint, J=5.5 Hz), 2.62 (2H, t, J=5.9 Hz), 2.90 (2H, t, J=5.9 Hz), 3.97 (2H, t, J=5.9 Hz), 4.04 (2H, t, J=5.9 Hz), 6.42 (1H, d, J=7.8 Hz), 7.37 (1H, d, J=7.8 Hz), 12.48 (1H, brs);

HRMS m/z calcd for C₁₁H₁₃N₃S₂ 251.0551. found 251.0553;

MS (EI) m/z: 251 [M⁺], 236, 223, 204, 190, 177, 164, 150, 136, 108, 60;

Anal. Calcd for C₁₁H₁₃N₃S₂: C, 52.56; H, 5.21; N, 16.72; S, 25.51. Found: C, 52.41; H, 5.37; N, 17.01, S, 25.62.

### (33c) 3-amino-4-(1,4-thiazepan-4-yl)thieno[2,3-b]pyridine-2-carboxamide

4-(1,4-thiazepan-4-yl)-2-thioxo-1,2-dihydropyridine-3-carbonitrile which was produced in Example 33 (33b) was used in place of 4-(isobutylamino)-2-thioxo-1,2-dihydropyridine-3-carbonitrile and the reaction was performed in a similar method as described in Example 5 (5c) and the title compound was obtained.

Pale yellow powder

Mp 171-174° C.;

IR (KBr) νₘₐₓ 3416, 3302, 3171, 1645, 1582, 1498, 1367, 1259, 1125, 481 cm⁻¹;

¹H NMR(DMSO-d₆, 500 MHz) δ 1.94-1.99 (2H, m), 2.76 (2H, t, J=5.9 Hz), 2.98 (2H, t, J=5.9 Hz), 3.29-3.32 (2H, m), 3.44-3.46 (2H, m), 7.07 (2H, brs), 7.20 (1H, d, J=5.4 Hz), 7.41 (2H, brs), 8.46 (1H, d, J=5.4 Hz);

HRMS m/z calcd for C₁₃H16ON₄S₂ 308.0765. found 308.0767;

MS (EI) m/z: 308 [M⁺], 276, 244, 230, 202, 188, 176, 148, 122, 78, 45;

Anal. Calcd for C₁₃H₁₆N₄OS₂.0.66H₂O: C, 48.75; H, 5.45; N, 17.49. Found: C, 48.44; H, 5.38; N, 17.79.

### Example 34

### 3-amino-4-(1-oxo-1,4-thiazepan-4-yl)thieno[2,3-b]pyridine-2-carboxamide

(Exemplified Compound No. 1-47)

3-amino-4-(1,4-thiazepan-4-yl)thieno[2,3-b]pyridine-2-carboxamide which was produced in Example 33 (33c) was used in place of 3-amino-4-(thiomorpholine-4-yl)thieno[2,3-b]pyridine-2-carboxamide and the reaction was performed in a similar method as described in Example 32 and the title compound was obtained.

Pale yellow powder

Mp 111-119° C.;

IR (KBr) νₘₐₓ 3432, 3321, 1648, 1590, 1499, 1368, 1035 cm⁻¹;

¹H NMR(CDCl₃, 500 MHz) δ 2.03-2.11 (1H, m), 2.59-2.67 (1H, m), 2.98-3.11 (2H, m), 3.19-3.30 (2H, m), 3.36-3.54 (3H, m), 3.91-3.96 (1H, m), 5.30 (2H, brs), 7.00 (1H, d, J=4.9 Hz), 7.15 (2H, brs), 8.53 (1H, d, J=4.9 Hz);

HRMS m/z calcd for C₁₃H₁₇O₂N₄S₂ 325.0793. found 325.0774;

MS (FAB) m/z: 325 [M+H]⁺, 273, 178, 165, 51;

### Example 35

### 3-amino-4-(3-phenylpiperidin-1-yl)thieno[2,3-b]pyridine-2-carboxamide

(Exemplified Compound No. 1-25)

### (35a) (2Z)-2-cyano-3-(3-phenylpiperidin-1-yl)but-2-enethioamide

3-phenylpiperidine was used in place of isobutylamine and the reaction was performed in a similar method as described in Example 5 (5a) and the title compound was synthesized.

Pale brown powder

Mp 159-162° C. (decomposition);

IR (KBr) νₘₐₓ 3307, 3184, 2190, 1600, 1533, 1412, 1260, 978, 850, 703 cm⁻¹;

¹H NMR(DMSO-d₆, 500 MHz) δ 1.65-1.94 (4H, m), 2.32 (3H, s), 2.85-2.91 (1H, m), 3.12-3.19 (2H, m), 3.63 (1H, d, J=11.2 Hz), 3.74 (1H, d, J=11.2 Hz), 7.22-7.33 (5H, m), 8.30 (1H, brs), 8.99 (1H, brs);

HRMS m/z calcd for C₁₆H₁₉N₃S 285.1299. found 285.1286;

MS (EI) m/z: 285 [M⁺], 251, 226, 186, 160, 135, 109, 104, 91, 59;

Anal. Calcd for C₁₆H₁₉N₃S: C, 67.33; H, 6.71; N, 14.72; S, 11.23. Found: C, 67.25; H, 6.81; N, 14.53, S, 11.04.

### (35b) 4-(3-phenylpiperidin-1-yl)-2-thioxo-1,2-dihydropyridine-3-carbonitrile

(2Z)-2-cyano-3-(3-phenylpiperidin-1-yl)but-2-enethioamide which was produced in Example 35 (35a) was used in place of (2Z)-2-cyano-3-(isobutylamino)but-2-enethioamide and the reaction was performed in a similar method as described in Example 5 (5b) and the title compound was obtained.

Pale brown powder

Mp 236-238° C. (decomposition);

IR (KBr) νₘₐₓ 3118, 2939, 2208, 1622, 1515, 1449, 1309, 1251, 1164, 974, 758, 700 cm⁻¹;

1H NMR(DMSO-d₆, 500 MHz) δ 1.64-1.98 (4H, m), 2.84-2.90 (1H, m), 3.18-3.22 (2H, m), 4.12-4.15 (2H, m), 6.55 (1H, d, J=7.3 Hz), 7.22-7.86 (5H, m), 7.46 (1H, d, J=7.3 Hz), 12.66 (1H, brs);

HRMS m/z calcd for C₁₇H₁₈N₃S 296.1222. found 296.1196;

MS (FAB) m/z: 296 [M+H]⁺, 273, 242, 165, 65, 51;

### (35c) 3-amino-4-(3-phenylpiperidin-1-yl)thieno[2,3-b]pyridine-2-carboxamide

4-(3-phenylpiperidin-1-yl)-2-thioxo-1,2-dihydropyridine-3-carbonitrile which was produced in Example 35 (35b) was used in place of 4-(isobutylamino)-2-thioxo-1,2-dihydropyridine-3-carbonitrile and the reaction was performed in a similar method as described in Example 5 (5c) and the title compound was obtained.

Pale yellow powder

Mp 267-269° C.;

IR (KBr) νₘₐₓ 3444, 3328, 3173, 2932, 1643, 1578, 1500, 1370, 1247, 1053, 966, 700 cm⁻¹;

¹H NMR(DMSO-d₆, 500 MHz) δ 1.58-1.73 (1H, m), 1.86-2.02 (3H, m), 2.73-2.80 (2H, m), 3.09-3.16 (1H, m), 3.38-3.41 (2H, m), 6.99 (2H, brs), 7.06 (1H, d, J=5.1 Hz), 7.12 (2H, brs), 7.20-7.33 (5H, m), 8.43 (1H, d, J=5.1 Hz);

HRMS m/z calcd for C₁₉H₂₀ON₄S 352.1358. found 352.1360;

MS (EI) m/z: 352 [M⁺], 334, 307, 274, 252, 233, 202, 176, 91, 77, 73;

Anal. Calcd for C₁₉H₂₀N₄OS.0.34H₂O: C, 63.64; H, 5.81; N, 15.62; S, 8.94. Found: C, 63.32; H, 5.57; N, 15.87, S, 8.69.

### Example 36

### 3-amino-4-(3-hydroxypiperidin-1-yl)thieno[2,3-b]pyridine-2-carboxamide

(Exemplified Compound No. 1-27)

### (36a) (2Z)-2-cyano-3-(3-hydroxypiperidin-1-yl)but-2-enethioamide

3-hydroxypiperidine was used in place of isobutylamine and the reaction was performed in a similar method as described in Example 5 (5a) and the title compound was synthesized.

Pale yellow powder

Mp 159-162° C. (decomposition);

IR (KBr) νₘₐₓ 3287, 2939, 2184, 1601, 1539, 1407, 1261, 1071, 859 cm⁻¹;

¹H NMR(DMSO-d₆, 400 MHz) δ 1.42-1.52 (2H, m), 1.74-1.86 (2H, m), 2.27 (3H, s), 3.03 (1H, dd, J=7.8, 13.3 Hz), 3.11-3.16 (1H, m), 3.44 (1H, dd, J=3.4, 13.3 Hz), 3.59-3.64 (1H, m), 5.01 (1H, d, J=4.7 Hz), 8.14 (1H, brs), 8.91 (1H, brs);

HRMS m/z calcd for C₁₀H₁₆ON₃S 226.1014. found 226.1024;

MS (FAB) m/z: 226 [M+H]⁺, 192, 171, 65;

Anal. Calcd for C₁₀H₁₅N₃OS.0.04H₂O: C, 53.14; H, 6.72; N, 18.59; S, 14.19. Found: C, 53.00; H, 6.52; N, 18.47, S, 14.15.

### (36b) 4-(3-hydroxypiperidin-1-yl)-2-thioxo-1,2-dihydropyridine-3-carbonitrile

(2Z)-2-cyano-3-(3-hydroxypiperidin-1-yl)but-2-enethioamide which was produced in Example 36 (36a) was used in place of (2Z)-2-cyano-3-(isobutylamino)but-2-enethioamide and the reaction was performed in a similar method as described in Example 5 (5b) and the title compound was obtained.

Brown powder

Mp 202-206° C.;

IR (KBr) νₘₐₓ 3119, 2945, 2208, 1625, 1522, 1251, 1173, 995, 962, 773 cm⁻¹;

¹H NMR(DMSO-d₆, 500 MHz) δ 1.46-1.53 (2H, m), 1.79-1.88 (2H, m), 3.29 (1H, dd, J=7.8, 13.2 Hz), 3.39-3.44 (1H, m), 3.60-3.71 (2H, m), 3.80 (1H, dd, J=3.4, 13.2 Hz), 6.48 (1H, d, J=7.8 Hz), 7.44 (1H, d, J=7.8 Hz), 12.59 (1H, brs).

### (36c) 3-amino-4-(3-hydroxypiperidin-1-yl)thieno[2,3-b]pyridine-2-carboxamide

4-(3-hydroxypiperidin-1-yl)-2-thioxo-1,2-dihydropyridine-3-carbonitrile which was produced in Example 36 (36b) was used in place of 4-(isobutylamino)-2-thioxo-1,2-dihydropyridine-3-carbonitrile and the reaction was performed in a similar method as described in Example 5 (5c) and the title compound was obtained.

Yellow powder

Mp 208-211° C.;

IR (KBr) νₘₐₓ 3325, 1633, 1594, 1501, 1374, 1243, 959 cm⁻¹;

¹H NMR(DMSO-d₆, 400 MHz) δ 1.60-2.02 (4H, m), 3.16-3.96 (5H, m), 6.38 (2H, brs), 6.99 (1H, d, J=5.1 Hz), 7.05 (2H, brs), 8.40 (1H, d, J=5.1 Hz);

HRMS m/z calcd for C₁₃H₁₆O₂N₄S 292.0994. found 292.1013;

MS (EI) m/z: 292 [M⁺], 274, 256, 252, 218, 201, 176, 148, 128, 122, 43;

Anal. Calcd for C₁₃H₁₆N₄O₂S.0.16H₂O: C, 52.89; H, 5.57; N, 18.98; S, 10.86. Found: C, 53.11; H, 5.55; N, 18.61, S, 10.86.

### Example 37

### tert-butyl 4-[3-amino-2-(aminocarbonyl)thieno[2,3-b]pyridin-4-yl]piperazine-1-carboxylate

(Exemplified Compound No. 3-75)

### (37a) tert-butyl 4-[(1Z)-3-amino-2-cyano-1-methyl-3-thioxoprop-1-enyl]piperazine-1-carboxylate

tert-butyl piperazine-1-carboxylate was used in place of propylamine, the reaction was performed in a similar method as described in Example 4 (4a) and the title compound was obtained. Yield 40%.

¹H NMR(DMSO-d₆, 400 MHz) δ 1.41 (9H, s), 2.27 (3H, s), 3.36-3.46 (8H, m), 8.36 (1H, s), 9.06 (1H, s).

### (37b) tert-butyl 4-(3-cyano-2-thioxo-1,2-dihydropyridin-4-yl)piperazine-1-carboxylate

tert-butyl 4-[(1Z)-3-amino-2-cyano-1-methyl-3-thioxoprop-1-enyl]piperazine-1-carboxylate which was produced in Example 37 (37a) was used in place of (2Z)-2-cyano-3-(dimethylamino)but-2-enethioamide and the reaction was performed in a similar method as described in Example 1 (1b) and the title compound was obtained.

Yield 69%.

¹H NMR(DMSO-d₆, 400 MHz) δ 2.49 (9H, s), 3.44-3.49 (4H, m), 3.61-3.66 (4H, m), 6.45 (1H, d, J=7.4 Hz), 6.49 (1H, d, J=7.4 Hz).

### (37c) tert-butyl 4-[3-amino-2-(aminocarbonyl)thieno[2,3-b]pyridin-4-yl]piperazine-1-carboxylate

tert-butyl 4-(3-cyano-2-thioxo-1,2-dihydropyridin-4-yl)piperazine-1-carboxylate which was produced in Example 37 (37b) was used in place of 4-(dimethylamino)-2-thioxo-1,2-dihydropyridine-3-carbonitrile and the reaction was performed in a similar method as described in Example 1 (1c) and the title compound was synthesized. Yield 90%.

Mp 198-203° C.;

IR (KBr) νₘₐₓ 3428, 3323, 3176, 2974, 1693, 1649, 1584, 1501, 1367, 1241, 1169, 1124, 976, 959, 825, 770 cm⁻¹;

¹H NMR(DMSO-d₆, 400 MHz) δ 1.42 (9H, s), 3.25 (4H, s), 3.29 (4H, s), 6.95 (2H, br s), 7.03 (1H, d, J=5.4 Hz), 7.12 (2H, br s), 8.45 (1H, d, J=5.4 Hz);

MS (FAB) m/z: 378 [M+H]⁺;

Anal. Calcd for C₁₇H₂₃N₅O₃S.0.3H₂O: C, 53.33; H, 6.21; N, 18.29; S, 8.37. Found: C, 53.14; H, 5.86; N, 18.06; S, 8.41.

### Example 38

### 3-amino-4-piperazin-1-ylthieno[2,3-b]pyridine-2-carboxamide dihydrochloride

(Exemplified Compound No. 3-1)

tert-butyl 4-[3-amino-2-(aminocarbonyl)thieno[2,3-b]pyridin-4-yl]piperazine-1-carboxylate (326 mg, 0.86 mmol) which was produced in Example 37 (37c) was suspended in 1,4-dioxane (10 mL) and was blended with 4N hydrochloric acid-dioxane (4 mL) and the mixture was stirred for two hours. After the solvent was evaporated, obtained yellow solid was dried under vacuum, and the title compound was obtained (316 mg, yield 100%).

Mp 270-280° C.;

IR (KBr) νₘₐₓ 3320, 3180, 2925, 2770, 2717, 1648, 1604, 1446, 1395, 1259, 1059, 973, 906, 797, 556, 540, 516 cm⁻¹;

¹H NMR(DMSO-d₆, 400 MHz) δ 3.58-4.28 (8H, br s), 7.125 (1H, d, J=5.1 Hz), 7.24 (2H, br s), 8.525 (1H, d, J=5.1 Hz), 9.45 (2H, br s);

MS (FAB) m/z: 278 [M+H]⁺;

Anal. Calcd for C₁₂H₁₅N₅OS.2HCl.H₂O: C, 39.14; H, 5.20; N, 19.02. Found: C, 38.99; H, 5.13; N, 18.77.

### Example 39

### 3-amino-4-(4-methylpiperazin-1-yl)thieno[2,3-b]pyridine-2-carboxamide

(Exemplified Compound No. 3-2)

### (39a) (2Z)-2-cyano-3-(4-methylpiperazin-1-yl)but-2-enethioamide

1-methylpiperazine was used in place of propylamine, the reaction was performed in a similar method as described in Example 4 (4a) and the title compound was obtained. Yield 85%.

¹H NMR(DMSO-d₆, 400 MHz) δ 2.18 (3H, s), 2.26 (3H, s), 2.38 (4H, m), 3.35 (4H, m), 8.29 (1H, br s), 8.99 (1H, br s).

### (39b) 3-amino-4-(4-methylpiperazin-1-yl)thieno[2,3-b]pyridine-2-carboxamide

(2Z)-2-cyano-3-(4-methylpiperazin-1-yl)but-2-enethioamide which was produced in Example 39 (39a) (301 mg, 1.34 mmol) and dimethylformamide dimethylacetal (0.53 mL, 4.01 mmol) were suspended in toluene (5 mL) and the mixture was stirred under heat reflux for three minutes. After cooling to room temperature, 1N aqueous solution of sodium hydroxide (3.50 mL) was added to the obtained residue by evaporating the solvent and it was heated for 30 minutes under reflux. After cooling to room temperature, 4N hydrochloric acid-dioxane solution (5 mL) was added and the pH value of solution was adjusted to around 3. The mixture was concentrated under reduced pressure and 2-chloroacetamide (126 mg, 1.35 mmol), 8N aqueous solution of sodium hydroxide (2.5 mL), dimethylformamide (5 mL) were added to the obtained residue and the mixture was stirred for three hours. Water (10 mL) was added to the reaction mixture and allowed to stand still for one week. The solid which resulted was filtered and the title compound was obtained (99 mg, yield 26%).

Mp 260-263° C.;

IR (KBr) νₘₐₓ 3502, 3424, 3322, 3161, 2939, 2801, 1653, 1588, 1502, 1451, 1371, 1344, 1288, 1246, 1199, 973, 819, 737, 683, 625, 476 cm⁻¹;

¹H NMR(DMSO-d₆, 400 MHz) δ 2.26 (3H, s), 2.40-2.72 (4H, br s), 2.85-3.22 (4H, br s), 6.89 (2H, br s), 7.00 (1H, d, J=5.5 Hz), 7.07 (2H, s), 8.41 (1H, d, J=5.5 Hz);

MS (FAB) m/z: 291 [M+H]⁺;

Anal. Calcd for C₁₃H₁₇N₅OS.0.3H₂O: C, 52.61; H, 5.98; N, 23.60; S, 10.80. Found: C, 52.62; H, 5.79; N, 23.63; S, 10.92.

### Example 40

### 3-amino-4-(4-ethyl piperazin-1-yl)thieno[2,3-b]pyridine-2-carboxamide

(Exemplified Compound No. 3-3)

### (40a) (2Z)-2-cyano-3-(4-ethyl piperazin-1-yl)but-2-enethioamide

1-ethyl piperazine was used in place of propylamine and the reaction was performed in a similar method as described in Example 4 (4a) and the title compound was obtained. Yield 67%.

¹H NMR(DMSO-d₆, 400 MHz) δ 1.00 (3H, t, J=7.0 Hz), 2.25 (3H, s), 2.36 (2H, q, J=7.0 Hz), 2.49 (4H, m), 3.36 (4H, m), 8.29 (1H, br s), 8.99 (1H, br s).

### (40b) 3-amino-4-(4-ethyl piperazin-1-yl)thieno[2,3-b]pyridine-2-carboxamide

(2Z)-2-cyano-3-(4-ethyl piperazin-1-yl)but-2-enethioamide (299 mg, 1.25 mmol) which was produced in Example 40 (40a) and dimethylformamide dimethylacetal (0.50 mL, 3.77 mmol) were suspended in toluene (5 mL) and the mixture was stirred under heat reflux for three minutes. After the reaction mixture was cooled to room temperature, 1N aqueous solution of sodium hydroxide (3.50 mL) was added to the obtained residue by evaporating the solvent, and heated under reflux for 40 minutes. After cooling to room temperature, 4N hydrochloric acid-dioxane solution (2 mL) was added and the pH value of solution was adjusted to around 4. The mixture was concentrated under reduced pressure and 2-chloroacetamide (118 mg, 1.27 mmol), 8N aqueous solution of sodium hydroxide (2.5 mL), dimethylformamide (5 mL) were added to the obtained residue and it was stirred for three hours. Water (10 mL) was added to the reaction mixture and the solid which resulted after allowing to stand still for one day was filtered and the title compound was obtained (123 mg, yield 32%).

Mp 218-219° C.;

IR (KBr) νₘₐₓ 3425, 3316, 3173, 2968, 2821, 1644, 1581, 1503, 1448, 1375, 1346, 1243, 1136, 975, 826, 770, 735, 542, 478 cm⁻¹;

¹H NMR(DMSO-d₆, 400 MHz) δ 1.04 (3H, t, J=7.0 Hz), 2.415 (2H, q, J=7.0 Hz), 2.46-3.24 (8H, br s), 6.89 (2H, s), 7.015 (1H, d, J=5.5 Hz), 7.08 (2H, s), 8.415 (1H, d, J=5.5 Hz);

MS (FAB) m/z: 306 [M+H]⁺;

Anal. Calcd for C₁₄H₁₉N₅OS.0.5H₂O: C, 53.48; H, 6.41; N, 22.27; S, 10.20. Found: C, 53.54; H, 6.05; N, 22.17; S, 10.27.

### Example 41

### 3-amino-4-(4-isopropyl piperazin-1-yl)thieno[2,3-b]pyridine-2-carboxamide

(Exemplified Compound No. 3-5)

### (41a) (2Z)-2-cyano-3-(4-isopropyl piperazin-1-yl)but-2-enethioamide

1-isopropyl piperazine was used in place of propylamine, the reaction was performed in a similar method as described in Example 4 (4a) and the title compound was obtained. Yield 67%.

¹H NMR(DMSO-d₆, 400 MHz) δ 0.955 (6H, d, J=6.7 Hz), 2.25 (3H, s), 2.36 (2H, q, J=7.0 Hz), 2.48 (4H, m), 2.65 (1H, m), 3.34 (4H, m), 8.25 (1H, br s), 8.94 (1H, br s).

### (41b) 3-amino-4-(4-isopropyl piperazin-1-yl)thieno[2,3-b]pyridine-2-carboxamide

(2Z)-2-cyano-3-(4-isopropyl piperazin-1-yl)but-2-enethioamide (299 mg, 1.18 mmol) which was produced in Example 41 (41a) and dimethylformamide dimethylacetal (0.47 mL, 3.56 mmol) were suspended in toluene (5 mL) and the mixture was stirred under heat reflux for three minutes. After the reaction mixture was cooled to room temperature, 1N aqueous solution of sodium hydroxide (3.50 mL) was added to the obtained residue by evaporating the solvent, and heated under reflux for 40 minutes. After cooling to room temperature, 1N hydrochloric acid (5 mL) was added and the pH value of solution was adjusted to around 4. The solid which resulted was removed by filtration and the obtained filtrate was concentrated under reduced pressure. 2-chloroacetamide (153 mg, 1.64 mmol), 8N aqueous solution of sodium hydroxide (5 mL), dimethylformamide (1.5 mL) were added to the residue and the mixture was stirred for three hours. Water (10 mL) was added to the reaction mixture and the solid which resulted was filtered and washed with diisopropyl ether (4×3 mL) and the title compound was obtained (137 mg, yield 28%).

Mp 240-243° C.;

IR (KBr) νₘₐₓ 3413, 3322, 3121, 2964, 2827, 1660, 1584, 1502, 1448, 1376, 1345, 1245, 1176, 1134, 982, 820, 766, 741, 646, 486 cm⁻¹;

¹H NMR(DMSO-d₆, 400 MHz) δ 1.015 (6H, d, J=6.3 Hz), 2.40-3.40 (9H, br s), 6.90 (2H, br s), 7.00 (1H, d, J=5.5 Hz), 7.06 (2H, s), 8.40 (1H, d, J=5.1 Hz);

MS (FAB) m/z: 319 [M+H]⁺;

Anal. Calcd for C₁₅H₂₁N₅OS.0.19H₂O: C, 55.80; H, 6.67; N, 21.69; S, 9.93. Found: C, 55.82; H, 6.53; N, 21.63; S, 10.06.

### Example 42

### 3-amino-4-(4-phenylpiperazin-1-yl)thieno[2,3-b]pyridine-2-carboxamide

(Exemplified Compound No. 3-9)

### (42a) (2Z)-2-cyano-3-(4-phenylpiperazin-1-yl)but-2-enethioamide

1-phenylpiperazine was used in place of propylamine, the reaction was performed in a similar method as described in Example 4 (4a) and the title compound was obtained. Yield 60%.

¹H NMR(DMSO-d₆, 400 MHz) δ 2.31 (3H, s), 3.27 (4H, m), 3.51 (4H, m), 6.78 (1H, t, J=7.1 Hz), 6.92 (2H, d, J=7.8 Hz), 7.21 (2H, m), 8.36 (1H, s), 9.05 (1H, s).

### (42b) 4-(4-phenylpiperazin-1-yl)-2-thioxo-1,2-dihydropyridine-3-carbonitrile

(2Z)-2-cyano-3-(4-phenylpiperazin-1-yl)but-2-enethioamide which was produced in Example 42 (42a) was used in place of (2Z)-2-cyano-3-(dimethylamino)but-2-enethioamide and the reaction was performed in a similar method as described in Example 1 (lb) and the title compound was obtained. Yield 77%.

¹H NMR(DMSO-d₆, 400 MHz) δ 3.31 (4H, m), 3.80 (4H, m), 6.535 (lH, d, J=6.6 Hz), 6.79 (1H, t, J=7.4 Hz), 6.93 (2H, d, J=7.8 Hz), 7.14-7.25 (2H, m), 7.50 (2H, dd, J=6.9, 7.4 Hz).

### (42c) 3-amino-4-(4-phenylpiperazin-1-yl)thieno[2,3-b]pyridine-2-carboxamide

4-(4-phenylpiperazin-1-yl)-2-thioxo-1,2-dihydropyridine-3-carbonitrile which was produced in Example 42 (42b) was used in place of 4-(dimethylamino)-2-thioxo-1,2-dihydropyridine-3-carbonitrile and the reaction was performed in a similar method as described in Example 1 (1c) and the title compound was synthesized.

Yield 75%.

Mp 250-252° C.;

IR (KBr) νₘₐₓ 3438, 3318, 3176, 2832, 1645, 1596, 1579, 1447, 1377, 1343, 1238, 1136, 1135, 978, 914, 831, 762, 733, 693, 626, 484 cm⁻¹;

¹H NMR(DMSO-d₆, 400 MHz) δ 2.89-3.71 (8H, br s), 6.70 (1H, t, J=7.1 Hz), 6.93 (2H, s), 6.99 (2H, d, J=9.0 Hz), 7.07 (2H, d, J=5.5 Hz), 7.10 (2H, s), 7.22 (2H, dd, J=7.4, 8.6 Hz), 8.44 (1H, d, J=5.5 Hz);

MS (FAB) m/z: 353 [M+H]⁺;

Anal. Calcd for C₁₈H₁₉N₅OS.0.26H₂O: C, 60.37; H, 5.49; N, 19.56; S, 8.95. Found: C, 60.18; H, 5.33; N, 19.55; S, 9.02.

### Example 43

### 3-amino-4-(4-benzylpiperazin-1-yl)thieno[2,3-b]pyridine-2-carboxamide

(Exemplified Compound No. 3-6)

### (43a) (2Z)-3-(4-benzylpiperazin-1-yl) -2-cyanobut-2-enethioamide

1-benzylpiperazine was used in place of propylamine, the reaction was performed in a similar method as described in Example 4 (4a) and the title compound was obtained. Yield 33%.

¹H NMR(DMSO-d₆, 400 MHz) δ 2.25 (3H, s), 2.44 (4H, m), 3.37 (4H, m), 3.50 (2H, s), 7.22-7.34 (5H, m), 8.30 (1H, s), 8.98 (1H, s).

### (43b) 4-(4-benzylpiperazin-4-yl)-2-thioxo-1,2-dihydropyridine-3-carbonitrile

(2Z)-3-(4-benzylpiperazin-1-yl)-2-cyanobut-2-enethioamide which was produced in Example 42 (42a) was used in place of (2Z)-2-cyano-3-(dimethylamino)but-2-enethioamide and the reaction was performed in a similar method as described in Example 1 (1b) and the title compound was obtained. Yield 78%.

¹H NMR(DMSO-d₆, 400 MHz) δ 2.49 (4H, m), 3.51 (2H, s), 3.61 (4H, m), 6.45 (1H, d, J=7.8 Hz), 7.20-7.34 (5H, m), 7.50 (1H, d, J=7.4 Hz).

### (43c) 3-amino-4-(4-benzylpiperazin-1-yl)thieno[2,3-b]pyridine-2-carboxamide

4-(4-benzylpiperazin-4-yl)-2-thioxo-1,2-dihydropyridine-3-carbonitrile which was produced in Example 42 (42b) was used in place of 4-(dimethylamino)-2-thioxo-1,2-dihydropyridine-3-carbonitrile and the reaction was performed in a similar method as described in Example 1 (1c) and the title compound was synthesized.

Yield 75%.

Mp 241-242° C.;

IR (KBr) νₘₐₓ 3435, 3398, 3322, 3128, 2828, 1656, 1585, 1503, 1452, 1373, 1348, 1250, 1232, 1132, 1061, 1009, 973, 826, 742, 699, 627, 551, 487 cm⁻¹;

¹H NMR(DMSO-d₆, 400 MHz) δ 2.42-3.22 (8H, br s), 3.55 (1H, s), 6.89 (2H, s), 7.02 (1H, d, J=5.5 Hz), 7.07 (2H, s), 7.20-7.34 (5H, s), 8.415 (1H, d, J=5.1 Hz);

MS (FAB) m/z: 367 [M+H]⁺;

Anal. Calcd for C₁₉H₂₁N₅OS: C, 62.10; H, 5.76; N, 19.06; S, 8.73. Found: C, 61.96; H, 5.63; N, 18.76; S, 8.75.

### Example 44

### 3-amino-4-(4-pyrimidin-2-ylpiperazin-1-yl)thieno[2,3-b]pyridine-2-carboxamide

(Exemplified Compound No. 3-69)

### (44a) (2Z)-2-cyano-3-(4-pyrimidin-2-ylpiperazin-1-yl)but-2-enethioamide

1-pyrimidin-2-ylpiperazin was used in place of propylamine, the reaction was performed in a similar method as described in Example 4 (4a) and the title compound was obtained. Yield 87%.

¹H NMR(DMSO-d₆, 400 MHz) δ 2.31 (3H, s), 3.51 (4H, m), 3.83 (4H, m), 6.66 (1H, t, J=4.7 Hz), 8.32 (1H, s), 6.92 (2H, d, J=4.7 Hz), 9.03 (1H, s).

### (44b) 4-(4-pyrimidin-2-ylpiperazin-1-yl)-2-thioxo-1,2-dihydropyridine-3-carbonitrile

(2Z)-2-cyano-3-(4-pyrimidin-2-ylpiperazin-1-yl)but-2-enethioamide which was produced in Example 44 (44a) was used in place of (2Z)-2-cyano-3-(dimethylamino)but-2-enethioamide and the reaction was performed in a similar method as described in Example 1 (1b) and the title compound was obtained. Yield 77%.

¹H NMR(DMSO-d₆, 400 MHz) δ 3.785 (4H, m), 3.89 (4H, m), 6.53 (1H, d, J=7.4 Hz), 6.69 (1H, t, J=4.3 Hz), 7.52 (1H, t, J=7.8 Hz), 7.50 (2H, d, J=4.7 Hz), 12.74 (1H, br s).

### (44c) 3-amino-4-(4-pyrimidin-2-ylpiperazin-1-yl)thieno[2,3-b]pyridine-2-carboxamide

(2Z)-2-cyano-3-(4-pyrimidin-2-ylpiperazin-1-yl)but-2-enethioamide which was produced in Example 44 (44b) was used in place of 4-(dimethylamino)-2-thioxo-1,2-dihydropyridine-3-carbonitrile and the reaction was performed in a similar method as described in Example 1 (1c) and the title compound was synthesized.

Yield 69%.

Mp 280° C. (decomposition);

IR (KBr) νₘₐₓ 3439, 3317, 3141, 2835, 1648, 1582, 1548, 1500, 1466, 1359, 1241, 1133, 974, 797, 486 cm^{31 1};

¹H NMR(DMSO-d₆, 400 MHz) δ 2.72-3.65 (8H, br s), 6.66 (1H, t, J=4.7 Hz), 6.99 (2H, br s), 7.05 (2H, d, J=5.1 Hz), 7.11 (2H, br s), 8.39 (2H, d, J=4.7 Hz), 8.43 (1H, d, J=5.5 Hz);

MS (FAB) m/z: 356 [M+H]⁺;

Anal. Calcd for C₁₆H₁₇N₇OS.0.24H₂O: C, 54.07; H, 4.82; N, 27.59; S, 9.02. Found: C, 53.59; H, 4.81; N, 27.03; S, 8.99.

### Example 45

### 3-amino-4-(4-pyridin-2-ylpiperazin-1-yl)thieno[2,3-b]pyridine-2-carboxamide

(Exemplified Compound No. 3-62)

### (45a) (2Z)-2-cyano-3-(4-pyridin-2-ylpiperazin-1-yl)but-2-enethioamide

1-pyridin-2-ylpiperazin was used in place of propylamine, the reaction was performed in a similar method as described in Example 4 (4a) and the title compound was obtained. Yield 92%.

¹H NMR(DMSO-d₆, 400 MHz) δ 2.32 (3H, s), 3.53 (4H, m), 3.65 (4H, m), 6.67 (1H, t, J=4.7 Hz), 6.83 (1H, d, J=8.9 Hz), 7.57 (1H, t, J=8.9 Hz), 8.14 (1H, d, J=4.7 Hz), 8.36 (1H, br s), 9.07 (1H, br s).

### (45b) 4-(4-pyridin-2-ylpiperazin-1-yl)-2-thioxo-1,2-dihydropyridine-3-carbonitrile

(2Z)-2-cyano-3-(4-pyridin-2-ylpiperazin-1-yl)but-2-enethioamide which was produced in Example 45 (45a) was used in place of (2Z)-2-cyano-3-(dimethylamino)but-2-enethioamide and the reaction was performed in a similar method as described in Example 1 (1b) and the title compound was obtained. Yield 43%.

¹H NMR(DMSO-d₆, 400 MHz) δ 3.70 (4H, m), 3.82 (4H, m), 6.53 (1H, d, J=7.4 Hz), 6.72 (1H, t, J=5.9 Hz), 6.90 (1H, d, J=8.2 Hz), 7.52 (1H, t, J=6.7 Hz), 7.64 (1H, t, J=7.4 Hz), 8.13 (1H, d, J=5.1 Hz), 12.73 (1H, br s).

### (45c) 3-amino-4-(4-pyridin-2-ylpiperazin-1-yl)thieno[2,3-b]pyridine-2-carboxamide

4-(4-pyridin-2-ylpiperazin-1-yl)-2-thioxo-1,2-dihydropyridine-3-carbonitrile which was produced in Example 45 (45b) was used in place of 4-(dimethylamino)-2-thioxo-1,2-dihydropyridine-3-carbonitrile and the reaction was performed in a similar method as described in Example 1 (1c) and the title compound was synthesized. Yield 77%.

Mp 266-270° C.;

IR (KBr) νₘₐₓ 3438, 3317, 3142, 2834, 1674, 1590, 1582, 1501, 1435, 1369, 1345, 1133, 974, 774, 741, 620, 487 cm⁻¹;

¹H NMR(DMSO-d₆, 400 MHz) δ 2.80-3.60 (8H, br s), 6.67 (1H, dd, J=5.1, 6.3 Hz), 6.89 (1H, d, J=8.2 Hz), 6.97 (2H, s), 7.06 (1H, d, J=5.1 Hz), 7.11 (2H, s), 7.55 (1H, t, J=5.1 Hz), 8.39 (1H, d, J=3.5 Hz), 8.44 (1H, d, J=5.1 Hz);

MS (FAB) m/z: 355 [M+H]⁺;

Anal. Calcd for C₁₇H₁₈N₆OS: C, 57.61; H, 5.12; N, 23.71; S, 9.05. Found: C, 57.25; H, 5.01; N, 23.35; S, 9.04.

### Example 46

### 3-amino-4-[4-(4-methylphenyl)piperazin-1-yl]thieno[2,3-b]pyridine-2-carboxamide

(Exemplified Compound No. 3-34)

### (46a) (2Z)-2-cyano-3-[4-(4-methylphenyl)piperazin-1-yl]but-2-enethioamide

1-(4-methylphenyl)piperazine was used in place of propylamine, the reaction was performed in a similar method as described in Example 4 (4a) and the title compound was obtained. Yield 70%.

¹H NMR(DMSO-d₆, 400 MHz) δ 2.20 (3H, s), 2.30 (3H, s), 3.20 (4H, m), 3.51 (4H, m), 6.83 (2H, d, J=8.6 Hz), 7.02 (2H, d, J=8.2 Hz), 8.35 (1H, s), 9.04 (1H, s).

### (46b) 4-[4-(4-methylphenyl)piperazin-1-yl]-2-thioxo-1,2-dihydropyridine-3-carbonitrile

(2Z)-2-cyano-3-[4-(4-methylphenyl)piperazin-1-yl]but-2-enethioamide which was produced in Example 46 (46a) was used in place of (2Z)-2-cyano-3-(dimethylamino)but-2-enethioamide and the reaction was performed in a similar method as described in Example 1 (1b) and the title compound was obtained. Yield 77%.

¹H NMR(DMSO-d₆, 400 MHz) δ 2.21 (3H, s), 3.22 (4H, m), 3.79 (4H, m), 6.45 (1H, d, J=7.4 Hz), 6.86 (2H, d, J=8.6 Hz), 7.05 (2H, d, J=8.2 Hz), 7.52 (1H, t, J=7.0 Hz), 12.73 (1H, br s).

### (46c) 3-amino-4-[4-(4-methylphenyl)piperazin-1-yl]thieno[2,3-b]pyridine-2-carboxamide

4-[4-(4-methylphenyl)piperazin-1-yl]-2-thioxo-1,2-dihydropyridine-3-carbonitrile which was produced in Example 46 (46b) was used in place of 4-(dimethylamino)-2-thioxo-1,2-dihydropyridine-3-carbonitrile, the reaction was performed in a similar method as described in Example 1 (1c) and the title compound was synthesized. Yield 72%.

Mp 270° C. (decomposition);

IR (KBr) νₘₐₓ 3433, 3310, 3143, 2829, 1667, 1611, 1580, 1513, 1449, 1371, 1345, 1237, 1134, 1124, 974, 956, 812, 617, 482 cm⁻¹;

¹H NMR(DMSO-d₆, 400 MHz) δ 2.20 (3H, s), 2.91-3.60 (8H, br s), 6.86-7.12 (9H, m), 8.42 (1H, d, J=5.5 Hz);

MS (FAB) m/z: 367 [M+H]⁺;

Anal. Calcd for C₁₉H₂₁N₅OS.0.24H₂O: C, 61.38; H, 5.82; N, 18.84; S, 8.62. Found: C, 61.58; H, 5.58; N, 18.84; S, 8.63.

### Example 47

### 3-amino-4-[4-(4-fluorophenyl)piperazin-1-yl]thieno[2,3-b]pyridine-2-carboxamide

(Exemplified Compound No. 3-12)

### (47a) (²Z)-²-cyano-3-[4-(4-fluorophenyl)piperazin-1-yl]but-2-enethioamide

1-(4-fluorophenyl) piperazine was used in place of propylamine, the reaction was performed in a similar method as described in Example 4 (4a) and the title compound was obtained. Yield 73%.

¹H NMR(DMSO-d₆, 400 MHz) δ 2.31 (3H, s), 3.21 (4H, m), 3.52 (4H, m), 6.83 (2H, dd, J=4.7, 9.0 Hz), 7.02 (2H, t, J=8.6 Hz), 8.37 (1H, s), 9.06 (1H, s).

### (47b) 4-[4-(4-fluorophenyl)piperazin-1-yl]-2-thioxo-1,2-dihydropyridine-3-carbonitrile

(2Z)-2-cyano-3-[4-(4-fluorophenyl)piperazin-1-yl]but-2-enethioamide which was produced in Example 47 (47a) was used in place of (2Z)-2-cyano-3-(dimethylamino)but-2-enethioamide and the reaction was performed in a similar method as described in Example 1 (1b) and the title compound was obtained. Yield 74%.

¹H NMR(DMSO-d₆, 400 MHz) δ 3.24 (4H, m), 3.80 (4H, m), 6.56 (1H, d, J=7.4 Hz), 6.98 (2H, dd, J=4.7, 9.4 Hz), 7.08 (2H, t, J=8.9 Hz), 7.52 (1H, d, J=7.4 Hz).

### (47c) 3-amino-4-[4-(4-fluorophenyl) piperazin-1-yl]thieno[2,3-b]pyridine-2-carboxamide

4-[4-(4-fluorophenyl)piperazin-1-yl]-2-thioxo-1,2-dihydropyridine-3-carbonitrile which was produced in Example 47 (47b) was used in place of 4-(dimethylamino)-2-thioxo-1,2-dihydropyridine-3-carbonitrile, and the reaction was performed in a similar method as described in Example 1 (1c) and the title compound was synthesized. Yield 62%.

Mp 275° C. (decomposition);

IR (KBr) νₘₐₓ 3443, 3324, 3180, 2832, 1646, 1583, 1558, 1509, 1450, 1373, 1345, 1234, 1136, 977, 959, 826, 716, 553 cm⁻¹;

¹H NMR(DMSO-d₆, 400 MHz) δ 2.93-3.75 (8H, br s), 6.96 (2H, s), 7.00-7.19 (7H, m), 8.48 (1H, d, J=5.1 Hz);

MS (FAB) m/z: 371 [M+H]⁺;

Anal. Calcd for C₁₈H₁₈FN₅OS.0.19H₂O: C, 57.67; H, 4.94; N, 18.68; S, 8.55. Found: C, 57.72; H, 4.72; N, 18.58; S, 8.53.

### Example 48

### 3-amino-4-(4-benzhydrylpiperazin-1-yl)thieno[2,3-b]pyridine-2-carboxamide

(Exemplified Compound No. 3-7)

### (48a) (2Z)-3-(4-benzhydrylpiperazin-1-yl)-2-cyanobut-2-enethioamide

1-benzhydrylpiperazine was used in place of propylamine, the reaction was performed in a similar method as described in Example 4 (4a) and the title compound was obtained. Yield 90%.

¹H NMR(DMSO-d₆, 400 MHz) δ 2.22 (3H, s), 2.38 (4H, m), 3.41 (4H, m), 4.34 (1H, s), 7.18 (2H, t, J=7.4 Hz), 7.28 (4H, t, J=7.8 Hz), 7.42 (4H, d, J=7.0 Hz), 8.31 (1H, s), 8.98 (1H, s).

### (48b) 4-(4-benzhydrylpiperazin-1-yl)-2-thioxo-1,2-dihydropyridine-3-carbonitrile

(2Z)-3-(4-benzhydrylpiperazin-1-yl)-2-cyanobut-2-enethioamide which was produced in Example 48 (48a) was used in place of (2Z)-2-cyano-3-(dimethylamino)but-2-enethioamide and the reaction was performed in a similar method as described in Example 1 (1b) and the title compound was obtained. Yield 84%.

¹H NMR(DMSO-d₆, 400 MHz) δ 2.44 (4H, m), 3.66 (4H, m), 4.38 (1H, s), 6.47 (1H, d, J=7.0 Hz), 7.23 (1H, d, J=7.0 Hz), 7.27-7.66 (10H, m).

### (48c) 3-amino-4-(4-benzhydrylpiperazin-1-yl)thieno[2,3-b]pyridine-2-carboxamide

4-(4-benzhydrylpiperazin-1-yl)-2-thioxo-1,2-dihydropyridine-3-carbonitrile which was produced in Example 48 (48b) was used in place of 4-(dimethylamino)-2-thioxo-1,2-dihydropyridine-3-carbonitrile and the reaction was performed in a similar method as described in Example 1 (1c) and the title compound was synthesized. Yield 63%.

Mp 225-233° C.;

IR (KBr) νₘₐₓ 3450, 3381, 3171, 2827, 1649, 1580, 1501, 1449, 1366, 1242, 1137, 979, 957, 835, 758, 706, 626, 473 cm⁻¹;

¹H NMR(DMSO-d₆, 400 MHz) δ 2.20-3.40 (8H, br s), 4.38 (1H, s), 6.89 (1H, s), 7.07 (2H, m), 7.20 (2H, t, J=7.4 Hz), 7.31 (5H, t, J=7.4 Hz), 7.47 (5H, d, J=7.4 Hz), 8.44 (1H, d, J=5.5 Hz);

MS (FAB) m/z: 444 [M+H]⁺;

Anal. Calcd for C₂₅H₂₅N₅OS.0.26H₂O: C, 66.99; H, 5.74; N, 15.62; S, 7.15. Found: C, 67.07; H, 5.57; N, 15.36; S, 7.09.

### Example 49

### 3-amino-4-[4-(4-methoxyphenyl)piperazin-1-yl]thieno[2,3-b]pyridine-2-carboxamide

(Exemplified Compound No. 3-47)

### (49a) (2Z)-2-cyano-3-[4-(4-methoxyphenyl)piperazin-1-yl]but-2-enethioamide

1-(4-methoxyphenyl) piperazine was used in place of propylamine, the reaction was performed in a similar method as described in Example 4 (4a) and the title compound was obtained. Yield 73%.

¹H NMR(DMSO-d₆, 400 MHz) δ 2.31 (3H, s), 3.13 (4H, m), 3.52 (4H, m), 3.69 (3H, s), 6.84 (2H, d, J=9.0 Hz), 6.92 (2H, d, J=9.3 Hz), 8.39 (1H, s), 9.07 (1H, s).

### (49b) 4-[4-(4-methoxyphenyl)piperazin-1-yl]-2-thioxo-1,2-dihydropyridine-3-carbonitrile

(2Z)-2-cyano-3-[4-(4-methoxyphenyl)piperazin-1-yl]but-2-enethioamide which was produced in Example 49 (49a) was used in place of (2Z)-2-cyano-3-(dimethylamino)but-2-enethioamide and the reaction was performed in a similar method as described in Example 1 (1b) and the title compound was obtained. Yield 59%.

¹H NMR(DMSO-d₆, 400 MHz) δ 3.14 (4H, m), 3.67 (3H, s), 3.77 (4H, m), 6.45 (1H, d, J=7.1 Hz), 6.86 (2H, d, J=9.4 Hz), 7.05 (2H, d, J=9.4 Hz), 7.52 (1H, t, J=7.4 Hz).

### (49c) 3-amino-4-[4-(4-methoxyphenyl)piperazin-1-yl]thieno[2,3-b]pyridine-2-carboxamide

4-[4-(4-methoxyphenyl)piperazin-1-yl]-2-thioxo-1,2-dihydropyridine-3-carbonitrile which was produced in Example 49 (49b) was used in place of 4-(dimethylamino)-2-thioxo-1,2-dihydropyridine-3-carbonitrile, the reaction was performed in a similar method as described in Example 1 (1c) and the title compound was synthesized. Yield 79%.

Mp 275° C. (decomposition);

IR (KBr) νₘₐₓ 3435, 3311, 3160, 2957, 2831, 1664, 1609, 1511, 1449, 1373, 1346, 1242, 1182, 1136, 1035, 976, 959, 826, 740, 605, 480 cm⁻¹;

¹H NMR(DMSO-d₆, 400 MHz) δ 2.95-3.56 (8H, br s), 3.69 (1H, s), 6.3 (1H, d, J=9.0 Hz), 6.93 (2H, s), 6.95 (1H, d, J=9.4 Hz), 7.07 (1H, d, J=5.5 Hz), 7.09 (2H, s), 8.45 (1H, d, J=5.5 Hz);

MS (FAB) m/z: 384 [M+H]⁺;

Anal. Calcd for C₁₉H₂₁N₅O₂S.0.28H₂O: C, 58.74; H, 5.59; N, 18.03; S, 8.25. Found: C, 58.57; H, 5.42; N, 18.03; S, 8.03.

### Example 50

### 3-amino-4-{4-[(2E)-3-phenylprop-2-enyl]piperazin-1-yl}thieno[2,3b]pyridine-2-carboxamide

(Exemplified Compound No. 3-8)

### (50a) (2Z)-2-cyano-3-{4-[(2E)-3-phenylpropane-2-enyl]piperazin-1-yl}but-2enethioamide

1-[(2E)-3-phenylpropane-2-enyl]piperazine was used in place of propylamine, the reaction was performed in a similar method as described in Example 4 (4a) and the title compound was obtained. Yield 38%.

¹H NMR(DMSO-d₆, 400 MHz) δ 2.27 (3H, s), 2.50 (4H, m), 3.14 (2H, d, J=5.9 Hz), 3.40 (4H, m), 3.69 (3H, s), 6.30 (1H, dt, J=6.6, 16.0 Hz), 6.56 (1H, d, J=16.0 Hz), 7.24 (1H, t, J=7.4 Hz), 7.32 (2H, t, J=7.0 Hz), 7.44 (2H, d, J=7.0 Hz), 832 (1H, s), 9.01 (1H, s).

### (50b) 4-{4-[(2E)-3-phenylpropane-2-enyl]piperazin-1-yl}-2-thioxo-1,2-dihydropyridine-3-carbonitrile

(2Z)-2-cyano-3-{4-[(2E)-3-phenylpropane-2-enyl]piperazin-1-yl}but-2-enethioamide which was produced in Example 50 (50a) was used in place of (2Z)-2-cyano-3-(dimethylamino)but-2-enethioamide and the reaction was performed in a similar method as described in Example 1 (1b) and the title compound was obtained. Yield 84%.

¹H NMR(DMSO-d₆, 400 MHz) δ 2.85-4.44 (10H, m), 6.28 (1H, dt, J=7.8, 14.8 Hz), 6.55 (1H, d, J=7.4 Hz), 6.79 (1H, d, J=14.9 Hz), 7.31 (1H, d, J=7.4 Hz), 7.37 (2H, t, J=7.4 Hz), 7.48 (2H, d, J=7.4 Hz), 7.57 (1H, t, J=6.7 Hz), 12.91 (1H, s).

### (50c) 3-amino-4-{4-[(2E)-3-phenylprop-2-enyl]piperazin-1-yl}thieno[2,3-b]pyridine-2-carboxamide

4-{4-[(2E)-3-phenylpropane-2-enyl]piperazin-1-yl}-2-thioxo-1,2-dihydropyridine-3-carbonitrile which was produced in Example 50 (50b) was used in place of 4-(dimethylamino)-2-thioxo-1,2-dihydropyridine-3-carbonitrile and the reaction was performed in a similar method as described in Example 1 (1c) and the title compound was synthesized. Yield 60%.

Mp 225-227° C.;

IR (KBr) νₘₐₓ 3433, 3319, 3184, 3024, 2832, 2811, 1649, 1578, 1501, 1448, 1369, 1347, 1129, 971, 823, 736, 693, 627, 470 cm⁻¹;

¹H NMR(DMSO-d₆, 400 MHz) δ 2.42-3.81 (8H, br s), 3.19 (1H, d, J=6.3 Hz), 6.32 (1H, dt, J=6.6, 16.0 Hz), 6.55 (1H, d, J=16.0 Hz), 6.90 (2H, s), 7.02 (1H, d, J=50 Hz), 7.07 (2H, s), 7.21 (1H, t, J=7.4 Hz), 7.30 (2H, t, J=7.4 Hz), 7.43 (2H, d, J=7.0 Hz), 8.41 (1H, d, J=5.1 Hz);

MS (FAB) m/z: 394 [M+H]⁺;

Anal. Calcd for C₂₁H₂₃N₅OS.0.46H₂O: C, 62.78; H, 6.00; N, 17.43; S, 7.98. Found: C, 62.45; H, 5.70; N, 17.32; S, 7.94.

### Example 51

### 3-amino-4-(4-benzoylpiperazin-1-yl)thieno[2,3-b]pyridine-2-carboxamide

(Exemplified Compound No. 3-73)

3-amino-4-piperazin-1-ylthieno[2,3-b]pyridine-2-carboxamide dihydrochloride (99.7 mg, 0.28 mmol) which was produced in Example 38 was suspended in 1,4-dioxane (5 mL) and was blended with benzoyl chloride (37 μL, 0.31 mmol) and triethylamine (44 μL, 0.31 mmol) and the mixture was stirred for 12 hours. After evaporating the solvent, water (15 mL) was added and allowed to stand for several hours, a brown solid which was generated was filtrated, dried under vacuum and the title compound was obtained (31.8 mg, yield 29%).

Mp >300° C.;

IR (KBr) νₘₐₓ 3437, 3325, 3182, 2923, 2856, 1635, 1598, 1567, 1495, 1411, 1284, 1244, 1013, 974, 720, 675, 482 cm⁻¹;

¹H NMR(DMSO-d₆, 400 MHz) δ 2.95-3.66 (8H, br s), 6.99 (2H, s), 7.07 (1H, s), 7.15 (2H, s), 7.42-7.57 (3H, m), 7.96 (2H, m), 8.48 (1H, s);

MS (FAB) m/z: 382 [M+H]⁺.

### Example 52

### 3-amino-4-[4-(4-chlorophenyl)piperazin-1-yl]thieno[2,3-b]pyridine-2-carboxamide

(Exemplified Compound No. 3-17)

### (52a) (2Z)-3-[4-(4-chlorophenyl)piperazin-1-yl]-2-cyanobut-2-enethioamide

1-(4-chlorophenyl) piperazine was used in place of propylamine, the reaction was performed in a similar method as described in Example 4 (4a) and the title compound was obtained. Yield 73%.

¹H NMR(DMSO-d₆, 400 MHz) δ 2.31 (3H, s), 3.29 (4H, m), 3.53 (4H, m), 6.95 (2H, d, J=9.0 Hz), 7.25 (2H, d, J=9.0 Hz), 8.39 (1H, s), 9.10 (1H, s).

### (52b) 4-[4-(4-chlorophenyl)piperazin-1-yl]-2-thioxo-1,2-dihydropyridine-3-carbonitrile

(2Z)-3-[4-(4-chlorophenyl)piperazin-1-yl]-2-cyanobut-2-enethioamide which was produced in Example 52 (52a) was used in place of (2Z)-2-cyano-3-(dimethylamino)but-2-enethioamide and the reaction was performed in a similar method as described in Example 1 (1b) and the title compound was obtained. Yield 74%.

¹H NMR(DMSO-d₆, 400 MHz) δ 3.31 (4H, m), 3.79 (4H, m), 6.52 (1H, d, J=7.4 Hz), 6.93 (2H, d, J=9.4 Hz), 7.23 (2H, d, J=9.0 Hz), 7.50 (1H, m);

### (52c) 3-amino-4-[4-(4-chlorophenyl) piperazin-1-yl]thieno[2,3-b]pyridine-2-carboxamide

4-[4-(4-chlorophenyl)piperazin-1-yl]-2-thioxo-1,2-dihydropyridine-3-carbonitrile which was produced in Example 52 (52b) was used in place of 4-(dimethylamino)-2-thioxo-1,2-dihydropyridine-3-carbonitrile, and the reaction was performed in a similar method as described in Example 1 (1c) and the title compound was synthesized. Yield 50%.

Mp 297° C. (decomposition);

IR (KBr) νₘₐₓ 3423, 3317, 3164, 2975, 2832, 1669, 1649, 1580, 1496, 1449, 1370, 1343, 1238, 1137, 1106, 976, 962, 819, 609, 487 cm⁻¹;

¹H NMR(DMSO-d₆, 400 MHz) δ 2.95-3.66 (8H, br s), 6.94 (2H, s), 7.01 (2H, d, J=9.0 Hz), 7.065 (1H, d, J=5.1 Hz), 7.11 (2H, s), 7.25 (2H, d, J=9.0 Hz), 8.445 (1H, d, J=5.4 Hz);

MS (FAB) m/z: 388 [M+H]⁺;

Anal. Calcd for C₁₈H₁₈CIN₅OS: C, 55.74; H, 4.68; N, 18.06; S, 8.27. Found: C, 55.43; H, 4.75; N, 17.98; S, 8.12.

### Example 53

### 3-amino-4-[4-(2-methoxyphenyl)piperazin-1-yl]thieno[2,3-b]pyridine-2-carboxamide

(Exemplified Compound No. 3-45)

### (53a) (2Z)-2-cyano-3-[4-(2-methoxyphenyl)piperazin-1-yl]but-2-enethioamide

1-(2-methoxyphenyl) piperazine was used in place of propylamine, the reaction was performed in a similar method as described in Example 4 (4a) and the title compound was obtained. Yield 68%.

¹H NMR(DMSO-d₆, 400 MHz) δ 2.31 (3H, s), 3.05 (4H, m), 3.51 (4H, m), 3.80 (3H, s), 6.85-7.04 (4H, m), 8.39 (1H, s), 9.08 (1H, s).

### (53b) 4-[4-(2-methoxyphenyl)piperazin-1-yl]-2-thioxo-1,2-dihydropyridine-3-carbonitrile

(2Z)-2-cyano-3-[4-(2-methoxyphenyl)piperazin-1-yl]but-2-enethioamide which was produced in Example 53 (53a) was used in place of (2Z)-2-cyano-3-(dimethylamino)but-2-enethioamide and the reaction was performed in a similar method as described in Example 1 (1b) and the title compound was obtained. Yield 76%.

¹H NMR(DMSO-d₆, 400 MHz) δ 3.08 (4H, m), 3.79 (7H, m), 6.44 (1H, d, J=7.4 Hz), 6.86 (2H, s), 7.05 (2H, s), 7.49 (1H, t, J=6.6 Hz), 12.96 (1H, s).

### (53c) 3-amino-4-[4-(2-methoxyphenyl) piperazin-1-yl]thieno[2,3-b]pyridine-2carboxamide

4-[4-(2-methoxyphenyl)piperazin-1-yl]-2-thioxo-1,2-dihydropyridine-3-carbonitrile which was produced in Example 53 (53b) was used in place of 4-(dimethylamino)-2-thioxo-1,2-dihydropyridine-3-carbonitrile and the reaction was performed in a similar method as described in Example 1 (1c) and the title compound was synthesized. Yield 71%.

Mp 294° C. (decomposition);

IR (KBr) νₘₐₓ 3435, 3311, 3160, 2957, 2831, 1664, 1609, 1511, 1449, 1373, 1346, 1242, 1182, 1136, 1035, 976, 959, 826, 740, 605, 480 cm⁻¹;

¹H NMR(DMSO-d₆, 400 MHz) δ 2.96-3.56 (8H, br s), 3.79 (3H, s), 6.86-7.00 (6H, m), 7.10 (3H, d, J=5.1 Hz), 8.445 (1H, d, J=5.1 Hz);

MS (FAB) m/z: 384 [M+H]⁺;

Anal. Calcd for C₁₉H₂₁N₅O₂S.0.1H₂O: C, 59.23; H, 5.55; N, 18.18; S, 8.32. Found: C, 58.94; H, 5.25; N. 18.29; S, 8.21.

### Example 54

### 3-amino-4-[4-(2-chlorophenyl)piperazin-1-yl]thieno[2,3-b]pyridine-2-carboxamide

(Exemplified Compound No. 3-15)

### (54a) (2Z)-3-[4-(2-chlorophenyl)piperazin-1-yl]-2-cyanobut-2-enethioamide

1-(2-chlorophenyl) piperazine was used in place of propylamine, the reaction was performed in a similar method as described in Example 4 (4a) and the title compound was obtained. Yield 71%.

¹H NMR(DMSO-d₆, 400 MHz) δ 2.31 (3H, s), 3.05 (4H, m), 3.51 (4H, m), 3.80 (3H, s), 6.85-7.04 (4H, m), 8.39 (1H, s), 9.08 (1H, s).

### (54b) 4-[4-(2-chlorophenyl)-piperazin-1-yl]-2-thioxo-1,2-dihydropyridine-3-carbonitrile

(2Z)-3-[4-(2-chlorophenyl)piperazin-1-yl]-2-cyanobut-2-enethioamide which was produced in Example 54 (54a) was used in place of (2Z)-2-cyano-3-(dimethylamino)but-2-enethioamide and the reaction was performed in a similar method as described in Example 1 (1b) and the title compound was obtained. Yield 37%.

¹H NMR(DMSO-d₆, 400 MHz) δ 3.11 (4H, m), 3.79 (4H, m), 6.44 (1H, d, J=7.4 Hz), 7.06 (1H, t, J=7.4 Hz), 7.16 (1H, d, J=7.8 Hz), 7.30 (1H, t, J=7.4 Hz), 742 (1H, d, J=7.8 Hz), 7.51 (1H, t, J=7.0 Hz).

### (54c) 3-amino-4-[4-(2-chlorophenyl) piperazin-1-yl]thieno[2,3-b]pyridine-2-carboxamide

4-[4-(2-chlorophenyl)-piperazin-1-yl]-2-thioxo-1,2-dihydropyridine-3-carbonitrile which was produced in Example 54 (54b) was used in place of 4-(dimethylamino)-2-thioxo-1,2-dihydropyridine-3-carbonitrile and the reaction was performed in a similar method as described in Example 1 (1c) and the title compound was synthesized. Yield 79%.

Mp 294-298° C. (decomposition);

IR (KBr) νₘₐₓ 3449, 3327, 3131, 2842, 1665, 1606, 1581, 1501, 1447, 1375, 1284, 1245, 1230, 1182, 1135, 1038, 976, 957, 826, 766, 627, 483 cm⁻¹;

¹H NMR(DMSO-d₆, 400 MHz) δ 2.95-3.62 (8H, br s), 6.98 (2H, s), 7.03-7.14 (4H, m), 7.25 (1H, d, J=7.8 Hz), 7.33 (1H, t, J=7.0 Hz), 7.42 (1H, d, J=7.8 Hz), 8.45 (1H, d, J=5.1 Hz);

MS (FAB) m/z: 388 [M+H]⁺;

Anal. Calcd for C₁₈H₁₈CIN₅OS: C, 55.74; H, 4.68; N, 18.06; S, 8.27. Found: C, 55.48; H, 4.75; N, 17.95; S, 8.04.

### Example 55

### tert-butyl 4-[3-amino-2-(aminocarbonyl)thieno[2,3-b]pyridin-4-yl]-1,4-diazepane-1-carboxylate

(Exemplified Compound No. 3-152)

### (55a) tert-butyl 4-[(1Z)-3-amino-2-cyano-1-methyl-3-thiooxoprop-1-enyl]-1,4-diazepane-1-carboxylate

N-Boc-homopiperazine was used in place of isobutylamine and the reaction was performed in a similar method as described in Example 5 (5a) and the title compound was synthesized.

Yellow powder

Mp 189-190° C.;

IR (KBr) νₘₐₓ 3284, 3196, 2188, 1686, 1526, 1415, 1280, 1167, 884 cm⁻¹;

¹H NMR(DMSO-d₆, 400 MHz) δ 1.38 (9H, s), 1.75-1.82 (2H, m), 2.27 (3H, s), 3.29-3.52 (8H, m), 8.80 (1H, brs), 9.02 (1H, brs);

HRMS m/z calcd for C₁₅H₂₅O₂N₄S 325.1798, found 325.1715;

MS (FAB) m/z: 325 [M⁺], 269, 252, 235, 201, 191, 65, 57;

Anal. Calcd for C₁₅H₂₄N₄O₂S: C, 55.53; H, 7.46; N, 12.27; S, 9.88. Found: C, 55.40; H, 7.48; N, 17.15; S, 9.68.

### (5b) tert-butyl 4-(3-cyano-2-thioxo-1,2-dihydropyridin-4-yl)-1,4-diazepane-1-carboxylate

tert-butyl 4-[(1Z)-3-amino-2-cyano-1-methyl-3-thiooxoprop-1-enyl]-1,4-diazepane-1-carboxylate which was produced in Example 55 (55a) was used in place of (2Z)-2-cyano-3-(isobutylamino)but-2-enethioamide and the reaction was performed in a similar method as described in Example 5 (5b), and the title compound was obtained.

White powder

Mp 203-205° C.;

IR (KBr) νₘₐₓ 2974, 2205, 1692, 1625, 1537, 1478, 1246, 1152, 929, 771 cm⁻¹;

¹H NMR(DMSO-d₆, 400 MHz) δ 1.27 (4.5 H, s), 1.33 (4.5 H, s), 1.71-1.85 (2H, m), 3.31-3.38 (2H, m), 3.50-3.60 (2H, m), 3.74-3.84 (2H, m), 3.87-4.04 (2H, m), 642 (1H, t, J=7.4 Hz), 7.88 (1H, d, J=7.4 Hz), 12.44 (1H, brs);

HRMS m/z calcd for C₁₆H₂O₂N₄S 335.1542, found 335.1541;

MS (FAB) m/z: 335 [M+H]⁺, 289, 279, 233, 200, 176, 165, 93, 83;

Anal. Calcd for C₁₆H₂₂N₄O₂S: C, 57.46; H, 6.63; N, 16.75; S, 9.59. Found: C, 57.10; H, 6.24; N, 16.66, S, 9.82.

### (55c) tert-butyl 4-[3-amino-2-(aminocarbonyl)thieno[2,3-b]pyridin-4-yl]-1,4-diazepane-1-carboxylate

tert-butyl 4-(3-cyano-2-thioxo-1,2-dihydropyridin-4-yl)-1,4-diazepane-1-carboxylate which was produced in Example 55 (55b) was used in place of 4-(isobutylamino)-2-thioxo-1,2-dihydropyridine-3-carbonitrile and the reaction was performed in a similar method as described in Example 5 (5c) and the title compound was obtained.

Pale yellow powder

Mp 177-178° C.;

IR (KBr) νₘₐₓ 3431, 3316, 3145, 2973, 1686, 1581, 1502, 1411, 1366, 1170, 929, 769 cm⁻¹;

¹H NMR(DMSO-d₆, 400 MHz) δ 1.40 (4.5H, s), 1.43 (4.5H, s), 1.93-2.02 (2H, m), 3.17-3.23 (4H, m), 3.47 (2H, t, J=6.3 Hz), 3.58-3.63 (2H, m), 7.00 (2H, brs), 706 (1H, d, J=5.1 Hz), 7.08 (2H, brs), 8.39 (1H, d, J=5.1 Hz);

HRMS m/z calcd for C₁₈H₂₆O₃N₅S 392.1757, found 392.1761;

MS (FAB) m/z: 391 [M⁺], 336, 319, 289, 230, 218, 202, 190, 176, 93;

Anal. Calcd for C₁₈H₂₅N₅O₃S: C, 55.22; H, 6.44; N, 17.89; S, 8.19. Found: C, 54.90; H, 6.40; N, 17.85; S, 8.26.

### Example 56

### 3-amino-4-(4-benzyl-1,4-diazepan-1-yl)thieno[2,3-b]pyridine-2-carboxamide

(Exemplified Compound No. 3-83)

### (56a) (2Z)-3-(4-benzyl-1,4-diazepan-1-yl)-2-cyanobut-2-enethioamide

1-benzylhomopiperazine was used in place of propylamine, the reaction was performed in a similar method as described in Example 4 (4a) and the title compound was obtained. Yield 88%.

¹H NMR(DMSO-d₆, 400 MHz) δ 1.86 (2H, m), 2.30 (3H, s), 2.57 (2H, t, J=5.1 Hz), 2.67-2.73 (2H, m), 3.50-3.54 (4H, m), 3.57, (2H, s), 7.21-7.32 (5H, m), 8.18 (1H, s), 8.84 (1H, s).

### (56b) 4-(4-benzyl-1,4-diazepan-1-yl)-2-thioxo-1,2-dihydropyridine-3-carbonitrile

(2Z)-3-(4-benzyl-1,4-diazepan-1-yl)-2-cyanobut-2-enethioamide which was produced in Example 56 (56a) was used in place of (2Z)-2-cyano-3-(dimethylamino)but-2-enethioamide and the reaction was performed in a similar method as described in Example 1 (1b) and the title compound was obtained. Yield 70%.

¹H NMR(DMSO-d₆, 400 MHz) δ 1.80-4.43 (12H, m), 6.38 (1H, d, J=7.4 Hz), 7.15-7.61 (6H, m).

### (56c) 3-amino-4-(4-benzyl-1,4-diazepan-1-yl)thieno [2,3-b]pyridine-2-carboxamide

4-(4-benzyl-1,4-diazepan-1-yl)-2-thioxo-1,2-dihydropyridine-3-carbonitrile which was produced in Example 56 (56b) was used in place of 4-(dimethylamino)-2-thioxo-1,2-dihydropyridine-3-carbonitrile and the reaction was performed in a similar method as described in Example 1 (1c) and the title compound was synthesized. Yield 75%.

Mp 194-195° C.;

IR (KBr) νₘₐₓ 3429, 3306, 3142, 2937, 2828, 1674, 1646, 1583, 1501, 1452, 1368, 1344, 1262, 1157, 1108, 1026, 925, 740, 699, 483 cm⁻¹;

¹H NMR(DMSO-d₆, 400 MHz) δ 1.86 (2H, m), 2.70 (2H, m), 2.79 (2H, m), 3.29 (2H, m), 3.35 (2H, m), 3.67 (2H, s), 7.04 (2H, s), 7.06 (2H, s), 7.15 (2H, s), 7.25 (1H, m), 7.31-7.37 (4H, m), 8.365 (1H, d, J=5.5 Hz);

MS (FAB) m/z: 382 [M+H]⁺;

Anal. Calcd for C₂₀H₂₃N₅OS: C, 62.97; H, 6.08; N, 18.36; S, 8.41. Found: C, 62.94; H, 5.75; N, 18.33; S, 8.30.

### Example 57

### 3-amino-4-(4-phenyl-1,4-diazepan-1-yl)thieno[2,3-b]pyridine-2-carboxamide

(Exemplified Compound No. 3-86)

### (57a) tert-butyl 4-phenyl-1,4-diazepane-1-carboxylate

The title compound was synthesized by performing a reaction in a similar method as described in Org. Lett., 4, 581-584 (2002).

Brown liquid

IR (film) νₘₐₓ 2974, 1694, 1599, 1506, 1415, 1237, 1169, 930, 748, 692 cm⁻¹;

¹H NMR(CDCl₃, 400 MHz) δ 1.37 (4.5H, s), 1.44 (4.5H, s), 1.93-2.00 (2H, m), 3.19 (1H, t, J=5.9 Hz), 3.80 (1H, t, J=5.9 Hz), 3.51-3.57 (6H, m), 6.63-6.69 (3H, m), 7.19 (2H, t, J=7.1 Hz);

HRMS m/z calcd for C₁H₂₄O₂N₂ 276.1835, found 276.1833;

MS (EI) m/z: 276 [M⁺], 220, 205, 175, 146, 132, 120, 94, 57;

Anal. Calcd for C₁₆H₂₄N₂O₂.0.14H₂O: C, 68.90; H, 8.77; N, 10.04. Found: C, 68.96; H, 8.75; N, 9.85.

### (57b) 1-phenyl-1,4-diazepane

4N hydrochloric acid-dioxane solution (75 mL) was added to methanol (20 mL) solution of tert-butyl 4-phenyl-1,4-diazepane-1-carboxylate (16.58 g, 60.0 mmol) which was produced in Example 57 (57a) and the mixture was stirred at room temperature for one hour. The reaction liquid was concentrated, and a sodium hydrogen carbonate aqueous solution (100 mL) was added to the obtained residual substance, and extracted with a mixed solvent (3×100 mL) of methylene chloride/2-propanol (4:1) and after the extract was dried over sodium sulfate, the solvent was evaporated under reduced pressure and the title compound (10.57 g, yield 100%) was obtained.

Yellow liquid

IR (film) νₘₐₓ 2931, 1598, 1506, 1394, 1245, 1035, 748, 692 cm⁻¹;

¹H NMR(CDCl₃, 500 MHz) δ 1.87-1.92 (2H, m), 2.83 (2H, t, J=5.9 Hz), 3.03 (2H, t, J=5.9 Hz), 3.54-3.59 (4H, m), 6.65 (1H, t, J=8.3 Hz), 6.70 (2H, d, J=8.3 Hz), 7.21 (2H, t, J=8.3 Hz);

HRMS m/z calcd for C₁₁H₁₆N₂ 176.1313, found 176.1318;

MS (EI) m/z: 176 [M⁺], 146, 134, 120, 106, 94, 77, 69, 43.

### (57c) (2Z)-2-cyano-3-(4-phenyl-1,4-diazepan-1-yl)but-2-enethioamide

1-phenyl-1,4-diazepane which was produced in Example 57 (57b) was used in place of isobutylamine and the reaction was performed in a similar method as described in Example 5 (5a) and the title compound was synthesized.

Pale yellow powder

Mp 151-153° C.;

IR (KBr) νₘₐₓ 3290, 2185, 1599, 1538, 1503, 1397, 1369, 1011, 873, 754 cm⁻¹;

¹H NMR(DMSO-d₆, 500 MHz) δ 1.91-1.96 (2H, m), 2.23 (3H, s), 3.50-3.55 (4H, m), 3.61-3.63 (2H, m), 3.71-3.73 (2H, m), 6.61 (1H, t, J=7.3 Hz), 6.76 (2H, d, J=7.3 Hz), 7.16 (2H, t, J=7.3 Hz), 8.33 (1H, brs), 9.00 (1H, brs);

HRMS m/z calcd for C₁₆H₂₁N₄S 301.1487, found 301.1465;

MS (FAB) m/z: 300 [M⁺], 267, 242, 195, 175;

Anal. Calcd for C₁₆H₂₀N₄S: C, 63.97; H, 6.71; N, 18.65; S, 10.67. Found: C, 63.76; H, 6.47; N, 18.75, S, 10.62.

### (57d) 4-(4-phenyl-1,4-diazepan-1-yl)-2-thioxo-1,2-dihydropyridine-3-carbonitrile

(2Z)-2-cyano-3-(4-phenyl-1,4-diazepan-1-yl)but-2-enethioamide which was produced in Example 57 (57c) was used in place of (2Z)-2-cyano-3-(isobutylamino)but-2-enethioamide and the reaction was performed in a similar method as described in Example 5 (5b) and the title compound was synthesized.

Brown powder

Mp 128-132° C.;

IR (KBr) νₘₐₓ 2954, 2205, 1626, 1504, 1248, 1136, 928, 750, 617 cm⁻¹;

¹H NMR(DMSO-d₆, 400 MHz) δ 1.90-1.96 (2H, m), 3.51 (2H, t, J=5.9 Hz), 3.71 (4H, q, J=5.9 Hz), 3.93 (2H, t, J=5.9 Hz), 6.40 (1H, d, J=7.4 Hz), 6.56 (1H, t, J=7.1 Hz), 6.74 (2H, d, J=7.1 Hz), 7.12 (2H, t, J=7.1 Hz), 7.83 (1H, d, J=12.47 (1H, brs);

HRMS m/z calcd for C₁₇H₁₈N₄S 310.1252, found 310.1224;

MS (EI) m/z: 310 [M⁺], 281, 251, 204, 165, 132;

Anal. Calcd for C₁₇H₁₈N₄S: C, 65.78; H, 5.84; N, 18.05; S, 10.33. Found: C, 65.58; H, 5.51; N, 18.03; S, 10.24.

### (57e) 3-amino-4-(4-phenyl-1,4-diazepan-1-yl)thieno[2,3-b]pyridine-2-carboxamide

4-(4-phenyl-1,4-diazepan-1-yl)-2-thioxo-1,2-dihydropyridine-3-carbonitrile which was produced in Example 57 (57d) was used in place of 4-(isobutylamino)-2-thioxo-1,2-dihydropyridine-3-carbonitrile and the reaction was performed in a similar method as described in Example 5 (5c) and the title compound was synthesized.

White powder

Mp 215-218° C.;

IR (KBr) νₘₐₓ 3340, 3316, 3143, 1645, 1598, 1504, 1369, 1233, 939, 752, 694 cm⁻¹;

¹H NMR(DMSO-d₆, 400 MHz) δ 2.10-2.15 (2H, m), 3.16-3.19 (2H, m), 3.26-3.29 (2H, m), 3.52 (2H, t, J=6.3 Hz), 3.75 (2H, t, J=6.3 Hz), 6.58 (1H, t, J=8.2 Hz), 6.74 (2H, d, J=8.2 Hz), 6.95 (2H, brs), 7.05 (1H, d, J=5.5 Hz), 7.05 (2H, brs), 7.14 (2H, t, J=8.2 Hz), 8.36 (1H, d, J=5.5 Hz);

HRMS m/z calcd for C₁₉H₂₁N₅OS 367.1467, found 367.1462;

MS (EI) m/z: 367 [M⁺], 335, 324, 275, 259, 242, 216, 202, 175, 146, 120;

Anal. Calcd for C₁₉H₂₁N₅OS.0.24H₂O: C, 61.38; H, 5.82; N, 18.84; S, 8.62. Found: C, 61.22; H, 5.64; N, 18.94; S, 8.49.

### Example 58

### 3-amino-4-[4-(4-methoxyphenyl)-1,4-diazepan-1-yl]thieno[2,3-b]pyridine-2-carboxamide

(Exemplified Compound No. 3-124)

### (58a) tert-butyl 4-(4-methoxyphenyl)-1,4-diazepane-1-carboxylate

The title compound was synthesized by performing a reaction in a similar method as described in Org. Lett., 4,581-584 (2002).

Brown liquid

IR (film) νₘₐₓ 2974, 1693, 1513, 1415, 1242, 1169, 1041, 930, 815 cm⁻¹;

¹H NMR(CDCl₃, 500 MHz) δ1.37 (4.5H, s), 1.44 (4.5H, s), 1.92-1.99 (2H, m), 3.20 (1H, t, J=5.9 Hz), 3.31 (1H, t, J=5.9 Hz), 3.47-3.57 (6H, m), 3.74 (3H, s), 6.65 (2H, d, J=9.3 Hz), 6.81 (2H, d, J=9.3 Hz);

HRMS m/z calcd for C₁₇H₂₆O₃N₂ 306.1943, found 306.1935;

MS (EI) m/z: 306 [M⁺], 250, 235, 205, 189, 162, 150, 121, 70, 57;

Anal. Calcd for C₁₇H₂₆N₂O₃.0.36H2O: C, 65.26; H, 8.61; N, 8.95. Found: C, 64.92; H, 8.29; N, 8.87.

### (58b) 1-(4-methoxyphenyl)-1,4-diazepane

tert-butyl 4-(4-methoxyphenyl)-1,4-diazepane-1-carboxylate which was produced in Example 58 (58a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2934, 1513, 1241, 1040, 814 cm⁻¹;

¹H NMR(CDCl₃, 400 MHz) δ 1.89 (2H, quint, J=5.1 Hz), 2.89 (2H, t, J=5.1 Hz), 3.02 (2H, t, J=5.1 Hz), 3.48-3.54 (4H, m), 3.75 (3H, s), 6.65 (2H, d, J=9.0 Hz), 6.82 (2H, d, J=9.0 Hz);

HRMS m/z calcd for C₁₂H₁₈ON₂ 206.1419, found 206.1424;

MS (EI) m/z: 206 [M⁺], 176, 164, 150, 136, 121, 109, 92, 77, 70, 43.

### (58c) (2Z)-2-cyano-3-[4-(4-methoxyphenyl)-1,4-diazepan-1-yl]but-2-enethioamide

1-(4-methoxyphenyl)-1,4-diazepane which was produced in Example 58 (58b) was used in place of isobutylamine and the reaction was performed in a similar method as described in Example 5 (5a) and the title compound was synthesized.

Pale yellow powder

Mp 157-158° C.;

IR (KBr) νₘₐₓ 3284, 3154, 2179, 1512, 1361, 1243, 1037, 821 cm⁻¹;

¹H NMR(DMSO-d₆, 400 MHz) δ 1.89-1.96 (2H, m), 2.24 (3H, s), 3.44 (2H, t, J=5.9 Hz), 3.49 (2H, t, J=5.9 Hz), 3.57-3.63 (4H, m), 3.64 (3H, s), 6.70 (2H, d, J=9.0 Hz), 6.76 (2H, d, J=9.0 Hz), 8.27 (1H, brs), 8.95 (1H, brs);

HRMS m/z calcd for C₁₇H₂₃ON₄S 331.1593, found 331.1589;

MS (FAB) m/z: 331 [M+H]⁺, 246, 228, 182, 63;

Anal. Calcd for C₁₇H₂₂N₄OS: C, 61.79; H, 6.71; N, 16.95; S, 9.70. Found: C, 61.51; H, 6.69; N, 16.99; S, 9.71.

### (58d) (2Z)-2-cyano-N-[(1E)-(dimethylamino) methylene]-3-[4-(4-methoxyphenyl)-1,4-diazepan-1-yl]but-2-enethioamide

(2Z)-2-cyano-3-[4-(4-methoxyphenyl)-1,4-diazepan-1-yl]but-2-enethioamide (476 mg, 1.44 mmol) which was produced in Example 58 (58c) was suspended in ethanol (15 mL) and was blended with N,N-dimethylformamide dimethylacetal (382 μL, 2.88 mmol) and the mixture was stirred at room temperature for one day. After the deposited solid was separated by filtration, it was washed with ethanol, the title compound (452 mg, yield 81%) was obtained.

Yellow powder

Mp 145-146° C.;

IR (KBr) νₘₐₓ 2926, 2178, 1610, 1512, 1324, 1294, 1187, 1012, 923, 669, 514 cm⁻¹;

¹H NMR(DMSO-d₆, 400 MHz) δ 1.94-2.00 (2H, m), 2.50 (3H, s), 2.93 (3H, s), 3.14 (3H, s), 3.45-3.49 (2H, m), 3.65 (3H, s), 3.69-3.79 (6H, m), 6.73 (2H, d, J=7.8 Hz), 6.78 (2H, d, J=7.8 Hz), 8.46 (1H, s);

HRMS m/z calcd for C₂₀H₂₈ON₅S 386.2014, found 386.2007;

MS (FAB) m/z: 386 [M+H]⁺, 369, 352, 273, 242, 196, 165, 65, 55;

Anal. Calcd for C₂₀H₂₇N₅OS: C, 62.31; H, 7.06; N, 18.17; S, 8.32. Found: C, 62.03; H, 6.88; N, 18.02; S, 8.50.

### (58e) 3-amino-4-[4-(4-methoxyphenyl)-1,4-diazepan-1-yl]thieno[2,3-b]pyridine-2-carboxamide

N,N-dimethylformamide (2.3 mL) solution of (2Z)-2-cyano-N-[(1E) -(dimethylamino)methylene]-3-[4-(4-methoxyphenyl)-1,4-diazepan-1-yl]but-2-enethioamide (443 mg, 1.15 mmol) which was produced in Example 58 (58d) was stirred at 80° C. for one hour. The reaction liquid was cooled to room temperature and was blended with 2-chloroacetamide (129 mg, 1.38 mmol) and 8M aqueous solution of sodium hydroxide (0.49 mL) and the mixture was stirred at room temperature for one hour. Water (5 mL) was added to the reaction liquid and the resulted solid was separated by filtration and the title compound (378 mg, yield 83%) was obtained.

Pale yellow powder

Mp 206-208° C.;

IR (KBr) νₘₐₓ 3446, 3328, 3168, 1578, 1511, 1370, 1240, 1037, 937, 816 cm⁻¹;

¹H NMR (DMSO-d₆, 400 MHz) δ 1.78-1.86 (2H, m), 2.89-2.95 (2H, m), 2.97-3.01 Hz), 6.51 (2H, d, J=9.4 Hz), 6.69 (2H, brs), 6.77-6.79 (2H, m), 8.10 (1H, d, J=5.5 Hz);

HRMS m/z calcd for C₂₀H₂₄O₂N₅S 398.1651, found 398.1635;

MS (FAB) m/z: 398 [M+H]⁺, 273, 257, 242, 226, 200, 165, 63;

Anal. Calcd for C₂₀H₂₃N₅O₂S.0.52H₂O: C, 59.04; H, 5.96; N, 17.21. Found: C, 58.73; H, 6.09; N, 17.52.

### Example 59

### 3-amino-4-[4-(4-nitrophenyl)-1,4-diazepan-1-yl]thieno[2,3-b]pyridine-2-carboxamide

(Exemplified Compound No. 3-97)

### (59a) tert-butyl 4-(4-nitrophenyl)-1,4-diazepane-1-carboxylate

4-fluoro nitrobenzene (282 mg, 2 mmol) and tert-butoxycarbonyl homopiperazine (801 mg, 4 mmol) were dissolved in dimethylsulfoxide (5 mL) and the mixture was stirred at 80° C. for one hour. Ethyl acetate (20 mL) and water (20 mL) were added to the reaction liquid and, after partitioning, the organic layer was washed with water (20 mLx5) and a saline solution (20 mL), and after drying over sodium sulfate, the solvent was evaporated under reduced pressure. After the obtained residue was blended with hexane and solidified, it was separated by filtration and purified and the title compound (620 mg, yield 96%) was obtained.

Yellow powder

IR (KBr) νₘₐₓ 1683, 1600, 1482, 1422, 1310, 1253, 1114, 983, 824 cm⁻¹;

¹H NMR(DMSO-d₆, 400 MHz) δ 1.16 (4.5H, s), 1.27 (4.5H, s), 1.71-1.83 (2H, m), 3.22 (1H, t, J=5.5 Hz), 3.29 (1H, t, J=7.4 Hz), 3.50 (1H, t, J=5.9 Hz), 356 (1H, t, J=5.9 Hz), 3.62-3.65 (2H, m), 3.70-3.77 (2H, m), 6.87 (2H, d, J=9.4 Hz), 8.00-8.04 (2H, m);

HRMS m/z calcd for C₁₆H₂₃N₃O₄ 321.1689, found 321.1682;

MS (FAB) m/z: 321 [M⁺], 266, 220, 200, 120.

### (59b) 1-(4-nitrophenyl)-1,4-diazepane

tert-butyl 4-(4-nitrophenyl)-1,4-diazepane-1-carboxylate which was produced in Example 59 (59a) was used in place of tert-butyl 4-phenyl-1,4-diazepane-1-carboxylate and the reaction was performed in a similar method as described in Example 57 (57b) and the title compound was obtained.

Yellow liquid

¹H NMR(DMSO-d₆, 400 MHz) δ 1.71-1.77 (2H, m), 2.61 (2H, t, J=5.9 Hz), 2.83 (2H, t, J=5.9 Hz), 3.56 (2H, t, J=5.9 Hz), 3.63 (2H, t, J=5.9 Hz), 6.79 (2H, d, J=9.4 Hz), 7.99 (2H, d, J=9.4 Hz).

### (59c) (2Z)-2-cyano-3-[4-(4-nitrophenyl)-1,4-diazepan-1-yl]but-2-enethioamide

1-(4-nitrophenyl)-1,4-diazepane which was produced in Example 59 (59b) was used in place of isobutylamine and the reaction was performed in a similar method as described in Example 5 (5a) and the title compound was synthesized.

Yellow powder

IR (KBr) νₘₐₓ 3190, 2183, 1596, 1516, 1315, 1115 cm⁻¹;

¹H NMR(DMSO-d₆, 400 MHz) δ 1.91-1.96 (2H, m), 2.22 (3H, s), 3.54-3.57 (2H, m), 3.52-3.65 (2H, m), 3.69-3.72 (2H, m), 3.90-3.92 (2H, m), 6.91 (2H, d, J=8.2 Hz), 8.03 (2H, d, J=8.2 Hz), 8.45 (1H, brs), 9.08 (1H, brs);

HRMS m/z calcd for C₁₆H₂₀O₂N₅S 346.1337, found 346.1342;

MS (FAB) m/z: 346 [M+H]⁺, 329, 273, 200, 165.

### (59d) 4-[4-(4-nitrophenyl)-1,4-diazepan-1-yl)-2-thioxo-1,2-dihydropyridine-3-carbonitrile

(2Z)-2-cyano-3-[4-(4-nitrophenyl)-1,4-diazepan-1-yl]but-2-enethioamide which was produced in Example 59 (59c) was used in place of (2Z)-2-cyano-3-(isobutylamino)but-2-enethioamide and the reaction was performed in a similar method as described in Example 5 (5b) and the title compound was obtained.

Brown powder

IR (KBr) νₘₐₓ 2204, 1596, 1515, 1312, 1114, 927, 752 cm⁻¹;

¹H NMR(DMSO-d₆, 400 MHz) δ 1.91-1.97 (2H, m), 3.65-3.70 (2H, m), 3.75-3.78 (2H, m), 3.88-3.90 (2H, m), 3.97-3.99 (2H, m), 6.43 (1H, d, J=7.8 Hz), 6.92 (1H, t, J=9.4 Hz), 7.86 (1H, d, J=7.8 Hz), 8.01 (2H, d, J=9.4 Hz);

HRMS m/z calcd for C₁₇H₁₈N₅O₂S 356.1182, found 356.1165;

MS (EI) m/z: 356 [M+H]⁺, 273, 246, 182, 120.

### (59e) 3-amino-4-[4-(4-nitrophenyl)-1,4-diazepan-1-yl]thieno[2,3-b]pyridine-2-carboxamide

4-[4-(4-nitrophenyl)-1,4-diazepan-1-yl]-2-thioxo-1,2-dihydropyridine-3-carbonitrile which was produced in Example 59 (59d) was used in place of 4-(isobutylamino)-2-thioxo-1,2-dihydropyridine-3-carbonitrile and the reaction was performed in a similar method as described in Example 5 (5c) and the title compound was obtained.

Yellow powder

Mp 244-248° C.;

IR (KBr) νₘₐₓ 3439, 1646, 1596, 1509, 1312, 1114, 824, 7534 cm⁻¹;

¹H NMR(DMSO-d₆, 400 MHz) δ 2.17-2.23 (2H, m), 3.18-3.24 (2H, m), 3.31-3.36 (2H, m), 3.72 (2H, t, J=5.9 Hz), 3.91-3.95 (2H, m), 6.93 (2H, d, J=9.4 Hz), 7.00 (2H, brs), 7.08 (1H, d, J=5.5 Hz), 7.12 (2H, brs), 8.07 (2H, t, J=9.4 Hz), 8.41 (1H, d, J=5.5 Hz);

HRMS m/z calcd for C₁₉H₂₁N₆O₃S 413.1396. found 413.1400;

MS (FAB) m/z: 413 [M+H]⁺, 338, 246, 182;

Anal. Calcd. for C₁₉H₂₀N₆O₃S.0.76H₂O: C, 53.55; H, 5.09; N, 19.72; S, 7.52. Found: C, 53.85; H, 4.86; N, 19.54, S, 7.14.

### Example 60

### 3-amino-4-[4-(4-chlorophenyl)-1,4-diazepan-1-yl]thieno[2,3-b]pyridine-2-carboxamide

(Exemplified Compound No. 3-94)

### (60a) tert-butyl 4-(4-chlorophenyl)-1,4-diazepane-1-carboxylate

The reaction was performed in a similar method as described in Org. Lett., 4,581-584(2002) and the title compound was synthesized.

Brown liquid

¹H NMR(DMSO-d₆, 400 MHz) δ 1.36 (4.5H, s), 1.43 (4.5H, s), 1.91-1.97 (2H, m), 3.19 (1H, t, J=6.3 Hz), 3.30 (1H, t, J=6.3 Hz), 3.48-3.56 (6H, m), 6.59 (2H, d, J=9.0 Hz), 7.121 (2H, d, J=3.5, 9.0 Hz).

### (60b) 1-(4-chlorophenyl)-1,4-diazepane

tert-butyl 4-(4-chlorophenyl)-1,4-diazepane-1-carboxylate which was produced in Example 60 (60a) was used in place of tert-butyl 4-phenyl-1,4-diazepane-1-carboxylate and the reaction was performed in a similar method as described in Example 57 (57b) and the title compound was obtained.

Yellow liquid

¹H NMR(CDCl₃, 400 MHz) δ 1.87 (2H, quint, J=5.9 Hz), 2.80 (2H, t, J=5.9 Hz), 3.00 (2H, t, J=5.9 Hz), 3.49 (2H, t, J=5.9 Hz), 3.53 (2H, t, J=5.9 Hz), 6.58 (2H, t, J=9.4 Hz), 7.11 (2H, d, J=9.4 Hz).

### (60c) (2Z)-3-[4-(4-chlorophenyl)-1,4-diazepan-1-yl]-2-cyanobut-2-enethioamide

1-(4-chlorophenyl)-1,4-diazepane which was produced in Example 60 (60b) was used in place of isobutylamine and the reaction was performed in a similar method as described in Example 5 (5a) and the title compound was synthesized.

Pale yellow powder

Mp 170-172° C. (decomposition);

IR (KBr) νₘₐₓ 3370, 3268, 3175, 2187, 1536, 1498, 1397, 813, 510 cm⁻¹;

¹H NMR(DMSO-d₆, 400 MHz) δ 1.88-1.94 (2H, m), 2.22 (3H, s), 3.48-3.53 (4H, m), 3.58-3.60 (2H, m), 3.70-3.72 (2H, m), 6.75 (2H, d, J=9.0 Hz), 7.14 (2H, d, J=9.0 Hz), 8.34 (1H, brs), 8.98 (1H, brs);

HRMS m/z calcd for C₁₆H₂₀N₄ClS 335.1097, found 335.1095;

MS (FAB) m/z: 335 [M+H]⁺, 273, 246, 211, 165, 63;

Anal. Calcd. for C₁₆H₁₉ClN₄S: C, 57.39; H, 5.47; N, 16.73; Cl, 10.59; S, 9.58. Found: C, 57.23; H, 5.47; N, 17.77; Cl, 10.59; S, 9.54.

### (60d) (2Z)-3-[4-(4-chlorophenyl)-1,4-diazepan-1-yl]-2-cyano-N-[(1E)-(dimethylamino)methylene]but-2-enethioamide

(2Z)-3-[4-(4-chlorophenyl)-1,4-diazepan-1-yl]-2-cyanobut-2-enethioamide which was produced in Example 60 (60c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 259-264° C.;

IR (KBr) νₘₐₓ 2926, 2176, 1610, 1499, 1328, 1291, 1194, 1012, 923, 820 cm⁻¹;

¹H NMR(DMSO-d₆, 400 MHz) δ 1.94-1.97 (2H, m), 2.46 (3H, s), 2.90 (3H, s) 3.13 (3H, s), 3.54 (2H, t, J=5.9 Hz), 3.69-3.76(4H, m), 3.79-3.81 (2H, m), 6.75 (2H, d, J=9.0 Hz), 7.11 (2H, d, J=9.0 Hz), 8.42 (1H, s);

HRMS m/z calcd for C₁₉H₂₅N₅ClS 390.1519, found 390.1524;

MS (FAB) m/z: 390 [M+H]⁺, 356, 318, 273, 208, 196, 180, 166, 90;

Anal. Calcd. for C₁₉H₂₄ClN₅S: C, 58.42; H, 6.20; N, 17.96; Cl, 9.09; S, 8.22. Found: C, 58.41; H, 6.17; N, 17.66, Cl, 8.91; S, 8.12.

### (60e) 3-amino-4-[4-(4-chlorophenyl)-1,4-diazepan-1-yl]thieno[2,3-b]pyridine-2-carboxamide

(2Z)-3-[4-(4-chlorophenyl)-1,4-diazepan-1-yl]-2-cyano-N-[(1E)-(dimethylamino)methylene]but-2-enethioamide which was produced in Example 60 (60d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder

Mp 92-96° C.;

IR (KBr) νₘₐₓ 3441, 3323, 2948, 1646, 1594, 1499, 1368, 939, 810 cm⁻¹;

¹H NMR(DMSO-d₆, 500 MHz) δ 2.11-2.16 (2H, m), 3.15-3.20 (2H, m), 3.26-3.28 (2H, m), 3.53 (2H, t, J=5.9 Hz), 3.75 (2H, t, J=4.4 Hz), 6.76 (2H, d, J=8.8 Hz), 6.99 (2H, brs), 7.07 (1H, d, J=5.4 Hz), 7.08 (2H, brs), 7.17 (2H, t, J=8.8 Hz), 8.40 (1H, d, J=5.4 Hz);

HRMS m/z calcd for C₁₉H₂₁N₅OClS 402.1155, found 402.1158;

MS (FAB) m/z: 402 [M+H]⁺, 385, 273, 258, 246, 211, 200, 93;

Anal. Calcd. for C₁₉H₂₀ClN₅OS.0.51H₂O: C, 55.51; H, 5.15; N, 17.04; Cl, 8.62. Found: C, 55.60; H, 5.09; N, 16.84; Cl, 8.76.

### Example 61

### 3-amino-4-[4-(2-chlorophenyl)-1,4-diazepan-1-yl]thieno[2,3-b]pyridine-2-carboxamide

(Exemplified Compound No. 3-92)

### (61a) tert-butyl 4-(2-chlorophenyl)-1,4-diazepane-1-carboxylate

The reaction was performed in a similar method as described in Org. Lett., 4, 581-584 (2002) and the title compound was synthesized.

Brown liquid

IR (film) νₘₐₓ 2976, 1692, 1482, 1366, 1160, 751 cm⁻¹;

¹H NMR(CDCl₃, 400 MHz) δ 1.46 (4.5H, s), 1.47 (4.5H, s), 1.97-2.04 (2H, m), 3.14-3.21 (4H, m), 3.53-3.64 (4H, m), 6.90 (1H, t, J=7.1 Hz), 7.04 (1H, d, J=7.1 Hz), 7.14 (1H, t, J=7.1 Hz), 7.32 (1H, d, J=7.1 Hz);

HRMS m/z calcd for C₁₆H₂₃N₂O₂³⁵Cl 310.1448, found 310.1472;

MS (EI) m/z: 310 [M⁺], 282, 253, 237, 209, 193, 180, 166, 154, 138, 125, 111, 70, 57.

### (61b) 1-(2-chlorophenyl)-1,4-diazepane

tert-butyl 4-(2-chlorophenyl)-1,4-diazepane-1-carboxylate which was produced in Example 61 (61a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2941, 2836, 1588, 1481, 1295, 1040, 752 cm⁻¹;

¹H NMR(CDCl₃, 400 MHz) δ 1.93 (2H, quint, J=5.9 Hz), 2.18 (1H, brs), 3.03-3.08 (4H, m), 3.23-3.29 (4H, m), 6.87 (1H, t, J=5.9 Hz), 7.07 (1H, d, J=5.9 Hz), 7.14 (1H, t, J=5.9 Hz), 7.31 (1H, d, J=5.9 Hz);

HRMS m/z calcd for C₁₁H₁₅N₂Cl 210.0924, found 210.0916;

MS (EI) m/z: 210 [M⁺], 180, 175, 168, 154, 146, 128, 111, 92, 77, 64.

### (61c) (2Z)-3-[4-(2-chlorophenyl)-1,4-diazepan-1-yl]-2-cyanobut-2-enethioamide

1-(2-chlorophenyl)-1,4-diazepane which was produced in Example 61 (61b) was used in place of isobutylamine and the reaction was performed in a similar method as described in Example 5 (5a) and the title compound was synthesized.

Pale yellow powder

Mp 123-124° C.;

IR (KBr) νₘₐₓ 3367, 3265, 3186, 2184, 1611, 1525, 1402, 1289, 1041, 766, 673 cm⁻¹;

¹H NMR(CDCl₃, 400 MHz) δ 2.12-2.17 (2H, m), 2.69 (3H, s), 3.23 (2H, t, J=5.9 Hz), 3.34-3.37 (2H, m), 3.91 (2H, t, J=5.9 Hz), 3.97-4.00 (2H, m), 6.70 (2H, brs), 6.97 (1H, dt, J=1.2, 8.2 Hz), 7.05 (1H, dd, J=1.2, 8.2 Hz), 7.19 (1H, dt, J=1.2, 8.2 Hz), 7.35 (1H, dd, J=1.2, 8.2 Hz);

HRMS m/z calcd for C₁₆H₂₀N₄ClS 335.1097, found 335.1098;

MS (FAB) m/z: 335 [M+H]⁺, 273, 246, 211;

Anal. Calcd. for C₁₆H₁₉ClN₄S.0.64H₂O: C, 55.48; H, 5.90; N, 16.17; Cl, 10.23; S, 9.26. Found: C, 55.69; H, 5.81; N, 16.19; Cl, 9.97; S, 9.35.

### (61d) 3-amino-4-[4-(2-chlorophenyl)-1,4-diazepan-1-yl]thieno[2,3-b]pyridine-2-carboxamide

(2Z)-3-[4-(2-chlorophenyl)-1,4-diazepan-1-yl]-2-cyanobut-2-enethioamide (64 mg, 0.19 mmol) which was produced in Example 61 (61c) and N,N-dimethylformamide dimethylacetal (54 mg, 0.45 mmol) were dissolved in ethanol (2 mL) and after stirring at room temperature for one hour, the solvent was evaporated. The residue was dissolved in N,N-dimethylformamide (0.3 mL) and the mixture was stirred at 80° C. for 15 minutes. The reaction mixture was cooled to room temperature and 8N aqueous solution of sodium hydroxide (0.08 mL) and 2-chloroacetamide (21 mg, 0.22 mmol) were added. After stirring at room temperature for one hour, the reaction mixture was partitioned with water and ethyl acetate and after the organic layer was dried over sodium sulfate, the solvent was evaporated under reduced pressure. A solid was separated by filtration after the residue was solidified with hydrous ethanol and 76 mg of the title compound was obtained (yield 31%).

Pale yellow powder

Mp 206-210° C.;

IR (KBr) νₘₐₓ 3435, 3318, 3168, 1645, 1584, 1501, 1369, 1041, 937, 761 cm⁻¹;

¹H NMR(DMSO-d₆, 400 MHz) δ 2.05-2.11 (2H, m), 3.28-3.31 (2H, m), 3.41-3.48 (6H, m), 6.97-7.01 (1H, m), 7.07 (2H, brs), 7.11 (2H, brs), 7.11-7.12 (1H, m), 7.25-7.27 (2H, m), 7.40 (1H, d, J=7.1 Hz), 8.40 (1H, d, J=5.5 Hz);

HRMS m/z calcd for C₁₉H₂₁N₅OClS 402.1155, found 402.1153;

MS (FAB) m/z: 402 [M+H]⁺, 246, 189, 182;

Anal. Calcd. for C₁₉H₂₀ClN₅OS.0.22H₂O: C, 56.23; H, 5.08; N, 17.25; Cl, 8.73; S, 7.90. Found: C, 56.18; H, 5.11; N, 17.08; Cl, 8.66; S, 7.62.

### Example 62

### 3-amino-4-[4-(4-cyanophenyl)-1,4-diazepan-1-yl]thieno[2,3-b]pyridine-2-carboxamide

(Exemplified Compound No. 3-98)

### (62a) tert-butyl 4-(4-cyanophenyl)-1,4-diazepane-1-carboxylate

The reaction was performed in a similar method as described in Example 59 (59a) using 4-cyano fluorobenzene (242 mg, 2 mmol) and tert-butoxycarbonyl homopiperazine (801 mg, 4 mmol) and the title compound (364 mg, yield 60%) was obtained.

Brown liquid

IR (film) νₘₐₓ 2975, 2214, 1691, 1606, 1521, 1417, 1365, 1240, 1178, 929, 819, 544 cm⁻¹;

¹H NMR(DMSO-d₆, 400 MHz) δ 1.16 (4.5H, s), 1.28 (4.5H, s), 1.68-1.85 (2H, m), 3.17 (1H, t, J=5.9 Hz), 3.25 (1H, t, J=5.5 Hz), 3.44-3.68 (6H, m), 6.81 (2H, d, J=9.4 Hz), 7.48 (2H, dd, J=3.5, 9.4 Hz);

HRMS m/z calcd for C₁₇H₂₃N₃O₂ 301.1790, found 301.1784;

MS (FAB) m/z: 302 [M+H]⁺, 301, 246, 228, 200, 120.

### (62b) 1-(4-cyanophenyl)-1,4-diazepane

tert-butyl 4-(4-cyanophenyl)-1,4-diazepane-1-carboxylate which was produced in Example 62 (62a) was used in place of tert-butyl 4-phenyl-1,4-diazepane-1-carboxylate and the reaction was performed in a similar method as described in Example 57 (57b) and the title compound was obtained.

Brown liquid

IR (film) νₘₐₓ 2935, 2211, 1606, 1521, 1404, 1178, 817, 544 cm⁻¹;

¹H NMR(CDCl₃, 400 MHz) δ 1.87 (2H, quint, J=5.9 Hz), 2.81 (2H, t, J=5.9 Hz), 3.01 (2H, t, J=5.9 Hz), 3.55 (2H, t, J=5.9 Hz), 3.61 (2H, t, J=5.9 Hz), 6.65 (2H, t, J=9.0 Hz), 8.43 (2H, d, J=9.0 Hz);

HRMS m/z calcd for C₁₂H₁₅N₃ 201.1266, found 201.1268;

MS (EI) m/z: 201 [M⁺], 171, 159, 145, 131, 116, 102.

### (62c) (2Z)-2-cyano-3-[4-(4-cyanophenyl)-1,4-diazepan-1-yl]but-2-enethioamide

1-(4-cyanophenyl)-1,4-diazepane which was produced in Example 62 (62b) was used in place of isobutylamine and the reaction was performed in a similar method as described in Example 5 (5a) and the title compound was synthesized.

Pale yellow powder

Mp 169-171° C. (decomposition);

IR (KBr) νₘₐₓ 3290, 2214, 217, 1604, 1519, 1408, 1179, 819, 543 cm⁻¹;

¹H NMR(DMSO-d₆, 400 MHz) δ 1.88-1.94 (2H, m), 2.21 (3H, s), 3.52 (2H, t, J=5.5 Hz), 3.58-3.63 (4H, m), 3.82 (2H, t, J=5.5 Hz), 6.85 (2H, d, J=9.0 Hz), 7.51 (2H, d, J=9.0 Hz), 8.38 (1H, brs), 9.02 (1H, brs);

HRMS m/z calcd for C₁₇H₂₀N₅S 326.1440, found 326.1436;

MS (FAB) m/z: 326 [M+H]⁺, 202, 171, 120.

### (62d) (2Z)-2-cyano-3-[4-(4-cyanophenyl)-1,4-diazepan-1-yl]-N-[(1E)-(dimethylamino)methylene]but-2-enethioamide

(2Z)-3-[4-(4-cyanophenyl)-1,4-diazepan-1-yl]-2-cyanobut-2-enethioamide which was produced in Example 62 (62c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 120-126° C.;

IR (KBr) νₘₐₓ 2926, 2211, 2180, 1606, 1519, 1178, 821, 546 cm⁻¹;

hu 1H NMR(DMSO-d₆, 400 MHz) δ 1.94-1.97 (2H, m), 2.44 (3H, s), 2.90 (3H, s) 3.14 (3H, s), 3.64 (2H, t, J=5.5 Hz), 3.71-3.77(4H, m), 3.90 (2H, t, J=5.5 Hz), 6.85 (2H, d, J=9.0 Hz), 7.48 (2H, d, J=9.0 Hz), 8.41 (1H, s);

HRMS m/z calcd for C₂₀H₂₅N₆S 381.1861, found 381.1856;

MS (FAB) m/z: 381 [M+H]⁺, 336, 257, 230, 202, 180, 90, 65;

Anal. Calcd. for C₂₀H₂₄N₆S.0.62H₂O: C, 61.33; H, 6.50; N. 21.46; S, 8.19. Found: C, 61.36; H, 6.35; N, 21.25, S, 8.11.

### (62e) 3-amino-4-[4-(4-cyanophenyl)-1,4-diazepan-1-yl]thieno[2,3-b]pyridine-2-carboxamide

(2Z)-2-cyano-3-[4-(4-cyanophenyl)-1,4-diazepan-1-yl]-N-[(1E)-(dimethylamino)methylene]but-2-enethioamide which was produced in Example 62 (62d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Yellow powder

Mp 151-153° C.;

IR (KBr) νₘₐₓ 3439, 3327, 2211, 1605, 1519, 1366, 1178, 938, 818, 544 cm⁻¹;

¹H NMR(DMSO-d₆, 400 MHz) δ 2.13-2.18 (2H, m), 3.17-3.19 (2H, m), 3.61 (2H, t, J=6.3 Hz), 3.84 (2H, t, J=5.1 Hz), 6.86 (2H, d, J=9.0 Hz), 6.96 (2H, brs), 7.05 (1H, d, J=5.5 Hz), 7.07 (2H, brs), 7.52 (2H, t, J=9.0 Hz), 8.37 (1H, d, J=5.5 Hz);

HRMS m/z calcd for C₂₀H₂₁N₆OS 393.1497, found 393.1501;

MS (FAB) m/z: 393 [M+H]⁺, 273, 246;

Anal. Calcd. for C₂₀H₂₀N₆OS.0.94H₂O: C, 58.67; H, 5.39; N, 20.53. Found: C, 58.99; H, 5.51; N, 20.22.

### Example 63

### 3-amino-4-[4-(4-trifluoromethylphenyl)-1,4-diazepan-1-yl]thieno[2,3-b]pyridine-2-carboxamide

(Exemplified Compound No. 3-121)

### (63a) tert-butyl 4-(4-trifluoromethylphenyl)-1,4-diazepane-1-carboxylate

The reaction was performed in a similar method as described in Org. Lett., 4, 581-584 (2002) and the title compound was synthesized.

White powder

IR (KBr) νₘₐₓ 2974, 1673, 1422, 1332, 1197, 1100, 987, 829 cm⁻¹;

¹H NMR(CDCl₃, 400 MHz) δ 1.33 (4.5H, s), 1.41 (4.5H, s), 1.93-1.99 (2H, m), 3.20 (1H, t, J=6.3 Hz), 3.31 (1H, t, J=5.9 Hz), 3.54-3.62 (6H, m), 6.67 (2H, d, J=9.0 Hz), 7.40 (2H, d, J=9.0 Hz);

HRMS m/z calcd for C₁₇H₂₃N₂O₂F₃ 344.1711, found 344.1718;

MS (FAB) m/z: 345 [M+H]⁺, 344, 289, 243, 214, 174, 120, 57;

Anal. Calcd. for C₁₇H₂₃F₃N₂O₂: C, 59.29; H, 6.73; N, 8.13. Found: C, 59.38; H, 6.45; N, 7.98.

### (63b) 1-(4-trifluoromethylphenyl)-1,4-diazepane

tert-butyl 4-(4-trifluoromethylphenyl)-1,4-diazepane-1-carboxylate which was produced in Example 63 (63a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2936, 1616, 1531, 1402, 1330, 1197, 1106, 818 cm⁻¹;

¹H NMR(CDCl₃, 400 MHz) δ 1.88 (2H, quint, J=6.3 Hz), 2.81 (2H, t, J=5.9 Hz), 3.01 (2H, t, J=5.1 Hz), 3.54-3.61 (4H, m), 6.67 (2H, t, J=9.0 Hz), 8.39 (2H, d, J=9.0 Hz);

HRMS m/z calcd for C₁₂H₁₅N₂F₃ 244.1187, found 244.1182;

MS (EI) m/z: 244 [M⁺], 225, 214, 202, 188, 174, 159, 145, 69, 43.

### (63c) (2Z)-2-cyano-3-[4-(4-trifluoromethylphenyl)-1,4-diazepan-1-yl]but-2-enethioamide

1-(4-trifluoromethylphenyl)-1,4-diazepane which was produced in Example 63 (63b) was used in place of isobutylamine and the reaction was performed in a similar method as described in Example 5 (5a) and the title compound was synthesized.

Pale yellow powder

Mp 266-269° C. (decomposition);

IR (KBr) νₘₐₓ 3373, 3267, 3171, 2185, 1614, 1527, 1330, 1104, 822 cm⁻¹;

¹H NMR(DMSO-d₆, 400 MHz) δ 1.91-1.95 (2H, m), 2.23 (3H, s), 3.51 (2H, t, J=5.5 Hz), 3.59-3.63 (4H, m), 3.79 (2H, t, J=5.1 Hz), 6.87 (2H, d, J=8.6 Hz), 7.42 (2H, d, J=8.6 Hz), 8.37 (1H, brs), 9.01 (1H, brs);

HRMS m/z calcd for C₁₇H₂₀N₄F₃S 369.1361, found 369.1359;

MS (FAB) m/z: 369 [M+H]⁺, 335, 245, 227, 200, 166, 63;

Anal. Calcd. for C₁₇H₁₉F₃N₄S 0.10H₂O: C, 55.15; H, 5.23; N, 15.13; F, 15.39. Found: C, 54.92; H, 5.26; N, 15.25, F, 15.59.

### (63d) (2Z)-2-cyano-N-[(1E)-(dimethylamino)methylene]-3-[4-(4-trifluoromethylphenyl)-1,4-diazepan-1-yl]but-2-enethioamide

(2Z)-3-[4-(4-trifluoromethylphenyl)-1,4-diazepan-1-yl]-2-cyanobut-2-enethioamide which was produced in Example 63 (63c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 251-253° C.;

IR (KBr) νₘₐₓ 2927, 2177, 1614, 1400, 1332, 1201, 1107, 925, 832 cm⁻¹;

¹H NMR(DMSO-d₆, 400 MHz) δ 1.95-1.99 (2H, m), 2.47 (3H, s), 2.89 (3H, s), 3.12 (3H, s), 3.63 (2H, t, J=6.3 Hz), 3.70-3.79(4H, m), 3.89 (2H, t, J=5.1 Hz), 6.90 (2H, d, J=9.0 Hz), 7.42 (2H, d, J=9.0 Hz), 8.44 (1H, s);

HRMS m/z calcd for C₂₀H₂₅N₆F₃S 424.1783, found 424.1783;

MS (FAB) m/z: 424 [M+H]⁺, 390, 379, 352, 245, 227, 200, 188, 90, 73;

Anal. Calcd. for C₂₀H₂₄F₃N₅S: C, 56.72; H, 5.71; N, 16.54; F, 13.46; S, 7.57. Found: C, 56.46; H, 5.60; N, 16.36, F, 13.32; S, 7.49.

### (63e) 3-amino-4-[4-(4-trifluoromethylphenyl)-1,4-diazepan-1-yl]thieno[2,3-b]pyridine-2-carboxamide

(2Z)-2-cyano-N-[(1E)-(dimethylamino)methylene]-3-[4-(4-trifluoromethylphenyl)-1,4-diazepan-1-yl]but-2-enethioamide which was produced in Example 63 (63d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder

Mp 204-208° C.;

IR (KBr) νₘₐₓ 3326, 1614, 1502, 1330, 1199, 1104, 939, 817 cm⁻¹;

¹H NMR(DMSO-d₆, 400 MHz) δ 2.15-2.19 (2H, m), 3.17-3.22 (2H, m), 3.30-3.32 (2H, m), 3.62 (2H, t, J=5.5 Hz), 3.84-3.86 (2H, m), 6.91 (2H, d, J=9.0 Hz), 7.00 (2H, brs), 7.09 (1H, d, J=5.5 Hz), 7.11 (2H, brs), 7.47 (2H, t, J=9.0 Hz), 8.41 (1H, d, J=5.5 Hz);

HRMS m/z calcd for C₂₀H₂₁ON₅F₃S 436.1419, found 436.1418;

MS (FAB) m/z: 436 [M+H]⁺, 419, 246, 214;

Anal. Calcd. for C₂₀H₂₀F₃N₅OS.0.98H₂O: C, 53.01; H, 4.88; N, 15.46; F, 12.58; S, 7.08. Found: C, 52.79; H, 4.61; N, 15.21; F, 12.97; S, 7.12.

### Example 64

### 3-amino-4-{4-[4-(methylthio)phenyl]-1,4-diazepan-1-yl}thieno[2,3-b]pyridine-2-carboxamide

(Exemplified Compound No. 3-131)

### (64a) tert-butyl 4-[4-(methylthio)phenyl]-1,4-diazepane-1-carboxylate

The reaction was performed in a similar method as described in Org. Lett., 4, 581-584 (2002) and the title compound was synthesized.

Brown liquid

IR (film) νₘₐₓ 2974, 1693, 1595, 1503, 1416, 1365, 1237, 1168, 1125, 930, 810 cm⁻¹;

¹H NMR(CDCl₃, 400 MHz) δ 1.35 (4.5H, s), 1.42 (4.5H, s), 1.91-1.96 (2H, m), 2.39 (3H, s), 3.19 (1H, t, J=5.9 Hz), 3.30 (1H, t, J=5.9 Hz), 3.49-3.54 (6H, m), 6.61 (2H, d, J=8.6 Hz), 7.22 (2H, d, J=8.6 Hz);

HRMS m/z calcd for C₁₇H₂₆O₂N₂S 322.1715, found 322.1714;

MS (FAB) m/z: 322 [M⁺], 266, 221, 178, 57;

Anal. Calcd. for C₁₇H₂₆N₂O₂S.0.28H₂O: C, 62.34; H, 8.17; N, 8.55. Found: C, 62.38; H, 8.41; N, 8.38.

### (64b) 1-[4-(methylthio)phenyl]-1,4-diazepane

tert-butyl 4-[4-(methylthio)phenyl]-1,4-diazepane-1-carboxylate which was produced in Example 64 (64a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2919, 1595, 1503, 1395, 1195, 809 cm⁻¹;

¹H NMR(CDCl₃, 400 MHz) δ 1.88 (2H, quint, J=5.9 Hz), 2.40 (3H, s), 2.82 (2H, t, J=5.9 Hz), 3.01 (2H, t, J=5.1 Hz), 3.47-3.57 (4H, m), 6.64 (2H, d, J=8.6 Hz), 8.25 (2H, d, J=8.6 Hz);

HRMS m/z calcd for C₁₂H₁₈N₂S 222.1191, found 222.1178;

MS (EI) m/z: 222 [M⁺], 192, 180, 166, 151, 137, 108, 91, 77, 70, 56, 43.

### (64c) (2Z)-2-cyano-3-{4-[4-(methylthio)phenyl]-1,4-diazepan-1-yl}but-2-enethioamide

1-(4-(methylsulphanylphenyl)-1,4-diazepane which was produced in Example 64 (64b) was used in place of isobutylamine and the reaction was performed in a similar method as described in Example 5 (5a) and the title compound was synthesized.

Pale yellow powder

Mp 159-160° C. (decomposition);

IR (KBr) νₘₐₓ 3341, 3154, 2178, 1594, 1501, 1392, 1244, 1011, 879, 811 cm⁻¹;

¹H NMR(DMSO-d₆, 400 MHz) δ 1.90-1.94 (2H, m), 2.23 (3H, s), 2.36 (3H, s), 3.49-3.54 (4H, m), 3.59-3.61 (2H, m), 3.70-3.73 (2H, m), 6.75 (2H, d, J=8.6 Hz), 7.16 (2H, d, J=8.6 Hz), 8.35 (1H, brs), 9.01 (1H, brs);

HRMS m/z calcd for C₁₇H₂₂N₄S₂ 346.1286, found 346.1245;

MS (EI) m/z: 346 [M⁺], 272, 247, 222, 205, 192, 178, 166, 151, 137, 123, 96, 68, 59, 42;

Anal. Calcd. for C₁₇H₂₂N₄S₂.0.08H₂O: C, 56.68; H, 6.42; N, 16.10; S, 18.43. Found: C, 58.47; H, 6.32; N, 15.98, S, 18.39.

### (64d) (2Z)-2-cyano-N-[(1E)-(dimethylamino)methylene]-3-{4-[4-(methylthio)phenyl]-1,4-diazepan-1-yl}but-2-enethioamide

(2Z)-3-{4-[4-(methylthio)phenyl]-1,4-diazepan-1-yl}-2-cyanobut-2-enethioamide which was produced in Example 64 (64c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 133-136° C.;

IR (KBr) νₘₐₓ 2921, 2177, 1610, 1501, 1396, 1325, 1291, 1195, 1011, 923, 815 cm⁻¹;

¹H NMR(DMSO-d₆, 400 MHz) δ 1.93-1.98 (2H, m), 2.35 (3H, s), 2.49 (3H, s), 2.92 (3H, s), 3.14 (3H, s), 3.53 (2H, t, J=5.9 Hz), 3.69-3.81 (6H, m), 6.75 (2H, d, J=9.0 Hz), 7.14 (2H, d, J=9.0 Hz), 8.46 (1H, s);

HRMS m/z calcd for C₂₀H₂₈N₅S₂ 402.1786, found 402.1770;

MS (FAB) m/z: 402 [M+H]⁺, 368, 330, 221, 192, 178, 166;

Anal. Calcd. for C₂₀H₂₇N₅S₂.0.2H₂O: C, 59.28; H, 6.82; N, 17.28; S, 15.83. Found: C, 59.53; H, 6.80; N, 17.00; S, 15.96.

### (64e) 3-amino-4-{4-[4-(methylthio)phenyl]-1,4-diazepan-1-yl}thieno[2,3-b]pyridine-2-carboxamide

(2Z)-2-cyano-N-[(1E)-(dimethylamino)methylene]-3-{4-[4-(methylthio)phenyl]-1,4-diazepan-1-yl}but-2-enethioamide which was produced in Example 64 (64d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in 58 embodiment (58e) and the title compound was obtained.

Pale yellow powder

Mp 186-188° C.;

IR (KBr) νₘₐₓ 3433, 3326, 3163, 1657, 1592, 1500, 1367, 1232, 940, 811 cm⁻¹;

¹H NMR(DMSO-d₆, 400 MHz) δ 2.09-2.14 (2H, m), 2.36 (3H, s), 3.16-3.18 (2H, m), 3.25-3.28 (2H, m), 3.52 (2H, t, J=6.3 Hz), 3.74 (2H, t, J=4.7 Hz), 6.73 (2H, d, J=9.0 Hz), 6.94 (2H, brs), 7.05 (1H, d, J=5.1 Hz), 7.06 (2H, brs), 7.17 (2H, t, J=9.0 Hz), 8.37 (1H, d, J=5.1 Hz);

HRMS m/z calcd for C₂₀H₂₄ON₅S₂ 414.1422, found 414.1414;

MS (FAB) m/z: 414 [M+H]⁺, 413, 397, 275, 230, 218, 192, 178, 166;

Anal. Calcd. for C₂₀H₂₃N₅OS₂: C, 58.08; H, 5.61; N, 16.93; S, 15.51. Found: C, 57.91; H, 5.66; N, 16.68; S, 15.42.

### Example 65

### 3-amino-4-[4-(3-toluyl)-1,4-diazepan-1-yl]thieno[2,3-b]pyridine-2-carboxamide

(Exemplified Compound No. 3-110)

### (65a) tert-butyl 4-(3-toluyl)-1,4-diazepane-1-carboxylate

The reaction was performed in a similar method as described in Org. Lett., 4,581-584(2002) and the title compound was synthesized.

Brown liquid

IR (film) νₘₐₓ 2974, 1695, 1602, 1498, 1415, 1175, 930, 692 cm⁻¹;

¹H NMR(CDCl₃, 400 MHz) δ 1.36 (4.5H, s), 1.44 (4.5H, s), 1.92-1.98 (2H, m), 2.28 (3H, s), 3.19 (1H, t, J=6.3 Hz), 3.29 (1H, t, J=6.3 Hz), 3.49-3.54 (6H, m), 6.46-6.48 (3H, m), 7.06 (1H, t, J=7.1 Hz);

HRMS m/z calcd for C₁₇H₂₆O₂N₂ 290.1994, found 290.1981;

MS (FAB) m/z: 290 [M⁺], 235, 217, 189, 120, 91, 70, 57;

Anal. Calcd. for C₁₇H₂₆N₂O₂.0.16H2O: C, 69.62; H, 9.05; N, 9.55. Found: C, 69.71; H, 9.36; N, 9.28.

### (65b) 1-(3-toluyl)-1,4-diazepane

tert-butyl 4-(3-toluyl)-1,4-diazepane-1-carboxylate which was produced in Example 65 (65a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2928, 1601, 1498, 1363, 1178, 765, 692 cm⁻¹;

¹H NMR(CDCl₃, 400 MHz) δ 1.88 (2H, quint, J=4.3 Hz), 2.29 (3H, s), 2.82 (2H, t, J=5.9 Hz), 3.02 (2H, t, J=5.1 Hz), 3.52-3.57 (4H, m), 6.47-6.51 (3H, m), 7.09 (2H, dd, J=1.9, 9.4 Hz);

HRMS m/z calcd for C₁₂H₁₈N₂ 190.1470, found 190.1456;

MS (EI) m/z: 190 [M⁺], 160, 148, 134, 122, 105, 91, 77, 65, 43.

### (65c) (2Z)-2-cyano-3-[4-(3-toluyl)-1,4-diazepan-1-yl]but-2-enethioamide

1-(3-toluyl)-1,4-diazepane which was produced in Example 65 (65b) was used in place of isobutylamine and the reaction was performed in a similar method as described in Example 5 (5a) and the title compound was synthesized.

Pale yellow powder

Mp 144-148° C.;

IR (KBr) νₘₐₓ 3151, 2188, 1601, 1542, 1345, 1174, 912, 766 cm⁻¹;

¹H NMR(DMSO-d₆, 400 MHz) δ 1.91-1.95 (2H, m), 2.22 (3H, s), 2.23 (3H, s), 3.48-3.54 (4H, m), 3.60-3.63 (2H, m), 3.68-3.71 (2H, m), 6.44 (1H, d, J=7.0 Hz), 6.55-6.57 (2H, m), 7.03 (1H, t, J=7.0 Hz), 8.34 (1H, brs), 9.01 (1H, brs);

HRMS m/z calcd for C₁₇H₂₃N₄S 315.1644, found 315.1645;

MS (FAB) m/z: 315 [M+H]⁺, 281, 256, 246, 173;

Anal. Calcd. for C₁₇H₂₂N₄S: C, 64.93; H, 7.05; N, 17.82; S, 10.20. Found: C, 64.65; H, 7.05; N, 17.73, S, 10.13.

### (65d) (2Z)-2-cyano-N-[(1E)-(dimethylamino)methylene]-3-[4-(3-toluyl)-1,4-diazepan-1-yl]but-2-enethioamide

(2Z)-3-[4-(3-toluyl)-1,4-diazepan-1-yl]-2-cyanobut-2-enethioamide which was produced in Example 65 (65c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 256-260° C. (decomposition);

IR (KBr) νₘₐₓ 2178, 1608, 1394, 1292, 1181, 1118, 773, 692 cm⁻¹;

¹H NMR(DMSO-d₆, 400 MHz) δ 1.94-1.99 (2H, m), 2.21 (3H, s), 2.49 (3H, s), 2.92 (3H, s), 3.14 (3H, s), 3.54 (2H, t, J=5.9 Hz), 3.68-3.77(6H, m), 6.43 (1H, d, J=7.8 Hz), 6.55-6.58 (2H, m), 7.02 (1H, t, J=7.8 Hz), 8.45 (1H, s);

HRMS m/z calcd for C₂₀H₂₈N₅S 370.2065found 370.2057;

MS (FAB) m/z: 370 [M+H]⁺, 354, 336, 325, 298, 281, 235, 208, 196, 180, 173, 160, 134, 115, 90, 58;

Anal. Calcd. for C₂₀H₂₇N₅S.0.06H₂O: C, 64.82; H, 7.38; N, 18.90; S, 8.65. Found: C, 64.85; H, 7.22; N, 18.61; S, 8.95.

### (65e) 3-amino-4-[4-(3-toluyl)-1,4-diazepan-1-yl]thieno[2,3-b]pyridine-2-carboxamide

(2Z)-2-cyano-N-[(1E)-(dimethylamino)methylene]-3-[4-(3-toluyl)-1,4-diazepan-1-yl]but-2-enethioamide which was produced in Example 65 (65d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder

Mp 209-212° C.;

IR (KBr) νₘₐₓ 3327, 3169, 2830, 1637, 1579, 1498, 1373, 1234, 1182, 942, 767 cm⁻¹;

¹H NMR(DMSO-d₆, 400 MHz) δ 2.10-2.15 (2H, m), 2.22 (3H, s), 3.14-3.18 (2H, m), 3.26-3.28 (2H, m), 3.52 (2H, t, J=6.3 Hz), 3.73 (2H, t, J=4.7 Hz), 6.41 (1H, d, J=7.8 Hz), 6.53-6.56 (2H, m), 6.97 (2H, brs), 7.01 (1H, t, J=7.8 Hz), 7.05 (1H, d, J=5.5 Hz), 7.06 (2H, brs), 8.37 (1H, d, J=5.5 Hz);

HRMS m/z calcd for C₂₀H₂₄ON₅S 382.1702, found 382.1699;

MS (FAB) m/z: 382 [M+H]⁺, 365, 248, 230, 218, 202, 176;

Anal. Calcd. for C₂₀H₂₃N₅OS.0.08H₂O: C, 62.73; H, 6.10; N, 18.29; S, 8.37. Found: C, 62.56; H, 6.08; N, 18.14; S, 8.32.

### Example 66

### 3-amino-4-{4-[4-(methylsulfinyl)phenyl]-1,4-diazepan-1-yl}thieno[2,3-b]pyridine-2-carboxamide

(Exemplified Compound No. 3-132)

3-amino-4-{4-[4-(methylthio)phenyl]-1,4-diazepan-1-yl}thieno[2,3-b]pyridine-2-carboxamide (57 mg, 0.14 mmol) which was produced in Example 64 (64e) was dissolved in methanol (5 mL), and was blended with an aqueous solution (1 mL) of sodium periodate (33 mg, 0.15 mmol) and heated under reflux for one hour. Water (10 mL) was added to the reaction liquid and after stirring for two hours, generated powder was separated by filtration and the title compound (53 mg, yield 89%) was obtained.

White powder

Mp 153-157° C.;

IR (KBr) νₘₐₓ 3439, 3324, 3182, 1646, 1592, 1505, 1367, 1093, 1037, 939, 814 cm⁻¹;

¹H NMR(DMSO-d₆, 400 MHz) δ 2.13-2.17 (2H, m), 2.67 (3H, s), 3.18-3.22 (2H, m), 3.29-3.31 (2H, m), 3.61 (2H, t, J=6.3 Hz), 3.84 (2H, t, J=6.3 Hz), 6.93 (2H, d, J=9.0 Hz), 6.94 (2H, brs), 7.09 (1H, d, J=5.1 Hz), 7.09 (2H, brs), 7.49 (2H, t, J=9.0 Hz), 8.40 (1H, d, J=5.1 Hz);

HRMS m/z calcd for C₂₀H₂₄O₂N₅S₂ 430.1371, found 430.1386;

MS (FAB) m/z: 430 [M+H]⁺, 412, 395, 242, 230, 204, 166, 65;

Anal. Calcd. for C₂₀H₂₃N₅O₂S₂.2.1H₂O: C, 51.40; H, 5.87; N, 14.98; S, 13.72. Found: C, 51.61; H, 5.87; N, 15.00; S, 13.52.

### Example 67

### 3-amino-4-[4-(2-methoxyphenyl)-1,4-diazepan-1-yl]thieno[2,3-b]pyridine-2-carboxamide

(Exemplified Compound No. 3-122)

### (67a) tert-butyl 4-(2-methoxyphenyl)-1,4-diazepane-1-carboxylate

The reaction was performed in a similar method as described in Org. Lett., 4,581-584(2002) and the title compound was synthesized.

Brown liquid

IR (film) νₘₐₓ 2975, 1694, 1503, 1415, 1242, 1165, 1029, 745 cm⁻¹;

¹H NMR(CDCl₃, 400 MHz) δ 1.14-1.46 (9H, m), 1.89-2.00 (2H, m), 3.18-3.31 (4H, m), 3.48-3.62 (4H, m), 3.83 (3H, s), 6.81-6.93 (4H, m);

HRMS m/z calcd for C₁₇H₂₆O₃N₂ 306.1943, found 306.1972;

MS (EI) m/z: 306 [M⁺], 249, 233, 205, 188, 176, 162, 150, 134, 120, 57.

### (67b) 1-(2-methoxyphenyl)-1,4-diazepane

tert-butyl 4-(2-methoxyphenyl)-1,4-diazepane-1-carboxylate which was produced in Example 67 (67a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2938, 1593, 1502, 1242, 1028, 744 cm⁻¹;

¹H NMR(CDCl₃, 400 MHz) δ 1.96 (2H, quint, J=5.9 Hz), 3.05 (2H, t, J=5.5 Hz), 3.11 (2H, t, J=5.5 Hz), 3.30-3.34 (4H, m), 3.84 (3H, s), 6.83-6.96 (4H, m);

HRMS m/z calcd for C₁₂H₁₈ON₂ 206.1419, found 206.1417;

MS (EI) m/z: 206 [M⁺], 176, 164, 150, 136, 120, 109, 91, 77, 43.

### (67c) (2Z)-2-cyano-3-[4-(2-methoxyphenyl)-1,4-diazepan-1-yl]but-2-enethioamide

1-(2-methoxyphenyl)-1,4-diazepane which was produced in Example 67 (67b) was used in place of isobutylamine and the reaction was performed in a similar method as described in Example 5 (5a) and the title compound was synthesized.

Yellow amorphous

IR (KBr) νₘₐₓ 3287, 3171, 2918, 2184, 1607, 1533, 1452, 1238, 1024, 751 cm⁻¹;

¹H NMR(DMSO-d₆, 400 MHz) δ 1.93-1.98 (2H, m), 2.33 (3H, s), 3.14-3.17 (2H, m), 3.25-3.37 (2H, m), 3.60-3.66 (4H, m), 3.75 (3H, s), 6.79-6.90 (4H, m), 8.23 (1H, brs), 8.91 (1H, brs);

HRMS m/z calcd for C₁₇H₂₃ON₄S 331.1593, found 331.1587;

MS (FAB) m/z: 331 [M+H]⁺, 315, 273, 200, 165, 63.

### (67d) 3-amino-4-[4-(2-methoxyphenyl)-1,4-diazepan-1-yl]thieno[2,3-b]pyridine-2-carboxamide

(2Z)-2-cyano-3-[4-(2-methoxyphenyl)-1,4-diazepan-1-yl]but-2-enethioamide which was produced in Example 67 (67c) was used in place of (2Z)-3-[4-(2-chlorophenyl)-1,4-diazepan-1-yl]-2-cyanobut-2-enethioamide and the reaction was performed in a similar method as described in Example 61 (61d) and the title compound was obtained.

Pale yellow powder

Mp 179-184° C.;

IR (KBr) νₘₐₓ 3440, 3314, 3141, 1581, 1500, 1371, 1235, 937, 751 cm⁻¹;

¹H NMR(DMSO-d₆, 400 MHz) δ 2.04-2.10 (2H, m), 3.25-3.40 (8H, m), 3.75 (3H, s), 6.80-6.93 (4H, m), 7.05 (2H, brs), 7.08 (2H, brs), 7.11 (1H, d, J=5.5 Hz), 8.39 (1H, d, J=5.5 Hz);

HRMS m/z calcd for C₂₀H₂₄O₂N₅S 398.1651, found 398.1653;

MS (FAB) m/z: 398 [M+H]⁺, 273, 246, 200, 63;

Anal. Calcd. for C₂₀H₂₃N₅O₂S.0.14H₂O: C, 60.05; H, 5.87; N, 17.51; S, 8.02. Found: C, 59.87; H, 5.87; N, 17.43; S, 7.85.

### Example 68

### 3-amino-4-[4-(3-methoxyphenyl)-1,4-diazepan-1-yl]thieno[2,3-b]pyridine-2-carboxamide

(Exemplified Compound No. 3-123)

### (68a) tert-butyl 4-(3-methoxyphenyl)-1,4-diazepane-1-carboxylate

The reaction was performed in a similar method as described in Orgy. Let., 4, 581-584 (2002) and the title compound was synthesized.

Brown liquid

AIR (film) νₘₐₓ 2973, 1694, 1612, 1500, 1416, 1165, 930, 753 cm⁻¹;

¹H NMR(CDCl₃, 400 MHz) δ 1.37 (4.5H, s), 1.44 (4.5H, s), 1.93-2.00 (2H, m), 3.19 (1H, t, J=6.3 Hz), 3.28-3.30 (1H, m), 3.49-3.55 (6H, m), 3.77 (3H, s), 6.23-6.25 (2H, m), 6.32 (1H, d, J=7.0 Hz), 7.11 (1H, t, J=7.0 Hz);

HRMS m/z calcd for C₁₇H₂₆O₃N₂ 306.1943, found 306.1937;

MS (EI) m/z: 306 [M⁺], 250, 235, 205, 188, 176, 162, 150, 121, 70, 57;

Anal. Calcd for C₁₇H₂₆N₂O₃.0.38H2O: C, 65.18; H, 8.61; N, 8.94. Found: C, 65.22; H, 8.59; N, 8.79.

### (68b) 1-(3-methoxyphenyl)-1,4-diazepane

tert-butyl 4-(3-methoxyphenyl)-1,4-diazepane-1-carboxylate which was produced in Example 68 (68a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2935, 1611, 1500, 1166, 1054, 923, 752, 688 cm⁻¹;

¹H NMR(CDCl₃, 400 MHz) δ 1.88 (2H, quint, J=5.9 Hz), 2.82 (2H, t, J=5.9 Hz), 3.01 (2H, t, J=5.9 Hz), 3.51-3.57 (4H, m), 3.78 (3H, s), 6.21-6.24 (2H, m), 6.32 (1H, dd, J=1.9, 9.4 Hz), 7.11 (1H, t, J=9.4 Hz);

HRMS m/z calcd for C₁₂H₁₈ON₂ 206.1420, found 206.1403;

MS (EI) m/z: 206 [M⁺], 164, 150, 138, 121, 70, 56.

### (68c) (2Z)-2-cyano-3-[4-(3-methoxyphenyl)-1,4-diazepan-1-yl]but-2-enethioamide

1-(3-methoxyphenyl)-1,4-diazepane which was produced in Example 68 (68b) was used in place of isobutylamine and the reaction was performed in a similar method as described in Example 5 (5a) and the title compound was synthesized.

Pale yellow powder

Mp 129-131° C.;

IR (KBr) νₘₐₓ 3349, 3154, 2939, 2187, 1611, 1541, 1344, 1167, 1058, 914, 749 cm⁻¹;

¹H NMR(DMSO-d₆, 400 MHz) δ 1.91-1.94 (2H, m), 2.23 (3H, s), 3.49-3.54 (4H, m), 3.59-3.62 (2H, m), 3.69 (3H, s), 3.69-3.73 (2H, m), 6.21 (1H, d, J=9.0 Hz), 6.25 (1H, s), 6.35 (1H, d, J=9.0 Hz), 7.05 (1H, t, J=9.0 Hz), 8.35 (1H, brs), 9.02 (1H, brs);

HRMS m/z calcd for C₁₇H₂₃ON₄S 331.1592, found 331.1575;

MS (FAB) m/z: 331 [M+H]⁺, 246, 200, 165, 63;

Anal. Calcd for C₁₇H₂₂N₄OS: C, 61.79; H, 6.71; N, 16.95; S, 9.70. Found: C, 61.72; H, 6.66; N, 16.85; S, 9.44.

### (68d) 4-[4-(3-methoxyphenyl)-1,4-diazepan-1-yl]-2-thioxo-1,2-dihydropyridine-3-carbonitrile

(2Z)-2-cyano-3-[4-(3-methoxyphenyl)-1,4-diazepan-1-yl]but-2-enethioamide (476 mg, 1.44 mmol) which was produced in Example 68 (68c) and N,N-dimethylformamide dimethylacetal (344 mg, 2.88 mmol) were mixed with ethanol (15 mL) and the mixture was stirred at room temperature for 18 hours. The crystal which was deposited was separated by filtration and 490 mg of the title compound was obtained (yield 27%).

Pale yellow powder

Mp 224-227° C. (decomposition);

IR (KBr) νₘₐₓ 2955, 2208, 1608, 1499, 1256, 1166, 1052, 929, 756, 687 cm⁻¹;

¹H NMR(DMSO-d₆, 500 MHz) δ 1.90-1.95 (2H, m), 3.51 (2H, t, J=5.4 Hz), 3.69 (3H, s), 3.71-3.73 (4H, m), 3.95 (2H, t, J=5.4 Hz), 6.20 (1H, d, J=7.8 Hz), 6.26 (1H, s), 6.37 (1H, t, J=7.8 Hz), 6.43 (1H, d, J=7.8 Hz), 7.05 (1H, t, J=7.8 Hz), 7.37 (1H, d, J=7.8 Hz), 12.51 (1H, brs);

HRMS m/z calcd for C₁₈H₂₁ON₄S 341.1436, found 341.1445;

MS (FAB) m/z: 341 [M+H]⁺, 273, 246, 200, 165, 63;

Anal. Calcd for C₁₈H₂₀N₄OS.0.12H₂O: C, 63.10; H, 5.95; N, 16.35; S, 9.36. Found: C, 62.84; H, 5.97; N, 16.35; S, 9.36.

### (68e) 3-amino-4-[4-(3-methoxyphenyl)-1,4-diazepan-1-yl]thieno[2,3-b]pyridine-2-carboxamide

4-[4-(3-methoxyphenyl)-1,4-diazepan-1-yl]-2-thioxo-1,2-dihydropyridine-3-carbonitrile which was produced in Example 68 (68d) was used in place of 4-(isobutylamino)-2-thioxo-1,2-dihydropyridine-3-carbonitrile and the reaction was performed in a similar method as described in Example 5 (5c) and the title compound was obtained.

Pale yellow powder

Mp 193-195° C.;

IR (KBr) νₘₐₓ 3426, 3319, 3145, 1611, 1499, 1372, 1228, 1167, 1054, 943, 822 cm⁻¹;

¹H NMR(DMSO-d6, 400 MHz) δ 2.11-2.16 (2H, m), 3.16-3.21 (2H, m), 3.26-3.30 (2H, m), 3.53 (2H, t, J=6.7 Hz), 3.70 (3H, s), 3.75 (2H, t, J=4.7 Hz), 6.22 (1H, dd, J=2.4, 8.2 Hz), 6.26 (1H, t, J=2.4 Hz), 6.37 (1H, dd, J=2.4, 8.2 Hz), 7.00 (2H, brs), 7.04-7.08 (2H, m), 7.09 (2H, brs), 8.40 (1H, d, J=5.5 Hz);

HRMS m/z calcd for C₂₀H₂₄O₂N₅S 398.1651, found 398.1639;

MS (FAB) m/z: 398 [M+H]⁺, 273, 246, 200, 63;

Anal. Calcd for C₂₀H₂₃N₅O₂S.0.16H₂O: C, 60.00; H, 5.87; N, 17.49; S, 8.01. Found: C, 59.86; H, 5.67; N, 17.29; S, 7.83.

### Example 69

### 3-amino-4-[4-(4-fluoro-3-methylphenyl)-1,4-diazepan-1-yl]thieno[2,3-b]pyridine-2-carboxamide

(Exemplified Compound No. 3-118)

### (69a) tert-butyl 4-(4-fluoro-3-methylphenyl)-1,4-diazepane-1-carboxylate

The reaction was performed in a similar method as described in Orgy. Let., 4, 581-584 (2002) and the title compound was synthesized.

Brown liquid

IR (film) νₘₐₓ 2975, 1694, 1509, 1416, 1366, 1232, 1170, 931, 762 cm⁻¹;

¹H NMR(CDCl₃, 400 MHz) δ 1.34 (4.5H, s), 1.42 (4.5H, s), 1.91-1.95 (2H, m), 2.20 (3H, s), 3.18 (1H, t, J=5.9 Hz), 3.29 (1H, t, J=5.9 Hz), 3.44-3.55 (6H, m), 6.37-6.44 (2H, m), 6.81 (1H, t, J=8.6 Hz);

HRMS m/z calcd for C₁₇H₂₅O₂N₂F 308.1900, found 308.1892;

MS (EI) m/z: 308 [M⁺], 253, 251, 207, 205, 164, 152, 138, 123, 109, 57;

Anal. Calcd for C₁₇H₂₅FN₂O₂: C, 66.21; H, 8.17; N, 9.08; F, 6.16. Found: C, 66.50; H, 7.29; N, 8.88; F, 6.18.

### (69b) 1-(4-fluoro-3-methylphenyl)-1,4-diazepane

tert-butyl 4-(4-fluoro-3-methylphenyl)-1,4-diazepane-1-carboxylate which was produced in Example 69 (69a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2929, 1615, 1509, 1228, 839, 797, 761 cm⁻¹;

¹H NMR(CDCl₃, 400 MHz) δ 1.88 (2H, quint, J=6.3 Hz), 2.23 (3H, s), 2.83 (2H, t, J=5.9 Hz), 3.02 (2H, t, J=5.5 Hz), 3.48-3.54 (4H, m), 6.42-6.48 (2H, m), 6.85 (1H, t, J=9.0 Hz);

HRMS m/z calcd for C₁₂H₁₇N₂F 208.1575, found 208.1353;

MS (EI) m/z: 208 [M⁺], 178, 166, 152, 138, 123, 109, 43;

### (69c) (2Z)-2-cyano-3-[4-(4-fluoro-3-methylphenyl)-1,4-diazepan-1-yl]but-2-enethioamide

1-(4-fluoro-3-methylphenyl)-1,4-diazepane which was produced in Example 69 (69b) was used in place of isobutylamine and the reaction was performed in a similar method as described in Example 5 (5a) and the title compound was synthesized.

Pale yellow powder

Mp 146-150° C. (decomposition);

IR (KBr) νₘₐₓ 3328, 3152, 2189, 1627, 1543, 1508, 1389, 1220, 1056, 917, 868, 801 cm⁻¹;

¹H NMR(DMSO-d₆, 400 MHz) δ 1.88-1.96 (2H, m), 2.18 (3H, s), 2.24 (3H, s), 3.48-3.53 (4H, m), 3.60-3.62 (2H, m), 3.66-3.69 (2H, m), 6.54-6.60 (1H, m), 6.64-6.67 (1H, m), 6.92 (1H, t, J=9.0 Hz), 8.35 (1H, brs), 9.01 (1H, brs);

HRMS m/z calcd for C₁₇H₂₂N₄FS 333.1536, found 333.1534;

MS (FAB) m/z: 333 [M+H]⁺, 299, 274, 207, 191, 178, 164, 123, 65, 51;

Anal. Calcd for C₁₇H₂₁FN₄S: C, 61.42; H, 6.37; N, 16.85; S, 9.65. Found: C, 61.18; H, 6.29; N, 16.71; S, 9.74.

### (69d) (2Z)-2-cyano-N-[(1E)-(dimethylamino)methylene]-3-[4-(4-fluoro-3-methylphenyl)-1,4-diazepan-1-yl]but-2-enethioamide

(2Z)-3-[4-(4-fluoro-3-methylphenyl)-1,4-diazepan-1-yl]-2-cyanobut-2-enethioamide which was produced in Example 69 (69c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 148-150° C.;

IR (KBr) νₘₐₓ 2923, 2178, 1609, 1509, 1324, 1292, 1118, 1015, 512 cm⁻¹;

¹H NMR(DMSO-d₆, 400 MHz) δ 1.94-1.99 (2H, m), 2.15 (3H, s), 2.47 (3H, s), 2.92 (3H, s), 3.14 (3H, s), 3.51 (2H, t, J=5.9 Hz), 3.72 (2H, t, J=5.4 Hz), 3.73-3.77 (4H, m), 6.55-6.59 (1H, m), 6.64-6.66 (1H, m), 6.80 (1H, t, J=9.3 Hz), 8.45 (1H, s);

HRMS m/z calcd for C₂₀H₂₇N₅FS 388.1971, found 388.1962;

MS (FAB) m/z: 388 [M+H]⁺, 354, 299, 273, 165, 120, 65, 51;

Anal. Calcd for C₂₀H₂₆FN₅S: C, 61.99; H, 6.76; N, 18.07; F, 4.90; S, 8.27. Found: C, 61.75; H, 6.57; N, 17.81; F, 4.77; S, 8.31.

### (69e) 3-amino-4-[4-(4-fluoro-3-methylphenyl)-1,4-diazepan-1-yl]thieno[2,3-b]pyridine-2-carboxamide

(2Z)-2-cyano-N-[(1E)-(dimethylamino)methylene]-3-[4-(4-fluoro-3-methylphenyl)-1,4-diazepan-1-yl]but-2-enethioamide which was produced in Example 69 (69d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

White powder

Mp 194-197° C.;

IR (KBr) νₘₐₓ 3414, 3326, 3172, 1637, 1579, 1507, 1374, 1209, 943 cm⁻¹;

¹H NMR(DMSO-d₆, 400 MHz) δ 2.10-2.18 (2H, m), 2.18 (3H, s), 3.15-3.21 (2H, m), 3.27-3.29 (2H, m), 3.50 (2H, t, J=6.3 Hz), 3.71 (2H, t, J=4,7 Hz), 6.52-6.56 (1H, m), 6.62-6.64 (1H, m), 6.91 (1H, t, J=9.0 Hz), 6.98 (2H, brs), 7.06 (1H, d, J=5.5 Hz), 7.07 (2H, brs), 8.38 (1H, d, J=5.5 Hz);

HRMS m/z calcd for C₂₀H₂₃ON₅FS 400.1607, found 400.1632;

MS (FAB) m/z: 400 [M+H]⁺, 383, 275, 236, 209;

Anal. Calcd for C₂₀H₂₂FN₅OS.0.14H₂O: C, 59.75; H, 5.59; N, 17.42; F, 4.73; S, 7.98. Found: C, 59.82; H, 5.58; N, 17.42; F, 4.45; S, 7.71.

### Example 70

### 3-amino-4-[4-(2-toluyl)-1,4-diazepan-1-yl]thieno[2,3-b]pyridine-2-carboxamide

(Exemplified Compound No. 3-109)

### (70a) tert-butyl 4-(2-toluyl)-1,4-diazepane-1-carboxylate

The reaction was performed in a similar method as described in J. Orgy. Chem. 68, 452-459 (2003) and the title compound was synthesized.

Brown liquid

IR (film) νₘₐₓ 2975, 1695, 1492, 1413, 1159, 762, 725 cm⁻¹;

¹H NMR(CDCl₃, 400 MHz) δ 1.47-1.48 (9H, m), 1.87-1.97 (2H, m), 2.29 (3H, s), 3.00-3.10 (4H, m), 3.52-3.60 (4H, m), 6.92 (1H, t, J=7.8 Hz), 7.01 (1H, d, J=7.8 Hz), 7.08-7.14 (2H, m);

HRMS m/z calcd for C₁₇H₂₆O₂N₂ 290.1994, found 290.1977;

MS (FAB) m/z: 290 [M⁺], 249, 233, 189, 166, 146, 130, 95, 51.

### (70b) 1-(2-toluyl)-1,4-diazepane

tert-butyl 4-(2-toluyl)-1,4-diazepane-1-carboxylate which was produced in Example 70 (70a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2938, 2831, 1598, 1492, 1458, 1213, 1163, 1114, 759, 724 cm⁻¹;

¹H NMR(CDCl₃, 400 MHz) δ 1.89 (2H, quint, J=5.9 Hz), 2.32 (3H, s), 3.02 (2H, t, J=4.3 Hz), 3.07 (2H, t, J=5.9 Hz), 3.12-3.17 (4H, m), 6.94 (1H, t, J=7.8 Hz), 7.07 (1H, d, J=7.8 Hz), 7.12-7.17 (2H, m);

HRMS m/z calcd for C₁₂H₁₈N₂ 190.1470, found 190.1443;

MS (EI) m/z: 190 [M⁺], 160, 148, 134, 118, 105, 91, 77, 65, 43.

### (70c) (2Z)-2-cyano-3-[4-(2-toluyl)-1,4-diazepan-1-yl]but-2-enethioamide

1-(2-toluyl)-1,4-diazepane which was produced in Example 70 (70b) was used in place of isobutylamine and the reaction was performed in a similar method as described in Example 5 (5a) and the title compound was synthesized.

Pale yellow powder

Mp 99-100° C.;

IR (KBr) νₘₐₓ 3286, 3173, 2184, 1599, 1521, 1410, 1294, 1220, 881, 765 cm⁻¹;

¹H NMR(DMSO-d₆, 500 MHz) 8 2.00-2.04 (2H, m), 2.24 (3H, s), 2.36 (3H, s), 3.01 (2H, t, J=5.4 Hz), 3.16-3.17 (2H, m), 3.66 (2H, t, J=5.4 Hz), 3.70-3.72 (2H, m), 6.95 (1H, t, J=6.8 Hz), 7.07 (1H, d, J=7.3 Hz), 7.12-7.17 (2H, m), 8.27 (1H, brs), 8.93 (1H, brs);

HRMS m/z calcd for C₁₇H₂₃N₄S 315.1644, found 315.1643;

MS (FAB) m/z: 315 [M+H]⁺, 281, 256, 189, 173, 65, 39;

Anal. Calcd for C₁₇H₂₂N₄S.0.56H₂O: C, 62.92; H, 7.18; N, 17.26; S, 9.88. Found: C, 62.65; H, 6.88; N, 17.11; S, 9.13.

### (70d) 3-amino-4-[4-(2-toluyl)-1,4-diazepan-1-yl]thieno[2,3-b]pyridine-2-carboxamide

(2Z)-2-cyano-3-[4-(2-toluyl)-1,4-diazepan-1-yl]but-2-enethioamide which was produced in Example 70 (70c) was used in place of (2Z)-3-[4-(2-chlorophenyl)-1,4-diazepan-1-yl]-2-cyanobut-2-enethioamide and the reaction was performed in a similar method as described in Example 61 (61d) and the title compound was obtained.

Pale yellow powder

Mp 206-209° C. (decomposition);

IR (KBr) νₘₐₓ 3427, 3308, 3142, 1583, 1493, 1374, 1228, 1053, 944, 767 cm⁻¹;

¹H NMR(DMSO-d₆, 400 MHz) δ 1.97-2.04 (2H, m), 2.23 (3H, s), 3.11 (2H, t, J=5.9 Hz), 3.22-3.25 (2H, m), 3.38-3.42 (2H, m), 6.84-6.88 (1H, m), 7.00-7.10 (7H, m), 8.34 (1H, d, J=5.5 Hz);

HRMS m/z calcd for C₂₀H₂₄ON₅S 382.1702, found 382.1701;

MS (FAB) m/z: 382 [M+H]⁺, 246, 185, 107;

Anal. Calcd for C₂₀H₂₃N₅OS: C, 62.97; H, 6.08; N, 18.36. Found: C, 62.79; H, 6.27; N, 18.20.

### Example 71

### 3-amino-4-[4-(3-fluoro-4-methylphenyl)-1,4-diazepan-1-yl]thieno[2,3-b]pyridine-2-carboxamide

(Exemplified Compound No. 3-117)

### (71a) tert-butyl 4-(3-fluoro-4-methylphenyl)-1,4-diazepane-1-carboxylate

The reaction was performed in a similar method as described in Orgy. Let., 4, 581-584 (2002) and the title compound was synthesized.

Brown liquid

IR (film) νₘₐₓ 2975, 1695, 1634, 1518, 1416, 1366, 1246, 1171, 1124, 930 cm⁻¹;

¹H NMR(CDCl₃, 400 MHz) δ 1.38 (4.5H, s), 1.45 (4.5H, s), 1.93-1.99 (2H, m), 2.14 (3H, s), 3.20 (1H, t, J=5.9 Hz), 3.30 (1H, t, J=5.9 Hz), 3.45-3.55 (6H, m), 6.33-6.36 (2H, m), 6.96 (1H, t, J=8.6 Hz);

HRMS m/z calcd for C₁₇H₂₅O₂N₂F 308.1900, found 308.1877;

MS (EI) m/z: 308 [M⁺], 253, 207, 178, 164, 152, 138, 123, 109, 57.

### (71b) 1-(3-fluoro-4-methylphenyl)-1,4-diazepane

tert-butyl 4-(3-fluoro-4-methylphenyl)-1,4-diazepane-1-carboxylate which was produced in Example 71 (71a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2929, 1634, 1518, 1459, 1119, 927, 822 cm⁻¹;

¹H NMR(CDCl₃, 400 MHz) δ 1.88 (2H, quint, J=5.9 Hz), 2.14 (3H, s), 2.82 (2H, t J=5.9 Hz), 3.01 (2H, t, J=5.9 Hz), 3.50 (2H, t, J=5.9 Hz), 5.53 (2H, t, J=5.9 Hz), 6.34-6.38 (2H, m), 6.97 (1H, t, J=9.0 Hz);

HRMS m/z calcd for C₁₂H₁₇N₂F 208.1576, found 208.1360;

MS (EI) m/z: 208 [M⁺], 178, 166, 152, 138, 123, 109, 44.

### (71c) (2Z)-2-cyano-3-[4-(3-fluoro-4-methylphenyl)-1,4-diazepan-1-yl]but-2-enethioamide

1-(3-fluoro-4-methylphenyl)-1,4-diazepane which was produced in Example 71 (71b) was used in place of isobutylamine and the reaction was performed in a similar method as described in Example 5 (5a) and the title compound was synthesized.

Yellow powder

Mp 155-157° C. (decomposition);

IR (KBr) νₘₐₓ 3163, 2184, 1632, 1516, 1350, 1173, 1120, 872 cm⁻¹;

¹H NMR(DMSO-d₆, 400 MHz) δ 1.90-1.94 (2H, m), 2.08 (3H, s), 2.25 (3H, s), 3.51-3.53 (4H, m), 3.59-3.61 (2H, m), 3.70-3.72 (2H, m), 6.50 (1H, dd, J=2.4, 8.8 Hz), 6.56 (1H, dd, J=2.4, 13.8 Hz), 7.02 (1H, t, J=8.8 Hz), 8.35 (1H, brs), 9.01 (1H, brs);

HRMS m/z calcd for C₁₇H₂₁N₄FSNa 355.1369, found 355.1354;

MS (ESI) m/z: 355.14 [M+Na]⁺;

Anal. Calcd for C₁₇H₂₁FN₄S: C, 61.42; H, 6.37; N, 16.85; F, 5.71. Found: C, 61.07; H, 6.21; N, 16.66, F, 6.06.

### (71d) (2Z)-2-cyano-N-[(1E)-(dimethylamino)methylene]-3-[4-(3-fluoro-4-methylphenyl)-1,4-diazepan-1-yl]but-2-enethioamide

(2Z)-3-[4-(3-fluoro-4-methylphenyl)-1,4-diazepan-1-yl]-2-cyanobut-2-enethioamide which was produced in Example 71 (71c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 254-256° C.;

IR (KBr) νₘₐₓ 2925, 2177, 1608, 1517, 1397, 1291, 1120, 1013, 906, 512 cm⁻¹;

¹H NMR(DMSO-d₆, 500 MHz) δ 1.94-1.98 (2H, m), 2.07 (3H, s), 2.49 (3H, s), 2.93 (3H, s), 3.15 (3H, s), 3.53 (2H, t, J=5.9 Hz), 3.71 (2H, t, J=5.4 Hz), 3.77-3.81 (4H, m), 6.51 (1H, dd, J=2.4, 8.3 Hz), 6.56 (1H, dd, J=2.4, 13.7 Hz), 7.01 (1H, t, J=8.3 Hz), 8.46 (1H, s);

HRMS m/z calcd for C₂₀H₂₇N₅FS 388.1971, found 388.1986;

MS (FAB) m/z: 388 [M+H]⁺, 354, 273, 242, 209, 166;

Anal. Calcd for C₂₀H₂₆FN₅S: C, 61.99; H, 6.76; N, 18.07; F, 4.90; S, 8.27. Found: C, 61.73; H, 6.87; N, 18.08; F, 4.92; S, 8.29.

### (71e) 3-amino-4-[4-(3-fluoro-4-methylphenyl)-1,4-diazepan-1-yl]thieno[2,3-b]pyridine-2-carboxamide

(2Z)-2-cyano-N-[(1E)-(dimethylamino)methylene]-3-[4-(3-fluoro-4-methylphenyl)-1,4-diazepan-1-yl]but-2-enethioamide which was produced in Example 71 (71d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder

Mp 79-83° C.;

IR (KBr) νₘₐₓ 3440, 3323, 3177, 2926, 1633, 1578, 1517, 1368, 1118, 943, 824 cm⁻¹;

¹H NMR(DMSO-d₆, 400 MHz) δ 2.07-2.15 (2H, m), 2.09 (3H, s), 3.15-3.20 (2H, m), 3.23-3.27 (2H, m), 3.51 (2H, t, J=6.3 Hz), 3.73 (2H, t, J=4,7 Hz), 6.47-6.54 (2H, m), 6.97 (2H, brs), 7.02 (1H, t, J=8.7 Hz), 7.06 (1H, d, J=5.5 Hz), 7.08 (2H, brs), 8.38 (1H, d, J=5.5 Hz);

HRMS m/z calcd for C₂₀H₂₃ON₅FS 400.1608, found 400.1559;

MS (FAB) m/z: 400 [M+H]⁺, 278, 246, 185, 83, 57;

Anal. Calcd for C₂₀H₂₂FN₅OS.0.56H₂O: C, 58.65; H, 5.69; N, 17.10; F, 4.64. Found: C, 58.93; H, 6.04; N, 17.04; F, 4.89.

### Example 72

### 3-amino-4-[4-(2,3-dihydro-1,4-benzodioxin-6-yl)-1,4-diazepan-1-yl]thieno[2,3-b]pyridine-2-carboxamide

(Exemplified Compound No. 3-88)

### (72a) tert-butyl 4-(2,3-dihydro-1,4-benzodioxin-6-yl)-1,4-diazepane-1-carboxylate

The reaction was performed in a similar method as described in Orgy. Let., 4,581-584(2002) and the title compound was synthesized.

Brown liquid

IR (film) νₘₐₓ 2974, 1692, 1511, 1416, 1284, 1170, 1072, 931 cm⁻¹;

¹H NMR(CDCl₃, 400 MHz) δ 1.39 (4.5H, s), 1.45 (4.5H, s), 1.92-1.99 (2H, m), 3.20 (1H, t, J=6.3 Hz), 3.30 (1H, t, J=5.9 Hz), 3.43-3.49 (4H, m), 3.53-3.55 (2H, m), 4.17-4.18 (2H, m), 4.22-4.23 (2H, m), 6.18-6.22 (2H, m), 6.72 (1H, d, J=9.8 Hz);

HRMS m/z calcd for C₁₈H₂₇O₄N₂ 335.1971, found 335.1974;

MS (FAB) m/z: 334 [M⁺], 278, 246, 235, 189, 145, 139, 83, 57; Anal. Calcd for C₁₈H₂₆N₃O₄.0.26H₂O: C, 63.76; H, 7.88; N, 8.27. Found: C, 63.75; H, 7.66; N, 8.02.

### (72b) 1-(2,3-dihydro-1,4-benzodioxin-6-yl)-1,4-diazepane

tert-butyl 4-(2,3-dihydro-1,4-benzodioxin-6-yl)-1,4-diazepane-1-carboxylate which was produced in Example 72 (72a) was used in place of tert-butyl 4-phenyl-1,4-diazepane-1-carboxylate and the reaction was performed in a similar method as described in Example 57 (57b) and the title compound was obtained.

Yellow liquid

IR (film) νₘₐₓ 2929, 1626, 1510, 1284, 1071, 822, 789 cm⁻¹;

¹H NMR(CDCl₃, 400 MHz) δ 1.87 (2H, quint, J=6.3 Hz), 2.82 (2H, t, J=5.9 Hz), 3.00 (2H, t, J=5.5 Hz), 3.45-3.50 (4H, m), 4.17-4.19 (2H, m), 4.22-4.24 (2H, m), 6.19-6.22 (2H, m), 6.72 (1H, d, J=9.4 Hz);

HRMS m/z calcd for C₁₃H₁₈O₂N₂ 234.1368, found 234.1373;

MS (EI) m/z: 234 [M⁺], 204, 192, 178, 166, 149, 136, 117, 79, 56, 43.

### (72c) (2Z)-2-cyano-3-[4-(2,3-dihydro-1,4-benzodioxin-6-yl)-1,4-diazepan-1-yl]but-2-enethioamide 1-(2,3-dihydro-1,4-benzodioxin-6-yl)-1,4-diazepane which was produced in Example 72 (72b) was used in place of isobutylamine and the reaction was performed in a similar method as described in Example 5 (5a) and the title compound was synthesized.

Yellow powder

Mp 114-116° C.;

IR (KBr) νₘₐₓ 3314, 3155, 2185, 1542, 1510, 1286, 1211, 1068, 869 cm⁻¹;

¹H NMR(DMSO-d₆, 400 MHz) δ 1.88-1.84 (2H, m), 2.25 (3H, s), 3.40-3.45 (2H, m), 3.46-3.52 (2H, m), 3.59 (4H, brs), 4.10-4.12 (2H, m), 4.16-4.18 (2H, m), 6.24-6.27 (2H, m), 6.65 (1H, d, J=9.5 Hz), 8.30 (1H, brs), 8.97 (1H, brs);

HRMS m/z calcd for C₁₈H₂₃O₂N₄S 359.1542, found 359.1540;

MS (FAB) m/z: 359 [M+H]⁺, 338, 273, 226, 182, 165, 120, 63;

Anal. Calcd for C₁₈H₂₂N₄O₂S: C, 59.30; H, 6.27; N, 15.37. Found: C, 59.04; H, 6.18; N, 15.71.

### (72d) (2Z)-2-cyano-3-[4-(2,3-dihydro-1,4-benzodioxin-6-yl)-1,4-diazepan-1-yl]-N-[(1E)-(dimethylamino)methylene]but-2-enethioamide

(2Z)-2-cyano-3-[4-(2,3-dihydro-1,4-benzodioxin-6-yl)-1,4-diazepan-1-yl]but-2-enethioamide which was produced in Example 72 (72c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder

Mp 127-129° C.;

IR (KBr) νₘₐₓ 3429, 2925, 2179, 1610, 1410, 1293, 1209, 1069, 513 cm⁻¹;

¹H NMR(DMSO-d₆, 400 MHz) δ 1.92-1.98 (2H, m), 2.50 (3H, s), 2.95 (3H, s), 3.15 (3H, s), 3.45 (2H, t, J=5.9 Hz), 3.67-3.72 (4H, m), 3.75-3.77 (2H, m), 4.11-4.13 (2H, m), 4.16-4.18 (2H, m), 6.26-6.29 (2H, m), 6.66 (1H, d, J=9.4 Hz), 8.48 (1H, s);

HRMS m/z calcd for C₂₁H₂₈O₂N₅S 414.1964, found 414.1974;

MS (FAB) m/z: 413 [M+H]⁺, 380, 342, 273, 235, 178, 65, 39;

Anal. Calcd for C₂₁H₂₇N₅O₂S: C, 60.99; H, 6.58; N, 16.94; S, 7.75. Found: C, 60.86; H, 6.47; N, 16.79; S, 7.62.

### (72e) 3-amino-4-[4-(2,3-dihydro-1,4-benzodioxin-6-yl)-1,4-diazepan-1-yl]thieno[2,3-b]pyridine-2-carboxamide

(2Z)-2-cyano-3-[4-(2,3-dihydro-1,4-benzodioxin-6-yl)-1,4-diazepan-1-yl]-N-[(1E)-(dimethylamino)methylene]but-2-enethioamide which was produced in Example 72 (72d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder

Mp 104-107° C.;

IR (KBr) νₘₐₓ 3440, 3324, 1645, 1580, 1510, 1368, 1069, 625 cm⁻¹;

¹H NMR(DMSO-d₆, 500 MHz) δ 2.07-2.13 (2H, m), 3.17-3.23 (2H, m), 3.24-3.29 (2H, m), 3.46 (2H, t, J=5.9 Hz), 3.67 (2H, t, J=4.9 Hz), 4.13-4.14 (2H, m), 4.18-4.20 (2H, m), 6.25-6.29 (2H, m), 6.69 (1H, d, J=8.8 Hz), 6.98 (2H, brs), 7.08 (1H, d, J=5.4 Hz), 7.09 (2H, brs), 8.41 (1H, d, J=5.4 Hz);

HRMS m/z calcd for C₂₁H₂₄O₃N₅S 426.1600, found 426.1619;

MS (FAB) m/z: 426 [M+H]⁺, 409, 182, 165, 120, 63;

Anal. Calcd for C₂₁H₂₃N₅O₃S.0.50H₂O: C, 58.05; H, 5.57; N, 16.12. Found: C, 57.96; H, 5.85; N, 15.92.

### Example 73

### 3-amino-4-{4-[4-(methylsulsulfonyl)phenyl]-1,4-diazepan-1-yl}thieno[2,3-b]pyridine-2-carboxamide

(Exemplified Compound No. 3-133)

3-amino-4-{4-[4-(methylthio)phenyl]-1,4-diazepan-1-yl}thieno[2,3-b]pyridine-2-carboxamide (68 mg, 0.16 mmol) which was produced in Example 64 (64e) was dissolved in methanol (5 mL) and was blended with an aqueous solution (5 mL) of oxone (216 mg, 0.35 mmol) and the mixture was stirred at room temperature for 18 hours. Water (10 mL) was added to the reaction liquid and the aqueous layer was extracted with a mixed solvent (3×10 mL) of methylene chloride/2-propanol (4:1). After the extract was dried over sodium sulfate, the solvent was evaporated under reduced pressure. The obtained residue was purified by silica gel column chromatography (ethyl acetate/methanol=20:1) and the title compound (16 mg, yield 22%) was obtained.

White powder

Mp 89-96° C.;

IR (KBr) νₘₐₓ 3330, 1694, 1593, 1557, 1293, 1139, 772, 537 cm⁻¹;

¹H NMR(CDCl₃, 400 MHz) δ 2.11-2.17 (2H, m), 3.00 (3H, s), 3.47 (2H, dd, J=5.9, 7.4 Hz), 3.55-3.61 (4H, m), 3.68 (2H, t, J=5.9 Hz), 5.83 (1H, brs), 6.69 (2H, d, J=9.0 Hz), 6.77 (1H, d, J=5.5 Hz), 6.88 (1H, brs), 7.70 (2H, t, J=9.0 Hz), 8.42 (1H, d, J=5.5 Hz);

HRMS m/z calcd for C₂₀H₂₄O₃N₅S₂ 446.1321, found 446.1307;

MS (FAB) m/z: 446 [M+H]⁺, 415, 273, 242, 165, 65.

### Example 74

### 3-amino-4-{4-[4-(dimethylamino)phenyl]-1,4-diazepan-1-yl}thieno[2,3-b]pyridine-2-carboxamide

(Exemplified Compound No. 3-104)

### (74a) tert-butyl 4-(4-dimethylaminophenyl)-1,4-diazepane-1-carboxylate

The reaction was performed in a similar method as described in J. Orgy. Chem. 65,1158-(2000) and the title compound was synthesized.

Pale brown liquid

IR (film) ν^{max} 2973, 1694, 1519, 1415, 1168, 930, 810 cm⁻¹;

¹H NMR(CDCl₃, 400 MHz) δ 1.38 (4.5H, s), 1.44 (4.5H, s), 1.93-1.98 (2H, m), 2.81 (6H, s), 3.20 (1H, t, J=6.3 Hz), 3.31 (1H, t, J=6.3 Hz), 3.45-3.57 (6H, m), 6.67 (2H, d, J=9.0 Hz), 6.76 (2H, d, J=9.0 Hz);

HRMS m/z calcd for C₁₈H₃₀O₂NS 320.2338, found 320.2338;

MS (FAB) m/z: 320 [M⁺], 263, 182, 165, 120, 63.

### (74b) 1-(4-dimethylaminophenyl)-1,4-diazepane

tert-butyl 4-(4-dimethylaminophenyl)-1,4-diazepane-1-carboxylate which was produced in Example 74 (74a) was used in place of tert-butyl 4-phenyl-1,4-diazepane-1-carboxylate and the reaction was performed in a similar method as described in Example 57 (57b) and the title compound was obtained.

Pale yellow powder

Mp 214-217° C. (decomposition);

IR (film) νₘₐₓ 2934, 1520, 1472, 1323, 1194, 945, 813 cm⁻¹;

¹H NMR(CDCl₃, 500 MHz) δ 2.32 (2H, quint, J=5.4 Hz), 2.84 (6H, s), 3.24 (2H, t, J=5.9 Hz), 3.33 (2H, t, J=4.9 Hz), 3.52 (2H, t, J=6.8 Hz), 3.73 (2H, t, J=4.9 Hz), 6.68 (2H, d, J=9.3 Hz), 6.76 (2H, d, J=9.3 Hz);

HRMS m/z calcd for C₁₃H₂₁N₃ 219.1737, found 219.1752;

MS (EI) m/z: 219 [M⁺], 189, 176, 163, 148, 134, 120.

### (74c) (2Z)-2-cyano-3-[4-(4-dimethylaminophenyl)-1,4-diazepan-1-yl]but-2-enethioamide

1-(4-dimethylaminophenyl)-1,4-diazepane which was produced in Example 74 (74b) was used in place of isobutylamine and the reaction was performed in a similar method as described in Example 5 (5a) and the title compound was synthesized.

Pale yellow powder

Mp 161-164° C. (decomposition);

IR (KBr) νₘₐₓ 3439, 3314, 3154, 2178, 1601, 1518, 1442, 1292, 1218, 1015, 877, 816 cm⁻¹;

¹H NMR(DMSO-d₆, 500 MHz) δ 1.91-1.95 (2H, m), 2.26 (3H, s), 2.74 (6H, s), 3.41 (2H, t, J=5.9 Hz), 3.51 (2H, t, J=5.9 Hz), 3.58-3.62 (4H, m), 6.67 (2H, d, J=9.8 Hz), 6.71 (2H, d, J=9.8 Hz), 8.28 (1H, brs), 8.96 (1H, brs);

HRMS m/z calcd for C₁₈H₂₆N₅S 344.1909, found 344.1901;

MS (EI) m/z: 343 [M⁺], 309, 218, 182, 65;

Anal. Calcd for C₁₈H₂₅N₅S: C, 62.94; H, 7.34; N, 20.39; S, 9.34. Found: C, 62.66; H, 7.06; N, 20.28; S, 9.11.

### (74d) 4-{4-[4-(dimethylamino)phenyl]-1,4-diazepan-1-yl}-2-thioxo-1,2-dihydropyridine-3-carbonitrile

(2Z)-2-cyano-3-[4-(4-dimethylaminophenyl)-1,4-diazepan-1-yl]but-2-enethioamide which was produced in Example 74 (74c) was used in place of (2Z)-2-cyano-3-(isobutylamino)but-2-enethioamide and the reaction was performed in a similar method as described in Example 5 (5b) and the title compound was obtained.

Pale brown powder

Mp 186-188° C.;

IR (KBr) ν_{max ,} 2948, 2204, 1625, 1517, 1243, 1142, 929, 810 cm⁻¹;

¹H NMR(DMSO-d₆, 500 MHz) δ 1.92-1.95 (2H, m), 2.73 (6H, s), 3.42 (2H, t, J=5.9 Hz), 3.63 (2H, t, J=4.9 Hz), 3.71 (2H, t, J=5.4 Hz), 3.93 (2H, t, J=5.4 Hz), 6.42 (1H, d, J=7.8 Hz), 6.66 (2H, d, J=9.3 Hz), 6.69 (2H, d, J=9.3 Hz), 7.35 (1H, d, J=7.8 Hz), 12.52 (1H, brs);

HRMS m/z calcd for C₁₉H₂₄N₅S 354.1753, found 354.1772;

MS (FAB) m/z: 353 [M⁺], 182, 165, 65.

### (74e) 3-amino-4-{4-[4-(dimethylamino)phenyl]-1,4-diazepan-1-yl}thieno[2,3-b]pyridine-2-carboxamide

4-[4-(4-dimethylaminophenyl)-1,4-diazepan-1-yl]-2-thioxo-1,2-dihydropyridine-3-carbonitrile which was produced in Example 74 (74d) was used in place of 4-(isobutylamino)-2-thioxo-1,2-dihydropyridine-3-carbonitrile and the reaction was performed in a similar method as described in Example 5 (5c) and the title compound was obtained.

White powder

Mp 174-176° C.;

IR (KBr) νₘₐₓ 3438, 3324, 2944, 1644, 1579, 1517, 1368, 1230, 940, 811 cm⁻¹;

¹H NMR(DMSO-d₆, 500 MHz) δ 2.07-2.12 (2H, m), 2.75 (6H, s), 3.20-3.25 (2H, m), 3.26-3.30 (2H, m), 3.47 (2H, t, J=5.9 Hz), 3.67 (2H, t, J=4.4 Hz), 6.71 (4H, s), 6.97 (2H, brs), 7.07-7.08 (3H, m), 8.40 (1H, d, J=5.4 Hz);

HRMS m/z calcd for C₂₁H₂₇ON₆S 411.1967, found 411.1945;

MS (FAB) m/z: 411 [M+H]⁺, 394, 273, 242, 200, 189, 175, 93;

Anal. Calcd for C₂₁H₂₆N₆OS.0.16H₂O: C, 61.01; H, 6.42; N, 20.33; S, 7.76. Found: C, 60.75; H, 6.34; N, 20.20; S, 7.94.

### Example 75

### 3-amino-6-methyl-4-(4-phenylpiperazin-1-yl)thieno[2,3-b]pyridine-2-carboxamide

(Exemplified Compound No. 3-76)

### (75a) (1-ethoxyethylidene)malononitrile

An acetic acid (1 mL) solution of malononitrile (4.41 g, 66.8 mmol) and orthotriethyl acetate (14.7 mL, 80.2 mmol) was stirred at 80° C. for one hour. The reaction liquid was cooled to room temperature and generated powder was separated by filtration and the title compound (7.14 g, yield 79%) was obtained.

White powder

¹H NMR(DMSO-d₆, 400 MHz) δ 1.32 (3H, t, J=7.0 Hz), 2.45 (3H, s), 4.42 (2H, q, J=7.0 Hz).

### (75b) [1-(4-phenylpiperazin-1-yl)ethylidene]malononitrile

1-phenylpiperazine (1.68 mL, 11 mmol) was added to an ethanol (40 mL) suspension of (1-ethoxyethylidene)malononitrile (1.36 g, 10 mmol) which was produced in Example 75 (75a) and the mixture was stirred at room temperature for 15 hours. The powder which was generated was separated by filtration and the title compound (1.74 g, yield 69%) was obtained.

White powder

Mp 188-190° C.;

IR (KBr) νₘₐₓ 2199, 1560, 1450, 1236, 998, 763 cm⁻¹;

¹H NMR(DMSO-d₆, 500 MHz) δ 2.32 (3H, s), 3.32-3.34 (4H, m), 3.88 (4H, brs), 6.78 (1H, t, J=8.6 Hz), 6.91 (2H, d, J=8.6 Hz), 7.22 (2H, t, J=8.6 Hz);

HRMS m/z calcd for C₁₅H₁₆N₄ 252.1375, found 252.1372;

MS (EI) m/z: 252 [M⁺], 210, 160, 146, 132, 126, 119, 105, 91, 77, 65;

Anal. Calcd for C₁₅H₁₆N₄: C, 71.40; H, 6.39; N, 22.21. Found: C, 71.16; H, 6.41; N, 22.25.

### (75c) [(2E)-3-(dimethylamino)-1-(4-phenylpiperazin-1-yl)but-2-enylidene]malononitrile

A xylene (10 mL) suspension of [1-(4-phenylpiperazin-1-yl)ethylidene]malononitrile which was produced in Example 75 (75b) (1.74 g, 6.9 mmol) was blended with dimethylacetamide dimethylacetal (5.0 mL, 34.5 mmol) and heated for four hours under reflux. The reaction liquid was concentrated and the obtained residue was purified by silica gel column chromatography (100% ethyl acetate) and the obtained solid was further washed with ether and the title compound (1.23 g, yield 55%) was obtained.

Yellow powder

Mp 181-184° C.;

IR (KBr) νₘₐₓ 2193, 1557, 1511, 1440, 1375, 1258, 1023, 765, 551 cm⁻¹;

¹H NMR(DMSO-d₆, 400 MHz) δ 2.18 (3H, s), 3.04 (6H, s), 3.24 (4H, t, J=5.1 Hz), 3.68 (4H, t, J=5.1 Hz), 4.53 (1H, s), 6.81 (1H, t, J=7.8 Hz), 6.95 (2H, d, J=7.8 Hz), 7.24 (2H, t, J=7.8 Hz);

HRMS m/z calcd for C₁₉H₂₃N₅ 321.1953, found 321.1938;

MS (EI) m/z: 321 [M⁺], 277, 256, 189, 160, 132, 72;

Anal. Calcd for C₁₉H₂₃N₅: C, 71.00; H, 7.21; N, 21.79. Found: C, 71.03; H, 7.21; N, 21.60.

### (75d) 6-methyl-2-oxo-4-(4-phenylpiperazin-1-yl)-1,2-dihydropyridine-3-carbonitrile

[(2E)-3-(dimethylamino)-1-(4-phenylpiperazin-1-yl)but-2-enylidene]malononitrile (1.22 g, 3.8 mmol) which was produced in Example 75 (75c) was dissolved in a mixed solvent (10 mL) of acetic acid/water (4:1) and heated for one hour under reflux. The reaction liquid was cooled to room temperature and water (10 mL) was added and powder which was generated was separated by filtration and the title compound (0.87 g, yield 85%) was obtained.

Yellow powder

Mp >270° C.;

IR (KBr) νₘₐₓ 2837, 2202, 1618, 1497, 1447, 1230, 1003, 755 cm⁻¹;

¹H NMR(DMSO-d₆, 400 MHz) δ 2.14 (3H, s), 3.27 (4H, t, J=5.1 Hz), 3.76 (4H, t, J=5.1 Hz), 6.02 (1H, s), 6.81 (1H, t, J=7.8 Hz), 6.96 (2H, d, J=7.8 Hz), 7.24 (2H, t, J=7.8 Hz), 11.40 (1H, brs);

HRMS m/z calcd for C₁₇H₁₈ON₄ 294.1481, found 294.1477;

MS (EI) m/z: 294 [M⁺], 276, 252, 189, 162, 132, 105, 91, 77;

Anal. Calcd for C₁₇H₁₈N₄O.0.16H₂O: C, 68.69; H, 6.21; N, 18.85. Found: C, 69.00; H, 6.16; N, 18.49.

### (75e) 2-chloro-6-methyl-4-(4-phenylpiperazin-1-yl)nicotinonitrile

N,N-dimethylaniline (75 μL, 0.53 mmol) and phosphorus oxychloride (1.6 mL, 17.4 mmol) were added to 1,4-dioxane (3 mL) solution of 6-methyl-2-oxo-4-(4phenylpiperazin-1-yl)-1,2-dihydropyridine-3-carbonitrile (223 mg, 0.76 mmol) which was produced in Example 75 (75d), and heated under reflux for two hours. After the reaction liquid was concentrated and a sodium hydrogen carbonate aqueous solution (10 mL) was added to the obtained residual substance, the aqueous layer was extracted with ethyl acetate (3×10 mL) and after the extract was dried over sodium sulfate, the solvent was evaporated under reduced pressure. The obtained residue was purified by silica gel column chromatography (hexane/ethyl acetate=2:1) and the title compound (117 mg, yield 49%) was obtained.

Yellow powder

Mp 139-140° C.;

IR (KBr) νₘₐₓ 2830, 2216, 1582, 1502, 1447, 1229, 989, 758, 695 cm⁻¹;

¹H NMR(CDCl₃, 400 MHz) δ 2.49 (3H, s), 3.36 (4H, t, J=5.1 Hz), 3.68 (4H, t, J=5.1 Hz), 6.57 (1H, s), 6.89-6.95 (3H, m), 7.29 (2H, dd, J=7.1, 8.6 Hz);

HRMS m/z calcd for C₁₇H₁₇N₄Cl 312.1142, found 312.1147;

MS (EI) m/z: 312 [M⁺], 294, 270, 206, 194, 179, 152, 132, 105, 91, 77;

Anal. Calcd for C₁₇H₁₇ClN₄: C, 65.28; H, 5.48; N, 17.91. Found: C, 65.11; H, 5.14; N, 17.69.

### (75f) 3-amino-6-methyl-4-(4-phenylpiperazin-1-yl)thieno[2,3-b]pyridine-2-carboxamide

2-mercaptoacetamide (about 70% purity) (48 mg, 0.52 mmol) and 8N aqueous solution of sodium hydroxide (0.1 mL) were added to N,N-dimethylformamide (0.6 mL) solution of 2-chloro-6-methyl-4-(4-phenylpiperazin-1-yl)nicotinonitrile (92 mg, 0.29 mmol) which was produced in Example 75 (75e) and the mixture was stirred at room temperature for one hour. Water was added to the reaction solution and the deposited crystal was separated by filtration and 90 mg of the title compound was obtained (yield 84%).

Pale yellow crystal

Mp 247-250° C.;

IR (KBr) νₘₐₓ 3435, 2828, 1651, 1581, 1494, 1365, 1223, 993, 762, 694 cm⁻¹;

¹H NMR(DMSO-d₆, 400 MHz) δ 2.50 (3H, s), 2.88-3.70 (8H, m), 6.80 (1H, t, J=7.4 Hz), 6.90 (2H, brs), 6.95 (1H, s), 7.00 (2H, d, J=7.4 Hz), 7.04 (2H, brs), 7.23 (2H, t, J=7.4 Hz);

HRMS m/z calcd for C₁₉H₂₁ON₅S 367.1467, found 367.1450;

MS (EI) m/z: 367 [M⁺], 262, 244, 230, 218, 190, 175, 132, 120, 104, 91, 77;

Anal. Calcd for C₁₉H₂₁N₅OS: C, 62.10; H, 5.76; N, 19.06. Found: C, 62.38; H, 5.84; N, 18.84.

### Example 76

### 3-amino-4-[4-(4-fluorophenyl)-1,4-diazepan-1-yl]thieno[2,3-b]pyridine-2-carboxamide

(Exemplified Compound No. 3-89)

### (76a) tert-butyl 4-(4-fluorophenyl)-1,4-diazepane-1-carboxylate

The reaction was performed in a similar method as described in Org. Lett., 4,581-584 (2002) and the title compound was synthesized.

White powder (yeild 45%)

Mp 88-89° C.;

IR (KBr) νₘₐₓ 2968, 2921,1682, 1515, 1418, 1244, 828 cm⁻¹;

¹H NMR(CDCl₃, 400 MHz) δ 1.36 (4.5H, s), 1.43 (4.5H, s), 1.90-2.01 (2H, m), 3.21 (1H, t, J=5.9 Hz), 3.32 (1H, t, J=5.9 Hz), 3.46-3.63 (6H, m), 6.61 (2H, dd, J=9.4, 4.3 Hz), 6.92 (2H, t, J=8.6 Hz);

MS (EI) m/z: 294 [M⁺], 238, 223, 193;

Anal. Calcd for C₁₆H₂₃FN₂O₂.0.14H₂O: C, 64.73; H, 7.90; N, 9.44; F, 6.40. Found: C, 64.77; H, 7.92; N, 9.36; F, 6.37.

### (76b) 1-(4-fluorophenyl)-1,4-diazepane

tert-butyl 4-(4-fluorophenyl)-1,4-diazepane-1-carboxylate which was produced in Example 76 (76a) was used in place of tert-butyl 4-phenyl-1,4-diazepane-

1-carboxylate and the reaction was performed in a similar method as described in Example 57 (57b) and the title compound was obtained.

Pale yellow oil (yeild 81%)

IR (film) νₘₐₓ 3322, 2935, 1611, 1513, 1228, 814 cm⁻¹;

¹H NMR(CDCl₃, 500 MHz) δ 1.89 (2H, quint, J=5.9 Hz), 2.83 (2H, t, J=5.9 Hz), 3.02 (2H, t, J=5.4 Hz), 3.51 (2H, t, J=5.4 Hz), 3.54 (2H, t, J=6.3 Hz), 6.61 (2H, dd, J=9.3, 4.4 Hz), 6.91 (2H, t, J=9.3 Hz);

MS (EI) m/z: 194 [M⁺], 164, 152, 138.

### (76c) (2Z)-2-cyano-3-[4-(4-fluorophenyl)-1,4-diazepan-1-yl]but-2-enethioamide

1-(4-fluorophenyl)-1,4-diazepane which was produced in Example 76 (76b) was used in place of isobutylamine and the reaction was performed in a similar method as described in Example 5 (5a) and the title compound was synthesized.

Yellow ocher powder (yeild 30%)

Mp 150-152° C.;

IR (KBr) νₘₐₓ 3356, 3269, 3178, 2177, 1615, 1537, 1507 cm⁻¹;

¹H NMR(DMSO-d₆, 400 MHz) δ 1.89-1.98 (2H, m), 2.23 (3H, s), 3.47-3.54 (4H, m), 3.57-3.63 (2H, m), 3.66-3.72 (2H, m), 6.74 (2H, dd, J=9.0, 4.3 Hz), 6.97 (2H, t, J=9.0 Hz), 8.31 (1H, bs), 8.96 (1H, bs);

MS (FAB) m/z: 319 [M+H]⁺, 273, 259, 242;

Anal. Calcd for C₁₆H₁₉FN₄S.0.4H₂O: C, 59.02; H, 6.13; N, 17.21. Found: C, 58.73; H, 5.84; N, 17.20.

### (76d) (2Z)-2-cyano-N-[(1E)-(dimethylamino)methylene]-3-[4-(4-fluorophenyl)-1,4-diazepan-1-yl]but-2-enethioamide

(2Z)-2-cyano-3-[4-(4-fluorophenyl)-1,4-diazepan-1-yl]but-2-enethioamide which was produced in Example 76 (76c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder (yeild 73%)

Mp 127-128° C. (decomposition);

IR (KBr) νₘₐₓ 2924, 2182, 1610, 1510, 1421, 1395, 1324, 1293 cm⁻¹;

¹H NMR(DMSO-d₆, 400 MHz) δ 1.93-2.02 (2H, m), 2.47 (3H, s), 2.92 (3H, s), 3.14 (3H, s), 3.53 (2H, t, J=5.9 Hz), 3.73 (2H, t, J=5.9 Hz), 3.78 (4H, bs), 6.76 (2H, dd, J=9.0, 4.3 Hz), 6.97 (2H, t, J=9.0 Hz), 8.45 (2H, s);

MS (FAB) m/z: 374 [M+H]⁺, 340, 273, 195;

Anal. Calcd for C₁₉H₂₄FN₅S: C, 61.10; H, 6.48; N, 18.75; F, 5.09; S, 8.59. Found: C, 61.01; H, 6.49; N, 18.50; F, 5.17; S, 8.56.

### (76e) 3-amino-4-[4-(4-fluorophenyl)-1,4-diazepan-1-yl]thieno[2,3-b]pyridine-2-carboxamide

(2Z)-2-cyano-N-[(1E)-(dimethylamino)methylene]-3-[4-(4-fluorophenyl)-1,4-diazepan-1-yl]but-2-enethioamide which was produced in Example 76 (76d) was used in place of (2Z)-2-cyano-N-[(lE)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale yellow powder (yeild 64%)

Mp 203-205° C.;

IR (KBr) νₘₐₓ 3453, 3324, 3179, 2948, 2838, 1646, 1579, 1510, 1369 cm⁻¹;

¹H NMR(DMSO-d₆, 500 MHz) δ 2.10-2.19 (2H, m), 3.16-3.24 (2H, m), 3.30 (2H, s), 3.49-3.56 (2H, m), 3.71-3.78 (2H, m), 6.76 (2H, dd, J=9.0, 4.4 Hz), 6.96-7.05 (4H, m), 7.06-7.12 (3H, m), 8.40 (1H, d, J=4.9 Hz);

MS (FAB) m/z: 386 [M+H]⁺, 369, 273;

Anal. Calcd for C₁₉H₂₀FN₅OS.1.35H₂O: C, 57.95; H, 5.81; N, 17.78; F, 4.82; S, 8.14. Found: C, 57.58; H, 5.41; N, 17.76; F, 4.73; S, 7.99.

### Example 77

### 3-amino-4-[4-(4-methylphenyl)-1,4-diazepan-1-yl]thieno[2,3-b]pyridine-2-carboxamide

(Exemplified Compound No. 3-111)

### (77a) tert-butyl 4-(4-methylphenyl)-1,4-diazepane-1-carboxylate

The reaction was performed in a similar method as described in Org. Lett., 4, 581-584 (2002) and the title compound was synthesized.

Colourless oil (yeild 36%)

IR (film) νₘₐₓ 2974, 2929, 1695, 1619, 1521, 1415, 1365, 1237, 1169 cm⁻¹;

¹H NMR(CDCl₃, 400 MHz) δ 1.38 (4.5H, s), 1.44 (4.5H, s), 1.91-2.01 (2H, m), 2.23 (3H, s), 3.18 (1H, t, J=5.9 Hz), 3.20 (1H, t, J=5.9 Hz), 3.46-3.59 (6H, m), 6.60 (2H, d, J=8.2 Hz), 7.00 (2H, d, J=8.2 Hz);

MS (EI) m/z: 290 [M⁺], 234, 146.

### (77b) 1-(4-methylphenyl)-1,4-diazepane

tert-butyl 4-(4-methylphenyl)-1,4-diazepane-1-carboxylate which was produced in Example 77 (77a) was used in place of tert-butyl 4-phenyl-1,4-diazepane-1-carboxylate and the reaction was performed in a similar method as described in Example 57 (57b) and the title compound was obtained.

Pale yellow oil (yeild 100%)

IR (film) νₘₐₓ 3318, 2923, 1618, 1520, 1394, 1363, 1189, 802 cm⁻¹;

¹H NMR(CDCl₃, 400 MHz) δ 1.89 (2H, quint, J=5.9 Hz), 2.23 (3H, s), 2.79-2.85 (2H, m), 2.99-3.05 (2H, m), 3.49-3.58 (4H, m), 6.60 (2H, d, J=8.2 Hz), 7.00 (2H, d, J=8.2 Hz);

MS (EI) m/z: 190 [M⁺], 160, 148, 134.

### (77c) 4-[4-(4-methylphenyl)-1,4-diazepan-1-yl]-2-thioxo-1,2-dihydropyridine-3-carbonitrile

1-(4-methylphenyl)-1,4-diazepane (463 mg, 2.39 mmol) which was produced in Example 77 (77b) and (2Z)-2-cyano-3-ethoxybut-2-enethioamide (J. Org. Chem., (1962), 27,2433-2439) (386 mg, 2.27 mmol) were dissolved in N,N-dimethylformamide (4.78 mL) and the mixture was stirred at room temperature for 30 minutes. Subsequently N,N-dimethylformamide dimethylacetal (302 μL, 2.27 mmol) was added and the reaction mixture was stirred at 60° C. for 30 minutes after stirring at room temperature for one hour. After cooling to room temperature, the reaction mixture was blended with ethyl acetate (10 mL) and water (50 mL) and the mixture was stirred. The deposited solid was separated by filtration and washed with ethyl acetate and water sequentially and 125 mg of the title compound was obtained. Yield 28% from 1-(4-methylphenyl)-1,4-diazepane.

Pale brown powder

Mp 220-223° C.;

IR (KBr) νₘₐₓ 3115, 2940, 2205, 1626, 1518, 1250, 928,798, 775 cm⁻¹;

¹H NMR(CDCl₃, 500 MHz) δ 2.11 (2H, quint, J=5.9 Hz), 2.25 (3H, s), 3.56 (2H, t, J=5.9 Hz), 3.71-3.76 (2H, m), 3.76-3.80 (2H, m), 4.10-4.15 (2H, m), 6.19 (1H), d, J=7.8 Hz), 6.65 (2H, d, J=8.2 Hz), 7.05 (2H, d, J=8.2 Hz), 7.20 (1H, d, J=7.8 Hz), 11.22 (1H, bs);

MS (EI) m/z: 324 [M⁺], 160, 146;

Anal. Calcd for C₁₈H₂₀N₄S.0.1H₂O: C, 66.27; H, 6.24; N, 17.17; S, 9.83. Found: C, 66.07; H, 6.19; N, 17.10; S, 9.60.

### (77d) 3-amino-4-[4-(4-methylphenyl)-1,4-diazepan-1-yl]thieno[2,3-b]pyridine-2-carboxamide

4-[4-(4-methylphenyl)-1,4-diazepan-1-yl]-2-thioxo-1,2-dihydropyridine-3-carbonitrile which was produced in Example 77 (77c) was used in place of 4-(isobutylamino)-2-thioxo-1,2-dihydropyridine-3-carbonitrile and the reaction was performed in a similar method as described in Example 5 (5c) and the title compound was obtained.

Pale brown powder (yeild 28%)

Mp 175-176° C.;

IR (KBr) νₘₐₓ 3429,3317, 3170, 3093, 2942, 2833, 1635, 1579, 1520, 1372 cm⁻¹;

¹H NMR(DMSO-d₆, 400 MHz) δ 2.07-2.16 (2H, m), 2.18 (3H, s), 3.15-3.22 (2H, m), 3.23-3.30 (2H, m), 3.51 (2H, t, J=6.3 Hz), 3.72 (2H, t, J=4.7 Hz), 6.66 (2H, d, J=8.2 Hz), 6.92-6.99 (4H, m), 7.02-7.09 (3H, m), 8.37 (1H, d, J=5.1 Hz);

MS (FAB) m/z: 382 [M+H]⁺, 365, 275;

Anal. Calcd for C₂₀H₂₃N₅OS: C, 62.97; H, 6.08; N, 18.36; S, 8.41. Found: C, 62.58; H, 6.02; N, 18.23; S, 8.29.

### Example 78

### 3-amino-4-[4-(3-chlorophenyl)-1,4-diazepan-1-yl]thieno[2,3-b]pyridine-2-carboxamide

(Exemplified Compound No. 3-93)

### (78a) tert-butyl 4-(3-chlorophenyl)-1,4-diazepane-1-carboxylate

The reaction was performed in a similar method as described in Org. Lett.,4,581-584(2002) and the title compound was synthesized.

Colourless oil (yeild 30%)

IR (film) νₘₐₓ 2975, 1694, 1594, 1493, 1416, 1365, 1237,1168 cm⁻¹;

¹H NMR(CDC₁₃, 400 MHz) δ 1.36 (4.5H, s), 1.44 (4.5H, s), 1.95 (2H, quint, J=6.3 Hz), 3.21 (1H, t, J=5.9 Hz), 3.31 (1H, t, J=5.9 Hz), 3.46-3.60 (6H, m), 6.54 (1H, dd, J=9.0, 2.0 Hz), 6.58-6.65 (2H, m), 7.08 (1H, t, J=8.6 Hz);

MS (EI) m/z: 310 [M⁺], 253, 166.

### (78b) 1-(3-chlorophenyl)-1,4-diazepane

tert-butyl 4-(3-chlorophenyl)-1,4-diazepane-1-carboxylate which was produced in Example 78 (78c) was used in place of tert-butyl 4-phenyl-1,4-diazepane-1-carboxylate and the reaction was performed in a similar method as described in Example 57 (57b) and the title compound was obtained.

Pale brown oil (yeild 100%)

IR (film) νₘₐₓ 3323, 2933, 1593,1494, 1362, 1102, 984, 759, 683 cm⁻¹;

¹H NMR(CDCl₃, 400 MHz) δ 1.89 (2H, quint, J=5.9 Hz), 2.79-2.86 (2H, m), 2.98-3.06 (2H, m), 3.52 (2H, t, J=5.5 Hz), 3.56 (2H, t, J=6.3 Hz), 6.56 (1H, dd, J=8.6, 2.7 Hz), 5.59-6.63 (1H, m), 6.67-6.64 (1H, m), 7.10 (1H, t, J=8.6 Hz);

MS (EI) m/z: 210 [M⁺], 168, 154.

### (78c) (2Z)-3-[4-(3-chlorophenyl)-1,4-diazepan-1-yl]-2-cyanobut-2-enethioamide

1-(3-chlorophenyl)-1,4-diazepane which was produced in Example 78 (78b) was used in place of isobutylamine and the reaction was performed in a similar method as described in Example 5 (5a) and the title compound was synthesized.

Slightly brown powder (yeild 62%)

Mp 133-134° C.;

IR (KBr) νₘₐₓ 3320, 3152, 2964, 2187, 1593, 1541, 1488, 1390, 1346 cm⁻¹;

¹H NMR(DMSO-d₆, 400 MHz) δ 1.88-1.97 (2H, m), 2.24 (3H, s), 3.48-3.57 (4H, m), 3.58-3.63 (2H, m), 3.70-3.75 (2H, m), 6.60 (1H, dd, J=8.2, 1.6 Hz), 6.70 (1H, d, J=8.2, 2.3 Hz), 6.75-6.78 (1H, m), 7.14 (1H, t, J=8.2 Hz), 8.36 (1H, bs), 9.01 (1H, s);

MS (FAB) m/z: 335 [M+H]⁺, 246, 200.

### (78d) (2Z)-3-[4-(3-chlorophenyl)-1,4-diazepan-1-yl]-2-cyano-N-[(1E) -(dimethylamino)methylene]but-2-enethioamide

(2Z)-3-[4-(3-chlorophenyl)-1,4-diazepan-1-yl]-2-cyanobut-2-enethioamide which was produced in Example 78 (78c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Yellow powder (yeild 89%)

Mp 129-130° C. (decomposition);

IR (KBr) νₘₐₓ 2924, 2925, 2177, 1609, 1397, 1325, 1290 cm⁻¹;

¹H NMR(DMSO-d₆, 400 MHz) δ 1.92-2.01 (2H, m), 2.49 (3H, s), 3.15 (3H, s), 3.32 (3H, s), 3.57 (2H, t, J=5.5 Hz), 3.72 (2H, t, J=5.5 Hz), 3.75-3.80 (2H, m), 3.80-3.84 (2H, m), 6.61 (1H, d, J=8.3 Hz), 6.73 (1H, dd, J=8.3, 2.0 Hz), 6.79 (1H, s), 7.14 (1H, t, J=8.3 Hz), 8.45 (1H, s);

MS (FAB) m/z: 390 [M+H]⁺, 356, 211, 180;

Anal. Calcd for C₁₉H₂₄CIN₅S: C, 58.52; H, 6.20; N, 17.96; Cl, 9.09; S, 8.22. Found: C, 58.26; H, 6.18; N, 17.83; F, 8.95; S, 8.22.

### (78e) 3-amino-4-[4-(3-chlorophenyl)-1,4-diazepan-1-yl]thieno[2,3-b]pyridine-2-carboxamide

(2Z)-3-[4-(3-chlorophenyl)-1,4-diazepan-1-yl]-2-cyano-N-[(1E) -(dimethylamino)methylene]but-2-enethioamide which was produced in Example 78 (78d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Slightly brown powder (yeild 61%)

Mp 213-215° C.;

IR (KBr) νₘₐₓ 3442, 3324, 3183, 2950, 2836, 1644, 1593, 1494, 1369 cm⁻¹;

¹H NMR(DMSO-d₆, 400 MHz) δ 2.10-2.20 (2H, m), 3.13-3.24 (2H, m), 3.25-3.31 (2H, m), 3.51-3.58 (2H, m), 3.77 (2H, t, J=4.4 Hz), 6.62 (1H, dd, J=8.3, 2.0 Hz), 6.73 (1H, dd, J=8.3, 2.0 Hz), 7.01 (1H, bs), 7.06-7.12 (3H, m), 7.17 (1H, t, J=8.3 Hz), 8.40 (1H, d, J=5.4 Hz);

MS (FAB) m/z: 402 [M+H]⁺, 385, 273;

Anal. Calcd for C₁₉H₂₀N₅ClOS.0.18H₂0: C, 56.33; H, 5.07; N, 17.29; Cl, 8.75; S, 7.91. Found: C, 56.33; H, 5.01; N, 17.38; Cl, 8.77; S, 7.68.

### Example 79

### 3-amino-4-{4-[4-(benzyloxy)phenyl]-1,4-diazepan-1-yl}thieno[2,3-b]pyridine-2-carboxamide

(Exemplified Compound No. 3-134)

### (79a) tert-butyl 4-[4-(benzyloxy)phenyl]-1,4-diazepane-1-carboxylate

The reaction was performed in a similar method as described in Org. Lett., 4, 581-584 (2002) and the title compound was synthesized.

Colourless oil (yeild 15%)

IR (film) νₘₐₓ 2974, 1693, 1512, 1416, 1237, 1168 cm⁻¹;

¹H NMR(CDCl₃, 400 MHz) δ 1.37 (4.5H, s), 1.44 (4.5H, s), 1.90-2.00 (1H, m), 3.17-3.23 (1H, m), 3.27-3.35 (1H, m), 3.41-3.61 (6H, m), 4.97 (2H, s), 6.62 (2H, d, J=8.8 Hz), 6.86 (2H, d, J=8.8 Hz), 7.26-7.44 (5H, m);

MS (EI) m/z: 382 [M⁺], 327, 291, 235.

### (79b) 1-[4-(benzyloxy)phenyl]-1,4-diazepane

tert-butyl 4-[4-(benzyloxy)phenyl]-1,4-diazepane-1-carboxylate which was produced in Example 79 (79a) was used in place of tert-butyl 4-phenyl-1,4-diazepane-1-carboxylate and the reaction was performed in a similar method as described in Example 57 (57b) and the title compound was obtained.

Pale brown oil (yeild 100%)

IR (film) νₘₐₓ 3329, 3033, 1512, 1455, 1237, 1025, 812 cm⁻¹;

¹H NMR(CDCl₃, 400 MHz) δ 1.88 (2H, quint, J=5.9 Hz), 2.82 (2H, t, J=5.9 Hz), 3.01 (2H, t, J=5.5 Hz), 3.46-3.54 (4H, m), 4.98 (2H, s), 6.62 (2H, d, J=9.0 Hz), 6.86 (2H, d, J=9.0 Hz), 7.25-7.43 (5H, m);

MS (EI) m/z: 282 [M⁺], 191, 148.

### (79c) 4-{4-[4-(benzyloxy)phenyl]-1,4-diazepan-1-yl}-2-thieno-1,2-dihydropyridine -3-carbonitrile

1-[4-(benzyloxy)phenyl]-1,4-diazepane which was produced in Example 79 (79b) was used in place of 1-(4-methylphenyl)-1,4-diazepane and the reaction was performed in a similar method as described in Example 77 (77c) and the title compound was obtained.

Pale brown powder (yeild 30%)

Mp 190-191° C.;

IR (KBr) νₘₐₓ 3129, 3045, 2953, 2205, 1625, 1511, 1455, 1240 cm⁻¹;

¹H NMR(DMSO-d₆, 400 MHz) δ 1.87-1.98 (2H, m), 3.45 (2H, t, J=5.9 Hz), 3.62-3.75 (4H, m), 3.89-3.96 (2H, m), 4.96 (2H, s), 6.41 (1H, d, J=7.8 Hz), 6.70 (2H, d, J=9.0 Hz), 6.83 (2H, d, J=9.0 Hz), 7.26-7.42 (6H, m);

MS (FAB) m/z: 417 [M+H]⁺, 325, 200;

Anal. Calcd for C₂₄H₂₄N₄OS.0.54H₂O: C, 67.62; H, 5.93; N, 13.14; S, 7.52. Found: C, 67.30; H, 5.97; N, 13.50; S, 7.39.

### (79d) 3-amino-4-{4-[4-(benzyloxy)phenyl]-1,4-diazepan-1-yl}thieno[2,3-b]pyridine-2-carboxamide

b 4-{4-[4-(benzyloxy)phenyl]-1,4-diazepan-1-yl}-2-thieno-1,2-dihydropyridine-3-carbonitrile which was produced in Example 79 (79c) was used in place of 4-(isobutylamino)-2-thioxo-1,2-dihydropyridine-3-carbonitrile and the reaction was performed in a similar method as described in Example 5 (5c) and the title compound was obtained.

Slightly brown powder (yeild 89%)

Mp 211-213° C.;

IR (KBr) νₘₐₓ 3430, 3320, 3167, 2945, 1646, 1579, 1511, 1367, 1230 cm⁻¹;

¹H NMR(DMSO-d₆, 500 MHz) δ 2.06-2.16 (2H, m), 3.15-3.22 (2H, m), 3.24-3.30 (2H, m), 3.44-3.51 (2H, m), 3.65-3.72 (2H, m), 4.99 (2H, s), 6.69 (2H, d, J=9.0 Hz), 6.86 (2H, d, J=9.0 Hz), 6.98 (2H, bs), 7.05 (1H, d, J =5.1 Hz), 7.07 (2H, bs), 7.26-7.43 (5H, m), 8.37 (1H, d, J=5.1 Hz);

MS (FAB) m/z: 474 [M+H]⁺, 242, 200;

Anal. Calcd for C₂₆H₂₇N₅O₂S.0.26H₂O: C, 65.29; H, 5.80; N, 14.64; S, 6.70. Found: C, 65.46; H, 6.02; N, 14.59; S, 6.51.

### Example 80

### 3-amino-4-[4-(3-nitrophenyl)-1,4-diazepan-1-yl]thieno[2,3-b]pyridine-2-carboxamide

(Exemplified Compound No. 3-96)

### (80a) tert-butyl 4-(3-nitrophenyl)-1,4-diazepane-1-carboxylate

The reaction was performed in a similar method as described in Org. Lett., 4, 581-584 (2002) and the title compound was synthesized.

Orange oil (yeild 25%)

IR (film) νₘₐₓ 2975, 1692, 1528, 1347, 1166, 735 cm⁻¹;

¹H NMR(CDC₁₃, 400 MHz) δ 1.33 (4.5H, s), 1.41 (4.5H, s), 1.98 (2H, quint, J=5.9 Hz), 3.21-3.26 (1H, m), 3.31-3.37 (1H, m), 3.56-3.66 (6H, m), 6.94 (1H, dd, J=8.2, 2.0 Hz), 7.30 (1H, t, J=8.2 Hz), 7.44-7.50;

MS (FAB) m/z: 321 [M⁺], 266, 248, 222.

### (80b) 1-(3-nitrophenyl)-1,4-diazepane

tert-butyl 4-(3-nitrophenyl)-1,4-diazepane-1-carboxylate which was produced in Example 80 (80a) was used in place of tert-butyl 4-phenyl-1,4-diazepane-1-carboxylate and the reaction was performed in a similar method as described in Example 57 (57b) and the title compound was obtained.

Reddish Orange Oil (yeild 100%)

IR (film) νₘₐₓ 2935, 2855, 1618, 1525, 1347, 735 cm⁻¹;

¹H NMR(CDCl₃, 500 MHz) δ 1.93 (2H, quint, J=5.9 Hz), 2.85 (2H, t, J=5.9 Hz), 3.06 (2H, t, J=5.4 Hz, 3.60 (2H, t, J=5.4 Hz), 3.64 (2H, t, J=5.9 Hz), 6.95 (1H, dd, J=8.3, 2.4 Hz), 7.31 (1H, t, J=8.3 Hz), 7.44-7.51 (2H, m);

MS (EI) m/z: 221 [M⁺], 179, 165.

### (80c) (2Z)-2-cyano-3-[4-(3-nitrophenyl)-1,4-diazepan-1-yl]but-2-enethioamide

1-(3-nitrophenyl)-1,4-diazepane which was produced in Example 80 (80b) was used in place of isobutylamine and the reaction was performed in a similar method as described in Example 5 (5a) and the title compound was synthesized.

Orange powder (yeild 71%)

Mp 157-158° C.;

IR (KBr) νₘₐₓ 3443, 3353, 3277, 3176, 2926, 2182, 1617, 1525, 1346 cm⁻¹;

¹H NMR(DMSO-d₆, 500 MHz) δ 1.92-2.01 (2H, m), 2.24 (3H, s), 3.53-3.58 (2H, m), 3.62-3.69 (4H, m), 3.81-3.88 (2H, m), 7.20-7.26 (1H, m), 7.40-7.50 (3H, m), 8.40 (1H, bs), 9.05 (1H, bs);

MS (FAB) m/z: 346 [M+H]⁺, 246, 200.

### (80d) 3-amino-4-[4-(3-nitrophenyl)-1,4-diazepan-1-yl]thieno[2,3-b]pyridine-2-carboxamide

(2Z)-2-cyano-3-[4-(3-nitrophenyl)-1,4-diazepan-1-yl]but-2-enethioamide which was produced in Example 80 (80c) was used in place of (2Z)-2-cyano-3-(propylamino)but-2-enethioamide and the reaction was performed in a similar method as described in Example 57 (57b) and the title compound was obtained.

Pale yellow powder (yeild 6%)

Mp 180-185° C.;

IR (KBr) νₘₐₓ 3450, 3324, 3169, 2953, 2842, 1646, 1579, 1524,1368, 1345 cm⁻¹;

¹H NMR(DMSO-d₆, 500 MHz) δ 2.16-2.24 (2H, m), 3.15-3.24 (2H, m), 3.29-3.38 (2H, m), (2H, m), 3.64 (2H, t, J=5.9 Hz), 3.84-3.89 (2H, m), 7.01 (2H, bs), 7.09 (1H, d, J=5.4 Hz), 7.10 (2H, bs), 7.21-7.26 (1H. m), 7.44 (2H, d, J=5.4 Hz), 8.41 (1H, d, J=5.4 Hz);

MS (FAB) m/z: 413 [M+H]⁺, 246, 200.

### Example 81

### 3-amino-4-[4-(3-chloro-4-fluorophenyl)-1,4-diazepan-1-yl]thieno[2,3-b]pyridine-2-carboxamide

(Exemplified Compound No. 3-91)

### (81a) tert-butyl 4-(3-chloro-4-fluorophenyl)-1,4-diazepane-1-carboxylate

The reaction was performed in a similar method as described in Org. Lett.,4,581-584(2002) and the title compound was synthesized.

Colourless oil (yeild 36%)

IR (film) νₘₐₓ 2976, 1693, 1508, 1417, 1237, 1168 cm⁻¹;

¹H NMR(CDCl₃, 400 MHz) δ 1.36 (4.5H, s), 1.43 (4.5H, s), 1.89-1.98 (2H, m), 3.21 (1H, t, J=5.9 Hz), 3.32 (1H, t, J=5.9 Hz), 3.41-3.61 (6H, m), 6.48 (1H, d, J=9.0, 3.3 Hz), 6.59-6.65 (1H, m), 6.96 (1H, t, J=9.0 Hz);

MS (FAB) m/z: 328 [M⁺], 273, 229, 189.

### (81b) 1-(3-chloro-4-fluorophenyl)-1,4-diazepane

tert-butyl 4-(3-chloro-4-fluorophenyl)-1,4-diazepane-1-carboxylate which was produced in Example 81 (81a) was used in place of tert-butyl 4-phenyl-1,4-diazepane-1-carboxylate and the reaction was performed in a similar method as described in Example 57 (57b) and the title compound was obtained.

Slightly brown oil (yeild 100%)

IR (film) νₘₐₓ 2934, 1611, 1508, 1240, 1047, 797 cm⁻¹;

¹H NMR(CDCl₃, 500 MHz) δ 1.88 (2H, quint, J=5.9 Hz), 2.83 (2H, t, J=5.9 Hz), 3.01 (2H, t, J=5.5 Hz), 3.48 (2H, t, J=5.5 Hz), 3.52 (2H, t, J=5.9 Hz), 6.49 (1H, dt, J=9.0, 3.1 Hz), 6.64 (1H, q, J=3.1 Hz), 6.97 (1H, t, J=9.0 Hz);

MS (EI) m/z: 228 [M⁺], 186, 172.

### (81c) (2Z)-3-[4-(3-chloro-4-fluorophenyl)-1,4-diazepan-1-yl]-2-cyanobut-2-enethioamide

1-(3-chloro-4-fluorophenyl)-1,4-diazepane which was produced in Example 81 (81b) was used in place of isobutylamine and the reaction was performed in a similar method as described in Example 5 (5a) and the title compound was synthesized.

Slightly yellow powder (yeild 63%)

Mp 148-150° C.;

IR (KBr) νₘₐₓ 3358, 3265, 3170, 2177, 1615, 1526, 1505, 1408 cm⁻¹;

¹H NMR(DMSO-d₆, 500 MHz) δ 1.89-1.96 (2H, m), 2.25 (3H, s), 3.49-3.56 (4H, m), 3.58-3.64 (2H, m), 3.68-3.75 (2H, m), 6.73 (1H, dt, J =9.0, 3.1 Hz), 6.90 (1H, q, J=3.1 Hz), 7.18 (1H, t, J=9.0 Hz), 8.37 (1H, bs), 9.02 (1H, bs);

MS (FAB) m/z: 353 [M+H]⁺, 200, 165.

### (81d) (2Z)-3-[4-(3-chloro-4-fluorophenyl)-1,4-diazepan-1-yl]-2-cyano-N-[(1E) -(dimethylamino)methylene]but-2-enethioamide

(2Z)-3-[4-(3-chloro-4-fluorophenyl)-1,4-diazepan-1-yl]-2-cyanobut-2-enethioamide which was produced in Example 81 (81c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Single yellow powder (yeild 45%)

Mp 133-135° C. (decomposition);

IR (KBr) νₘₐₓ 2924, 2177, 1609, 1506, 1398, 1326, 1289 cm⁻¹;

¹H NMR(DMSO-d₆, 400 MHz) δ 1.91-2.00 (2H, m), 2.47 (3H, s), 2.91 (3H, s), 3.14 (3H, s) 3.50-3.58 (2H, m), 3.68-3.87 (6H, m), 6.73 (1H, dt, J=9.0, 3.1 Hz), 6.90 (1H, q, J=3.1 Hz), 7.15 (1H, t, J=9.0 Hz), 8.43 (1H, s);

MS (FAB) m/z: 408 [M+H]⁺, 374,273.

### (81e) 3-amino-4-[4-(3-chloro-4-fluorophenyl)-1,4-diazepan-1-yl]thieno[2,3-b]pyridine-2-carboxamide

(2Z)-3-[4-(3-chloro-4-fluorophenyl)-1,4-diazepan-1-yl]-2-cyano-N-[(1E) -(dimethylamino)methylene]but-2-enethioamide which was produced in Example 81 (81d) was used in place of (2Z)-2-cyano-N-[(1E)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Pale brown powder (yeild 54%)

Mp 181-184° C.;

IR (KBr) νₘₐₓ 3326, 3095, 2834, 1636, 1579, 1506, 1373 cm⁻¹;

¹H NMR(DMSO-d₆, 400 MHz) δ 2.09-2.19 (2H, d), 3.13-3.22 (2H, m), 3.24-3.32 (2H, m), 3.51 (2H, t, J=5.9 Hz), 3.71-3.77 (2H, m), 6.71 (1H, dt, J=9.0, 3.1 Hz), 6.85 (1H, q, J=3.1 Hz), 6.99 (2H, bs), 7.06 (1H, d, J=5.5 Hz), 7.09 (2H, bs), 7.18 (1H, t, J=9.0 Hz), 8.38 (1H, d, J=5.5 Hz);

MS (FAB) m/z: 420 [M+H]⁺, 273, 176;

Anal. Calcd for C₁₉H₁₉CIFN₅OS: C, 54.35; H, 4.65; N, 16.68; Cl, 8.44; F, 4.52; S, 7.64. Found: C, 54.04; H, 4.43; N, 16.30; Cl, 8.33; F, 4.86; S, 8.03.

### Example 82

### 3-amino-4-[4-(4-bromophenyl)-1,4-diazepan-1-yl]thieno[2,3-b]pyridine-2-carboxamide

(Exemplified Compound No. 3-95)

### (82a) tert-butyl 4-(4-bromophenyl)-1,4-diazepane-1-carboxylate

The reaction was performed in a similar method as described in Org. Lett.,4,581-584(2002) and the title compound was synthesized.

Colourless oil (yeild 27%)

IR (film) νₘₐₓ 2975, 1693, 1591, 1498, 1416, 1237, 1167 cm⁻¹;

¹H NMR(CDCl₃, 500 MHz) δ 1.37 (4.5H, s), 1.43 (4.5H, s), 1.90-1.99 (2H, m), 3.20 (1H, t, J=5.9 Hz), 3.31 (1H, t, J=5.9 Hz), 3.48-3.58 (6H, m), 6.56 (2H, d, J=8.8 Hz), 7.27 (2H, d, J=8.8 Hz);

MS (FAB) m/z: 355 [M+H]⁺, 255, 189.

### (82b) 1-(4-bromophenyl)-1,4-diazepane

tert-butyl 4-(4-bromophenyl)-1,4-diazepane-1-carboxylate which was produced in Example 82 (82a) was used in place of tert-butyl 4-phenyl-1,4-diazepane-1-carboxylate and the reaction was performed in a similar method as described in Example 57 (57b) and the title compound was obtained.

Slightly brown oil (yeild 90%)

IR (film) νₘₐₓ 2931, 1591, 1498, 1396, 1362, 1190, 806 cm₋₁;

¹H NMR(CDCl₃, 500 MHz) δ 1.88 (2H, quint, J=5.9 Hz), 2.82 (2H, t, J=5.9 Hz), 3.01 (2H, t, J=5.4 Hz), 3.51, t, J=5.4 Hz), 3.55 (2H, t, J=5.9 Hz), 6.56 (2H, d, J=8.8 Hz), 7.26 82H, d, J=8.8 Hz);

MS (EI) m/z: 254 [M⁺], 212, 198.

### (82c) (2Z)-3-[4-(4-bromophenyl)-1,4-diazepan-1-yl]-2-cyanobut-2-enethioamide

1-(4-bromophenyl)-1,4-diazepane which was produced in Example 82 (82b) was used in place of isobutylamine and the reaction was performed in a similar method as described in Example 5 (Sa) and the title compound was synthesized.

Slightly yellow powder (yield 72%)

Mp 152-155° C.;

IR (KBr) νₘₐₓ 3371, 3276, 3174, 2957, 2185, 1589, 1536,1496, 1408, 1357 cm⁻¹;

¹H NMR(DMSO-d₆, 500 MHz) δ 1.87-1.97 (2H, m), 2.24 (3H, s), 3.48-3.57 (4H, m), 3.58-3.64 (2H, m), 3.70-3.77 (2H, m), 6.74 (2H, d, J=8.8 Hz), 7.28 (2H, d, J=8.8 Hz), 8.38 (1H, bs), 9.03 (1H, bs);

MS (FAB) m/z: 379 [M+H]⁺, 273, 182.

### (82d) (2Z)-3-[4-(4-bromophenyl)-1,4-diazepan-1-yl]-2-cyano-N-[(1E) -(dimethylamino)methylene]but-2-enethioamide

(2Z)-3-[4-(4-bromophenyl)-1,4-diazepan-1-yl]-2-cyanobut-2-enethioamide which was produced in Example 82 (82c) was used in place of (2Z)-2-cyano-3-[4-(4-methoxyphenyl)-1,4-diazepan-1-yl]but-2-enethioamide and the reaction was performed in a similar method as described in Example 58 (58d) and the title compound was obtained.

Single yellow powder (yeild 84%)

Mp 143-146° C. (decomposition);

IR (KBr) νₘₐₓ 2925, 2176, 1611, 1497, 1397, 1328, 1290 cm⁻¹;

¹H NMR(DMSO-d₆, 400 MHz) δ 1.91-2.00 (2H, m), 2.47 (3H, s), 2.92 (3H, s), 3.15 (3H, s), 3.55 (2H, t, J=5.9 Hz), 3.69-3.85 (6H, m), 6.74 (2H, d, J=8.8 Hz), 7.26 (2H, d, J=8.8 Hz), 8.46 (1H, s);

MS (FAB) m/z: 434 [M+H]⁺, 400, 273.

### (82e) 3-amino-4-[4-(4-bromophenyl)-1,4-diazepan-1-yl]thieno[2,3-b]pyridine-2-carboxamide

(2Z)-3-[4-(4-bromophenyl)-1,4-diazepan-1-yl]-2-cyano-N-[(1E) -(dimethylamino)methylene]but-2-enethioamide which was produced in Example 82 (82d) was used in place of (2Z)-2-cyano-N-[(lE)-(dimethylamino)methylene]-3-[4-(4-methoxyphenyl)-1,4-diazepan-1-yl]but-2-enethioamide and the reaction was performed in a similar method as described in Example 58 (58e) and the title compound was obtained.

Slightly brown powder (yeild 59%)

Mp 170-173° C.;

IR (KBr) νₘₐₓ 3442, 3322, 3180, 2948, 2838, 1645, 1588, 1498, 1367 cm⁻¹;

¹H NMR(DMSO-d₆, 400 MHz) δ 2.13 (2H, quint, J =5.5 Hz), 3.13-3.21 (2H, m), 3.23-3.30 (2H, m), 3.52 (2H, t, J=6.3 H), 3.71-3.77 (2H, m), 6.72 (2H, d, J=9.0 Hz), 6.98 (2H, bs), 7.05 81H, d, J=5.5 Hz), 7.08 (2H, bs), 7.27 (2H, d, J=9.0 Hz), 8.38 (1H, d, J=5.5 Hz);

MS (FAB) m/z: 446 [M+H]⁺, 429, 273.

### Example 83

### 3-amino-4-[4-(4-ethoxyphenyl)-1,4-diazepan-1-yl]thieno[2,3-b]pyridine-2-carboxamide

(Exemplified Compound No. 3-127)

### (83a) tert-butyl 4-(4-ethoxyphenyl)-1,4-diazepane-1-carboxylate

The reaction was performed in a similar method as described in Org. Lett.,4,581-584(2002) and the title compound was synthesized.

Colourless oil (yeild 27%)

IR (film) νₘₐₓ 2976, 1694, 1513, 1416, 1240,1169 cm⁻¹;

¹H NMR(CDCl₃, 500 MHz) δ 1.37 (3H, t, J=6.8 Hz), 1.38(4.5H, s), 1.44 (4.5H, s), 1.91-2.00 (2H, m), 3.21 (1H, t, J =5.9 Hz), 3.32 (1H, t, J=5.9 Hz), 3.45-3.60 (6H, m), 3.96 (2H, q, J=6.8 Hz), 6.64 (2H, d, J=9.3 Hz), 6.81 (2H, d, J=9.3 Hz);

MS (FAB) m/z: 320 [M⁺], 264, 219.

### (83b) 1-(4-ethoxyphenyl)-1,4-diazepane

tert-butyl 4-(4-ethoxyphenyl)-1,4-diazepane-1-carboxylate which was produced in Example 83 (83a) was used in place of tert-butyl 4-phenyl-1,4-diazepane-1-carboxylate and the reaction was performed in a similar method as described in Example 57 (57b) and the title compound was obtained.

Brown Oil (yeild 98%)

IR (film) νₘₐₓ 2931, 1513, 1239, 1050, 812 cm⁻¹;

¹H NMR(CDCl₃, 400 MHz) δ 1.37 (3H, t, J=7.0 Hz), 1.88 (2H, quint, J=5.9 Hz), 2.82 (2H, t, J=5.5H), 3.01 (2H, t, J=5.5 Hz), 3.46-3.55 (4H, m), 3.95 (2H, q, J=7.0 Hz), 6.62 (2H, d, J=9.4 Hz), 6.79 (2H, d, J=9.4 Hz);

MS (EI) m/z: 220 [M⁺], 178, 164.

### (83c) 4-[4-(4-ethoxyphenyl)-1,4-diazepan-1-yl]-2-thioxa-1,2-dihydropyridine-3-carbonitrile

1-(4-ethoxyphenyl)-1,4-diazepane which was produced in Example 83 (83b) was used in place of 1-(4-methylphenyl)-1,4-diazepane and the reaction was performed in a similar method as described in Example 77 (77c) and the title compound was obtained.

Slightly brown powder (yeild 25%)

Mp 204-208° C.;

IR (KBr) νₘₐₓ 3118, 2970, 2207, 1625, 1511, 1244 cm⁻¹;

¹H NMR(CDCl₃, 400 MHz) δ 1.38 (3H, t, J=7.0 Hz), 2.11 (2H, quint, J=5.9 Hz), 3.50 (2H, t, J=5.9 Hz), 3.69-3.78 (4H, m), 3.96 (2H, q, J=7.0 Hz), 4.07-4.13 (2H, m), 6.20 (1H, d, J=7.6 Hz), 6.66 (2H, d, J=9.2 Hz), 6.81 (2H, d, J=9.2 Hz), 7.22 (1H, d, J=7.6 Hz),11.97 (1H, bs);

MS (FAB) m/z: 355 [M+H]⁺, 273;

Anal. Calcd for C₁₉H₂₂N₄OS.0.16H₂O: C, 63.86; H, 6.38; N, 15.68; S, 8.97. Found: C, 63.86; H, 6.26; N, 15.66; S, 8.85.

### (83d) 3-amino-4-[4-(4-ethoxyphenyl)-1,4-diazepan-1-yl]thieno[2,3-b]pyridine-2-carboxamide

4-[4-(4-ethoxyphenyl)-1,4-diazepan-1-yl]-2-thioxa-1,2-dihydropyridine-3-carbonitrile which was produced in Example 83 (83c) was used in place of 4-(isobutylamino)-2-thioxo-1,2-dihydropyridine-3-carbonitrile and the reaction was performed in a similar method as described in Example 5 (5c) and the title compound was obtained.

Slightly brown powder (yeild 84%)

Mp 181-183° C.;

IR (KBr) νₘₐₓ 3423, 3330, 3116, 2973, 1586, 1512, 1369, 1234 cm⁻¹;

¹H NMR(DMSO-d₆, 500 MHz) δ 1.28 (3H, t, J=7.0 Hz), 2.08-2.16 (2H, m), 3.18-3.24 (2H, m), 3.26-3.31 (2H, m), 3.40 82H, t, J=5.9 Hz), 3.70 82H, t, J=4.7 hz), 3.92 (2H, q, J=7.0 Hz), 6.71 (2H, d, J =8.8 Hz), 6.80 (2H, d, J=8.8 Hz), 6.99 (2H, bs), 7.05-7.12 (3H, m), 8.40 (1H, d, J=5.4 Hz);

MS (FAB) m/z: 412 [M+H]⁺, 75;

Anal. Calcd for C₂₁H₂₅N₅O₂S.0.42H₂O: C, 60.19; H, 6.21; N, 16.71; S, 7.65. Found: C, 60.49; H, 6.32; N, 16.93; S, 7.35.

### Example 84

### 3-amino-4-[4-(3,4-dimethylphenyl)-1,4-diazepan-1-yl]thieno[2,3-b]pyridine-2-carboxamide

(Exemplified Compound No. 3-116)

### (84a) tert-butyl 4-(3,4-dimethylphenyl)-1,4-diazepane-1-carboxylate

The reaction was performed in a similar method as described in Org. Lett., 4, 581-584 (2002) and the title compound was synthesized.

Colourless oil (Yield 17%)

IR (film) νₘₐₓ 3366, 2972, 2931, 1695, 1616, 1511, 1415, 1242, 1169 cm⁻¹;

¹H NMR (CDCl₃, 500 MHz) δ 1.36 (4.5H, s), 1.45 (4.5H, s), 1.93-2.02 (2H, m), 2.15 (3H, s), 2.22 (3H, s), 3.20 (1H, t, J=5.9 Hz), 3.30 (1H, d, J=5.9 Hz), 3.47-3.59 (6H, m), 6.46 (1H, d, J=8.3 Hz), 6.51 (1H, s), 6.96 (1H, d);

MS (FAB) m/z: 304 [M⁺], 249, 203.

### (84b) 1-(3,4-dimethylphenyl)-1,4-diazepane

tert-butyl 4-(3,4-dimethylphenyl)-1,4-diazepane-1-carboxylate which was produced in Example 84 (84a) was used in place of tert-butyl 4-phenyl-1, 4-diazepane-1-carboxylate and the reaction was performed in a similar method as described in Example 57 (57b) and the title compound was obtained.

Pale brown prism crystal (Yield 100%)

IR (KBr) νₘₐₓ 2939, 2860, 2384, 1615, 1512, 1459, 1411, 1280 cm⁻¹;

¹H NMR (CDCI₃, 400 MHz) δ 1.88 (2H, quint, J=5.9 Hz), 2.15 (3H, s), 2.21 (3H, s), 2.82 (2H, t, J=5.9 Hz), 3.01 (2H, t, J=5.5 Hz), 3.49-3.56 (4H, m), 6.45 (1H, dd, J=8.2, 2.7 Hz), 6.50 (1H, d, J=2.7 Hz), 6.94 (1H, d, J -8.2 Hz);

MS (EI) m/z: 204 [M⁺], 174, 162, 148.

### (84c) 4-[4-(3,4-dimethylphenyl)-1,4-diazepan-1-yl]-2-thioxa-1,2-dihydropyridine-3-carbonitrile

1-(3,4-dimethylphenyl)-1,4-diazepane which was produced in Example 84 (84b) was used in place of 1-(4-methylphenyl)-1,4-diazepane and the reaction was performed in a similar method as described in Example 77 (77c) and the title compound was obtained.

Pale brown powder (Yield 31%)

Mp 251-253° C.;

IR (KBr) νₘₐₓ 3121, 2937, 2205, 1625, 1511, 1459, 1254 cm⁻¹;

¹H NMR (DMSO-d₆, 400 MHz) δ 1.93 (2H, quint, J=5.5 Hz), 2.07 (3H, s), 2.14 (3H, s), 3.48 (2H, t, J=5.9 Hz), 3.65-3.74 (4H, m), 3.91-3.98 (2H, m), 6.43 (1, d, J=7.6 Hz), 6.50 (1H, dd, J=8.4, 2.7 Hz), 6.60 (1H, d, J=2.7 Hz), 6.90 (1H, d, J=8.4 Hz), 7.37 (1H, d, J=7.6 Hz), 12.44 (1H, bs);

MS (FAB) m/z: 339 [M+H]⁺, 273, 174;

Anal. Calcd for Cl₁₉H₂₂N₄S.0.24H₂O: C, 66.57; H, 6.61; N, 16.34; S, 9.35. Found: C, 66.47; H, 6.44; N, 16.34; S, 9.15.

### (84d) 3-amino-4-[4-(3,4-dimethylphenyl)-1,4-diazepan-1-yl]thieno[2,3-b]pyridine-2-carboxamide

4-[4-(3,4-dimethylphenyl)-1,4-diazepan-1-yl]-2-thioxa-1,2-dihydropyridine-3-carbonitrile which was produced in Example 84 (84c) was used in place of 4-(isobutylamino)-2-thioxo-1,2-dihydropyridine-3-carbonitrile and the reaction was performed in a similar method as described in Example 5 (5c) and the title compound was obtained.

Slightly brown powder (Yield 82%)

Mp 195-197° C.;

IR (KBr) νₘₐₓ 3432, 3324, 3174, 2934, 2829, 1642, 1579, 1507, 1369 cm⁻¹;

¹H NMR (DMSO-d₆, 500 MHz) δ2.08-2.15 (2H, m), 2.10 (3H,s), 2.16 (3H, s), 3.15-3.22 (2H, m), 3.25-3.31 (2H, m), 3.52 (2H, t, J=5.9 Hz), 3.69-3.76 (2m, 6.50 (1H, dd, J=8.3, 2.4 Hz), 6.60 (1H, d, J=2.4 Hz), 6.92 (1H, d, J=8.3 Hz), 6.99 (2H, bs), 7.04-7.13 (3H, m), 8.40 (1H, d, J=5.4 Hz);

MS (FAB) m/z: 396 [M+H]⁺, 246, 185;

Anal. Calcd for C₂₁H₂₅N₅OS: C, 63.77; H, 6.37; N, 17.71; S, 8.11. Found: C, 63.45; H, 6.33; N, 17.54; S, 8.11.

### Example 85

### 3-amino-4-[4-(3,4-difluorophenyl)-1,4-diazepan-1-yl]thieno[2,3-b]pyridine-2-carboxamide

(Exemplified Compound No. 3-90)

### (85a) tert-butyl 4-(3,4-difluorophenyl)-1,4-diazepane-1-carboxylate

The reaction was performed in a similar method as described in Org. Lett., 4, 581-584 (2002) and the title compound was synthesized.

Colourless oil (Yield 29%)

IR (film) νₘₐₓ 2976, 1691, 1521, 1417, 1234, 1168, 777 cm⁻¹;

¹H NMR (CDCl₃, 400 MHz) δ 1.36 (4.5H, s), 1.43 (4.5H, s), 1.89-1.98 (2H, m), 3.21 (1H, t, J=5.9 Hz), 3.31 (1H, t, J=5.9 Hz), 3.41-3.58 (6H, m), 6.27-6.33 (1H, m), 6.38-6.47 (1H, m), 6.96 (1H, q, J=9.0 Hz);

MS (FAB) m/z: 312 [M+], 257, 211.

### (85b) 1-(3,4-difluorophenyl)-1,4-diazepane

tert-butyl 4-(3,4-difluorophenyl)-1,4-diazepane-1-carboxylate which was produced in Example 85 (85a) was used in place of tert-butyl 4-phenyl-1,4-diazepane-1-carboxylate and the reaction was performed in a similar method as described in Example 57 (57b) and the title compound was obtained.

Brown oil (Yield 98%)

IR (film) νₘₐₓ 2935, 2838, 1631, 1597, 1520, 1275, 777 cm⁻¹;

¹H NMR (CDCl₃, 500 MHz) ν 1.88 (2H, m, J=5.9 Hz), 2.83 (2H, t, J=5.9 Hz), 3.01 (2H, t, J=5.4 Hz), 3.48 (2H, t, J=5.4 Hz), 3.52 (2H, t, J=5.9 Hz), 6.28-6.34 (1H, m), 6.44 (1H, ddd, J=14.2, 6.8, 2.9 Hz), 6.98 (1H, q, J=9.3 Hz);

MS (EI) m/z: 212 [M+], 170, 156.

### (85c) 4-[4-(3,4-difluorophenyl)-1,4-diazepan-1-yl]-2-thioxo-1,2-dihydropyridine-3-carbonitrile

1-(3,4-difluorophenyl)-1,4-diazepane which was produced in Example 85 (85b) was used in place of 1-(4-methylphenyl)-1,4-diazepane and the reaction was performed in a similar method as described in Example 77 (77c) and the title compound was obtained.

Slightly brown powder (Yield 27%)

Mp 250-251° C.;

IR (KBr) νₘₐₓ 3122, 2959, 2206, 1626, 1520, 1247, 777 cm⁻¹;

¹H NMR (DMSO-d₆, 500 MHz) δ 1.93 (2H, quint, J=5.9 Hz), 3.51 (2H, m), 3.68-3.76 (4H, m), 3.92-3.98 (2H, m), 6.43 (1H, d, J=7.8 Hz), 6.52-6.57 (1H, m), 6.83 (1H, ddd, J=14.7, 6.8, 2.9 Hz), 7.17 (1H, q, J=9.8 Hz), 7.37 (1H, d, J=7.8 Hz), 12.15 (1H, bs);

MS (FAB) m/z: 347 [M+H]⁺, 273, 242;

Anal. Calcd for C₁₇H₁₆F₂N₄S.0.2 H₂O: C, 58.34; H, 4.72; F, 10.86; N, 16.01; S, 9.16. Found: C, 58.48; H, 4.73; F, 10.59; N, 16.13; S, 9.04.

### (85d) 3-amino-4-[4-(3,4-difluorophenyl)-1,4-diazepan-1-yl]thieno[2,3-b]pyridine-2-carboxamide

4-[4-(3,4-difluorophenyl)-1,4-diazepan-1-yl]-2-thioxo-1,2-dihydropyridine-3-carbonitrile which was produced in Example 85 (85c) was used in place of 4-(isobutylamino) -2-thioxo-1,2-dihydropyridine-3-carbonitrile and the reaction was performed in a similar method as described in Example 5 (5c) and the title compound was obtained.

Slightly brown powder (Yield 83%)

Mp 199-201° C.;

IR (KBr) νₘₐₓ 3454, 3324, 3171, 2953, 2839, 1650, 1582, 1519, 1369 cm⁻¹;

¹NMR(DMSO-d₆, 500 MHz) δ 2.10-2.18 (2H, m), 3.14-3.23 (2H, m), 3.24-3.32 (2H, m), 3.52 (2H, t, J=6.4 Hz), 3.75 (2H, t, J=5.0 Hz), 6.49-6.56 (1H, m), 6.78 (1H, ddd, J=14.7, 6.8, 2.9 Hz), 7.00 (2H, bs), 7.08 (1H, d, J=5.2 Hz), 7.10 (2H, bs), 7.20 (1H, q, J=9.8 Hz), 8.41 (1H, d, J=5.2 Hz);

MS (FAB) m/z: 404 [M+H]⁺, 387, 246, 200;

Anal. Calcd for C₁₉H₁₉F2N₅O₂S.0.24H₂O: C, 55.96; H, 4.82; F, 9.32; N, 17.17; S, 7.86. Found: C, 55.97; H, 4.76; F, 9.25; N, 17.24; S, 7.76.

### Example 86

### 3-amino-4-{4-[3-(trifluoromethyl)phenyl]-1,4-diazepan-1-yl}thieno[2,3-b]pyridine-2-carboxamide

(Exemplified Compound No. 3-120)

### (86a) tert-butyl 4-[3-(trifluoromethyl)phenyl]-1,4-diazepane-1-carboxylate

The reaction was performed in a similar method as described in Org. Lett., 4,581-584(2002) and the title compound was synthesized.

Colourless oil (Yield 13%)

IR (film) νₘₐₓ 2976, 1695, 1612, 1463, 1417, 1321, 1164, 1124 cm⁻¹;

¹H NMR (CDCl₃, 500 MHz) δ 1.33 (4.5H, s), 1.43 (4.5H, s), 1.93-2.01 (2H, m), 3.23 (8H, t, J=5.9 Hz), 3.34 (1H, t, J=5.9 Hz), 3.54-3.64 (6H, m), 6.80-6.94 (3H, m), 7.23-7.32 (1H, m);

MS (FAB) m/z: 344 [M+], 289, 243.

### (86b) 1-[3-(trifluoromethyl)phenyl]-1,4-diazepane

tert-butyl 4-[3-(trifluoromethyl)phenyl]-1,4-diazepane-1-carboxylate which was produced in Example 86 (86a) was used in place of tert-butyl 4-phenyl-1,4-diazepane -1-carboxylate and the reaction was performed in a similar method as described in Example 57 (57b) and the title compound was obtained.

Brown oil (Yield 89%)

IR (film) νₘₐₓ 2938, 1691, 1611, 1506, 1457, 1321, 1162, 1121 cm⁻¹;

¹H NMR (CDCl₃, 500 MHz) δ 1.91 (2H, t, J=5.9 Hz), 2.84 (2H, t, J=5.9 Hz), 3.04 (2H, t, J=5.4 Hz), 3.57 (2H, t, J=5.4 Hz), 3.60 (2H, t, J=6.4 Hz), 6.80-691 (3H, m), 7.25-7.31 (1H, m);

MS (EI) m/z: 244 [M+], 202, 188.

### (86c) 2-thioxo-4-{4-[3-(trifluoromethyl)phenyl]-1,4-diazepan-1-yl}-1,2-dihydropyridine-3-carbonitrile

1-[3-(trifluoromethyl)phenyl]-1,4-diazepane which was produced in Example 86 (86b) was used in place of 1-(4-methylphenyl)-1,4-diazepane and the reaction was performed in a similar method as described in Example 77 (77c) and the title compound was obtained.

Slightly brown powder (Yield 27%)

Mp 247-248° C.;

IR (KBr) νₘₐₓ 2414, 3129, 2964, 2207, 1625, 1523, 1320, 1120 cm⁻¹;

¹H NMR (DMSO-d₆, 500 MHz) 6 1.92-2.00 (2H, m), 3.60 (2H, t, J=5.9 Hz), 3.75 (2H, t, J=5.9 Hz), 3.80 (2H, t, J=5.4 Hz), 3.08 (2H, t, J=5.4 Hz), 6.45 (1H, d, J=7.8 Hz), 6.90 (1H, d, J=7.3 Hz), 6.99 (1H, s), 7.07 (1H, dd, J=8.3, 2.0 Hz), 7.33-7.40 (2H, m), 11.53 (1H, bs);

MS (FAB) m/z: 379 [M+H]⁺, 273, 226;

Anal. Calcd for C₁₈H₁₇F₃N₄S.0.2H₂O: C, 56.69; H, 4.59; N, 14.67; S, 8.39. Found: C, 56.60; H, 4.58; N, 14.67; S, 8.39.

### (86d) 3-amino-4-{4-[3-(trifluoromethyl)phenyl]-1,4-diazepan-1-yl}thieno[2,3-b]pyridine-2-carboxamide

2-thioxo-4-{4-[3-(trifluoromethyl)phenyl]-1,4-diazepan-1-yl}-1,2-dihydropyridine -3-carbonitrile which was produced in Example 86 (86c) was used in place of 4-(isobutylamino)-2-thioxo-1,2-dihydropyridine-3-carbonitrile and the reaction was performed in a similar method as described in Example 5 (5c) and the title compound was obtained.

Slightly brown powder (Yield 84%)

Mp 195-197° C.;

IR (KBr) νₘₐₓ 3427, 3326, 3167, 1639, 1580, 1504, 1371, 1121 cm⁻¹;

¹H NMR (DMSO-d₆, 500 MHz) δ 2.14-2.23 (2H, m), 3.15-3.25 (2H, m), 3.26-3.36 (2H, m), 3.60 (2H, t, J=5.9 Hz), 3.84 (2H, t, J=4.6 Hz), 6.91 (1H, d, J=7.3Hz), 6.95-6.99 (1H, m), 7.01 (1H, bs), 7.06 (1H, dd, J=8.3, 2.0 Hz), 7.09 (1H, d, J=5.2 Hz), 7.10 (1H, bs), 7.38 81H, t, J=8.3 Hz), 8.41 (1H, d, J=5.2 Hz);

MS (FAB) m/z: 436 [M+H]⁺, 419, 240.

### Example 87

### 3-amino-4-{4-[4-(trifluoromethoxy)phenyl]-1,4-diazepan-1-yl}thieno[2,3-b]pyridine-2-carboxamide

(Exemplified Compound No. 3-130) (87a) tert-butyl 4-[4-(trifluoromethoxy)phenyl]-1,4-diazepane-1-carboxylate

The reaction was performed in a similar method as described in Org. Lett., 4, 581-584 (2002) and the title compound was synthesized.

Slightly brown oil (Yield 41%)

IR (film) νₘₐₓ 2976, 1692, 1516, 1417, 1268, 1163 cm⁻¹;

¹H NMR (CDCl₃, 400 MHz) δ 1.33 (4.5H, s), 1.42 (4.5H, s), 1.90-1.98 (2H, m), 3.22 (1H, t, J=5.9 Hz), 3.32 (1H, t, J=5.5 Hz), 3.48-3.60 (6H, m), 6.61 (2H, d, J=9.2 Hz), 7.04 (2H, d, J -9.2 Hz);

MS (FAB) m/z: 360 [M⁺], 305, 259.

### (87b) 1-[4-(trifluoromethoxy)phenyl]-1,4-diazepane

tert-butyl 4-[4-(trifluoromethoxy)phenyl]-1,4-diazepane-1-carboxylate which was produced in Example 87 (87a) was used in place of tert-butyl 4-phenyl-1,4-diazepane-1-carboxylate and the reaction was performed in a similar method as described in Example 57 (57b) and the title compound was obtained.

Green oil (Yield 57%)

IR (film) νₘₐₓ 2936, 1610, 1516, 1265, 1231, 1205, 1156, 805 cm⁻¹;

¹H NMR (CDCl₃, 500 MHz) δ 1.89 (2H, quint, J=5.9 Hz), 2.83 (2H, t, J=5.9 Hz), 3.03 (2H, t, J=5.4 Hz), 3.53 (2H, t, J=5.4 Hz), 3.56 (2H, t, J=5.9 Hz), J=9.3 Hz), 7.05 (2H, d,J=9.3 Hz);

MS (EI) m/z: 260 [M+], 218, 204.

### (87c) 2-thioxa-4-{4-[4-(trifluoromethoxy)phenyl]-1,4-diazepan-1-yl}-1,2-dihydropyridine -3-carbonitrile

1-[4-(trifluoromethoxy)phenyl]-1,4-diazepane which was produced in Example 87 (87b) was used in place of 1-(4-methylphenyl)-1,4-diazepane and the reaction was performed in a similar method as described in Example 77 (77c) and the title compound was obtained.

Slightly brown powder (Yield 19%)

Mp 240-242° C.;

IR (KBr) νₘₐₓ 3122, 2957, 2210, 1627, 1514, 1245, 1150 cm⁻¹;

¹H NMR (DMSO-d₆, 500 MHz) δ1.91-1.98 (2H, m), 3.55 (2H, t, J=5.9 Hz), 3.70-3.78 (4H, m), 3.97 (2H, t, J=5.4 Hz), 6.44 (1H, d, J=7.8 Hz), 6.83 (2H, d, J=9.3

Hz), 7.13 (2H, d, J=9.3 Hz), 7.37 (1H, d, J=7.8 Hz), 12.53 (1H, bs);

MS (FAB) m/z: 395 [M+H]⁺, 175;

Anal. Calcd for C₁₈H₁₇F₃N₄OS: C, 54.81; H, 4.34; N, 14.21; S, 8.13. Found: C, 54.74; H, 4.06; N, 14.12; S, 8.08.

### (87d) 3-amino-4-{4-[4-(trifluoromethoxy)phenyl]-1,4-diazepan-1-yl}thieno[2,3-b]pyridine-2-carboxamide

2-thioxa-4-{4-[4-(trifluoromethoxy)phenyl]-1,4-diazepan-1-yl}-1,2-dihydropyridine -3-carbonitrile which was produced in Example 87 (87c) was used in place of 4-(isobutylamino)-2-thioxo-1,2-dihydropyridine-3-carbonitrile and the reaction was performed in a similar method as described in Example 5 (5c) and the title compound was obtained.

lightly brown powder (Yield 79%)

Mp 163-165° C.;

IR (KBr) νₘₐₓ 3413, 3325, 3176, 2947, 2836, 1637, 1579, 1515, 1372 cm⁻¹;

¹H NMR (DMSO-d₆, 500 MHz) δ 2.11-2.19 (2H, m), 3.16-3.24 (2H, m), 3.26 -3.33 (2H, m), 3.55 (2H, t, J=5.9 Hz), 3;79 (2H, t, J=4.6 Hz), 6.82 (2H, d, J=7.3 Hz), 7.00 (2H, bs), 7.08 (1H, d, J=5.4 Hz), 7.10 (2H, bs), 7.15 (2H, d, J=9.3 (1H, d, J=5.4 Hz);

MS (FAB) m/z: 452 [M+H]⁺, 435, 216;

Anal. Calcd for C₂₀H₂₀F₃N₅O₂S: C, 53.21; H, 4.47; F, 12.62; N, 15.51; S, 7.10.

### Example 88

### b 3-amino-4-[4-(2,3,5,6-tetrafluoropyridin-4-yl)-1,4-diazepan-1-yl]thieno[2,3-b]pyridine-2-carboxamide

(Exemplified Compound No. 3-145)

### (88a) tert-butyl 4-(2,3,5,6-tetrafluoropyridin-4-yl)-1,4-diazepane-1-carboxylate

Pentafluoropyridine (878 μL, 8 mmol), tert-butyl 1,4-diazepane-1-carboxylate (1,577 μL, 8 mmol) and triethylamine (1,227 μL, 8.8 mmol) were dissolved in methylene chloride (40 mL) and the mixture was stirred at room temperature for one hour. The reaction liquid was blended with a saturated sodium hydrogen carbonate aqueous solution (50 mL) and partitioned and the aqueous layer was extracted with methylene chloride (2×25 mL) and then the organic layer was combined and the solvent was evaporated under reduced pressure after drying over anhydrous sodium sulfate. Residual substance was purified by silica gel column chromatography (eluent: hexane/ethyl acetate =5/1) and 2.54 g (91%) of the title compound was obtained.

White powder

Mp 55-59° C.;

IR (KBr) νₘₐₓ 2985, 1690, 1636, 1523, 1476, 1170, 1143 cm⁻¹;

1H NMR (CDCl₃, 400 MHz) δ 1.44 (4.5H, s), 1.46 (4.5H, s), 1.90-2.03 (1H, m), 3,47 (1H, m, J=5.9 Hz, 3.53)1H, t, J=5.9 Hz), 3.71-3.56 (6H, m);

MS (FAB) m/z: 350 [M+H]⁺, 294, 250.

### (88b) 1-(2,3,5,6-tetrafluoropyridin-4-yl)-1,4-diazepane

tert-butyl 4-(2,3,5,6-tetrafluoropyridin-4-yl)-1,4-diazepane-1-carboxylate which was produced in Example 88 (88a) was used in place of tert-butyl 4-phenyl -1,4-diazepane-1-carboxylate and the reaction was performed in a similar method as described in Example 57 (57b) and the title compound was obtained.

White powder (yield 97%)

Mp 58-61° C.;

IR (KBr) νₘₐₓ 3356, 2932, 2852, 1638, 1534, 1473, 1126, 1068, 932 cm⁻¹;

¹H NMR (CDCl₃, 400 MHz) δ 1.92 (2H, quint, J=5.9 Hz), 2.96 (2H, t, J=5.9 Hz) 3.05 (2H, t, J=5.5 Hz), 3.62-3.71 (6H, m);

MS (EI) m/z: 249 [M+], 207, 193.

### (88c) 4-[4-(2,3,5,6-tetrafluoropyridin-4-yl]-1,4-diazepan-1-yl)-2-thioxa-1,2-dihydropyridine-3-carbonitrile

1-(2,3,5,6-tetrafluoropyridin-4-yl)-1,4-diazepane which was produced in Example 88 (88b) was used in place of 1-(4-methylphenyl)-1,4-diazepane and the reaction was performed in a similar method as described in Example 77 (77c) and the title compound was obtained.

Pale brown powder (Yield 22%)

Mp 267-269° C.;

IR (KBr) ₘₐₓ 3121, 2961, 2210, 1628, 1522, 1471, 1250, 1135, 962 cm⁻¹;

¹H NMR (DMSO-d₆, 400 MHz) δ 1.97-2.08 (2H, m), 3.58-3.66 (2H, m), 3.76-3.85 (2H, m), 3.89-3.96 (2H, m), 3.97-4.05 (2H, m), 6.50 (1H, d, J=7.6 Hz), 7.42 (1H, d, J=7.6 Hz), 12.51 (1H, bs);

MS (FAB) m/z: 384 [M+H]⁺, 200;

Anal. Calcd for C₁₆H₁₃F4N₅S: C, 50.13; H, 3.42; N, 18.27; S, 8.36. Found: C, 50.08; H, 3.66; N, 18.35; S, 8.37.

### (88d) 3-amino-4-[4-(2,3,5,6-tetrafluoropyridin-4-yl)-1,4-diazepan-1-yl]thieno[2,3-b]pyridine-2-carboxamide

4-[4-(2,3,5,6-tetrafluoropyridin-4-yl)-1,4-diazepan-1-yl]-2-thioxa-1,2-dihydropyridine -3-carbonitrile which was produced in Example 88 (88c) was used in place of 4-(isobutylamino)-2-thioxo-1,2-dihydropyridine-3-carbonitrile and the reaction was performed in a similar method as described in Example 5 (5c) and the title compound was obtained.

Slightly yellow powder (Yield 85%)

Mp 228-231° C.;

IR (KBr) 67 ₘₐₓ 3441, 3325, 3175, 2924, 1641, 1583, 1469, 1371, 1121, 962 cm⁻¹;

¹H NMR (DMSO-d₆, 500 MHz) δ 2.14-2.23 (2H, m), 3.26-3.35 (2H, m), 3.39-3.48 (2H, m), 3.75-3.82 (2H, m), 3.87-3.94 (2H, m), 7.04 (2H, bs), 7.10 (1H, d, J=5.4 Hz), 7.11 (2H, bs), 8.44 (1H, d, J=5.4 Hz);

MS (FAB) m/z: 441 [M+H]⁺, 329, 273;

Anal. Calcd for C₁₈H₁₆F4N₆OS: C, 49.09; H, 3.66; F, 17.25; N, 19.08; S, 7.28. Found: C, 49.02; H, 3.72; F, 16.85; N, 19.13; S, 7.21.

### Example 89

### 3-amino-4-(4-quinazolin-4-yl-1,4-diazepan-1-yl)thieno[2,3-b]pyridine -2-carboxamide

(Exemplified Compound No. 3-147)

### (89a) tert-butyl 4-quinazolin-4-yl-1,4-diazepane-1-carboxylate

4-chloroquinazoline (Helv. Chim. Acta, (2001), 84,1112-1118) was used in place of pentafluoropyridine and the reaction was performed in a similar method as described in Example 88 (88a) and the title compound was obtained.

Colourless oil (Yield 91%)

Mp 55-59° C.;

IR (film) νₘₐₓ 2975, 2929, 2869, 1693, 1566, 1506, 1346, 1167 cm⁻¹;

¹H NMR (CDCl₃, 400 MHz) δ 1.34 (4.5H, s), 1.42 (4.5H, s), 2.03-2.21 (2H, m), 3.47 (1H, t, J=5.9 Hz), 3.56 (1H, t, J=5.5 Hz), 3.65-3.77 (2H, m), 3.90-4.10 (4H, m), 7.41 (1H, t, J=8.2 Hz), 7.71 (1H, t, J=8.2 Hz), 7.86 (1H, d, J=8.2 Hz), 7.95 (1H, d, J=8.2 Hz), 8.64 (1H, s);

MS (FAB) m/z: 329 [M+H]⁺, 273.

### (89b) 4-(1,4-diazepan-1-yl) quinazoline

tert-butyl 4-quinazolin-4-yl-1,4-diazepane-1-carboxylate which was produced in Example 89 (89a) was used in place of tert-butyl 4-phenyl-1,4-diazepane-1-carboxylate and the reaction was performed in a similar method as described in Example 57 (57b) and the title compound was obtained.

Slightly brown oil (Yield 76%)

IR (film) νₘₐₓ 3296, 2937, 1684, 1613, 1567, 1505, 1346 cm⁻¹;

¹H NMR (CDCl₃, 400 MHz) δ 2.03 (2H, m), 2.96-3.02 (2H, m), 3.17-3.22 (2H, m), 3.98-4.04 (4H, m), 7.33-7.39 (1H, m), 7.64-7.70 (1H, m), 7.80-7.84 (1H, m), 7.92-7.97 (1H, m), 8.60 (1H,s);

MS (EI) m/z: 228 [M+], 185, 172, 159.

### (89c) 4-(4-quinazolin-4-yl-1,4-diazepan-1-yl)-2-thioxo-1,2-dihydropyridine-3-carbonitrile

4-(1,4-diazepan-1-yl)quinazoline produced in Example 89 (89b) was used in place of 1-(4-methylphenyl)-1,4-diazepane and the reaction was performed in a similar method as described in Example 77 (77c) and the title compound was obtained.

Brown powder (Yield 19%)

Mp 221-223° C.;

IR (KBr) ν^{max} 3431, 3111, 2926, 2204, 1623, 1566, 1504, 1344, 1242 cm⁻¹;

¹H NMR (DMSO-d₆, 400 MHz) δ 2.14-2.22 (2H, m), 3.93 (2H, t, J=5.9 Hz), 3.98 (2H, t, J=5.5 Hz), 4.13 (4H, bs), 6.47 (1H, d, J=7.4 Hz), 7.35 (1H, d, J=8.2 Hz), 7.45-7.52 (1H, m), 7.70-7.79 (2H, m), 8.02 (1H, d, J=8.2 Hz), 8.50 (1H, s), 12.49 (1H, bs);

MS (FAB) m/z: 363 [M+H]⁺, 257, 229;

Anal. Calcd for C₁₉H₁₈N₆S: C, 60.09; H, 5.29; N, 22.13. Found: C, 60.18; H, 5.38; N, 22.09.

### (89d) 3-amino-4-(4-quinazolin-4-yl-1,4-diazepan-1-yl)thieno[2,3-b]pyridine-2-carboxamide

4-(4-quinazolin-4-yl-1,4-diazepan-1-yl)-2-thioxo-1,2-dihydropyridine-3-carbonitrile which was produced in Example 89 (89c) was used in place of 4-(isobutylamino)-2-thioxo-1,2-dihydropyridine-3-carbonitrile and the reaction was performed in a similar method as described in Example 5 (5c) and the title compound was obtained.

Slightly yellow powder (Yield 85%)

Mp 228-231° C.;

IR (KBr) νₘₐₓ 3439, 3321, 3181, 2957, 1648, 1567, 1501, 1344 cm⁻¹;

¹H NMR (DMSO-d₆, 400 MHz) δ 2.24-2.36 (2H, m), 3.17-3.30 (2H, m), 3.50-3.59 (2H, m), 4.03-4.13 (2H, m), 4.18-4.29 (2H, m), 7.00 (2H, bs), 7.06 (1H, d, J=5.1 Hz), 7.10 (2H, bs), 7.46 (1H, ddd, J=8.6, 6.0, 2,3 Hz), 7.70-7.78 (2H, m), 8.10 (1H, d, J=8.2 Hz), 8.39 (1H, d, J=5.1 Hz), 8.50 (1H, s);

MS (FAB) m/z: 420 [M+H]⁺, 273, 246, 200.

### Example 90

### 3-amino-4-[4-(4-methyl-3-nitrophenyl)-1,4-diazepan-1-yl]thieno[2,3-b]pyridine-2-carboxamide

(Exemplified Compound No. 3-119)

### (90a) tert-butyl 4-(4-methyl-3-nitrophenyl)-1,4-diazepane-1-carboxylate

The reaction was performed in a similar method as described in Org. Lett., 4, 581-584 (2002) and the title compound was synthesized.

Orange powder (Yield 41%)

Mp 103-106° C.;

IR (KBr) vmax. 2978, 1685, 1528, 1415, 1334 cm-⁻¹;

¹H NMR (CDCl₃, 500 MHz) δ 1.35 (4.5H, s), 1.42 (4.5H, s), 1.93-2.00 (2H, m), 2.45 (3H, s), 3.23 (1H, t, J=5.9 Hz), 3.33 (1H, t, J=5.9 Hz), 3.53-3.62 (6H, m), 6.82 (1H, dd, J=8.8, 2.9 Hz, 7.13 (1H, d, J=8.8 Hz, 7.26 (1H, d, J=2.9 Hz);

MS (FAB) m/z: 336 [M+H]⁺, 280, 234, 189;

Anal. Calcd for C₁₇H₂₅N₃O₄.0.3H₂O: C, 59.91; H, 7.57; N, 12.33. Found: C, 59.94; H, 7.29; N, 12.01.

### (90b) 1-(4-methyl-3-nitrophenyl)-1,4-diazepane

tert-butyl 4-(4-methyl-3-nitrophenyl)-1,4-diazepane-1-carboxylate which was produced in Example 90 (90a) was used in place of tert-butyl 4-phenyl-1,4-diazepane-1-carboxylate and the reaction was performed in a similar method as described in Example 57 (57b) and the title compound was obtained.

Red oil (Yield 100%)

IR (liquid) νₘₐₓ 2930, 1628, 1527, 1346 cm-⁻¹;

1H NMR (CDCl₃, 500 MHz) δ 1.91 (2H, t, J=5.9 Hz), 2.45 (3H, s), 2.83 (2H, t, J=5.9 Hz), 3.04 (2H, t, J=5.5 Hz), 3.55 (2H, t, J=5.5 Hz), 3.59 (2H, t, J=5.9 Hz), 6.81 (1H, dd, J=8.3, 2.9 Hz), 7.12 (1H, d, J=8.3 Hz), 7.26 (1H, d, J=2.9 Hz);

MS (EI) m/z: 235 [M⁺], 193, 179.

### (90c) 4-[4-(4-methyl-3-nitrophenyl)-1,4-diazepan-1-yl]-2-thioxo-1,2-dihydropyridine-3-carbonitrile

b 1-(4-methyl-3-nitrophenyl)-1,4-diazepane which was produced in Example 90 (90b) was used in place of 1-(4-methylphenyl)-1,4-diazepane and the reaction was performed in a similar method as described in Example 77 (77c) and the title compound was obtained.

Brown powder (Yield 31%)

Mp 277-278° C.;

IR (KBr) νₘₐₓ 3437, 3124, 2961, 2206, 1625, 1525, 1341, 1252 cm⁻¹;

¹H NMR (DMSO-d₆, 400 MHz) δ 1.94 (2H, m), 2.32 (3H, s), 3.57 (2H, t, J=5.9 Hz), 3.70-3.81 (4H, m), 3.92-3.99 (2H, m), 6.42 (1H, d, J=7.8 Hz), 7.05 (1H, dd, J=8.6, 2.7 Hz), 7.22 (1H, d, J=8.6 Hz), 7.24 (1H, d, J=2.7 Hz), 7.35 (1H, d, J=7.8 Hz), 12.47 (1H, bs);

MS (FAB) m/z: 370 [M+H]⁺, 259, 242;

Anal. Calcd for C₁₈H₁₉N₅O₂S.0.26H₂O: C, 57.79; H, 5.26; N, 18.72; S, 8.57. Found: C, 57.78; H, 5.16; N, 18.73; S, 8.51.

### (90d) 3-amino-4-[4-(4-methyl-3-nitrophenyl)-1,4-diazepan-1-yl]thieno[2,3-b]pyridine-2-carboxamide

4-[4-(4-methyl-3-nitrophenyl)-1,4-diazepan-1-yl]-2-thioxo-1,2-dihydropyridine-3-carbonitrile which was produced in Example 90 (90c) was used in place of 4-(isobutylamino)-2-thioxo-1,2-dihydropyridine-3-carbonitrile and the reaction was performed in a similar method as described in Example 5 (5c) and the title compound was obtained.

Orange powder (Yield 91%)

Mp 114-117° C.;

IR (KBr) νₘₐₓ 3439, 3323, 3181, 2927, 2846, 1650, 1579, 1525, 1366 cm⁻¹;

¹H NMR (DMSO-d₆, 500 MHz) 8 2.13-2.21 (2H, m), 2.36 (3H,s), 3.14-3.23 (2H, m), 3.27-3.34 (2H, m), 3.59 (2H, t, J=5.9 Hz), 3.81 (2H, t, J=4.8 Hz), 7.01 (2H, bs), 7.0-7.15 84H, m), 7.24-7.29 (2H, m), 8.41 (1H, d, J=5.4 Hz);

MS (FAB) m/z: 427 [M+H]⁺, 410, 273, 246;

Anal. Calcd for C₂₀H₂₂N₆O₃S.0.5H₂O: C, 55.16; H, 5.32; N, 19.30. Found: C, 54.87; H, 5.49; N, 19.58.

### Example 91

### 3-amino-4-[4-(4-isopropoxyphenyl)-1,4-diazepan-1-yl]thieno[2,3-b]pyridine-2-carboxamide

(Exemplified Compound No. 3-129)

### (91a) tert-butyl 4-(4-isopropoxyphenyl)-1,4-diazepane-1-carboxylate

The reaction was performed in a similar method as described in Org. Lett., 4, 581-584 (2002) and the title compound was synthesized.

Colourless oil (Yield 14%)

IR (film) νₘₐₓ 2974, 1694, 1511, 1415, 1366, 1237, 1168, 1122 cm⁻¹;

¹H NMR (CDCl₃, 400 MHz) δ 1.28 (6H, d, J=5.9 Hz), 1.37 (4.5H, s), 1.43 (4.5H, s), 1.90-2.00 (2H, m), 3.20 (2H, t, J=5.9 Hz), 3.31 (2H, t, J=5.5 Hz), 3.40-3.61 (6H, m), 4.35 (1H, sept, J=5.9 Hz), 6.61 (2H, d, J=9.0 Hz), 6.78 (2H, d, J=9.0 Hz);

MS (EI) m/z: 334 [M⁺], 278, 236.

### (91b) 1-(4-isopropoxyphenyl)-1,4-diazepane

tert-butyl 4-(4-isopropoxyphenyl)-1,4-diazepane-1-carboxylate which was produced in Example 91 (91a) was used in place of tert-butyl 4-phenyl-1,4-diazepane-1-carboxylate and the reaction was performed in a similar method as described in Example 57 (57b) and the title compound was obtained.

Slightly orange prism crystal (Yield 89%)

Mp 62-64° C.;

IR (film) νₘₐₓ 2974, 2932, 1511, 1237, 1114, 957, 815 cm⁻¹;

¹H NMR (CDCl₃, 400 MHz) δ 1.29 (6H, d, J=5.9 Hz), 1.88 (2H, quint, J=5.9 Hz), 2.83 (2H, t, J=5.9 Hz), 3.01 (2H, t, J=5.5 Hz), 3.49 (2H, t, J=5.5 Hz), 3.52 (2H, t, J=5.9 Hz), 4.35 (1H, sept, J=5.9 Hz), 6.61 (2H, d, J=9.0 Hz), 6.79 (2H, d, J=9.0 Hz);

MS (EI) m/z: 234 [M⁺], 191, 178.

### (91c) 4-[4-(4-isopropoxyphenyl)-1,4-diazepan-1-yl]-2-thioxo-1,2-dihydropyridine-3-carbonitrile

1-(4-isopropoxyphenyl)-1,4-diazepane which was produced in Example 91 (91b) was used in place of 1-(4-methylphenyl)-1,4-diazepane and the reaction was performed in a similar method as described in Example 77 (77c) and the title compound was obtained.

Slightly brown powder (Yield 32%)

Mp 216-218° C.;

IR (KBr) νₘₐₓ 3440, 3128, 3046, 2974, 2205, 1625, 1511, 1511, 1241 cm⁻¹;

¹H NMR (CDCl₃, 500 MHz) δ 1.30 (6H, d, J=5.9 Hz), 2.11 (2H, quint, J=5.9 Hz), 3.51 (2H, t, J=5.9 Hz), 3.73 (2H, t, J=5.4 Hz), 3.76 (2H, t, J=5.9 Hz), 4.12 (2H, t, J=5.4 Hz), 4.39 (1H, sept, J=5.9 Hz), 6.21 (1H, d, J=7.8 Hz), 6.67 (2H, d, T=8.8 Hz), 6.82 (2H, d, J=8.8 Hz), 7.23 (1H, d, J=7.8 Hz), 1184 (1H, bs);

MS (EI) m/z: 368 [M+], 325, 148;

Anal. Calcd for C₂₀H₂4N₄OS.0.15H₂O: C, 64.71; H, 6.60; N, 15.09; S, 8.64. Found: C, 64.59; H, 6.46; N, 15.09; S, 8.47.

### (91d) 3-amino-4-[4-(4-isopropoxyphenyl)-1,4-diazepan-1-yl]thieno[2,3-b]pyridine-2-carboxamide

4-[4-(4-isopropoxyphenyl)-1,4-diazepan-1-yl]-2-thioxo-1,2-dihydropyridine -3-carbonitrile which was produced in Example 91 (91c) was used in place of 4-(isobutylamino)-2-thioxo-1,2-dihydropyridine-3-carbonitrile and the reaction was performed in a similar method as described in Example 5 (5c) and the title compound was obtained.

Slightly yellow powder 79%

Mp 173-175° C.;

IR (KBr) νₘₐₓ 3441, 3324, 2973, 1644, 1579, 1510, 1370, 1235 cm⁻¹;

¹ H NMR (DMSO-d₆, 400 MHz) δ 1.21 (6H, d, J=5.9 Hz), 2.07-2.16 (2H, m), 3.16-3.24 (2H, m), 3.25-3.33 (2H, m), 3.47 (2H, t, J=5.9 Hz), 3.69 (2H, t, J=4.7 Hz), 4.37 (1H, sept, J=5.9 Hz), 6.68 (2H, d, J=9.0 Hz), 6.76 (2H, d, J=9.0 Hz), 6.97 (2H, bs), 7.03-7.11 (3H, m), 8.37 (1H, d, J=5.5 Hz);

MS (EI) m/z: 425 [M+], 382, 275, 183;

Anal. Calcd for C₂₂H₂₇N₅ O₂S: C, 62.09; H, 6.40; N, 16.46; S, 7.54. Found: C, 61.83; H, 6.23; N, 16.32; S, 7.38.

### Example 92

### 3-amino-4-[4-(4-tert-butylphenyl)-1,4-diazepan-1-yl]thieno[2,3-b]pyridine-2-carboxamide

(Exemplified Compound No. 3-115)

### (92a) tert-butyl 4-(4-tert-butylphenyl)-1,4-diazepane-1-carboxylate

The reaction was performed in a similar method as described in Org. Lett., 4,581-584(2002) and the title compound was synthesized.

White powder (Yield 9%)

Mp 62-64° C.;

IR (KBr) νₘₐₓ 2962, 1686, 1520, 1420, 1364, 1246, 1170 cm⁻¹;

¹H NMR (CDCl₃, 400 MHz) δ 1.27 (9H, s), 1.35 (4.5H, s), 1.43 (4.5H, s), 1.91-2.00 (1H, m), 3.21 (1H, t, J=6.3 Hz), 3.32 (1H, t, J=5.9 Hz), 3.48-3.59 (6H, m), 6.62 (2H, d, J=8.8 Hz), 7.20 (2H, d, J=8.8 Hz);

MS (EI) m/z: 332 [M⁺], 276, 261.

### (92b) 1-(4-tert-butylphenyl)-1,4-diazepane

tert-butyl 4-(4-tert-butylphenyl)-1,4-diazepane-1-carboxylate which was produced in Example 92 (92a) was used in place of tert-butyl 4-phenyl-1,4-diazepane-1-carboxylate and the reaction was performed in a similar method as described in Example 57 (57b) and the title compound was obtained.

Pale brown oil (Yield 97%)

IR (film) νₘₐₓ 2959, 1614, 1520, 1363, 1201, 812, 552 cm⁻¹;

¹H NMR (CDCl₃, 400 MHz) δ 1.28 (9H, s), 1.89 (2H, quint, J=5.9 Hz), 2.84 (2H, t, J=5.9 Hz), 3.02 (2H, t, J=5.5 Hz), 3.52 (2H, t, J=5.5 Hz), 3.54 (2H, t, J=5.9 Hz), 6.63 (2H, d, J=8.4 Hz), 7.21 (2H, d, J=8.4 Hz);

MS (EI) m/z: 232 [M+], 217, 190, 176.

### (92c) 4-[4-(4-tert-butylphenyl)-1,4-diazepan-1-yl]-2-thioxo-1,2-dihydropyridine-3-carbonitrile

1-(4-tert-butylphenyl)-1,4-diazepane which was produced in Example 92 (92b) was used in place of 1-(4-methylphenyl)-1,4-diazepane and the reaction was performed in a similar method as described in Example 77 (77c) and the title compound was obtained.

Pale brown powder (Yield 21%)

Mp 257-258° C.;

IR (KBr) νₘₐₓ 3121, 3041, 2958, 2205, 1625, 1519, 1459, 1247 cm⁻¹;

¹H NMR (CDCl₃, 400 MHz) δ 1.28 (9H, s), 2.12 (2H, quint, J=5.9 Hz), 3.56 (2H, t, J=5.9 Hz), 3.71-3.81 (4H, m), 4.13 (2H, t, J=5.1 Hz), 6.20 (1H, d, J=7.6 Hz), 6.66 (2H, d, J=8.6 Hz), 7.22 (1H, d, J=7.6 Hz), 7.24 (2H, d, J=8.6 Hz), 11.94 (1H, bs);

MS (EI) m/z: 366 [M+], 351, 188.

### (92d) 3-amino-4-[4-(4-tert-butylphenyl)-1,4-diazepan-1-yl]thieno[2,3-b]pyridine-2-carboxamide

4-[4-(4-tert-butylphenyl)-1,4-diazepan-1-yl]-2-thioxo-1,2-dihydropyridine-3-carbonitrile which was produced in Example 92 (92c) was used in place of 4-(isobutylamino)-2-thioxo-1,2-dihydropyridine-3-carbonitrile and the reaction was performed in a similar method as described in Example 5 (5c) and the title compound was obtained.

Slightly yellow powder (Yield 72%)

Mp 231-232° C.;

IR (KBr) νₘₐₓ 3440, 3324, 3182, 2957, 1645, 1579, 1579, 1519, 1364 cm⁻¹;

¹H NMR (DMSO-d₆, 400 MHz) δ 1.21 (9H, s), 2.06-2.17 (2H, m), 3.22-3.16 (4H, m), 6.69 (2H, d, J=9.0), 6.89 (2H, bs), 7.03-7.09 (3H, m), 7.17 (2H, d, J=9.0 Hz), 8.38 (1H, d, J=5.5 Hz);

MS (EI) m/z: 423 [M+], 391, 275;

Anal. Calcd for C₂₃H₂₉N₅OS.0.27H₂0: C, 64.48; H, 6.95; N, 16.35; S, 7.48. Found: C, 64.17; H, 6.64; N, 16.31; S, 7.43.

### Example 93

### 3-amino-4-[4-(4-ethylphenyl)-1,4-diazepan-1-yl]thieno[2,3-b]pyridine-2-carboxamide

(Exemplified Compound No. 3-112)

(93a) tert-butyl 4-(4-ethylphenyl)-1,4-diazepane-1-carboxylate

The reaction was performed in a similar method as described in Org. Lett., 4,581-584(2002) and the title compound was synthesized.

Colourless oil (Yield 11%)

IR (film) νₘₐₓ 2964, 1695, 1519, 1415, 1236, 1169 cm⁻¹;

¹H NMR (CDCl₃, 400 MHz) δ 1.19 (3H, t, J=7.6 Hz), 1.37 (4.5H, s), 1.44 (4.5H, s), 1.92-2.01 (2H, m), 2.53 (2H, q, J=7.6 Hz), 3.19 (1H, t, J=6.3 Hz), 3.30 (1H, t, J=5.9 Hz), 3.47-3.59 (6H, m), 6.62 (2H, d, J=8.4 Hz), 7.02 (2H, d, J=8.4 Hz

MS (FAB) m/z: 304 [M⁺], 248, 160.

### (93b) 1-(4-ethylphenyl)-1,4-diazepane

tert-butyl 4-(4-ethylphenyl)-1,4-diazepane-1-carboxylate which was produced in Example 93 (93a) was used in place of tert-butyl 4-phenyl-1,4-diazepane-1-carboxylate and the reaction was performed in a similar method as described in Example 57 (57b) and the title compound was obtained.

Pale brown oil (Yield 100%)

IR (film) νₘₐₓ 2929, 1616, 1519, 1189, 813 cm⁻¹;

¹H NMR (CDCl₃, 400 MHz) δ 1.19 (3H, t, J=7.6 Hz), 1.88 (2H, quint, J=5.9 Hz), 2.54 (2H, q, J=7.6 Hz), 2.82 (2H, t, J=5.9 Hz), 3.01 (2H, t, J -5.5 Hz), 3.49-3.57 (2H, m), 6.62 (2H, d, J=8.8 Hz), 7.03 (2H, d, J=8.8 Hz);

MS (EI) m/z: 204 [M+], 162, 148.

### (93c) 4-[4-(4-ethylphenyl)-1,4-diazepan-1-yl]-2-thioxo-1,2-dihydropyridine-3-carbonitrile

1-(4-ethylphenyl)-1,4-diazepane which was produced in Example 93 (93b) was used in place of 1-(4-methylphenyl)-1,4-diazepane and the reaction was performed in a similar method as described in Example 77 (77c) and the title compound was obtained.

Slightly brown powder (Yield 22%)

Mp 226-229° C.;

IR (KBr) νₘₐₓ 3122, 2959, 2205, 1625, 1517, 1457, 1246 cm⁻¹;

¹H NMR(CDCl₃, 400 MHz) δ 1.20 (2H, t, J=7.6 Hz), 2.11 (2H, quint, J=5.9 Hz), 2.55 (2H, q, J=7.6 Hz), 3.55 (2H, t, J=5.9 Hz), 3.74 (2H, t, J=5.9 Hz), 3.77 (2H, t J=5.1 Hz), 4.12 (2H, t, J=5.1 Hz), 6.19 (1H, d, J=7.6 Hz), 6.65 (2H, d, J=8.6 Hz), 7.06 (2H, d, J=8.6 Hz), 7.20 (1H, d, J=7.6 Hz), 11.58 (1H, bs);

MS (EI) m/z: 338 [M⁺],323, 174, 160.

### (93d) 3-amino-4-[4-(4-ethylphenyl)-1,4-diazepan-1-yl]thieno[2,3-b]pyridine-2-carboxamide

4-[4-(4-ethylphenyl)-1,4-diazepan-1-yl]-2-thioxo-1,2-dihydropyridine-3-carbonitrile which was produced in Example 93 (93c) was used in place of 4-(isobutylamino)-2-thioxo-1,2-dihydropyridine-3-carbonitrile, and the reaction was performed in a similar method as described in Example 5 (5c) and the title compound was obtained.

White powder (Yield 82%)

Mp 186-188° C.;

IR (KBr) νₘₐₓ 3439, 3324, 3178, 2958, 1643, 1579, 1518, 1369 cm⁻¹;

¹H NMR(DMSO-d₆, 400 MHz) δ 1.14 (3H, t, J=7.4 Hz), 2.07-2.17 (2H, m), 2.49 (2H, q, J=7.4 Hz), 3.15-3.35 (4H, m), 3.53 (2H, t, J=5.9 Hz), 3.74 (2H, t, J=4.7 Hz), 6.70 (2H, d, J=8.4 Hz), 6.96 (1H, bs), 7.02 (2H, d, J=8.4 Hz), 7.04-7.14 (3H, m), 8.40 (1H, d, J=5.5 Hz);

MS (EI) m/z: 395 [M⁺],377, 189;

Anal. Calcd for C₂₁H₂₅N₅OS: C, 63.77; H, 6.37; N, 17.71; S, 8.11. Found: C, 63.56; H, 6.25; N, 17.68; S, 8.10.

### Example 94

### 4-[4-(4-acetylphenyl)-1,4-diazepan-1-yl]-3-aminothieno [2,3-b]pyridine-2-carboxamide

(Exemplified Compound No. 3-99)

### (94a) tert-butyl 4-(4-acetylphenyl)-1,4-diazepane-1-carboxylate

4-fluoroacetophenone was used in place of 4-fluoronitrobenzene and the reaction was performed in a similar method as described in Example 59 (59a) and the title compound was obtained.

Yellow liquid

IR (film) νₘₐₓ 2974, 1596, 1523, 1416, 1363, 1284, 1237, 1191, 929, 819, 756 cm⁻¹;

¹H NMR(CDCl₃, 400 MHz) δ 1.36 (4.5H, s), 1.42 (4.5H, s), 1.94-2.01 (2H, m), 2.50 (1H, s), 3.21 (1H, t, J=5.9 Hz), 3.32 (1H, t, J=5.9 Hz), 3.58-3.64 (6H, m), 6.66 (2H, d, J=8.6 Hz), 7.83 (2H, t, J=8.6 Hz);

HRMS m/z calcd for C₁₈H₂₆O₃N₂ 318.1944, found 318.1934;

MS (EI) m/z: 318 [M⁺],261, 247, 217, 188, 174, 162, 132, 105, 91, 57, 41.

### (94b) 1-[4-(1,4-diazepan-1-yl)phenyl]ethanone

tert-butyl 4-(4-acetylphenyl)-1,4-diazepane-1-carboxylate which was produced in Example 94 (94a) was used in place of tert-butyl 4-phenyl-1,4-diazepane-1-carboxylate and the reaction was performed in a similar method as described in Example 57 (57b) and the title compound was obtained.

Brown liquid

IR (KBr) νₘₐₓ 2931, 1658, 1597, 1523, 1403, 1360, 1285, 1191, 820 cm⁻¹;

¹H NMR(CDCl₃, 500 MHz) δ 1.91 (2H, quint, J=5.9 Hz), 2.50 (3H, s), 2.83 (2H, t, J=5.9 Hz), 3.04 (2H, t, J=5.4 Hz), 3.61 (2H, t, J=5.4 Hz), 3.66 (2H, t, J=6.4 Hz), 6.68 (2H, d, J=8.8 Hz), 7.85 (2H, d, J=8.8 Hz);

HRMS m/z calcd for C₁₃H₁₈ON₂ 218.1420.1313, found 218.1422;

MS (EI) m/z: 218 [M^{+],} 203, 176, 162, 148, 132, 70, 43.

### (94c) (2Z)-3-[4-(4-acetylphenyl)-1,4-diazepan-1-yl]-2-cyanobut-2-enethioamide

1-[4-(1,4-diazepan-1-yl)phenyl]ethanone which was produced in Example 94 (94b) was used in place of isobutylamine and the reaction was performed in a similar method as described in Example 5 (5a) and the title compound was obtained.

Yellow powder

Mp 173-177° C.;

IR (KBr) νₘₐₓ 3310, 3178, 2183, 1593, 1524, 1407, 1354, 1289, 1194, 825, 594 cm⁻¹;

¹H NMR(DMSO-d₆, 400 MHz) δ 1.89-1.96 (2H, m), 2.23 (3H, s), 2.42 (3H, s), 3.49-3.54 (2H, m), 3.60-3.66 (4H, m), 3.83-3.86 (2H, m), 6.81 (2H, d, J=9.0 Hz), 7.79 (2H, d, J=9.0 Hz), 7.16 (2H, t, J=7.3 Hz), 8.39 (1H, brs), 9.04 (1H, brs);

HRMS m/z calcd for C₁₈H₂₃ON₄S 343.1592, found 343.1595;

MS (FAB) m/z: 343 [M+H]⁺, 326, 273, 246, 219, 165, 120, 65;

Anal. Calcd for C₁₈H₂₂N₄OS.0.40H₂O: C, 61.83; H, 6.57; N, 16.02; S, 9.17. Found: C, 61.52; H, 6.35; N, 15.90, S, 9.52.

### (94d) 4-[(4-acetylphenyl)-1,4-diazepan-1-yl]-2-thioxo-1,2-dihydropyridine-3-carbonitrile

(2Z)-3-[4-(4-acetylphenyl)- 1,4-diazepan-1-yl]-2-cyanobut-2-enethioamide which was produced in Example 94 (94c) (504 mg, 1.47 mmol) and N,N-dimethylformamide dimethylacetal (175 mg, 1.47 mmol) were dissolved in N,N-dimethylformamide (2 mL) and the mixture was stirred at 60° C. for 80 minutes. After 1N hydrochloric acid was added to make the reaction solution slightly acidic, ethyl acetate (10 mL) and water (20 mL) were added. The deposited solid was separated by filtration and the title compound was obtained (518 mg, 38%).

Pale brown powder

Mp 265-270° C.;

IR (KBr) νₘₐₓ 2940, 2206, 1625, 1593, 1521, 1355, 1288, 1248, 1238, 1192, 1143, 929, 817 cm⁻¹;

¹H NMR(DMSO-d₆, 500 MHz) δ 1.91-1.97 (2H, m), 2.42 (3H, s), 3.64 (2H, t, J=5.9 Hz), 3.76 (2H, t, J=5.9 Hz), 3.86 (2H, t, J=5.4 Hz), 3.98 (2H, t, J=5.4 Hz), 6.44 (1H, d, J=7.8 Hz), 6.85 (1H, d, J=8.8 Hz), 7.87 (1H, t, J=7.8 Hz), 7.77 (2H, t, J=8.8 Hz), 12.57 (1H, brs);

HRMS m/z calcd for C₁₉H₂₁ON₄S 353.1436, found 353.1425;

MS (EI) m/z: 352 [M⁺], 337, 323, 218, 204, 174, 162, 132, 105, 91, 77, 43;

Anal. Calcd for C₁₉H₂₀N₄OS.0.33H₂O: C, 63.66; H, 5.81; N, 15.63; S, 8.95. Found: C, 63.58; H, 5.80; N, 15.63, S, 8.74.

### (94e) 4-[4-(4-acetylphenyl)-1,4-diazepan-1-yl]-3-aminothieno[2,3-b]pyridine-2-carboxamide

4-[(4-acetylphenyl)-1,4-diazepan-1-yl]-2-thioxo-1,2-dihydropyridine-3-carbonitrile which was produced in Example 94 (94d) was used in place of 4-(isobutylamino)-2-thioxo-1,2-dihydropyridine-3-carbonitrile and the reaction was performed in a similar method as described in Example 5 (5c), and the title compound was obtained.

Pale brown powder

Mp 279-282° C. (decomposition);

IR (KBr) νₘₐₓ 3439, 3326, 3183, 1653, 1595, 1364, 1279, 1193, 939, 820 cm⁻¹;

¹H NMR(DMSO-d₆, 500 MHz) δ 2.16-2.18 (2H, m), 2.44 (3H, s), 3.19-3.20 (2H, m), 3.30-3.32 (2H, m), 3.66 (2H, t, J=6.3 Hz), 3.87 (2H, t, J=4.9 Hz), 6.84 (2H, d, J=8.8 Hz), 6.99 (2H, brs), 7.08 (1H, d, J=5.4 Hz), 8.10 (2H, brs), 7.80 (2H, d, J=8.8 Hz), 8.41 (1H, d, J=5.4 Hz);

MS (EI) m/z: 409 [M⁺].

### Example 95

### 3-amino-4-[3-methyl-4-(3-methylphenyl)piperazin-1-yl]thieno[2,3-b]pyridine-2-carboxamide

(Exemplified Compound No. 3-77)

### (95a) 4-[3-methyl-4-(3-methylphenyl)piperazin-1-yl]-2-thioxo-1,2-dihydropyridine -3-carbonitrile

2-methyl-1-(3-methylphenyl) piperazine was used in place of 1-(4-methylphenyl)-1,4-diazepane and the reaction was performed in a similar method as described in Example 77 (77c) and the title compound was obtained.

Pale brown powder (yield 23%)

Mp 250-251° C.;

IR (KBr) νₘₐₓ 3122, 3039, 2970, 2208, 1621, 1496, 1447, 1248, 1011 cm⁻¹;

¹H NMR(DMSO-d₆, 400 MHz) δ 0.99 (3H, d, J=6.3 Hz), 2.25 (3H, s), 3.14-3.23 (1H, m), 3.37-3.52 (2H, m), 3.67 (1H, dd, J=13.3, 3.5 Hz), 3.91-3.99 (1H, m), 4.01-4.15 82H, m), 6.53 (2H, d, J=7.6 Hz), 6.58 (1H, d, J=7.4 Hz), 6.65-6.71 (2H, m), 7.09 (1H, t, J=7.4 Hz), 7.49 (1H, d, J=7.6 Hz);

MS (FAB) m/z: 325 [M+H]⁺, 200;

Anal. Calcd for C₁₈H₂₀N₄S.0.32H₂O: C, 65.47; H, 6.30; N, 16.97; S, 9.71. Found: C, 65.24; H, 6.38; N, 17.19; S, 9.78.

### (95b) 3-amino-4-[3-methyl-4-(3-methylphenyl)piperazin-1-yl]thieno[2,3-b]pyridine -2-carboxamide

4-[3-methyl-4-(3-methylphenyl)piperazin-1-yl]-2-thioxo-1,2-dihydropyridine -3-carbonitrile which was produced in Example 95 (95a) was used in place of 4-(isobutylamino)-2-thioxo-1,2-dihydropyridine-3-carbonitrile and the reaction was performed in a similar method as described in Example 5 (5c) and the title compound was obtained.

Slightly Brown powder (yield 81%)

Mp 215-217° C.;

IR (KBr) νₘₐₓ 3449, 3325, 3179, 2972, 2836, 1644, 1580, 1501, 1370 cm⁻¹;

¹H NMR(DMSO-d₆, 400 MHz) δ 0.99-1.14 (3H, m), 2.27 (3H, s), 2.62-3.51 (7H, m), 6.58-6.69 (1H, m), 6.74-6.85 (2H, m), 6.96 (2H, bs), 7.06-7.19 (4H, m), 8.46 (1H,d, J=5.5 Hz);

MS (EI) m/z: 382 [M+H]⁺, 200;

Anal. Calcd for C₂₀H₂₃N₅OS.0.16: C, 62.50; H, 6.12; N, 18.22; S, 8.34. Found: C, 62.49; H, 6.19; N, 18.15; S, 8.16.

### Example 9

### 3-amino-4-(4-pyridin-3-yl-1,4-diazepan-1-yl)thieno[2,3-b]pyridine-2-carboxamide

(Exemplified Compound No. 3-140)

### (96a) (2Z)-2-cyano-3-(4-pyridin-3-yl-1,4-diazepan-1-yl)but-2-enethioamide

1-pyridin-3-yl-1,4-diazepane (J. Med. Chem., (2000), 43, 2217-2226) was used in place of isobutylamine and the reaction was performed in a similar method as described in Example 5 (5a) and the title compound was obtained. Yield 83%.

Mp 162-164° C.;

IR (KBr) νₘₐₓ 3300, 3164, 2187, 1583, 1530, 796, 707 cm⁻¹;

¹H NMR (DMSO-d₆, 400 MHz) δ 1.91-1.97 (2H, m), 2.24 (3H, s), 3.51-3.63 (6H, m), 3.76-3.79 (2H, m), 7.11-7.15 (2H, m), 7.82-7.84 (1H, m), 8.14 (1H, brs), 8.35 (1H, br), 9.00 (1H, br);

MS (FAB) m/z: 302 [M+H]⁺;

Anal. Calcd for C₁₅H₁₉N₅S.0.1 H₂O: C, 59.42; H, 6.38; N, 23.10; S, 10.57. Found: C, 59.53; H, 6.48; N, 22.88; S, 10.57.

### (96b) 3-amino-4-(4-pyridin-3-yl-1,4-diazepan-1-yl)thieno[2,3-b]pyridine-2-carboxamide

(2Z)-2-cyano-3-(4-pyridin-3-yl-1,4-diazepan-1-yl)but-2-enethioamide which was produced in Example 96 (96a) (485 mg, 1.61 mmol) and N,N-dimethylformamide dimethylacetal (211 mg, 1.77 mmol) were dissolved in N,N-dimethylformamide (3 mL) and the mixture was stirred at room temperature for one hour. Furthermore, it was stirred from 100° C. to 120° C. for 1.5 hours. The reaction solution was cooled to room temperature and 8N aqueous solution of sodium hydroxide (0.3 mL) and 2-chloroacetamide (196 mg, 2.09 mmol) were added. Water (3 mL) was added after the mixture was stirred at room temperature for three hours. The deposited solid was separated by filtration and washed with water and ethanol and 239 mg of a solid was obtained. The obtained solid was recrystallized from ethanol (2 mL) and 195 mg of the title compound was obtained (yield 33%).

Mp 214-216° C.;

IR (KBr) νₘₐₓ 3444, 3324, 3166, 1650, 1579, 1495, 1368, 794, 709 cm⁻¹;

¹H NMR (DMSO-d₆, 400 MHz) δ 2.14-2.19 (2H, m), 3.16-3.22 (2H, m), 3.27-3.81 (2H, m), 3.55-3.58 (2H, m), 3.79-3.81 (2H, m), 6.97 (2H, brs), 7.07 (1H, d, J=5.5 Hz), 7.09 (2H, brs), 7.10-7.16 (2H, m), 7.83 (1H, dd, J=2.0, 4.3 Hz), 8.16 (1H, d, J =2.0 Hz), 8.35 (1H, d, J=5.5 Hz);

MS (FAB) m/z: 369 [M+H]⁺.

### Example 97

### 3-amino-4-[4-(1,3-thiazol-2-yl)-1,4-diazepan-1-yl]thieno[2,3-b]pyridine-2-carboxamide

(Exemplified Compound No. 3-136)

### (97a) 1-(1,3-thiazol-2-yl)-1,4-diazepane dihydrochloride

2-bromothiazole (8.20 g, 50 mmol) and homopiperazine (10.00 g, 100 mmol) were stirred in n-butanol (100 mL) under heat reflux for 24 hours. The reaction mixture was allowed to stand at room temperature for 24 hours and the deposited solid was separated by filtration. 1N aqueous solution of sodium hydroxide (30 mL) was added to the residue which was obtained by concentrating the filtrate under reduced pressure and the aqueous layer was extracted with methylene chloride (3×100 mL). The extract was concentrated under reduced pressure after drying over sodium sulfate. The residue was dissolved in methanol (30 mL) and 4N hydrochloric acid-1,4-dioxane solution (30 mL) was added. The deposited hydrochloride was separated by filtration and washed with 1,4-dioxane and acetone and 9.43 g (yield 74%) of the title compound was obtained.

IR (neat) νₘₐₓ 1603, 1581, 743 cm⁻¹;

¹H NMR (CDCl₃, 400MHz) δ 2.13-2.19 (2H, m), 3.18-3.25 (2H, m), 3.30-3.36 (2H, m), 3.64-3.67 (2H, m), 3.96-3.99 (2H, m), 6.98 (1H, d, J=3.9 Hz), 7.35 (1H, d, J=3.9 Hz);

MS (EI) m/z: 183 [M⁺], 83;

Anal. Calcd for C₈H₁₃N₃S.2(HCl).0.1(H₂O): C, 37.24; H, 5.94; N, 16.29; S, 12.43; Cl, 27.48. Found: C, 37.33; H, 5.88; N, 16.24; S, 12.37; Cl, 27.27.

### (97b) 1-(1,3-thiazol-2-yl)-1,4-diazepane

After 1-(1,3-thiazol-2-yl)-1,4-diazepane dihydrochloride (5.11 g, 19.9 mmol) which was produced in Example 97 (97a) was mixed with 1N aqueous solution of sodium hydroxide (60 mL), the mixture was extracted with methylene chloride (3×50 mL). The extract was dried over sodium sulfate and concentrated under reduced pressure and 3.60 g (yield 99%) of the title compound was obtained.

IR (neat) νₘₐₓ 3304, 1534, 1139, 614 cm⁻¹;

¹H NMR (CDCl₃, 400 MHz) δ 1.90-1.96 (2H, m), 2.89-2.91 (2H, m), 3.04-3.07 (2H m), 3.66-3.70 (4H, m), 6.44 (1H, d, J=3.5 Hz), 7.15 (1H, d, J=3.5 Hz);

MS (EI) m/z: 183 [M⁺], 83;

Anal. Calcd for C₈H₁₃N₃S: C, 52.43; H, 7.15; N, 22.93; S, 17.50. Found: C, 52.15; H, 7.46; N, 22.66; S, 10.50.

### (97c) (2Z)-2-cyano-3-[4-(1,3-thiazol-2-yl)-1,4-diazepan-1-yl]but-2-enethioamide

1-(1,3-thiazol-2-yl)-1,4-diazepane (3.60 g) which was produced in Example 97 (97b) was used in place of isobutylamine and the reaction was performed in a similar method as described in Example 5 (5a) and the title compound was obtained (4.56 g). Yield 91%.

Mp 149-152° C.;

IR (KBr) νₘₐₓ 3276, 3130, 2182, 1531, 885, 615 cm⁻¹;

¹H NMR (DMSO-d₆, 400 MHz) δ 1.95-2.00 (2H, m), 2.27 (3H, s), 3.57-3.69 (6H, m), 3.80-3.84 (2H, m), 6.77 (1H, d, J=3.5 Hz), 7.12 (1H, d, J=3.5 Hz), 8.42 (1H, br), 9.06 (1H, br);

MS (FAB) m/z: 308 [M+H]⁺;

Anal. Calcd for C₁₃H₁₇N₅S₂: C, 50.79; H, 5.57; N, 22.78; S, 20.86. Found: C, 50.52; H, 5.64; N, 22.44; S, 21.10.

### (97d) 3-amino-4-[4-(1,3-thiazol-2-yl)-1,4-diazepan-1-yl]thieno[2,3-b]pyridine-2-carboxamide

(2Z)-2-cyano-3-[4-(1,3-thiazol-2-yl)-1,4-diazepan-1-yl]but-2-enethioamide (394 mg, 1.28 mmol) which was produced in Example 97 (97c) and N,N-dimethylformamide dimethylacetal (168 mg, 1.41 mmol) were dissolved in N,N-dimethylformamide (2 mL) and the mixture was stirred at room temperature for one hour. Furthermore, it was stirred at 100° C. for one hour. The reaction solution was cooled to room temperature and 8N aqueous solution of sodium hydroxide (0.3 mL) and 2-chloroacetamide (156 mg, 1.66 mmol) were added. Water (2 mL) was added after the mixture was stirred at room temperature overnight. The deposited solid was separated by filtration and 215 mg of a solid was obtained. The obtained solid was recrystallized from 95% ethanol (15 mL) and 150 mg of the title compound was obtained (yield 31%).

Mp 243-245° C.;

IR (KBr) νₘₐₓ 3432, 3310, 3154, 1648, 1580, 1509, 1375, 933, 614 cm⁻¹;

¹H NMR (DMSO-d₆, 400 MHz) δ 2.48-2.50 (2H, m), 3.19-3.37 (4H, m), 3.62 (2H, t, J=5.9 Hz), 3.93-3.95 (2H, m), 6.73 (1H, d, J=3.5 Hz), 7.00 (2H, brs), 7.06 (1H, d, J=5.5 Hz), 7.10 (2H, brs), 7.13 (1H, d, J=3.5 Hz), 8.39 (1H, d, J=3.5 Hz);

MS (EI) m/z: 374 [M⁺];

Anal. Calcd for C₁₆H₁₈N₆OS₂.1.1 H₂O: C, 48.74; H, 5.16; N, 21.31; S, 16.26. Found: C, 48.98; H, 5.03; N, 21.22; S, 15.89.

### Example 98

### 3-amino-4-[4-(6-methoxypyridin-3-yl)-1,4-diazepan-1-yl]thieno[2,3-b]pyridine-2-carboxamide

(Exemplified Compound No. 3-144)

### (98a) (2Z)-2-cyano-3-[4-(6-methoxypyridin-3-yl)-1,4-diazepan-1-yl]but-2-enethioamide

1-(6-methoxypyridin-3-yl)-1,4-diazepane (J. Med.Chem., (2000), 43,2217-2226) was used in place of isobutylamine and the reaction was performed in a similar method as described in Example 5 (5a) and the title compound was obtained. Yield 66%.

Mp 159-161° C.;

IR (KBr) νₘₐₓ 3306, 3186, 2187, 1643, 1530, 1504, 1041 cm⁻¹;

¹H NMR (DMSO-d₆, 400 MHz) δ 1.90-1.97 (2H, m), 2.25 (3H, s), 3.44-3.45 (4H, m), 3.60-3.68 (4H, m), 3.74 (3H, s), 6.66 (1H, d, J=9.0 Hz), 7.28 (1H, dd, J=3.1, 9.0 Hz), 7.67 (1H, d, J=3.1 Hz), 8.31 (1H, br), 8.97 (1H, br);

MS (El) m/z: 331 [M⁺];

Anal. Calcd for C₁₆H₂₁N₅SO.0.1 H₂O: C, 57.67; H, 6.41; N, 21.02; S, 9.62. Found: C, 57.74; H, 6.50; N, 20.84; S, 9.49.

### (98b) 3-amino-4-[4-(6-methoxypyridin-3-yl)-1,4-diazepan-1-yl]thieno[2,3-b]pyridine-2-carboxamide

(2Z)-2-cyano-3-[4-(6-methoxypyridin-3-yl)-1,4-diazepan-1-yl]but-2-enethioamide which was produced in Example 98 (98a) (220 mg, 0.66 mmol) and N,N-dimethylformamide dimethylacetal (83 mg, 0.70 mmol) were dissolved in N,N-dimethylformamide (2 mL) and the mixture was stirred at room temperature for two hours. Furthermore, it was stirred at 100° C. for one hour. The reaction solution was cooled to room temperature and 8N aqueous solution of sodium hydroxide (0.2 mL) and 2-chloroacetamide (80 mg, 0.86 mmol) were added. The mixture was partitioned with water (50 mL) and ethyl acetate (50 mL) after stirring at room temperature for one hour. The organic layer was washed with a saturated saline solution (30 mL) and the solvent was evaporated under reduced pressure after drying over sodium sulfate. The residue was purified by silica gel chromatography (100% ethyl acetate) and was recrystallized from ethanol and 96 mg of the title compound was obtained (yield 36%).

Mp 170-172° C.;

IR (KBr) νₘₐₓ 3440, 3323, 3183, 1645, 1579, 1499, 1370, 1033, 939, 819 cm⁻¹;

¹H NMR (DMSO-d₆, 400 MHz) δ 2.10-2.16 (2H, m), 3.17-3.31 (4H, m), 3.49-3.52 (2H, m), 3.70-3.72 (2H, m), 3.75 (3H, s), 6.67 (1H, d, J=9.0 Hz), 6.98 (2H, br), 7.06 (1H, d, J=5.5 Hz), 7.07 (2H, br), 7.27 (1H, dd, J=3.1, 9.0 Hz), 7.67 (1H, d, J=3.1 Hz), 8.38 (1H, d, J=5.5 Hz);

(FAB) m/z: 399 [M+H]⁺;

Anal. Calcd for C₁₉H₂₂N₆SO₂.0.4 H₂O: C, 56.25; H, 5.66; N, 20.72; S, 7.90. Found: C, 55.97; H, 5.44; N, 21.11; S, 7.86.

### Example 99

### 3-amino-4-(4-pyridin-2-yl-1,4-diazepan-1-yl)thieno[2,3-b]pyridine-2-carboxamide

(Exemplified Compound No. 3-139)

### (99a) tert-butyl 2-pyridin-2-yl-1,4-diazepane-1-carboxylate

Synthesis was performed with reference to a method described in J. Org. Chem., (2001), 66, 7729-7737 as follows.

Potassium tert-butoxide (1.68 g, 15 mmol), tris(dibenzylideneacetone)dipalladium (0 valent) (92 mg, 0.1 mmol), 1,3-bis(2,6-diisopropylphenyl)imidazole-2-ylidene) hydrochloride (85 mg, 0.2 mmol) were mixed in 1,4-dioxane (20 mL), and 1,4-dioxane (10 mL) solution of 2-bromopyridine (2.37 g, 15 mmol) and N-Boc-homopiperazine (2.00 g, 10 mmol) was added dropwise to the mixture. After stirring at room temperature for 12 hours, water (100 mL) and ethyl acetate (100 mL) were added to the reaction mixture and insolubles were removed with Celite. After the liquid was partitioned into the organic layer and aqueous layer, the organic layer was washed with a saturated saline solution (50 mL) and dried over sodium sulfate. The residue which was obtained by evaporating the solvent under reduced pressure was purified by silica gel column chromatography (hexane/ethyl acetate=4:1) and 2.27 g of the title compound was obtained (yield 82%).

IR (neat) νₘₐₓ 1694, 1597, 1494, 1240, 1169, 928, 770 cm⁻¹;

¹H NMR (DMSO-d₆, 400 MHz) δ 1.38, (4.5H, s), 1.43, (4.5H, s), 1.92-1.99 (2H, m), 3.21-3.34 (2H, m), 3.54-3.78 (6H, m), 6.47-6.52 (2H, m), 7.38-7.42 (1H, m), 8.10 -8.12 (1H, m);

MS (FAB) m/z: 278 [M+H]⁺.

### (99b) 1-pyridin-2-yl-1,4-diazepane

tert-butyl 2-pyridin-2-yl-1,4-diazepane-1-carboxylate (2.17 g, 7.8 mmol) which was produced in Example 99 (99a) was dissolved in methylene chloride (8 mL) and trifluoroacetic acid (8 mL) was added under ice-cooling. The reaction mixture was concentrated under reduced pressure after stirring at room temperature for three hours. 1N aqueous solution of sodium hydroxide (30 mL) was added to the residue and the aqueous layer was extracted with methylene chloride (3×40 mL). The extract was dried over sodium sulfate and the solvent was evaporated under reduced pressure and 1.34 g (yield 97%) of the title compound was obtained as oil.

IR (neat) νₘₐₓ 3308, 1597, 1496, 1440, 769 cm⁻¹;

¹H NMR (CDCl₃, 400 MHz) δ 1.86-1.92 (2H, m), 2.83-2.86 (2H, m), 3.01-3.04 (2H, m), 3.69-3.74 (4H, m), 6.46-6.50 (2H, m), 7.40 (1H, ddd, J=2.0, 7.1, 8.6 Hz), 8.12 (1H, ddd, J=0.8, 2.0, 5.1 Hz);

MS (EI) m/z: 177 [M⁺], 121.

### (99c) (2Z)-2-cyano-3-(4-pyridin-2-yl-1,4-diazepan-1-yl)but-2-enethioamide

1-pyridin-2-yl-1,4-diazepane which was produced in Example 99 (99b) was used in place of isobutylamine and the reaction was performed in a similar method as described in Example 5 (5a) and the title compound was obtained. Yield 79%.

Mp 155-157° C.;

IR (KBr) νₘₐₓ 3398, 3288, 3184, 2185, 1598, 1540, 1494, 1439, 773 cm⁻¹;

¹H NMR (DMSO-d₆, 400 MHz) δ 1.88-1.94 (2H, m), 2.24 (3H, s), 3.55-3.70 (6H, m), 3.89-3.92 (2H, m), 6.57 (1H, dd, J=5.0, 7.0 Hz), 6.72 (1H, d, J=8.6 Hz), 7.50 (1H, ddd, J=2.0, 7.0, 8.6 Hz), 8.07 (1H, dd, J=2.0, 5.0 Hz), 8.34 (1H, br), 9.01 (1H, br);

MS (FAB) m/z: 302 [M+H]⁺.

### (99d) 4-(4-pyridin-2-yl-1,4-diazepan-1-yl)-2-thioxo-1,2-dihydropyridine-3-carbonitrile

(2Z)-2-cyano-3-(4-pyridin-2-yl-1,4-diazepan-1-yl)but-2-enethioamide (0.90 g, 3.0 mmol) which was produced in Example 99 (99c) and N,N-dimethylformamide dimethylacetal (0.39 g, 3.3 mmol) were dissolved in N,N-dimethylformamide (5 mL) and the mixture was stirred at room temperature for one hour and further at 100° C. for one hour. The reaction solution was cooled to room temperature and partitioned with isopropyl ether (20 mL) and 1N aqueous solution of sodium hydroxide (10 mL). The aqueous layer was made acidic (pH=4) with 1N hydrochloric acid and was neutralized by adding a saturated sodium bicarbonate aqueous solution. The deposited crystal was separated by filtration and washed with water and ethanol and a crude product of the title compound (0.37 g) was obtained.

Mp 220-224° C.;

¹H NMR (DMSO-d₆, 400 MHz) δ 1.88-1.94 (2H, m), 3.63-3.66 (2H, m), 3.76-3.79 (2H, m), 3.88-3.98 (4H, m), 6.41 (1H, d, J=7.8 Hz), 6.53 (1H, dd, J=5.0, 7.0 Hz), 6.71 (1H, d, J=8.6 Hz), 7.81-7.35 (1H, m), 7.46 (1H, ddd, J=2.0, 7.0, 8.6 Hz), 8.03 (1H, dd, J=2.0, 5.0 Hz), 12.50 (1H, br).

### (99e) 3-amino-4-(4-pyridin-2-yl-1,4-diazepan-1-yl)thieno[2,3-b]pyridine-2-carboxamide

A crude product (0.37 g) of 4-(4-pyridin-2-yl-1,4-diazepan-1-yl)-2-thioxo -1,2-dihydropyridine-3-carbonitrile which was produced in Example 99 (99d) was dissolved in N,N-dimethylformamide (4 mL) and 8N aqueous solution of sodium hydroxide (0.4 mL) and 2-chloroacetamide (0.14 g, 1.5 mmol) were added. Water (4 mL) was added after the mixture was stirred at room temperature for one hour. The deposited solid was separated by filtration and washed with water and ethanol and 0.33 g of a solid was obtained. The obtained solid was heated in 80% ethanol (5 mL) and the mixture was stirred, and after it was cooled, the solid was separated by filtration and 0.30 g of the title compound was obtained. Yield 27% from (2Z) -2-cyano-3-(4-pyridin-2-yl-1,4-diazepan-1-yl)but-2-enethioamide.

Mp 237-239° C.;

IR (KBr) ν^{max} 3444, 3325, 3167, 1650, 1596, 1496, 1371, 942, 770 cm⁻¹;

¹H NMR (DMSO-d₆, 400 MHz) δ 2.11-2.17 (2H, m), 3.17-3.32 (4H, m), 3.70 (2H, t, J=6.3 Hz), 3.96-4.03 (2H, m), 6.57 (1H, dd, J=5.1, 7.0 Hz), 6.69 (1H, d, J=8.6 Hz), 7.01 (2H, brs), 7.07 (1H, d, J=5.1 Hz), 7.10 (2H, brs), 7.51 (1H, ddd, J=2.0, 7.0, 8.6 Hz), 8.09 (1H, dd, J=2.0, 5.1 Hz), 8.39 (1H, d, J=5.1 Hz);

MS (FAB) m/z: 369 [M+H]⁺;

Anal. Calcd for C₁₈H₂₀N₆SO.0.3 H₂O: C, 57.83; H, 5.55; N, 22.48; S, 8.58. Found:C, 57.99; H, 5.35; N, 22.43; S, 8.59.

### Example 100

### 3-amino-4-(4-pyridin-4-yl-1,4-diazepan-1-yl)thieno[2,3-b]pyridine-2-carboxamide

(Exemplified Compound No. 3-141)

### (100a) tert-butyl 4-pyridin-4-yl-1,4-diazepane-1-carboxylate

4-bromopyridine hydrochloride was used in place of 2-bromopyridine with 2.1 equivalent of potassium tert-butoxide, and the reaction was performed in a similar method as described in Example 99 (99a). The residual substance which was obtained by post-treatment of the reaction was purified by silica gel column chromatography (Chromatorex NH, Fuji Silysia) (100% ethyl acetate) and the title compound was obtained. Yield 34%.

Mp 124-129° C.;

IR (KBr) νₘₐₓ 1674, 1597, 1167, 987 cm⁻¹;

¹H NMR (CDCl₃, 400 MHz) δ 1.36 (4.5H, s), 1.42 (4.5H, s), 1.92-1.99 (2H, m), 3.21-3.34 (2H, m), 3.52-3.59 (4H, m), 6.50 (2H, brd, J=6.5 Hz), 8.18-8.20 (2H, m);

MS (EI) m/z: 277 [M⁺], 220;

Anal. Calcd for C₁₅H₂₃N₃O₂: C, 64.95; H, 8.36; N, 15.15. Found: C, 64.78; H, 8.46; N, 15.03.

### (100b) Pyridin-4-yl-1,4-diazepane

tert-butyl 4-pyridin-4-yl-1,4-diazepane-1-carboxylate (0.42 g, 1.5 mmol) which was produced in Example 100 (100a) was dissolved in methanol (3 mL) and 4N hydrochloric acid-1,4-dioxane solution (3 mL) was added. The reaction mixture was concentrated under reduced pressure after stirring at room temperature for two hours. 1N aqueous solution of sodium hydroxide (10 mL) was added to the residue and the aqueous layer was extracted with methylene chloride (3×20 mL). The extract was dried over sodium sulfate and the solvent was evaporated under reduced pressure and 0.26 g (yield 97%) of the title compound was obtained as oil.

IR (neat) νₘₐₓ 3270, 1600, 1518, 804 cm⁻¹;

¹H NMR (CDCl₃, 400 MHz) δ 1.86-1.92 (2H, m), 2.83 (2H, t, J=5.9 Hz), 3.02 (2H, t, J=5.3 Hz), 3.53-3.61 (4H, m), 6.51 (2H, d, J=6.6 Hz), 8.20 (2H, d, J=6.6 Hz);

MS (FAB) m/z: 178 [M+H]⁺.

### (100c) (2Z)-2-cyano-3-(4-pyridin-4-yl-1,4-diazepan-1-yl)but-2-enethioamide

1-pyridin-4-yl-1,4-diazepane which was produced in Example 100 (100b) was used in place of isobutylamine and the reaction was performed in a similar method as described in Example 5 (5a) and the title compound was obtained. Yield 57%.

Amorphous solid

IR (KBr) νₘₐₓ 3172, 2185, 1600, 1538, 1520, 1411 cm⁻¹;

¹H NMR (DMSO-d₆, 400 MHz) δ 1.89-1.94 (2H, m), 2.23 (3H, s), 3.52-3.62 (6H, m), 3.78-3.80 (2H, m), 6.70 (2H, d, J=6.7 Hz), 8.09 (2H, d, J=6.7 Hz), 8.41 (1H, br), 9.04 (1H, br);

MS (FAB) m/z: 302 [M+H]⁺.

### (100d) 3-amino-4-(4-pyridin-4-yl-1,4-diazepan-1-yl)thieno[2,3-b]pyridine-2-carboxamide

(2Z)-2-cyano-3-(4-pyridin-4-yl-1,4-diazepan-1-yl)but-2-enethioamide which was produced in Example 100 (100c) (200 mg, 0.66 mmol) and N,N-dimethylformamide dimethylacetal (87 mg, 0.73 mmol) were dissolved in N,N-dimethylformamide (3 mL) and after stirring at room temperature for two hours, further stirred at 100° C. for one hour. The reaction solution was cooled to room temperature and 8N aqueous solution of sodium hydroxide (0.2 mL) and 2-chloroacetamide (93 mg, 0.99 mmol) were added. Water (4 mL) was added after the mixture was stirred at room temperature for one hour. The deposited solid was separated by filtration after allowing to stand at room temperature for 15 hours and washed with water and then dried, and 55 mg of a crude product was obtained. The crude product was suspended in 80% aqueous ethanol (3 mL), and heated and the mixture was stirred and after it was cooled, the solid was separated by filtration and purified and 30 mg of the title compound was obtained (yield 12%).

Mp 288-291° C. (decomposition);

IR (KBr) νₘₐₓ 3334, 1650, 1597, 1573, 1510, 1367 cm⁻¹;

¹H NMR (DMSO-d₆, 400 MHz) δ 2.13-2.18 (2H, m), 3.15-3.30 (4H, m), 3.56-3.59 (2H, m), 3.79-3.81 (2H, m), 6.68 (2H, d, J=6.7 Hz), 6.98 (2H, br), 7.06 (1H, d, J=5.1 Hz), 7.09 (2H, br), 8.09 (2H, d, J=6.7 Hz), 8.38 (1H, d, J=5.1 Hz);

MS (El) m/z: 368 [M⁺], 108;

Anal. Calcd for C₁₈H₂₀N₆OS.0.3 H₂O: C, 57.83; H, 5.55; N, 22.48; S, 8.58. Found: C, 57.76; H, 5.48; N, 22.46; S, 8.50.

### Example 101

### 3-amino-4-[3-(methoxymethyl) piperidin-1-yl]thieno[2,3-b]pyridine -2-carboxamide

(Exemplified Compound No. 1-21)

### (101a) tert-butyl 3-(methoxymethyl)piperidine-1-carboxylate

Sodium hydride (55% oily, 0.26 g, 6 mmol) was suspended in tetrahydrofuran (5 mL) and N,N-dimethylformamide (3 mL) solution of tert-butyl 3-(hydroxymethyl)piperidine-1-carboxylate (Bioorg.Med.Chem.Lett., 8, (1998), 1595-1600) (1.08 g, 5 mmol) was added under ice-cooling, and further methyl iodide (0.85 g, 6 mmol) was added. Water (50 mL) was added to the reaction mixture after stirring at room temperature for three days, the aqueous layer was extracted with ethyl acetate (50 mL). The extract was dried over sodium sulfate and the solvent was evaporated under reduced pressure. The residue was purified by silica gel column chromatography (hexane/ethyl acetate=10:1) and 1.03 g of the title compound was obtained (yield 90%).

IR (neat) νₘₐₓ 1696, 1422, 1151 cm⁻¹;

¹H NMR (CDCl₃, 400 MHz) δ 1.16-1.51 (2H, m), 1.46 (9H, s), 1.60-1.82 (3H, m), 2.64 (1H, br), 2.82 (1H, brt, J=13.3 Hz), 3.24 (2H, d, J=5.9 Hz), 3.32 (3H, s), 3.87 (1H, dt, J=4.0, 13.3 Hz), 3.94 (1H, m);

MS (El) m/z: 229 [M⁺], 114;

Anal. Calcd for C₁₂H₂₃NO₃: C, 62.85; H, 10.11; N, 6.11. Found: C, 62.90; H, 9.77; N, 6.04.

### (101b) 3-(methoxymethyl)piperidine

tert-butyl 3-(methoxymethyl)piperidine-1-carboxylate (1.03 g, 4.5 mmol) which was produced in Example 101 (101a) was dissolved in methylene chloride (4 mL) and trifluoroacetic acid (2 mL) was added under ice-cooling. The reaction mixture was concentrated under reduced pressure after stirring at room temperature for two hours. 1N aqueous solution of sodium hydroxide (20 mL) was added to the residue and the aqueous layer was extracted with methylene chloride (3×30 mL). The extract was dried over sodium sulfate and the solvent was evaporated under reduced pressure and 0.50 g of the title compound was obtained (yield 86%) as oil.

IR (neat) νₘₐₓ 3312, 1467, 1450, 1270, 1128, 1097 cm⁻¹;

¹H NMR (CDCl₃, 400 MHz) δ 1.06-1.16 (1H, m), 1.40-1.51 (1H, m), 1.63-1.80 (3H, m), 2.33 (1H, t, J=11.7 Hz), 2.55 (1H, dt, J=2.7, 11.7 Hz), 3.00 (1H, brd, J=12.1 Hz), 3.11 (1H, brd, J=12.1 Hz), 3.20 (2H, d, J=6.3 Hz), 3.31 (3H, s);

MS (EI) m/z: 129 [M⁺], 114.

### (101c) (2Z)-2-cyano-3-[3-(methoxymethyl)piperidin-1-yl]but-2-enethioamide

3-(methoxymethyl)piperidine which was produced in Example 101 (101b) was used in place of isobutylamine and the reaction was performed in a similar method as described in Example 5 (5a) and the title compound was obtained. Yield 81%.

Mp 151-152° C.;

IR (KBr) νₘₐₓ 3380, 3255, 3157, 2183, 1605, 1531, 1410, 1261 cm⁻¹;

¹H NMR (DMSO-d₆, 400 MHz) δ 1.25-1.35 (1H, m), 1.48-1.59 (1H, m), 1.66-1.76 (2H, m), 1.85-1.94 (1H, m), 2.26 (3H, s), 2.91-2.97 (1H, m), 3.03-3.10 (1H, m), 3.16-3.24 (2H, m), 3.22 (3H, s), 3.50-3.63 (2H, d, J=6.3 Hz), 8.18 (1H, br), 8.90 (1H, br);

MS (EI) m/z: 253 [M⁺], 220;

Anal. Calcd for C₁₂H₁₉N₃OS.0.1 H₂O: C, 56.49; H, 7.58; N, 16.47; S, 12.57. Found: C, 56.62; H, 7.41; N, 16.39; S, 12.60.

### (101d) 4-[3-(methoxymethyl)piperidin-1-yl]-2-thioxo-1,2-dihydropyridine-3-carbonitrile

(2Z)-2-cyano-3-[3-(methoxymethyl)piperidin-1-yl]but-2-enethioamide which was produced in Example 101 (101c) (0.39 g, 1.5 mmol) and N,N-dimethylformamide dimethylacetal (0.20 g, 1.7 mmol) were dissolved in N,N-dimethylformamide (3 mL) and the mixture was stirred at room temperature for one hour, and further stirred at 100° C. for one hour. The reaction solution was cooled to room temperature and water (50 mL) was added. The aqueous layer was extracted with ethyl acetate (3×50 mL) and the extract was dried over sodium sulfate and concentrated under reduced pressure. The residue was purified by silica gel column chromatography (methylene chloride/methanol=20:1) and 125 mg of the title compound was obtained (yield 31%).

Mp 154-156° C.;

IR (KBr) νₘₐₓ 2208, 1622, 1550, 1517, 1249 cm⁻¹;

¹H NMR (DMSO-d₆, 400 MHz) δ 1.26-1.93 (5H, m), 3.04 (1H, dd, J=10.2, 13.3 Hz), 3.18-3.27 (3H, m), 3.24 (3H, s), 3.96-4.02 (2H, m), 6.46 (1H, d, J=7.8 Hz) , 7.46 (1H, d, J=7.8 Hz);

MS (EI) m/z: 263 [M⁺], 248, 218;

Anal. Calcd for C₁₃H₁₇N₃OS.0.1 H₂O: C, 58.89; H, 6.54; N, 15.85; S, 12.09. Found: C, 58.93; H, 6.60; N, 16.00; S, 12.07.

### (101e) 3-amino-4-[3-(methoxymethyl)piperidin-1-yl]thieno[2,3-b]pyridine-2-carboxamide

4-[3-(methoxymethyl) piperidin-1-yl]-2-thioxo-1,2-dihydropyridine-3-carbonitrile (120 mg, 0.46 mmol) which was produced in Example 101 (101d) was dissolved in N,N-dimethylformamide (1 mL) and 8N aqueous solution of sodium hydroxide (0.2 mL) and 2-chloroacetamide (55 mg, 0.59 mmol) were added. The mixture was blended with water (50 mL) and ethyl acetate (50 mL) and partitioned after stirring at room temperature for one hour. After the organic layer was washed with a saturated saline solution (30 mL), it was dried over sodium sulfate and concentrated under reduced pressure. Ether was added to the residue and solidified, and the deposited solid was separated by filtration and further washed with ether and 138 mg of the title compound was obtained (yield 95%).

Mp 229-230° C.;

IR (KBr) νₘₐₓ 3432, 3320, 3144, 1653, 1577, 1501, 1375, 1097 cm⁻¹;

¹H NMR (DMSO-d₆, 400 MHz) δ 1.03-1.15 (1H, m), 1.73-1.84 (3H, m), 2.07-2.19 (1H, m), 2.35-3.44 (4H, m), 3.22 (3H, s), 6.97 (2H, br), 7.00 (1H, d, J=5.1 Hz), 7.09 (2H, br), 8.40 (1H, d, J=5.1 Hz);

MS (EI) m/z: 320 [M⁺];

Anal. Calcd for C₁₅H₂₀N₄O₂S: C, 56.23; H, 6.29; N, 17.49; S, 10.01. Found: C, 56.21; H, 6.45; N, 17.37; S, 9.80.

### Example 102

### 3-amino-4-(1,4-diazepan-1-yl)thieno[2,3-b]pyridine-2-carboxamide dihydrochloride

(Exemplified Compound No. 3-78)

3-amino-4-(4-tert-butoxycarbonyl-1,4-diazepan-1-yl)thieno[2,3-b]pyridine-2-carboxamide (0.77 g, 2.0 mmol) which was produced in Example 55 (55c) was dissolved in 1,4-dioxane (4 mL) and 4N hydrochloric acid/1,4-dioxane (4 mL) was added. The reaction mixture was concentrated under reduced pressure after stirring at room temperature for four hours and the 0.72 g of the title compound was obtained.

Mp >295° C.;

IR (KBr) νₘₐₓ3457, 3371, 3291, 1654, 1614, 1579 cm⁻¹;

¹HNMR (DMSO-d₆, 400MHz) δ 2.12-2.19 (2H, m), 3.20-3.26 (2H, m), 3.36-3.42 (2H, m), 3.58-3.64 (2H, m), 3.80-3.87 (2H, m), 7.11 (1H, d, J=6.5 br), 8.44 (1H, d, J=6.5 Hz), 9.46 (2H, br);

MS (FAB) m/z: 292 [M+H]⁺;

Anal. Calcd for C₁₃H₁₇N₅OS.2(HCl).0.3 H₂O: C, 42.24; H, 5.34; N, 18.94; S, 8.67; Cl, 19.18. Found: C, 42.39; H, 5.22; N, 19.01; S, 8.53; Cl, 18.93.

### Example 103

### 4-(4-acetyl-1,4-diazepan-1-yl)-3-aminothieno[2,3-b]pyridine-2-carboxamide

(Exemplified Compound No. 3-149)

3-amino-4-(1,4-diazepan-1-yl)thieno[2,3-b]pyridine-2-carboxamide dihydrochloride (182 mg, 0.5 mmol) which was produced in Example 102 was suspended in tetrahydrofuran (3 mL) and blended with triethylamine (0.15 mL) and acetic acid anhydride (77 mg, 0.75 mmol) and the mixture was stirred at room temperature for two hours. A saturated sodium bicarbonate aqueous solution (20 mL) was added to the reaction mixture and the aqueous layer was extracted with methylene chloride (2×30 mL). After the extract was dried over sodium sulfate, the solvent was evaporated under reduced pressure and 55 mg of the title compound (yield 33%) was obtained when the obtained solid was recrystallized from ethanol (4 mL).

Mp 214-215° C.;

IR (KBr) νₘₐₓ3425, 3333, 3188, 1632, 1581, 1370, 938 cm⁻¹;

¹H NMR (DMSO-d₆, 400MHz) δ 1.97-2.08 (2H, m), 2.02 (1.5H, s), 2.05 (1.5H, s), 3.15-3.24 (4H, m), 3.55-3.62 (2H, m), 3.69-3.77 (2H, m), 7.01 (2H, br), 7.04 (0.5H, d, J=5.5 Hz), 7.07 (0.5H, d, J=5.5 Hz), 7.09 (2H, br), 8.38 (0.5H, d, J=5.5 Hz), 8.41 (0.5H, d, J=5.5 Hz);

MS (FAB) m/z: 334 [M+H]⁺;

Anal. Calcd for C₁₅H₁₉N₅O₂S.0.1 H₂O: C, 53.75; H, 5.77; N, 20.89; S, 9.57. Found: C, 53.89; H, 5.75; N, 20.73; S, 9.42.

### Example 104

3-amino-4-[4-(methylsulfonyl)-1,4-diazepan-1-yl]thieno[2,3-b]pyridine-2-carboxamide

(Exemplified Compound No. 3-151)

3-amino-4-(1,4-diazepan-1-yl)thieno[2,3-b]pyridine-2-carboxamide dihydrochloride which was produced in Example 102 (182 mg, 0.5 mmol) and potassium carbonate (221 mg, 1.6 mmol) were suspended in tetrahydrofuran (5 mL), and the resultant solution was blended with methanesulfonic acid anhydride (105 mg, 0.6 mmol) and the mixture was stirred at room temperature for 15 hours. Water (20 mL) and ethyl acetate (30 mL) were added to the reaction mixture and partitioned and the aqueous layer was extracted with ethyl acetate (30 mL). After the combined organic layer was dried over sodium sulfate, the solvent was evaporated under reduced pressure and 72 mg of the title compound (yield 39%) was obtained when the obtained residue was purified by silica gel column chromatography (methylene chloride/methanol=15:1).

Mp 214-217° C.;

IR (KBr) νₘₐₓ 3424, 3327, 3155, 1650, 1582, 1371, 1323, 1147, 934 cm⁻¹; ¹H NMR (DMSO-d₆, 400MHz) δ 2.02-2.07 (2H, m), 2.96 (3H, s), 3.26-3.84 (2H, m), 3.44-3.47 (2H, m), 3.58-3.61 (2H, m), 7.04 (2H, br), 7.10 (1H, d, J=5.5 Hz), 701 (2H, br), 8.44 (1H, d, J=5.5 Hz);

MS (FAB) m/z: 370 [M+H]⁺;

Anal. Calcd for C₁₄H₁₉N₅O₃S₂: C, 45.51; H, 5.18; N, 18.96; S, 17.36. Found: C, 45.38; H, 5.08; N, 18.64; S, 17.04.

### Example 105

### 3-amino-4-[4-(6-chloropyridazin-3-yl)-1,4-diazepan-1-yl]thieno[2,3-b]pyridine-2-carboxamide

(Exemplified Compound No. 3-148)

### (105a) (2Z)-3-[4-(6-chloropyridazin-3-yl)-1,4-diazepan-1-yl]-2-cyanobut-2-enethioamide

1-(6-chloropyridazin-3-yl)-1,4-diazepane (J. Med. Chem., (2002), 45,4011-4017) was used in place of isobutylamine; and the reaction was performed in a similar method as described in Example 5 (5a) and the title compound was obtained. Yield 60%.

Amorphous solid

IR (KBr) νₘₐₓ 3289, 3185, 2191, 1587, 1531, 1453, 1429 cm⁻¹; ¹H NMR (DMSO-d₆, 400MHz) δ 6 1.89-1.95 (2H, m), 2.25 (3H, s), 3.59-3.66 (4H, m), 3.74-3.77 (2H, m), 3.97-4.00 (2H, m), 7.31 (1H, d, J=9.8 Hz), 7.51 (1H,d, J=9.8 Hz), 8.40 (1H, br), 9.04 (1H, br);

MS (FAB) m/z: 337 [M+H]⁺.

### (105b) 3-amino-4-[4-(6-chloropyridazin-3-yl)-1,4-diazepan-1-yl]thieno[2,3-b]pyridine-2-carboxamide

(2Z)-3-[4-(6-chloropyridazin-3-yl)-1,4-diazepan-1-yl]-2-cyanobut-2-enethioamide (0.75 g, 2.2 mmol) which was produced in Example 105 (105a) and N,N-dimethylformamide dimethylacetal (0.28 g, 2.3 mmol) were dissolved in N,N-dimethylformamide (5 mL) and the mixture was stirred at room temperature for one hour, and further stirred at 100° C. for two hours. The reaction solution was cooled to room temperature and 8N aqueous solution of sodium hydroxide (0.5 mL) and 2-chloroacetamide (0.27 g, 2.9 mmol) were added. Water (30 mL) was added after the mixture was stirred at room temperature for one hour. The aqueous layer was extracted with ethyl acetate (2×50 mL) and the extract was concentrated under reduced pressure after drying over sodium sulfate. The obtained residue was purified by silica gel column chromatography (methylene chloride/methanol=20:1) and the obtained crystal was further washed with ethanol and 0.15 g of the title compound was obtained (yield 17%).

Mp 232-234° C.;

IR (KBr) νₘₐₓ 3320, 1645, 1584, 1501, 1450, 1372, 939 cm^{31 1}; ¹H NMR (DMSO-d₆, 400MHz) δ 2.13-2.19 (2H, m), 3.18-3.23 (2H, m), 3.30-3.35 (2H, m), 3.75-3.78 (2H, m), 4.00-4.05 (2H, m), 7.00 (2H, br), 7.05 (1H, d, J=5.1 Hz), 7.09 (2H, br), 7.26 (1H, d, J=9.6 Hz), 7.50 (1H, d, J=9.6 Hz), 8.38 (1H, d, J=5.1 Hz);

MS (FAB) m/z: 404 [M+H]⁺;

Anal. Calcd for C₁₇H₁₈N₇OSCl. 0.3 H₂O: C, 49.89; H, 4.58; N, 23.95; S, 7.83; Cl, 8.66. Found: C, 49.93; H, 4.52; N, 23.93; S, 8.09; Cl, 8.39.

### Example 106

### 3-amino-4-[4-(5-methylpyridin-2-yl)-1,4-diazepan-1-yl]thieno[2,3-b]pyridine-2-carboxamide

(Exemplified Compound No. 3-143)

### (106a) tert-butyl-4-(5-methylpyridin-2-yl)-1,4-diazepane-1-carboxylate

2-bromo-5-methylpyridine was used in place of 2-bromopyridine and the reaction was performed in a similar method as described in Example 99 (99a). The obtained crude product was purified by silica gel column chromatography (hexane/ethyl acetate=5:1) and the title compound was obtained at yield 81%.

IR (neat) νₘₐₓ 1694, 1612, 1503, 1415, 1170, 929 cm⁻¹;

¹HNMR (CDCl₃, 400MHz) δ 1.39 (4.5H, s), 1.44 (4.5H, s), 1.92-1.98 (2H, m), 2.19 (3H, s), 3.20-3.33 (2H, m), 3.54-3.76 (6H, m), 6.45 (1H, d, J=8.6 Hz), 7.26 (1H, dd, J=2.0, 8.6 Hz), 7.96 (1H,d, J=2.0 Hz);

MS (EI) m/z: 291 [M⁺], 135;

Anal. Calcd for C₁₆H₂₅N₃O. 0.4 H₂O: C, 64.36; H, 8.71; N, 14.07. Found: C, 64.35; H, 8.96; N, 13.98.

### (106b) 1-(5-methylpyridin-2-yl)-1,4-diazepane

The reaction was performed in a similar method as described in Example 100 (100b) and the title compound was obtained at yield 97%.

IR (neat) νₘₐₓ 3307, 1613, 1503, 1409, 804 cm⁻¹;

¹H NMR (CDCl1₃, 400MHz) δ 1.85-1.91 (2H, m), 2.17 (3H, s), 2.82-2.85 (2H, m), 3.01-3.03 (2H, m), 3.68-3.72 (4H, m), 6.43 (1H, d, J=8.6 Hz), 7.26 (1H, dd, J=2.0, 8.6 Hz), 7.97 (1H, d, J=2.0 Hz);

MS (FAB) m/z: 192 [M+H]⁺, 135.

### (106c) (2Z)-2-cyano-3-[(4-(5-methylpyridin-2-yl)-1,4-diazepan-1-yl)but-2-enethioamide

1-(5-methylpyridin-2-yl)-1,4-diazepane which was produced in Example 106 (106b) was used in place of isobutylamine and the reaction was performed in a similar method as described in Example 5 (5a) and the title compound was obtained. Yield 79%.

Mp 140-145° C.;

IR (KBr) νₘₐₓ 3274, 3131, 2181, 1611, 1528, 1501, 1411, 885 cm⁻¹; ¹H NMR (DMSO-d₆, 400 MHz) δ 1.87-1.93 (2H, m), 2.12 (3H, s), 2.24 (3H, s), 3.53-3.67 (6H, m), 3.86-3.89 (2H, m), 6.65 (1H, d, J=8.6 Hz), 7.34 (1H, dd, J=2.0, 86 Hz), 7.91 (1H, d, J=2.0 Hz), 8.82 (1H, br), 8.99 (1H, br);

MS (FAB) m/z: 316 [M+H]⁺;

Anal. Calcd for C₁₆H₂₁N₅S.0.3 H₂O: C, 59.90; H, 6.79; N, 21.83; S, 9.95. Found: C, 59.96; H, 6.62; N, 21.55; S, 10.19.

### (106d) 3-amino-4-[4-(5-methylpyridin-2-yl)-1,4-diazepan-1-yl]thieno[2,3-b]pyridine-2-carboxamide

(2Z)-2-cyano-3-[(4-(5-methylpyridin-2-yl)-1,4-diazepan-1-yl)but-2-enethioamide (1.26 g, 4.0 mmol) which was produced in Example 106 (106c) and N,N-dimethylformamide dimethylacetal (0.52 g, 4.4 mmol) were dissolved in N,N-dimethylformamide (6 mL) and the mixture was stirred at room temperature for one hour, and further stirred at 100° C. for one hour. The reaction solution was cooled to room temperature and 8N aqueous solution of sodium hydroxide (1 mL) and 2-chloroacetamide (0.49 g, 5.2 mmol) were added. Water (6 mL) was added after the mixture was stirred at room temperature for two hours. The deposited solid was separated by filtration, and 0.87 g of a crude product was obtained when washed with water and ethanol. The crude product was suspended in 80% aqueous ethanol (10 mL), and heated and the mixture was stirred and after it was cooled, the solid was separated by filtration and purified and 0.66 g of the title compound was obtained (yield 43%).

Mp 205-208° C.;

IR (KBr) νₘₐₓ 3440, 3316, 3155, 1649, 1609, 1579, 1499, 1371, 939 cm⁻¹;

¹H NMR (DMSO-d₆, 400MHz) δ 2.08-2.15 (2H, m), 2.13 (3H, s), 3.15-3.28 (4H, m), 3.64-3.67 (2H, m), 3.92-3.98 (2H, m), 6.60 (1H, d, J=8.6 Hz), 6.98 (2H, br), 7.4 (1H, d, J=5.1 Hz), 7.07 (2H, br), 7.34 (1H, dd, J=2.3, 8.6 Hz), 7.90 (1H, d, J=2.0 Hz), 8.36 (1H, d, J=5.1 Hz);

MS (EI) m/z: 382 [M⁺]; p Anal. Calcd for C₁₉H₂₂N₆SO: C, 59.66; H, 5.80; N, 21.97; S, 8.38. Found: C, 59.46; H, 5.93; N, 21.78; S, 8.41.

### Example 107

### 3-amino-4-methoxythieno[2,3-b]pyridine-2-carboxamide

(Exemplified Compound No. 1-1)

### (107a) 2-chloro-4-methoxynicotinonitrile

A phosphorus oxychloride (10 mL) solution of 4-methoxy-2-oxo-1,2-dihydropyridine -3-carbonitrile (1.50 g, 10 mmol) which was synthesized by a method of Ogawa et al. (Heterocycles, 36,145-148,1993) was heated under reflux for two hours. The reaction liquid was concentrated and a sodium hydrogen carbonate aqueous solution (10 mL) was added to the obtained residual substance and the mixture was extracted with ethyl acetate (3×10 mL), and the solvent was evaporated under reduced pressure after the extract was dried over sodium sulfate. The obtained residue was powderized with ether and the title compound was obtained (1.31 g, yield 78%).

Pale yellow powder

¹H NMR (DMSO-d₆, 400 MHz) δ 4.05 (3H, s), 7.40 (1H, d, J=4.3 Hz), 8.55 (1H, d, J=4.3 Hz).

### (107b) 3-amino-4-methoxythieno[2,3-b]pyridine-2-carboxamide

2-mercaptoacetamide (109 mg, 1.2 mmol) and 8M aqueous solution of sodium hydroxide (0.4 mL) were added to N,N-dimethylformamide (2 mL) solution of 2-chloro-4-methoxynicotinonitrile (169 mg, 1.0 mmol) which was produced in Example 107 (107a), and the mixture was stirred at room temperature for one hour. Water (5 mL) was added to the reaction liquid and the resulted solid was separated by filtration and the title compound was obtained (92 mg, yield 41%).

Yellow powder

Mp 238-241° C.;

IR (KBr) νₘₐₓ 3482, 3325, 3149, 1667, 1613, 1583, 1504, 1375, 1289, 1044 cm⁻¹; ¹H NMR (DMSO-d₆, 400 MHz) δ 4.01 (3H, s), 6.95 (2H, brs), 6.98 (1H, d, J=5.9 Hz), 7.05 (2H, brs), 8.46 (1H, d, J=5.9 Hz);

HRMS m/z calcd for C₉H₉N₃O₂S 223.0415. Found. 223.0416;

MS (EI) m/z: 223 [M⁺], 205, 178, 150, 137, 122, 104, 77, 66, 45;

Anal. Calcd for C₉H₉N₃O₂S: C, 48.42; H, 4.06; N, 18.82; S, 14.36. Found: C, 48.11; H, 4.32; N, 18.76, S, 14.18.

### Example 108

### 3-amino-4-ethoxythieno[2,3-b]pyridine-2-carboxamide

(Exemplified Compound No. 1-2)

### (108a) 2-chloro-4-ethoxynicotinonitrile

4-methoxy-2-oxo-1,2-dihydropyridine-3-carbonitrile, which was synthesized by a method of Ogawa et al. (Heterocycles, 36,145-148,1993) using triethyl orthoacetate in place of trimethyl orthoacetate, was used and the reaction was performed in a similar method as described in Example 107 (107a) and the title compound was synthesized.

Pale yellow powder

¹H NMR (DMSO-d₆, 500 MHz) δ1.39 (3H, t, J=6.8 Hz), 4.36 (2H, q, J=6.8 Hz), 7.39 (1H, d, J=6.4 Hz), 8.52 (1H, d, J=6.4 Hz).

### (108b) 3-amino-4-ethoxythieno[2,3-b]pyridine-2-carboxamide

2-chloro-4-ethoxynicotinonitrile was used in place of 2-chloro-4-methoxynicotinonitrile and the reaction was performed in a similar method as described in Example 107 (107b) and the title compound was synthesized.

Pale yellow powder

Mp 241-243° C.;

IR (KBr) νₘₐₓ 3446, 3331, 1645, 1584, 1506, 1375, 1294, 1046 cm⁻¹;

¹H NMR (DMSO-d₆, 400 MHz) δ 1.44 (3H, t, J=7.1 Hz), 4.30 (2H, q, J=7.1 Hz), 6.84 (2H, brs), 6.95 (1H, d, J=5.9 Hz), 7.03 (2H, brs), 8.41 (1H, d, J=5.9 Hz); HRMS m/z calcd for C ₁₀H₁₁O₂N₃S 237.0572. Found 237.0574;

MS (EI) m/z: 237 [M⁺], 219, 205, 192, 176, 164, 148, 137, 120, 104;

Anal. Calcd for C₁₀H₁₁N₃O₂S: C, 50.62; H, 4.67; N, 17.71; S, 13.51. Found: C, 50.62; H, 4.69; N, 17.97, S, 13.52.

### Example 109

### {[3-amino-2-(aminocarbonyl)thieno[2,3b]pyridin-4-yl loxy}acetic acid

(Exemplified Compound No. 1-5)

### (109a) 2-chloro-4-oxo-1,4-dihydropyridine-3-carbonitrile

Concentrated hydrochloric acid (5 mL) was added to acetic acid (5 mL) solution of 2-chloro-4-methoxynicotinonitrile (0.98 g, 5.81 mmol) which was produced in Example 107 (107a), and heated under reflux for two hours. Water (10 mL) was added to the reaction liquid and extracted with ethyl acetate (10 mL), and the solvent was evaporated under reduced pressure after the extract was dried over sodium sulfate. The obtained residue was powderized with ether and the title compound was obtained (0.35 g, yield 39%).

White powder

Mp 214-217° C.;

¹H NMR (DMSO-d₆, 400 MHz) δ 6.96 (1H, d, J=5.9 Hz), 8.25 (1H, d, J=5.9 Hz). (108b) { [3-amino-2-(aminocarbonyl)thieno[2,3-b]pyridin-4-yl]oxy}acetic acid

Potassium carbonate (152 mg, 1.1 mmol) and bromoacetate tert-butyl ester (162 μL, 1.1 mmol) were added to N, N-dimethylacetamide (5 mL) solution of 2-chloro -4-oxo-1,4-dihydropyridine-3-carbonitrile (155 mg, 1.0 mmol) which was produced in Example 109 (109a) and the mixture was stirred at room temperature for five hours. Ethyl acetate (20 mL) and water (20 mL) were added to the reaction liquid and partitioned. The organic layer was washed with water (5×20 mL), a saline solution (20 mL) and the solvent was evaporated under reduced pressure after drying over sodium sulfate. The obtained residue was dissolved in N,N-dimethylformamide (2 mL) and was blended with 2-mercaptoacetamide (118 mg, 1.3 mmol) and 8N aqueous solution of sodium hydroxide (0.4 mL) and the mixture was stirred at room temperature for one hour. After 1N hydrochloric acid (4 mL) was added to the reaction liquid, it was concentrated under reduced pressure. The obtained residue was purified by reverse-phase chromatography (100% water) and the title compound was obtained (78 mg, yield 29%).

Yellow powder

Mp 248-249° C. (decomposition);

IR (KBr) νₘₐₓ 3462, 3340, 3169, 1647, 1591, 1507, 1376, 1084 cm⁻¹;

¹H NMR (DMSO-d₆, 400 MHz) δ 4.97 (2H, s), 6.93 (1H, d, J=5.9 Hz), 7.04 (2H, brs), 7.07 (2H, brs), 8.44 (1H, d, J=5.9 Hz);

HRMS m/z calcd for C₁₀H₉O₄N₃S 267.0314. Found 267.0325;

MS (FAB) m/z: 267 [M⁺], 251, 205, 187, 69, 55;

Anal. Calcd for C₁₀H₉N₃O₄S. 1.96H₂O: C, 39.70; H, 4.30; N, 13.89; S, 10.60. Found: C, 39.39; H, 3.99; N, 13.89; S, 10.46.

### Example 110

### 3-amino-4-(benzyloxy)thieno[2,3-b]pyridine-2-carboxamide

(Exemplified Compound No. 1-4)

Benzyl bromide was used in place of bromoacetic acid tert-butyl ester and the reaction was performed in a similar method as described in Example 109 (109b) and the title compound was synthesized.

White powder

Mp 216-221° C.;

IR (KBr) νₘₐₓ 3490, 3324, 3151, 1651, 1580, 1502, 1370, 1290, 1039, 741 cm⁻¹;

¹H NMR (DMSO-d₆, 400 MHz) δ5.44 (2H, s), 6.89 (2H, brs), 7.04 (1H, d, J=5.5 Hz), 7.07 (2H, brs), 7.36-7.55 (5H, m), 8.42 (1H, d, J=5.5 Hz);

HRMS m/z calcd for C₁₅H₁₃O₂N₃S 299.0728. Found 299.0730;

MS (FAB) m/z: 299 [M⁺], 283, 273, 257, 200, 193, 165, 91, 65;

Anal. Calcd for C₁₅H₁₃N₃O₂S.0.12H₂O: C, 59.75; H, 4.43; N, 13.94; S, 10.63. Found: C, 60.09; H, 4.36; N, 13.63; S, 10.26.

### Example 111

### 3-amino-4-isopropoxythieno[2,3-b]pyridine-2-carboxamide

(Exemplified Compound No. 1-3)

### (111a) 2,4-dichloronicotinonitrile

A phosphorus oxychloride (0.7 mL) solution of 2-chloro-4-oxo-1,4-dihydropyridine -3-carbonitrile (238 mg, 1.54 mmol) which was produced in Example 109 (109a) was heated for two hours under reflux. The reaction liquid was concentrated and a sodium hydrogen carbonate aqueous solution (10 mL) was added to the obtained residual substance and extracted with ethyl acetate (10 mL) twice, and the solvent was evaporated under reduced pressure after drying over sodium sulfate. The obtained residue was powderized in hexane and the title compound was obtained (167 mg, yield 63%).

White powder

Mp 107-109° C.;

IR (KBr) νₘₐₓ 3072, 2235, 1559, 1538, 1444, 1406, 1368, 1220, .820 cm⁻¹;

¹H NMR (DMSO-d₆, 400 MHz) δ 7.91 (1H, d, J=5.5 Hz), 8.65 (1H, d, J=5.5 Hz);

HRMS m/z calcd for C₆H₂N₂Cl₂ 171.9595, found 171.9598;

MS (EI) m/z: 172 [M⁺], 137, 110, 101, 75, 62, 51.

### (111b) 2-chloro-4-isopropoxynicotinonitrile

sodium hydride (48 mg, 1.1 mmol) and N,N-dimethylacetamide (1 mL) solution of 2,4-dichloronicotinonitrile (173 mg, 1.00 mmol) which was produced in Example 111 (lila) were added to THF (1 mL) solution of 2-propanol (84 μL, 1.1 mmol) at 0C and the mixture was stirred at room temperature for one hour. Water (3 mL) was added to the reaction liquid and generated powder was separated by filtration and the title compound was obtained (125 mg, yield 63%).

White powder

IR (KBr) νₘₐₓ 3096, 2985, 2235, 1580, 1550, 1469, 1390, 1316, 1262, 1102, 985, 840 cm⁻¹;

¹H NMR (DMSO-d₆, 400 MHz) δ 1.36 (6H, d, J=5.9 Hz), 4.99 (1H, quint, J=5.9 Hz), 7.43 (1H, d, J=6.4 Hz), 8.49 (1H, d, J=6.4 Hz);

HRMS m/z calcd for C₉H₉ON₂Cl 196.0404. Found 196.0401;

MS (EI) m/z: 196 [M⁺], 181, 154, 145, 126, 119, 111, 93, 71, 57, 44. (11c) 3-amino-4-isopropoxythieno[2,3-b]pyridine-2-carboxamide 2-chloro-4-isopropoxynicotinonitrile which was produced in Example 111 (111b) was used in place of 2-chloro-4-methoxynicotinonitrile and the reaction was performed in a similar method as described in Example 107 (107b) and the title compound was obtained.

White powder

Mp 238-240° C. (decomposition);

IR (KBr) νₘₐₓ 3485, 3322, 3137, 1672, 1616, 1582, 1506, 1378, 1287, 1107, 995 cm −1;

¹H NMR (DMSO-d₆, 400 MHz) δ 1.40 (6H, d, J=6.3 Hz), 4.94 (1H, quint, J=6.3 Hz), 6.83 (2H, brs), 6.98 (1H, d, J=5.9 Hz), 7.02 (2H, brs), 8.39 (1H, d, J=5.9 Hz);

HRMS m/z calcd for C₁₀H₁₃O₂N₃S 251.0728. Found 251.0742;

MS (EI) m/z: 251 [M⁺], 209, 192, 180, 164, 137, 120, 103, 92, 66, 52, 42.

### Example 112

### 3-amino-4-(cycloheptyloxy)thieno[2,3-b]pyridine-2-carboxamide

(Exemplified Compound No. 1-8)

### (112a) 2-chloro-4-(cycloheptyloxy)nicotinonitrile

Cycloheptyl alcohol was used in place of 2-propanol and the reaction was performed in a similar method as described in Example 111 (111b) and the title compound was synthesized.

Colourless liquid

IR (film) νₘₐₓ 2933, 2233, 1575, 1464, 1307, 998, 821 cm⁻¹;

¹H NMR (CDCl₃, 500 MHz) δ 1.47-2.05 (12H, m), 4.64-4.69 (1H, m), 6.79 (1H, d, J=6.3 Hz), 8.34 (1H, d, J=6.3 Hz);

HRMS m/z calcd for C₁₃H₁₅ON₂³⁵Cl 250.0873. Found 250.0880;

MS (EI) m/z: 250 [M⁺], 215, 172, 155, 137, 119, 97, 81, 55, 42;

Anal. Calcd for C₁₃H₁₅ClN₂O.0.36H₂O: C, 60.71; H, 6.16; N, 10.89. Found: C, 61.03; H, 6.51; N, 10.93.

### (112b) 3-amino-4-(cycloheptyloxy)thieno[2,3-b]pyridine-2-carboxamide

2-chloro-4-(cycloheptyloxy)nicotinonitrile which was produced in Example 112 (112a) was used in place of 2-chloro-4-methoxynicotinonitrile and the reaction was performed in a similar method as described in Example 107 (107b) and the title compound was synthesized.

White powder

Mp 179-180° C.;

IR (KBr) νₘₐₓ 3491, 3329, 3163, 2927, 1654, 1582, 1504, 1371, 1288, 1012 cm⁻¹;

¹H NMR (DMSO-d₆, 400 MHz) δ 1.46-1.70 (8H, m), 1.81-1.90 (2H, m), 2.01-2.08 (2H, m), 4.83-4.89 (1H, m), 6.81 (2H, brs), 6.94 (1H, d, J=5.5 Hz), 7.03 (2H, brs), 8.38 (1H, d, J=5.5 Hz);

HRMS m/z calcd for C₁₅H₁₉O₂N₃S 305.1198. Found: 305.1197;

MS (EI) m/z: 305 [M⁺], 209, 192, 164, 120, 97, 55, 41;

Anal. Calcd for C₁₅H₁₉N₃O₂S: C, 58.99; H, 6.27; N, 13.76; S, 10.50. Found: C, 58.81; H, 6.33; N, 13.60; S, 10.30.

### Example 113

### 3-amino-4-(cyclohexyloxy)thieno[2,3-b]pyridine-2-catboxamide

(Exemplified Compound No. 1-7)

### (113a) 2-chloro-4-(cyclohexyloxy)nicotinonitrile

Cyclohexyl alcohol was used in place of 2-propanol and the reaction was performed in a similar method as described in Example 111 (111b) and the title compound was synthesized.

Colourless liquid

IR (film) νₘₐₓ 2941, 2862, 2233, 1578, 1465, 1305, 1016, 987, 824 cm⁻¹;

¹H NMR (CDCl₃, 500 MHz) δ 1.42-1.96 (10H, m), 4.50-4.56 (1H, m), 6.84 (1H, d, J=6.4 Hz), 8.35 (1H, d, J=6.4 Hz);

HRMS m/z calcd for C₁₂H₁₃ON₂Cl 236.0717. Found 236.0721;

MS (EI) m/z: 236 [M⁺], 207, 195, 181, 155, 119, 83, 67, 55, 42;

Anal. Calcd for C₁₂H₁₃ClN₂O.0.12H₂O: C, 60.34; H, 5.59; N, 11.73; Cl, 14.84. Found: C, 60.06; H, 5.85; N, 11.50; Cl, 15.24.

### (113b) 3-amino-4-(cyclohexyloxy)thieno [2,3-b]pyridine-2-carboxamide 2-chloro-4-(cyclohexyloxy)nicotinonitrile which was produced in Example 113 (113a) was used in place of 2-chloro-4-methoxynicotinonitrile and the reaction was performed in a similar method as described in Example 107 (107b) and the title compound was synthesized.

White powder

Mp 206-208° C.;

IR (KBr) νₘₐₓ 3492, 3330, 3159, 2935, 1654, 1580, 1504, 1368, 1286, 1041, 1020, 992 cm⁻¹;

¹H NMR (DMSO-d₆, 400 MHz) δ 1.28-1.77 (8H, m), 1.94-2.03 (2H, m), 4.70-4.76 (1H, m), 6.81 (2H, brs), 7.02 (1H, d, J=5.9 Hz), 7.04 (2H, brs), 8.38 (1H, d, J=5.9 Hz);

HRMS m/z calcd for C₁₄H₁₇O₂N₃S 291.1041. Found 291.1040;

MS (EI) m/z: 291 [M⁺], 209, 192, 164, 137, 120, 55, 41;

Anal. Calcd for C₁₄H₁₇N₃O₂S.0.36H₂O: C, 56.45; H, 6.00; N, 14.11; S, 10.76. Found: C, 56.75; H, 5.74; N, 14.07; S, 10.46.

### Example 114

### 3-amino-4-(ethylthio)thieno[2,3-b]pyridine-2-carboxamide

(Exemplified Compound No. 1-10)

### (114a) 2-chloro-4-(ethylthio)nicotinonitrile

N,N-dimethylacetamide (1 mL) solution of sodium thioethoxide (93 mg, 1.1 mmol) was added to N,N-dimethylacetamide (1 mL) solution of 2,4-dichloronicotinonitrile (173 mg, 1.00 mmol) which was produced in Example 111 (111a) at 0° C. and the mixture was stirred at room temperature for one hour. Water (3 mL) was added to the reaction liquid, and generated powder was separated by filtration and further purified by silica gel column chromatography (hexane/ethyl acetate=3:1) and the title compound was obtained (44 mg, yield 22%).

Pale yellow powder

Mp 97-98° C.;

IR (KBr) νₘₐₓ 2226, 1556, 1518, 1431, 1379, 1199, 819 cm⁻¹;

¹H NMR (CDCl₃, 500 MHz) δ 1.46 (3H, t, J=7.3 Hz), 3.12 (2H, q, J=7.3 Hz), 7.11 (1H, d, J=5.9 Hz), 8.33 (1H, d, J=5.9 Hz);

HRMS m/z calcd for C₈H₇N₂³⁵ClS 198.0018. Found 198.0011;

MS (EI) m/z: 198 [M⁺], 183, 170, 165, 147, 134, 126, 108, 98, 76, 69, 64, 46.

### (114b) 3-amino-4-(ethylthio)thieno[2,3-b]pyridine-2-carboxamide

2-chloro-4-(ethylthio)nicotinonitrile which was produced in Example 114 (114a) was used in place of 2-chloro-4-methoxynicotinonitrile and the reaction was performed in a similar method as described in Example 107 (107b) and the title compound was obtained.

Pale yellow powder

Mp 230-235° C.;

IR (KBr) νₘₐₓ 3460, 3297, 2141, 1666, 1589, 1494, 1365, 625 cm⁻¹; ¹H NMR (DMSO-d₆, 400 MHz) δ 1.35 (3H, t, J=7.4 Hz), 3.23 (2H, q, J=7.4 Hz), 3.23 (2H, q, J=7.4 Hz), 6.94 (2H, brs), 7.20 (2H, brs), 7.29 (1H, d, J=5.5 Hz), 8.39 (1H, d, J=5.5 Hz);

HRMS m/z calcd for C₁₀H₁₁ON₃S₂ 253.0344. Found 253.0333;

MS (EI) m/z: 253 [M⁺], 236, 221, 208, 203, 193, 176, 148, 135, 104, 76, 45.

### Example 115

### 3-amino-4-(cyclohexylthio)thieno[2,3-b]pyridine-2-carboxamide

(Exemplified Compound No. 1-13)

Mercaptocyclohexane (116 mg, 1.00 mmol) and 8N aqueous solution of sodium hydroxide (0.19 mL) were added under ice-cooling to N,N-dimethylacetamide (1 mL) solution of 2,4-dichloronicotinonitrile (173 mg, 1.00 mmol) which was produced in Example 111 (111a) and the mixture was stirred for one hour. The reaction mixture was partitioned with water and methylene chloride, and the organic layer was concentrated under reduced pressure after drying over sodium sulfate. The residue was purified by silica gel column chromatography (hexane/ethyl acetate=4:1) and a mixture (187 mg) of 2-chloro-4-(ethylthio)nicotinonitrile and 2,4-di(cyclohexylthio)nicotinonitrile was obtained.

The obtained mixture (181 mg) and 2-mercaptoacetamide (66 mg) were dissolved in N,N-dimethylformamide (1.4 mL) and blended with 8N aqueous solution of sodium hydroxide (0.31 mL) and the mixture was stirred at room temperature for one hour. The solid which was deposited by adding water to the reaction mixture was separated by filtration and furthermore washed with ether and ethanol and 23 mg of the title compound was obtained (yield 7%).

Yellow powder

Mp 195-197° C.;

IR (KBr) νₘₐₓ 3450, 3310, 3154, 2929, 1662, 1585, 1495, 1363, 829, 620 cm⁻¹;

¹H NMR (DMSO-d₆, 400 MHz) δ 1.39-2.04 (1OH, m), 3.60-3.71 (1H, m), 7.06 (2H, brs), 7.19 (2H, brs), 7.38 (1H, d, J=5.5 Hz), 8.41 (1H, d, J=5.5 Hz);

HRMS m/z calcd for C₁₄H₁₇ON₃S₂ 307.0813. Found 307.0819;

MS (EI) m/z: 307 [M⁺], 289, 262, 225, 208, 180, 136, 104, 83, 55, 41.

### Preparation Examples

### Preparation Example 1 Powder

Powdered pharmaceutical composition can be obtained by mixing 5 g of a compound of Example 94, 895 g of lactose and 100 g of corn starch in a blender.

### Preparation Example 2 Granule

After mixing 5 g of a compound of Example 74, 865 g of lactose and 100 g of low substituted hydroxypropyl cellulose, 300 g of 10% hydroxypropyl cellulose aqueous solution is added and blended. Granules can be obtained by extruding this with an extrusion granulating machine followed by drying.

### Preparation Example 3 Tablet

Tablets can be obtained by mixing 5 g of a compound of Example 66, 90 g of lactose, 34 g of corn starch, 20 g of crystalline cellulose and 1 g of magnesium stearate in a blender and punching with a tabletting machine.

### Test Examples

### Test Example 1 Osteoblast differentiation test

ST2 cells (available from Institute of Physical and Chemical Research) which are stroma cells derived from mouse marrow were used. In this test, α-MEM medium (available from GIBCO BRL Cat. No. 10370-021) with which 10% (v/v) inactivated fetal bovine serum (available from Hyclone Company, FBS) and 1% (v/v) Penicillin-Streptomycin, Liquid (available from GIBCO BRL Cat. No. 15140-122) are mixed (hereinbelow abbreviated as 10% -FBS-αMEM) was used. Every culture in this test was conducted in a CO₂ incubator (37° C., 95% humidity, 5% CO₂).

The cells mentioned above were harvested with 2 mL of 0.25% trypsin solution (available from GIBCO BRL Cat. No. 15050-065) and after they were dispersed by adding 10 mL of 10% -FBS-αMEM, the cells were collected by centrifugal separation (25° C., 800 rpm, 5 minutes). A cell suspension containing 40,000 cells/mL was prepared from the collected cells using 10% -FBS-αMEM.

The cell suspension was dispensed to the wells of a 96 well microplate (Falcon company) in an amount of 100 μL per well so as to achieve 4,000 cells/well and cultured for 24 hours. Compounds were dispensed to the wells in final concentrations of 0.01, 0.03, 0.1, 0.3 μg/ml except the following control group. DMSO was dispensed to the wells of the control group in a final concentration of 0.1% (v/v). Alkaline phosphatase (ALP) activity was measured for each group after culturing for four days.

The measurement of ALP activity was performed as follows. That is, after the whole medium was removed from each well of the culturing plate, each well was washed twice by dispensing and removing 100 μL of Dulbecco's phosphate buffer (available from GIBCO BRL Cat. No. 14190-144). Cell lysing solution containing 10 mM MgCl₂ and 2%(v/v) TritonX-100 (Sigma company) was prepared and 50 μL/well of the cell lysing solution was dispensed and the mixture was stirred at room temperature for five minutes. An ALP substrate solution containing 50 mM of diethanolamine (Wako Pure Chemical Industries, Ltd. Cat. No. 099-03112) and 20 mM of p-nitrophenyl phosphite (Wako Pure Chemical Industries, Ltd. Cat. No. 147-02343) was prepared and 50 μL/well of the ALP substrate solution was dispensed and after allowing to stand at room temperature for 10 minutes, absorbance was measured using a microplate reader (Bio-rad company). Alkaline phosphatase activity increase rate (%) in the group blended with test compounds was calculated assuming the observed value of the control group of each plate to be 100% and evaluated as the osteoblast differentiation promoting activity.

In this test, the compounds of Examples 14, 16, 17, 24, 29, 57, 58, 59, 64, 65, 66, 68, 72, 74, 76, 77, 83, 84, 93, 94, 96, 97, 98, 99, 101 and 106 exhibited an alkaline phosphatase activity increase rate of more than 150% at 0.03 μg/mL.

### Test Example 2 Osteoclast formation suppression test

18 day-old ICR mice were purchased from Japan SLC, Inc. and used in the following experiments. The mice were sacrificed by cervical dislocation and the right and left femurs and tibias were extracted. After the tissue around the extracted femurs and tibias was removed, they were finely minced with scissors. 10 mL of 15%-FBS-αMEM was added to the minced femurs and tibias and the supernatant was collected after agitation for one minute and filtering with a cell strainer (Becton Dickinson company). A cell suspension containing 500,000 cells/mL was prepared with 15%-FBS-αMEM. The cell suspension was dispensed to the wells of a 96 well microplate (Falcon company) in an amount of 100 μL per well so as to achieve 50,000 cells/well and cultured for 24 hours. Active form vitamin D3 (Sigma company, Cat.No.D1530) was dispensed to the wells in final concentrations of 20 nM. Compounds were dispensed to the wells in final concentrations of 0.01, 0.03, 0.1, 0.3 μg/ml except the following control group. DMSO was dispensed to the wells of the control group in a final concentration of 0.1% (v/v). Tartrate-resistant acid phosphatase (TRAP) activity was measured for each group after culturing for five days.

The measurement of TRAP activity was performed as follows. That is, after the whole medium was removed from each well of the culturing plate, each well was washed twice by dispensing and removing 100 μL of Dulbecco's phosphate buffer (available from GIBCO BRL Cat. No. 14190-144). After retaining with a mixture of acetone-ethanol (1:1) for one minute, the retention liquid was removed and the wells were stained at 37° C. with Leukocyte acid phosphatase kit (Sigma company, Cat. No. 387-A) for 30 minutes. The staining solution was removed, then 100 μL of 10% sodium dodecylsulfate (Wako Pure Chemical Industries, Ltd. Cat. No. 191-07145) was dispensed and the mixture was stirred for five minutes, and then absorbance was measured using a microplate reader (Bio-rad company). TRAP activity decrease rate (%) in the group blended with test compounds was calculated assuming the observed value of the control group of each plate to be 100% and evaluated as the osteoclast formation suppression activity.

In this test, the compounds of Examples 57, 65, 66, 72, 83, 94, 97, 99, 101 and 106 exhibited an excellent osteoclast formation suppression effect.

### Test Example 3 Effect on bone mineral density

8 week-old female F344 rats were purchased from Charles River Laboratories and used in the following experiment. After ketamine (12.5 mg/ml)/xylazine (2.5 mg/ml) liquid mixture was intraperitoneally injected in an amount of 0.25 ml/100 g for anesthesia, ovariectomy or a sham operation was performed. From the next day of the operation, 0.5% carboxymethyl cellulose sodium salt solution (Wako Pure Chemical Industries, Ltd. Cat. No.039-01335) in which a test compound was suspended was orally administered once per day and six days a week. Six weeks after the administration started, the animals were painlessly killed by removing the whole blood from the aorta abdominalis under ketamine xylazine anesthesia and the right and left femurs were extracted.

After soft tissue was removed from the extracted femurs, the bone density was measured by DXA device DCS-600R (Aloka Co., Ltd.). The bone density was determined for the whole femur as well as three evenly divided parts, i.e., proximal, midshaft and distal end.

In this test, the compound of Example 57 significantly increased the bone density at 10 mg/kg.

### Test Example 4 Effect on Fracture Healing

12 week-old female F344 rats were purchased from Charles River Laboratories and used in the following experiment. An operation for fracturing bone was performed following a method of Li et al. (J. Bone Miner. Res 1999, 14: 969-979) under ketamine xylazine anesthesia. From the next day of the operation, 0.5% carboxymethyl cellulose sodium salt solution (Wako Pure Chemical Industries, Ltd. Cat. No. 039-01335) in which a test compound was suspended was orally administered once per day and six days a week. 39 days after the administration started, the animals were painlessly killed by removing the whole blood from the aorta abdominalis under ketamine xylazine anesthesia and the femurs were extracted.

After soft tissue was removed from the extracted femurs, they were measured by bone strength measuring apparatus MZ-500D (Maruto Instrument Co., Ltd.). 3-point bending tests were performed and determined by maximum load.

In this test, the compound of Example 57 significantly improved fracture healing.

### INDUSTRIAL APPLICABILITY

Since the compound having a general formula (I) of the present invention or pharmacologically acceptable salt thereof has effects for promoting osteogenesis, suppressing bone resorption and/or improving bone density, it is useful as a pharmaceutical composition {particularly a pharmaceutical composition for prevention or treatment of osteopathy [for example, osteoporosis (for example, postmenopausal osteoporosis, senile osteoporosis or secondary osteoporosis caused by the use of steroids or immunosuppressants), osteopenia or bone destruction associated with rheumatoid arthritis, Paget's disease of bone, bone fracture or dysostosis due to dwarfism] or osteoarthritis}.

1. A compound having the following general formula (I) 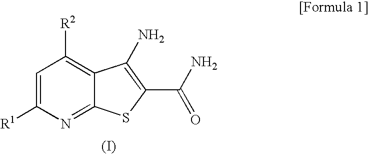
[wherein
R¹ represents a hydrogen atom, a cyclopropyl group or a C₁-C₆ alkyl group,
R² represents R^{a}S—, R^{a}O—, R^{a}NH—, R^{a}(R^{b})N— or a group 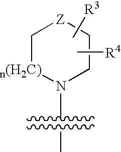
wherein R^{a} and R^{b} are the same or different and independently represent a C₁-C₆ alkyl group which may be substituted with one or more groups selected from Substituent Group α and Substituent Group γ; a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms,
R³ and R⁴ are the same or different and independently represent a hydrogen atom; a group selected from Substituent Group α, Substituent Group β and Substituent Group γ; a C₁-C₆ alkyl group substituted with one or more groups selected from Substituent Group γ; or a C₁-C₆ alkoxy group substituted with one or more groups selected from Substituent Group γ,
or when R³ and R⁴ are bonded to adjacent carbon atoms, R³ and R⁴ together with the carbon atoms to which they are bonded may form a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms,
Z represents a single bond; a double bond; an oxygen atom; a sulfur atom; sulfinyl; sulfonyl; or a group having the formula R⁵N<;
R⁵ represents a hydrogen atom; a C₁-C₆ alkyl group which may be substituted with one or more groups selected from Substituent Group α and Substituent Group γ; a C₂-C₆ alkenyl group which may be substituted with one or more groups selected from Substituent Group α and Substituent Group γ; a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group 65 ; a 5- to 7-membered heteroaryl group which may be substituted with one or more groups selected from Substituent Group α, Substituent Group β and Substituent Group γ and which contains 1 to 3 sulfur, oxygen and/or nitrogen atoms; a formyl group; a C₂-C₇ alkylcarbonyl group which may be substituted with one or more groups selected from Substituent Group a and Substituent Group γ; a 5- to 7-membered heterocyclylcarbonyl group which may be substituted with one or more groups selected from Substituent Group α, Substituent Group β and Substituent Group γ and which contains 1 to 3 sulfur, oxygen and/or nitrogen atoms; a C₇-C₁₁ arylcarbonyl group which may be substituted with one or more groups selected from Substituent Group α, Substituent Group β and Substituent Group γ; a 5- to 7-membered heteroarylcarbonyl group which may be substituted with one or more groups selected from Substituent Group α, Substituent Group β and Substituent Group γ and which contains 1 to 3 sulfur, oxygen and/or nitrogen atoms; a C₁-C₆ alkylsulfonyl group which may be substituted with one or more groups selected from Substituent Group α and Substituent Group γ; a C₆-C₁₀ arylsulfonyl group which may be substituted with one or more groups selected from Substituent Group α, Substituent Group β and Substituent Group γ; a 5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms; a C₂-C₇ alkoxycarbonyl group which may be substituted with one or more groups selected from Substituent Group α and Substituent Group γ; a C₇-C₁₁ aryloxycarbonyl group which may be substituted with one or more groups selected from Substituent Group α, Substituent Group β and Substituent Group γ; or a group having the formula R^{c}(R^{d})N—CO— (wherein R^{c} and R^{d} are the same or different and independently represent a hydrogen atom or a C₁-C₆ alkyl group which may be substituted with one or more groups selected from Substituent Group α and Substituent Group γ),
n represents an integer of 1 to 4,
Substituent Group α represents the group consisting of a halogen atom; a nitro group; a cyano group; a hydroxy group; a group having the formula R⁶—CO—, the formula R^{e}(R^{f})N—, the formula R^{e}(R^{f})N—CO— or the formula R^{e}(R^{f})N—SO₂— (wherein R⁶ represents a hydrogen atom, a C₁-C₆ alkyl group, a C₁-C₆ halogenated alkyl group, a C₃-C₈ cycloalkyl group, a hydroxy group, a C₁-C₆ alkoxy group, a C₆-C₁₀ aryl group or a C₆-C₁₀ aryloxy group and R^{e} and R^{f} are the same or different and independently represent a hydrogen atom; a C₁-C₆ alkyl group; a C₁-C₆ alkoxy group; a C₆-C₁₀ aryl group; a 5- to 7-membered heteroaryl group which contains 1 to 3 sulfur, oxygen and/or nitrogen atoms; a formyl group; a C₂-C₇ alkylcarbonyl group; a C₂-C₇ alkoxycarbonyl group; a C₇-C₁₁ arylcarbonyl group; a 5- to 7-membered heteroarylcarbonyl group which contains 1 to 3 sulfur, oxygen and/or nitrogen atoms; a C₁-C₆ alkylsulfonyl group; a C₆-C₁₀ arylsulfonyl group; or a 5- to 7-membered heteroarylsulfonyl group which contains 1 to 3 sulfur, oxygen and/or nitrogen atoms, or alternatively R^{e} and R^{f} together with the nitrogen atom to which they are bonded form a 4- to 7-membered heterocyclyl group which contains 1 to 3 sulfur, oxygen and/or nitrogen atoms (wherein the heterocyclyl group may have 1 or 2 substituent groups selected from a hydroxy group and a methyl group)); a hydroxyimino group; a C₁-C₆ alkoxyimino group; a C₁-C₆ alkoxy group; a C₃-C₈ cycloalkyloxy group; a C₁-C₆ halogenated alkoxy group; a C₁-C₆ alkylthio group; a C₁-C₆ alkylsulfinyl group; and a C₁-C₆ alkylsulfonyl group,
Substituent Group β represents the group consisting of a C₁-C₆ alkyl group which may be substituted with one or more groups selected from Substituent Group α; and a C₁-C₆ alkyl group substituted with a 5- to 7-membered heterocyclyl group which may be substituted with one or more groups selected from Substituent Group α and a C₁-C₆ alkyl group and which contains 1 to 3 sulfur, oxygen and/or nitrogen atoms, and Substituent Group γ represents the group consisting of a C₁-C₆ alkoxy group substituted with one or more groups selected from Substituent Group α; a C₁-C₆ alkylthio group substituted with one or more groups selected from Substituent Group α; a C₃-C₈ cycloalkyl group which may be substituted with one or more groups selected from Substituent Group α and Substituent Group β; a 5- to 7-membered heterocyclyl group which may be substituted with one or more groups selected from Substituent Group α and Substituent Group β and which contains 1 to 3 sulfur, oxygen and/or nitrogen atoms; a C₆-C₁₀ aryl group which may be substituted with one or more groups selected from Substituent Group α and Substituent Group β; a 5- to 7-membered heteroaryl group which may be substituted with one or more groups selected from Substituent Group α and Substituent Group β and which contains 1 to 3 sulfur, oxygen and/or nitrogen atoms; a C₃-C₈ cycloalkyloxy group which may be substituted with one or more groups selected from Substituent Group α and Substituent Group β; a 5- to 7-membered heterocyclyloxy group which may be substituted with one or more groups selected from Substituent Group α and Substituent Group β and which contains 1 to 3 sulfur, oxygen and/or nitrogen atoms; a C₆-C₁₀ aryloxy group which may be substituted with one or more groups selected from Substituent Group α and Substituent Group β; a 5- to 7-membered heteroaryloxy group which may be substituted with one or more groups selected from Substituent Group α and Substituent Group β and which contains 1 to 3 sulfur, oxygen and/or nitrogen atoms; and a C₆-C₁₀ aryl-C₁-C₆ alkoxy group in which the aryl moiety may be substituted with one or more groups selected from Substituent Group α and Substituent Group β] or a pharmacologically acceptable salt thereof.

2. The compound or pharmacologically acceptable salt thereof according to claim 1 wherein R¹ is a hydrogen atom, a cyclopropyl group or a C₁-C₄ alkyl group.

3. The compound or pharmacologically acceptable salt thereof according to claim 1 wherein R¹ is a hydrogen atom, methyl, ethyl, propyl or cyclopropyl.

4. The compound or pharmacologically acceptable salt thereof according to claim 1 wherein R¹ is a hydrogen atom or methyl.

5. The compound or pharmacologically acceptable salt thereof according to claim 1 wherein R² is a group R^{a}(R^{b})N—, and R^{a} and R^{b} are the same or different and independently represent a C₁-C₆ alkyl group which may be substituted with one or more groups selected from Substituent Group α and Substituent Group γ.

6. The compound or pharmacologically acceptable salt thereof according to claim 1 wherein R^{a} is a C₁-C₆ alkyl group which may be substituted with one group selected from Substituent Group α and Substituent Group γ, R^{b} is a C₁-C₆ alkyl group, and Substituent Group α is the group consisting of a C₁-C₆ alkoxy group, and Substituent Group γ is the group consisting of a C₁-C₆ alkoxy group substituted with one or more groups selected from Substituent Group α; a C₆-C₁₀ aryloxy group which may be substituted with one or more groups selected from Substituent Group α and Substituent Group β; and a 5- to 7-membered heteroaryloxy group which may be substituted with one or more groups selected from Substituent Group α and Substituent Group β and which contains 1 to 3 sulfur, oxygen and/or nitrogen atoms.

7. The compound or pharmacologically acceptable salt thereof according to claim 1 wherein R² is a group 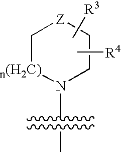
wherein R⁴ is a hydrogen atom or together with R³ forms a C₃-C₈ cycloalkyl group which may be substituted with one or more groups selected from Substituent Group α, Substituent Group β and Substituent Group γ; a 5- to 7-membered heterocyclyl group which may be substituted with one or more groups selected from Substituent Group α, Substituent Group β and Substituent Group γ and which contains 1 to 3 sulfur, oxygen and/or nitrogen atoms; a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms.

8. The compound or pharmacologically acceptable salt thereof according to claim 7 wherein R² is a group 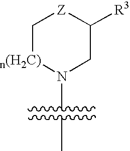
wherein Z represents a single bond, an oxygen atom, a sulfur atom or a group having the formula R⁵N<, R⁵ represents a C₆-C₁₀ aryl group which may be substituted with one or more groups selected from Substituent Group α, Substituent Group β and Substituent Group γ; a 5- to 7-membered heteroaryl group which may be substituted with one or more groups selected from Substituent Group α, Substituent Group β and Substituent Group γ and which contains 1 to 3 sulfur, oxygen and/or nitrogen atoms; a formyl group; a C₂-C₇ alkylcarbonyl group which may be substituted with one or more groups selected from Substituent Group α and Substituent Group γ; a C₁-C₆ alkylsulfonyl group which may be substituted with one or more groups selected from Substituent Group γ and Substituent Group γ; a C₆-C₁₀ arylsulfonyl group which may be substituted with one or more groups selected from Substituent Group α, Substituent Group β and Substituent Group γ; a 5- to 7-membered heteroarylsulfonyl group which may be substituted with one or more groups selected from Substituent Group α, Substituent Group β and Substituent Group γ and which contains 1 to 3 sulfur, oxygen and/or nitrogen atoms; a C₂-C₇ alkoxycarbonyl group which may be substituted with one or more groups selected from Substituent Group α and Substituent Group γ; or a group having the formula R^{c}(R^{d})N—CO—, and n is an integer of 1 to 3.

9. The compound or pharmacologically acceptable salt thereof according to claim 8 wherein R³ is a C₁-C₆ alkoxy group; a C₁-C₆ alkyl group which may be substituted with one or more groups selected from Substituent Group α; a C₁-C₆ alkoxy group substituted with one or more groups selected from Substituent Group α; a C₆-C₁₀ aryloxy group which may be substituted with one or more groups selected from Substituent Group α, Substituent Group β and Substituent Group γ; a C₁-C₆ alkyl group substituted with one or more groups selected from Substituent Group γ; or a C₁-C₆ alkoxy group substituted with one or more groups selected from Substituent Group γ, Z is a single bond, and n is 2.

10. The compound or pharmacologically acceptable salt thereof according to claim 8 wherein R³ is a hydrogen atom, Z is a sulfur atom, and n is 1.

11. The compound or pharmacologically acceptable salt thereof according to claim 8 wherein R³ is a hydrogen atom, Z is a group having the formula R⁵N<, R⁵ represents a C₆-C₁₀ aryl group which may be substituted with one or more groups selected from Substituent Group α, Substituent Group β and Substituent Group γ; or a 5- to 7-membered heteroaryl group which may be substituted with one or more groups selected from Substituent Group α, Substituent Group β and Substituent Group γ and which contains 1 to 3 sulfur, oxygen and/or nitrogen atoms: n is 2.

12. The compound or pharmacologically acceptable salt thereof according to claim 11 wherein the compound is selected from the following:
3-amino-4-[(3S)-3-(methoxymethyl)piperidin-1-yl]thieno[2,3-b]pyridine-2-carboxamide,
3-amino-4-[(3S)-3-(methoxymethyl)piperidin-1-yl]-6-methylthieno[2,3-b]pyridine-2-carboxamide,
3-amino-4-{3-[3-(2-hydroxyethoxy)propyl]piperidin-1-yl}thieno[2,3 -b]pyridine-2-carboxamide,
3-amino-4-{(3S)-[(2-methoxyethoxy)methyl]piperidin-1-yl}thieno[2,3-b]pyridine-2-carboxamide,
3-amino-4-{(3S)-3-[(3-methoxypropoxy)methyl]piperidin-1-yl}thieno[2,3-b]pyridine-2-carboxamide,
3-amino-4-(3-{[2-(dimethylamino)-2-oxoethoxy]methyl}piperidin-1-yl)thieno[2,3-b]pyridine-2-carboxamide,
3-amino-4-(3-{3-[2-(dimethylamino)-2-oxoethoxy]propyl}piperidin-1-yl)thieno[2,3-b]pyridine-2-carboxamide,
4-[4-(4-acetylphenyl)-1,4-diazepan-1-yl]-3-aminothieno[2,3-b]pyridine-2-carboxamide,
3-amino-4-[4-(4-propionylphenyl)-1,4-diazepan-1-yl]thieno[2,3-b]pyridine-2-carboxamide,
3-amino-4-{4-[4-(dimethylamino)phenyl]-1,4-diazepan-1-yl}thieno[2,3-b]pyridine-2-carboxamide,
3-amino-4-(4-{4-[(dimethylamino)carbonyl]phenyl}-1,4-diazepan-1-yl)thieno[2,3-b]pyridine-2-carboxamide,
4-[4-(5-acetylpyridin-2-yl)-1,4-diazepan-1-yl]-3-aminothieno[2,3-b]pyridine-2-carboxamide,
3-amino-4-(4-{4-[(dimethylamino)carbonyl]phenyl}-1,4-diazepan-1-yl)-6-methylthieno[2,3-b]pyridine-2-carboxamide,
3 -amino-4-{4-[4-(2-methoxyethyl)phenyl]-1,4-diazepan-1-yl}thieno[2,3-b]pyridine-2-carboxamide,
3-amino-4-(4-{4-[2-(dimethylamino)-2-oxoethyl]phenyl}-1,4-diazepan-1-yl)thieno[2,3-b]pyridine-2-carboxamide,
3-amino-4-(4-{4-[3-(dimethylamino)-3-oxopropyl]phenyl}-1,4-diazepan-1-yl)thieno[2,3-b]pyridine-2-carboxamide,
3-amino-4-{4-[4-(azetidin-1-ylcarbonyl)phenyl]-1,4-diazepan-1-yl}thieno[2,3-b]pyridine-2-carboxamide,
3-amino-4-{4-[4-(morpholin-4-ylcarbonyl)phenyl]-1,4-diazepan-1-yl}thieno[2,3-b]pyridine-2-carboxamide,
3-amino-4-(4-{4-[2-(dimethylamino)ethyl]phenyl}-1,4-diazepan-1-yl)thieno[2,3-b]pyridine-2-carboxamide,
3-amino-4-{4-[4-(2-hydroxyethyl)phenyl]-1,4-diazepan-1-yl}thieno[2,3-b]pyridine-2-carboxamide,
3-amino-4-{4-[3-(2-hydroxyethyl)phenyl]-1,4-diazepan-1-yl}thieno[2,3-b]pyridine-2-carboxamide,
3-amino-4-{4-[4-(3-hydroxypropyl)phenyl]-1,4-diazepan-1-yl}thieno[2,3-b]pyridine-2-carboxamide,
3-amino-4-{4-[4-(1-hydroxyethyl)phenyl]-1,4-diazepan-1-yl}thieno[2,3-b]pyridine-2-carboxamide,
3-amino-4-{4-[4-(2-oxopropyl)phenyl]-1,4-diazepan-1-yl}thieno[2,3-b]pyridine-2-carboxamide,
3-amino-4-{4-[4-(N-hydroxyethaneimidoyl)phenyl]-1,4-diazepan-1-yl}thieno[2,3-b]pyridine-2-carboxamide,
3-amino-4-(4-{4-[(2-methyl-1,3-dioxolan-2-yl)methyl]phenyl}-1,4-diazepan-1-yl)thieno[2,3-b]pyridine-2-carboxamide,
3-amino-4-[4-(2-methyl-1-oxo-2,3-dihydro-1H-isoindol-5-yl)-1,4-diazepan-1-yl]thieno[2,3-b]pyridine-2-carboxamide,
3-amino-4-[4-(1,3-benzoxazol-6-yl)-1,4-diazepan-1-yl]thieno[2,3-b]pyridine-2-carboxamide,
3-amino-4-[4-(4-hydroxyimino-3,4-dihydro-2H-chromen-7-yl)-1,4-diazepan-1-yl]thieno[2,3-b]pyridine-2-carboxamide,
4-[4-(4-acetyl-1,3-thiazol-2-yl)-1,4-diazepan-1-yl]-3-aminothieno[2,3-b]pyridine-2-carboxamide,
4-[4-(5-acetylthiophen-2-yl)-1,4-diazepan-1-yl]-3-aminothieno[2,3-b]pyridine-2-carboxamide, and
3-amino-4-(4-{4-[(dimethylamino)carbonyl]-1,3-thiazol-2-yl}-1,4-diazepan-1-yl)thieno[2,3-b]pyridine-2-carboxamide.

13. A pharmaceutical composition which comprises the compound or pharmacologically acceptable salt thereof according to claim 1 as an active ingredient.

14. A pharmaceutical composition according to claim 13, for prevention or treatment of osteopathy or osteoarthritis.

15. The pharmaceutical composition according to claim 14, for prevention or treatment of osteopathy wherein the osteopathy is osteoporosis, osteopenia or bone destruction associated with rheumatoid arthritis, Paget's disease of bone, bone fracture or dysostosis due to dwarfism.

16. The pharmaceutical composition according to claim 15, for prevention or treatment of osteoporosis wherein the osteoporosis is postmenopausal osteoporosis, senile osteoporosis or secondary osteoporosis caused by the use of steroids or immunosuppressants.

17-26. (canceled)

27. A method for promoting osteogenesis, suppressing bone resorption and/or improving bone density comprising administering an effective amount of the compound or pharmacologically acceptable salt thereof according to claim 1 to a mammal or a bird.

28. The method according to claim 27 for prevention or treatment of osteopathy or osteoarthritis.

29. The method according to claim 28 for prevention of osteopathy wherein the osteopathy is osteoperosis, osteopenia or bone destruction associated with rheumatoid arthritis, Paget's disease of bone, bone fracture or dystosis due to dwarfism.

30. The method according to claim 29 for prevention or treatment of osteoporosis wherein the osteoporosis is postmenopausal osteoporosis, senile osteoporosis or secondary osteoporosis caused by the use of steroids or immunosuppressants.

31. The method according to claim 27 comprising administering the compound or salt thereof to a mammal wherein the mammal is a human, horse, cow or pig.

32. The method according to claim 30 wherein the mammal is a human.

33. The method according to claim 27 comprising administering the compound or salt thereof to a bird wherein the bird is a chicken.

34. The method according to claim 27 wherein the compound is selected from the following:
3-amino-4-[(3S)-3-(methoxymethyl)piperidin-1-yl]thieno[2,3-b]pyridine-2-carboxamide,
3-amino-4-[(3S)-3-(methoxymethyl)piperidin-1-yl]-6-methylthieno[2,3-b]pyridine-2-carboxamide,
3-amino-4-{3-[3-(2-hydroxyethoxy)propyl]piperidin-1-yl}thieno[2,3-b]pyridine-2-carboxamide,
3-amino-4-{(3S)-[(2-methoxyethoxy)methyl]piperidin-1-yl}thieno[2,3-b]pyridine-2-carboxamide,
3-amino-4-{(3S)-3-[(3-methoxypropoxy)methyl]piperidin-1-yl}thieno[2,3-b]pyridine-2-carboxamide,
3-amino-4-(3-{[2-(dimethylamino)-2-oxoethoxy]methyl}piperidin-1-yl)thieno[2,3-b]pyridine-2-carboxamide,
3-amino-4-(3-{3-[2-(dimethylamino)-2-oxoethoxy]propyl}piperidin-1-yl)thieno[2,3-b]pyridine-2-carboxamide,
4-[4-(4-acetylphenyl)-1,4-diazepan-1-yl]-3-aminothieno[2,3-b]pyridine-2-carboxamide,
3-amino-4-[4-(4-propionylphenyl)-1,4-diazepan-1-yl]thieno[2,3-b]pyridine-2-carboxamide,
3-amino-4-{4-[4-(dimethylamino)phenyl]-1,4-diazepan-1-yl}thieno[2,3-b]pyridine-2-carboxamide,
3-amino-4-(4-{4-[(dimethylamino)carbonyl]phenyl}-1,4-diazepan-1-yl)thieno[2,3-b]pyridine-2-carboxamide,
4-[4-(5 -acetylpyridin-2-yl)-1,4-diazepan-1-yl]-3-aminothieno[2,3-b]pyridine-2-carboxamide,
3-amino-4-(4-{4-[(dimethylamino)carbonyl]phenyl}-1,4-diazepan-1-yl)-6-methylthieno[2,3-b]pyridine-2-carboxamide,
3-amino-4-{4-[4-(2-methoxyethyl)phenyl]-1,4-diazepan-1-yl}thieno[2,3-b]pyridine-2-carboxamide,
3-amino-4-(4-{4-[2-(dimethylamino)-2-oxoethyl]phenyl}-1,4-diazepan-1-yl)thieno[2,3 -b]pyridine-2-carboxamide,
3-amino-4-(4-{4-[3-(dimethylamino)-3-oxopropyl]phenyl}-1,4-diazepan-1-yl)thieno[2,3-b]pyridine-2-carboxamide,
3-amino-4-{4-[4-(azetidin-1-ylcarbonyl)phenyl]-1,4-diazepan-1-yl}thieno[2,3-b]pyridine-2-carboxamide,
3-amino-4-{4-[4-(morpholin-4-ylcarbonyl)phenyl]-1,4-diazepan-1-yl}thieno[2,3-b]pyridine-2-carboxamide,
3-amino-4-(4-{4-[2-(dimethylamino)ethyl]phenyl}-1,4-diazepan-1-yl)thieno[2,3-b]pyridine-2-carboxamide,
3-amino-4-{4-[4-(2-hydroxyethyl)phenyl]-1,4-diazepan-1-yl}thieno[2,3-b]pyridine-2-carboxamide,
3-amino-4-{4-[3-(2-hydroxyethyl)phenyl]-1,4-diazepan-1-yl}thieno[2,3-b]pyridine-2-carboxamide,
3-amino-4-{4-[4-(3-hydroxypropyl)phenyl]-1,4-diazepan-1-yl}thieno[2,3-b]pyridine-2-carboxamide,
3-amino-4-{4-[4-(1-hydroxyethyl)phenyl]-1,4-diazepan-1-yl}thieno[2,3-b]pyridine-2-carboxamide,
3-amino-4-{4-[4-(2-oxopropyl)phenyl]-1,4-diazepan-1-yl}thieno[2,3-b]pyridine-2-carboxamide,
3-amino-4-{4-[4-(N-hydroxyethaneimidoyl)phenyl]-1,4-diazepan-1-yl}thieno[2,3-b]pyridine-2-carboxamide,
3-amino-4-(4-{4-[(2-methyl-1,3-dioxolan-2-yl)methyl]phenyl}-1,4-diazepan-1-yl)thieno[2,3-b]pyridine-2-carboxamide,
3-amino-4-[4-(2-methyl-1-oxo-2,3-dihydro-1H-isoindol-5-yl)-1,4-diazepan-1-yl]thieno[2,3-b]pyridine-2-carboxamide,
3-amino-4-[4-(1,3-benzoxazol-6-yl)-1,4-diazepan-1-yl]thieno[2,3-b]pyridine-2-carboxamide,
3-amino-4-[4-(4-hydroxyimino-3,4-dihydro-2H-chromen-7-yl)-1,4-diazepan-1-yl]thieno[2,3-b]pyridine-2-carboxamide,
4-[4-(4-acetyl-1,3-thiazol-2-yl)-1,4-diazepan-1-yl]-3-aminothieno[2,3-b]pyridine-2-carboxamide,
4-[4-(5-acetylthiophen-2-yl)-1,4-diazepan-1-yl]-3-aminothieno[2,3-b]pyridine-2-carboxamide, and
3-amino-4-(4-{4-[(dimethylamino)carbonyl]-1,3-thiazol-2-yl}-1,4-diazepan-1-yl)thieno[2,3-b]pyridine-2-carboxamide.
